(19) United States
(12) Patent Application Publication
(10) Pub. No.: US 2023/0151026 A1
(43) Pub. Date: May 18, 2023

(54) MULTI-LAYER BODY FOR DIFFUSE TRANSILLUMINATION

(71) Applicant: Merck Patent GmbH, Darmstadt (DE)

(72) Inventors: Amir Hossain PARHAM, Darmstadt (DE); Christian EHRENREICH, Darmstadt (DE); Jens ENGELHART, Darmstadt (DE)

(21) Appl. No.: 17/915,514

(22) PCT Filed: Mar. 30, 2021
(86) PCT No.: PCT/EP2021/058222

§ 371 Date: Sep. 29, 2022

(30) Foreign Application Priority Data
Apr. 2, 2020 (WO) 20167658.2

(51) Int. Cl. C07D 513/14; C07D 498/14; C07D 471/14; C07D 487/14; C07D 519/00; H10K 50/11; H10K 85/60

(52) U.S. Cl. CPC C07D 513/14; C07D 498/14; C07D 471/14; C07D 487/14; C07D 519/00; H10K 50/11; H10K 85/657; H10K 85/6572; H10K 2101/20

(57) ABSTRACT

The present invention relates to compounds suitable for use in electronic devices, and to electronic devices, especially organic electroluminescent devices, comprising these compounds.

The present invention relates to materials for use in electronic devices, especially in organic electroluminescent devices, and to electronic devices, especially organic electroluminescent devices comprising these materials.

Emitting materials used in organic electroluminescent devices (OLEDs) are frequently phosphorescent organometallic complexes. In general terms, there is still a need for improvement in OLEDs, especially also in OLEDs which exhibit triplet emission (phosphorescence), for example with regard to efficiency, operating voltage and lifetime. The properties of phosphorescent OLEDs are not just determined by the triplet emitters used. More particularly, the other materials used, such as matrix materials, are also of particular significance here. Improvements to these materials can thus also lead to improvements in the OLED properties. Suitable matrix materials for OLEDs are, for example, aromatic lactams as disclosed, for example, in WO 2011/116865, WO 2011/137951, WO 2013/064206 or KR 2015-037703.

It is an object of the present invention to provide compounds which are suitable for use in an OLED, especially as matrix material for phosphorescent emitters or as electron transport material, and which lead to improved properties therein.

It has been found that, surprisingly, this object is achieved by particular compounds described in detail hereinafter that are of good suitability for use in OLEDs. These OLEDs especially have a long lifetime, high efficiency and relatively low operating voltage. The present invention therefore provides these compounds and electronic devices, especially organic electroluminescent devices, comprising these compounds.

The present invention provides a compound of formula (1)

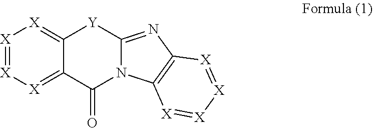

where the symbols used are as follows:
X is the same or different at each instance and is CR or N, where there is at least one instance of two adjacent X groups that are a group of the following formula (2), and the other symbols X are the same or different at each instance and are CR or N:

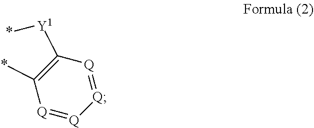

Y, Y¹ is the same or different at each instance and is an NR, NAr, CR₂, SiR₂, BAr, C═O, O or S;
Q is the same or different at each instance and is N or CR;
Ar is the same or different at each instance and is an aromatic or heteroaromatic ring system which has 5 to 60 aromatic ring atoms and may be substituted in each case by one or more R radicals;
R is the same or different at each instance and is H, D, F, Cl, Br, I, B(OR¹)₂, CHO, C(═O)R¹, CR¹═C(R¹)₂, CN, C(═O)OR¹, C(═O)N(R¹)₂, Si(R¹)₃, N(R¹)₂, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R¹C═CR¹—, —C≡C—, Si(R¹)₂, C═O, C═S, C═NR¹, —C(═O)O—, —C(═O)NR¹—, NR¹, P(═O)(R¹), —O—, —S—, SO or SO₂, or an aromatic or heteroaromatic ring system which has 5 to 60 aromatic ring atoms, preferably 5 to 40 aromatic ring atoms, and may be substituted in each case by one or more R¹ radicals, where two or more R radicals may be joined to one another and may form a ring;
R¹ is the same or different at each instance and is H, D, F, Cl, Br, I, B(OR²)₂, CHO, C(═O)R², CR²═C(R²)₂, CN, C(═O)OR², C(═O)N(R²)₂, Si(R²)₃, N(R²)₂, NO₂, P(═O)(R²)₂, OSO₂R², OR², S(═O)R², S(═O)₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more CH₂ groups in the abovementioned groups may be replaced by —R²C═CR²—, —C≡C—, Si(R²)₂, C═O, C═S, C═NR², —C(═O)O—, —C(═O)NR²—, NR², P(═O)(R²), —O—, —S—, SO or SO₂ and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, where two or more R¹ radicals may be joined to one another and may form a ring;
R² is the same or different at each instance and is H, D, F or an aliphatic, aromatic or heteroaromatic organic radical having 1 to 20 carbon atoms, in which one or more hydrogen atoms may also be replaced by D or F; at the same time, two or more R² substituents may be joined to one another and may form a ring.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An aromatic ring system in the context of this invention contains 6 to 60 carbon atoms, preferably 6 to 40 carbon atoms, in the ring system. A heteroaromatic ring system in the context of this invention contains 2 to 60 carbon atoms, preferably 2 to 40 carbon atoms, and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. These shall likewise be understood to mean systems in which two or more aryl or heteroaryl groups are joined directly to one another, for example biphenyl, terphenyl, bipyridine or phenylpyridine. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferred aromatic or heteroaromatic ring systems are simple aryl or heteroaryl groups and groups in which two or more aryl or heteroaryl groups are joined directly to one another, for example biphenyl or bipyridine, and also fluorene or spirobifluorene.

An electron-rich heteroaromatic ring system is characterized in that it is a heteroaromatic ring system containing no electron-deficient heteroaryl groups. An electron-deficient heteroaryl group is a six-membered heteroaryl group having at least one having at least one nitrogen atom or a five-membered heteroaryl group having at least two heteroatoms, one of which is a nitrogen atom and the other is oxygen, sulfur or a substituted nitrogen atom, where further aryl or heteroaryl groups may also be fused onto these groups in each case. By contrast, electron-rich heteroaryl groups our five-membered heteroaryl groups having exactly one heteroatom selected from oxygen, sulfur and substituted nitrogen, to which may be fused further aryl groups and/or further electron-rich five-membered heteroaryl groups. Thus, examples of electron-rich heteroaryl groups are pyrrole, furan, thiophene, indole, benzofuran, benzothiophene, carbazole, dibenzofuran, dibenzothiophene or indenocarbazole.

In the context of the present invention, the term “alkyl group” is used as an umbrella term both for linear and branched alkyl groups and for cyclic alkyl groups. Analogously, the terms “alkenyl group” and “alkynyl group” are used as umbrella terms both for linear or branched alkenyl or alkynyl groups and for cyclic alkynyl groups.

In the context of the present invention, an aliphatic hydrocarbyl radical or an alkyl group or an alkenyl or alkynyl group which may contain 1 to 40 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group OR¹ having 1 to 40 carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group SR¹ having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, Cl or CN, more preferably F or CN.

An aromatic or heteroaromatic ring system which has 5-60 aromatic ring atoms and may also be substituted in each case by the abovementioned R² radicals or a hydrocarbyl radical and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from a combination of these systems.

The wording that two or more radicals together may form a ring system, in the context of the present description, should be understood to mean, inter alia, that the two radicals are joined to one another by a chemical bond with formal elimination of two hydrogen atoms. This is illustrated by the following scheme:

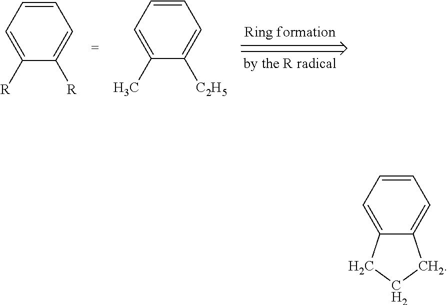

In addition, however, the abovementioned wording shall also be understood to mean that, if one of the two radicals is hydrogen, the second radical binds to the position to which the hydrogen atom was bonded, forming a ring. This will be illustrated by the following scheme:

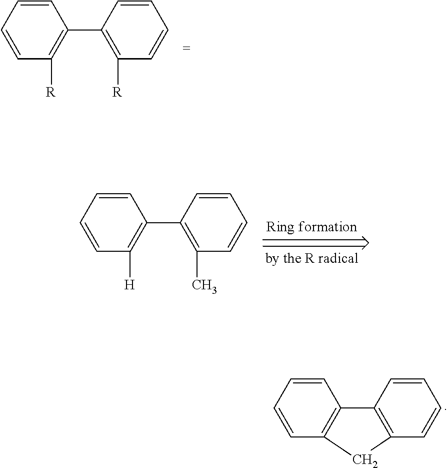

In a preferred embodiment of the invention, in each of the lateral aromatic six-membered rings in the compound of the formula 1, there is up to one identical or different instance of two adjacent X groups that are a group of the formula (2); preferably, exactly two adjacent X groups per formula (1) are a group of the formula (2).

If two adjacent X groups are a group of the formula (2), each of the X groups is a C corresponding to the positions identified by * in formula (2). Together with formula (2), the result is therefore a five-membered ring fused on to the formula (1) which is formed from the two X groups and formula (2).

This results in the preferred compounds of the formulae (3) to (6):

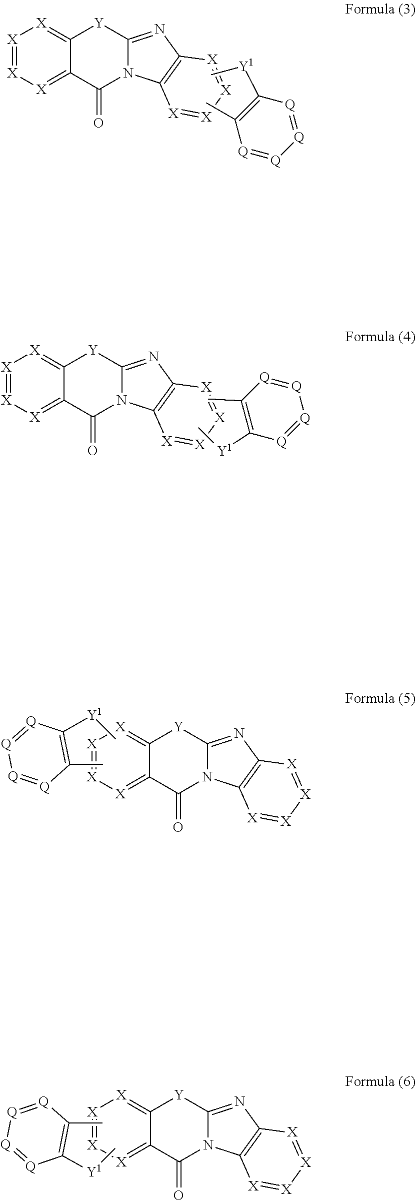

where the symbols used have the definitions given above, with the proviso that X is the same or different at each instance and is N or CR, and that two adjacent X groups are C and form a fused-on five-membered ring together with the group containing Y¹.

In a preferred embodiment of the invention, not more than two symbols X per cycle are N, more preferably not more than one symbol X.

In a preferred embodiment of the invention, not more than two symbols Q per cycle are N.

In a preferred embodiment of the invention, X, where it is CR or N, is CR. In a further preferred embodiment, Q is CR.

Further preferred embodiments are shown by the following formulae (7) to (10):

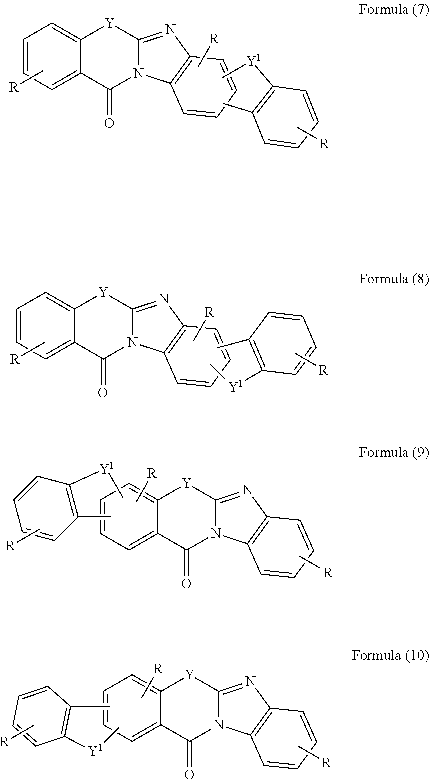

where the symbols used have the definitions given above and the aromatic systems may be substituted identically or differently by one or more R groups as shown.

In a preferred embodiment of the invention, the Y¹ group in the formulae (3) and (4) is in the para position to the nitrogen atoms, and in formulae (5) and (6) is in the para position to the keto group or to Y.

In a preferred embodiment of the invention, the Y¹ group in the formulae (7) and (8) is in the para position to the nitrogen atoms, and in formulae (9) and (10) is in the para position to the keto group or to Y.

In a further preferred embodiment of the invention, the compound is selected from compounds of the formulae (11) to (14):

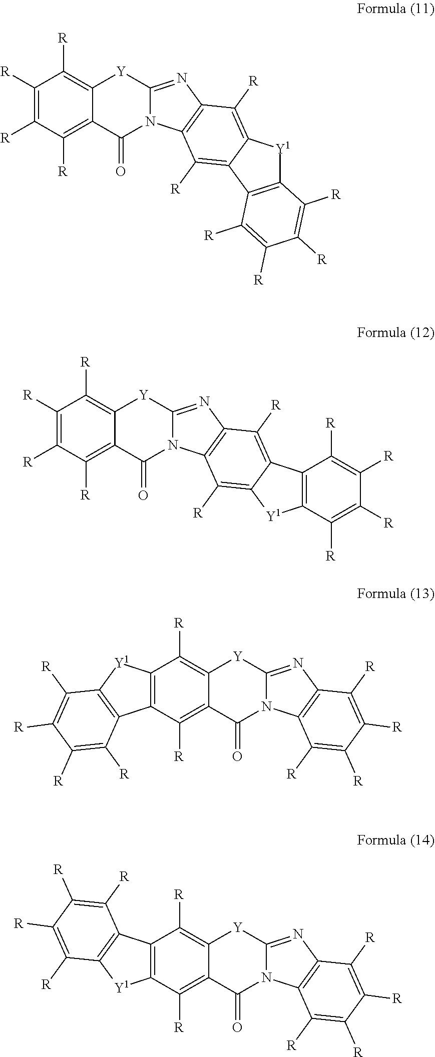

where the symbols used have the definitions given above.

In a preferred embodiment of the invention, not more than 3 R groups in the formulae (11) to (14) are not H or D, preferably not more than 2 R groups.

In a further preferred embodiment, the compound is a compound of the formulae (15) to (18):

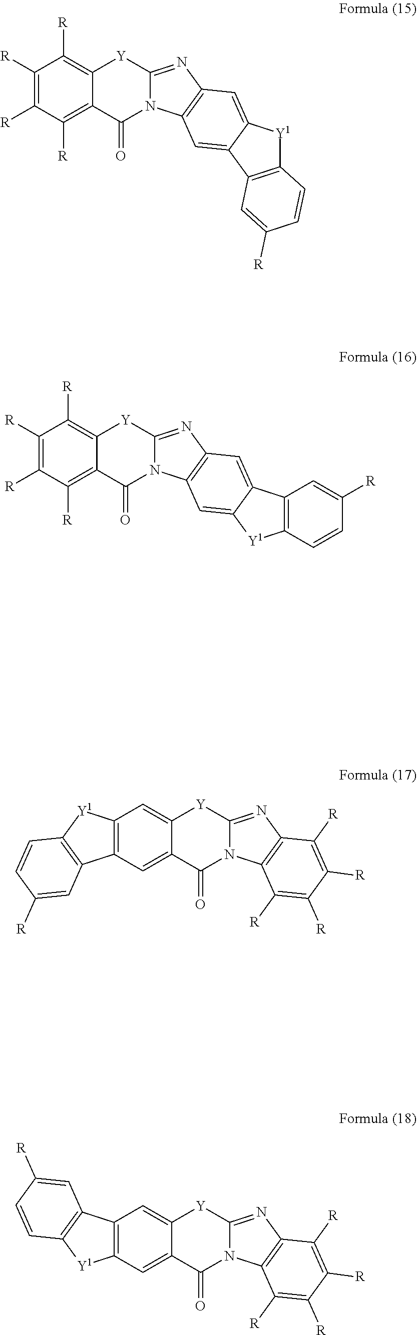

where the symbols used have the definitions given above.

In a preferred embodiment of the invention, Y and Y¹ are the same or different and are CR₂, NR, NAr, O or S, more preferably S, O, NAr or NR.

In a particularly preferred embodiment, Y and Y¹ are the same or different and are S, O or NAr.

In a further particularly preferred embodiment, Y¹ is NAr; in particular, Y¹ is NAr and Y is NAr, O or S.

More preferably, the abovementioned preferences for Y¹ and Y occur simultaneously in the abovementioned formulae, especially in the formulae (15) to (18), and so particular preference is given to the compounds of the following formulae (15-1) to (18-1):

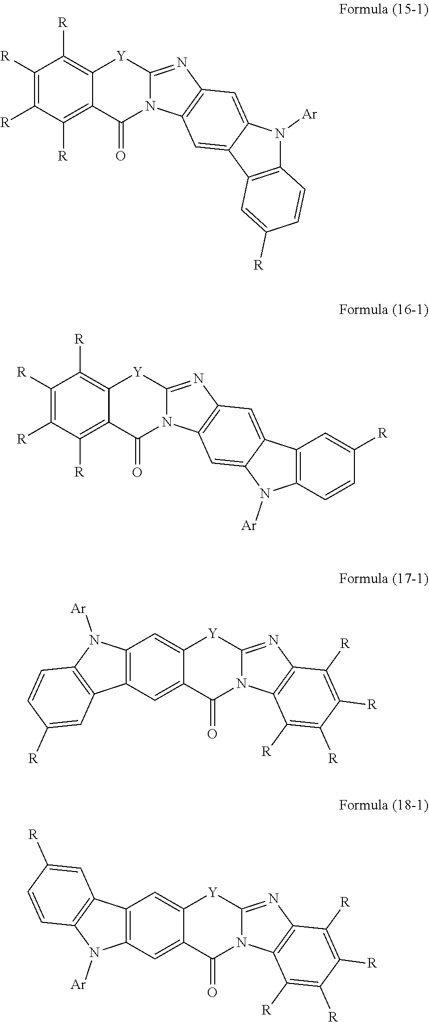

where the symbols used have the definitions given above.

There follows a description of preferred substituents R, Ar, R¹ and R². In a particularly preferred embodiment of the invention, the preferences specified hereinafter for R, Ar, R¹ and R² occur simultaneously and are applicable to the structures of the formula (1) and to all preferred embodiments detailed above.

In a preferred embodiment, Ar is an aromatic ring system which has 6 to 30 aromatic ring atoms and may be substituted by one or more R radicals, or a heteroaromatic ring system which has 6 to 30 aromatic ring atoms and may be substituted by one or more R radicals. In a particularly preferred embodiment of the invention, Ar is an aromatic or heteroaromatic ring system which has 6 to 24 aromatic ring atoms and may be substituted by one or more preferably nonaromatic R radicals.

Suitable aromatic or heteroaromatic ring systems Ar are the same or different at each instance and are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, phenanthrene, triphenylene or a combination of two or three of these groups, each of which may be substituted by one or more R radicals, preferably nonaromatic R radicals.

Further preferred embodiments of Ar, when these represent an aromatic ring system, are selected from the group consisting of pyridine, pyrimidine, pyrazine, pyridazine, triazine, quinoline, quinazoline and benzimidazole or a combination of these groups with one of the abovementioned groups. When Ar is a heteroaryl group, especially triazine, pyrimidine, quinazoline or carbazole, preference may also be given to aromatic or heteroaromatic R radicals on this heteroaryl group.

Ar here is an aromatic or heteroaromatic ring system, preferably the same or different at each instance and selected from the groups of the following formulae Ar-1 to Ar-76:

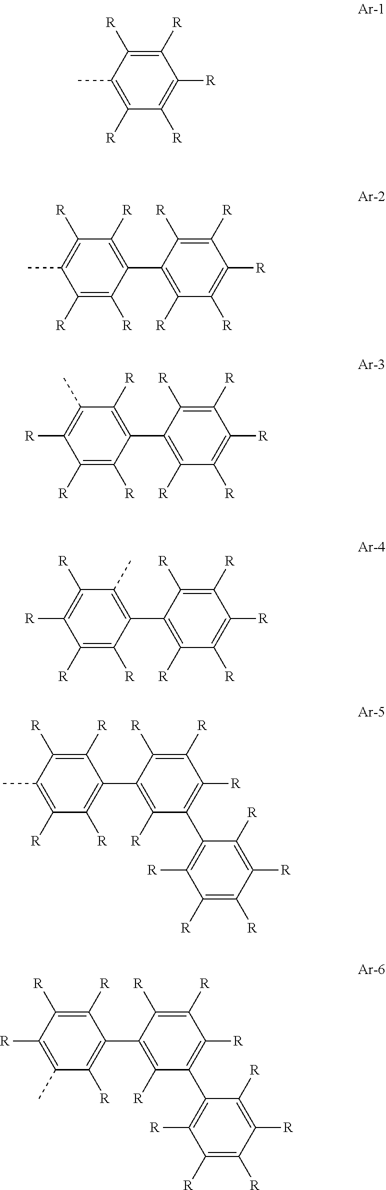

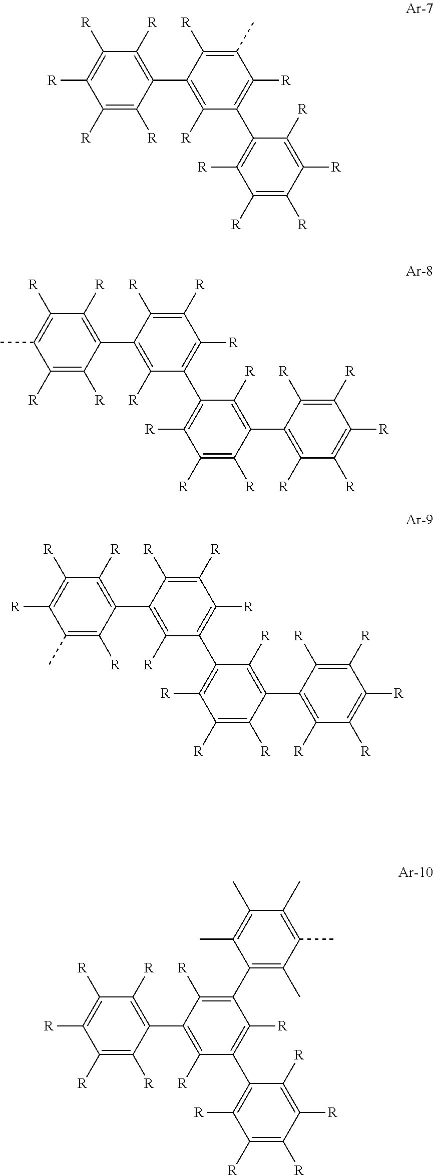

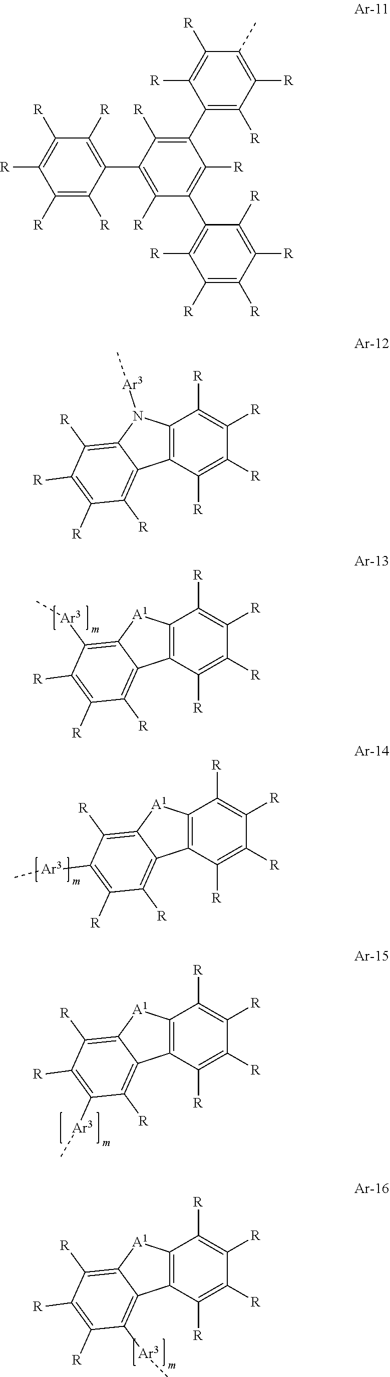

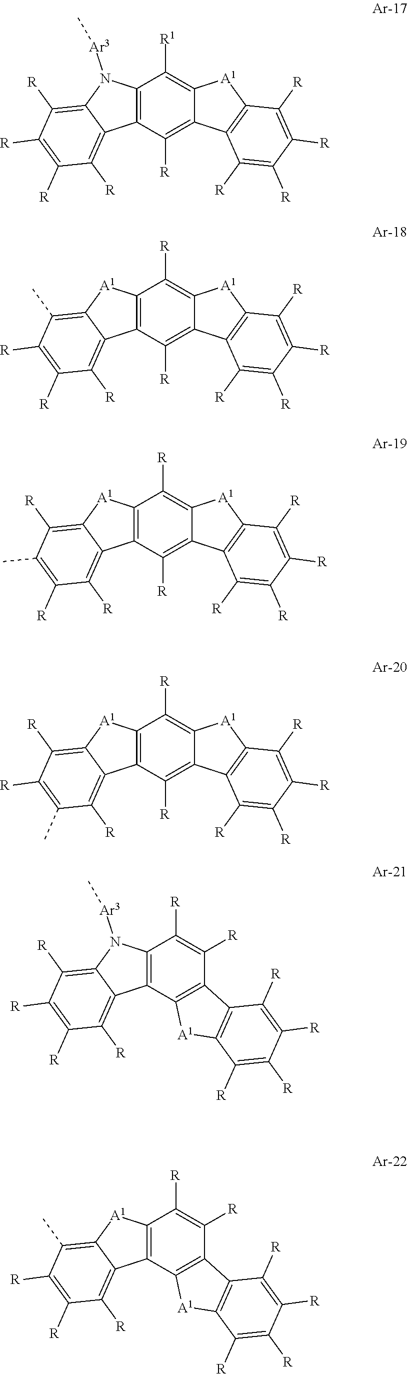

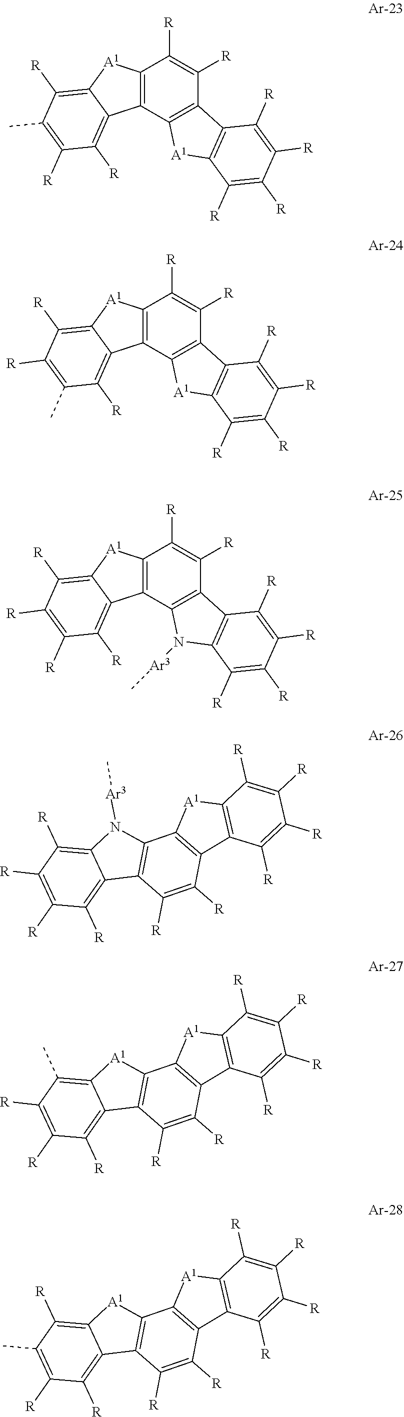

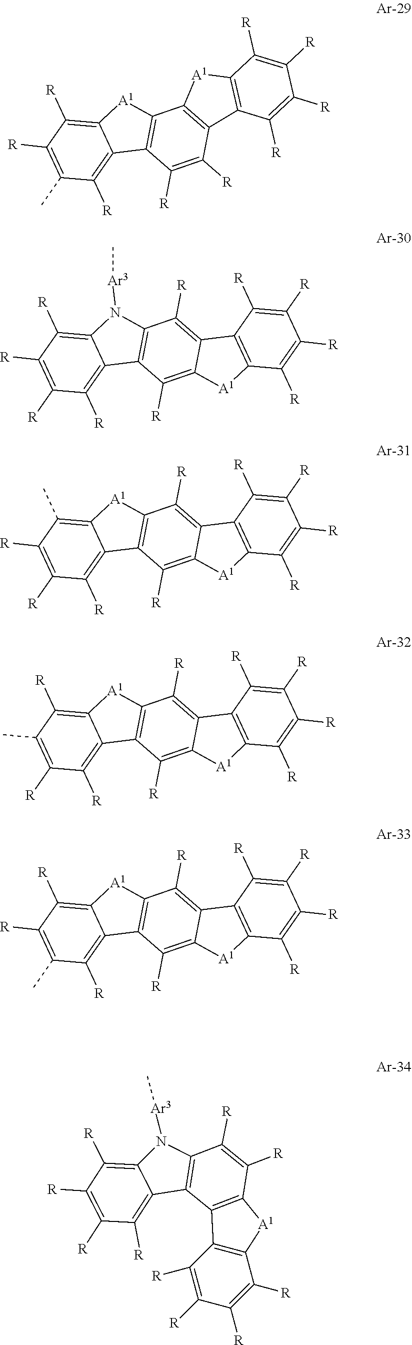

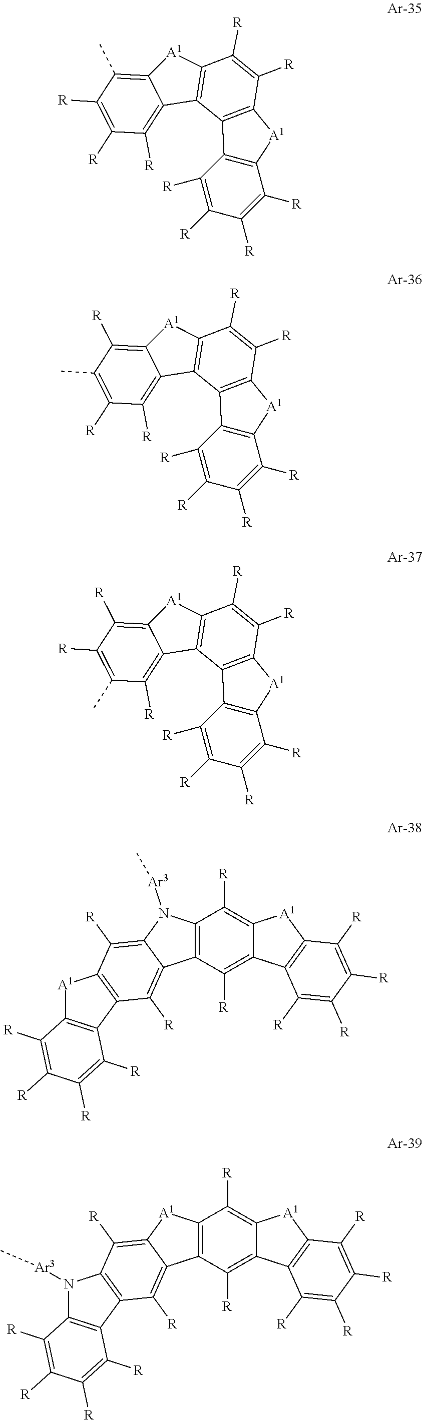

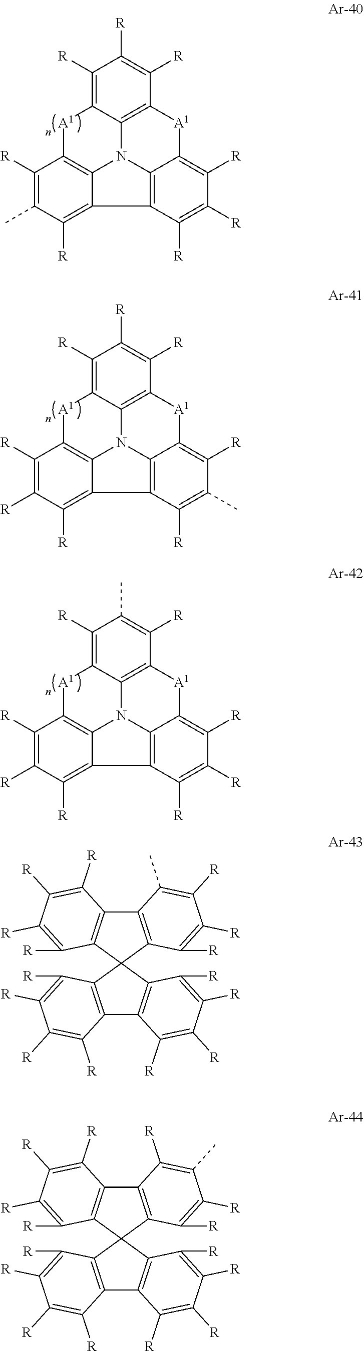

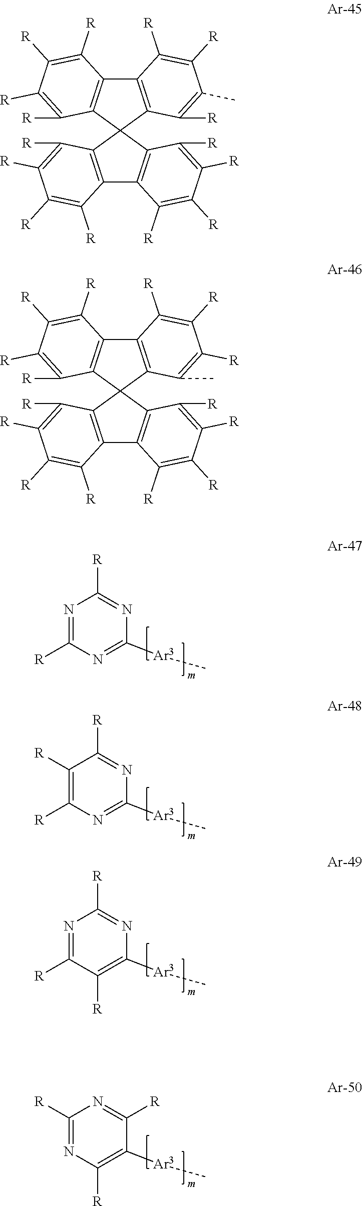

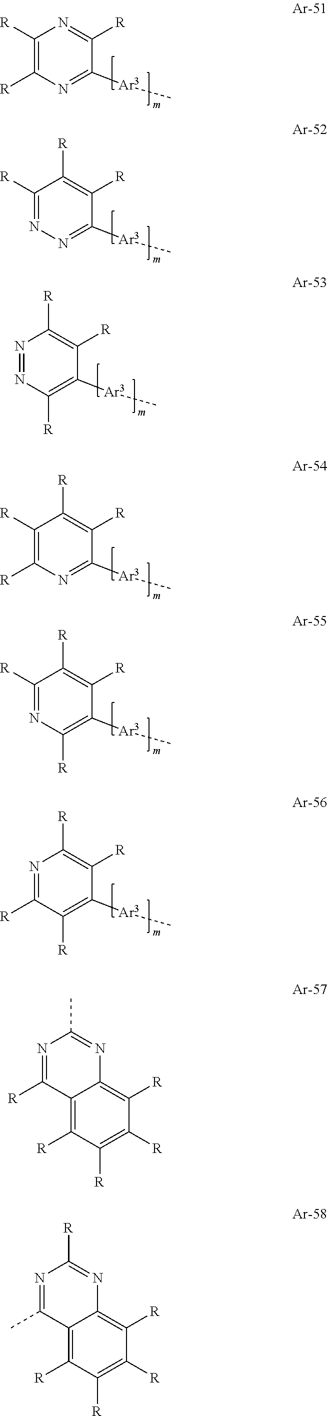

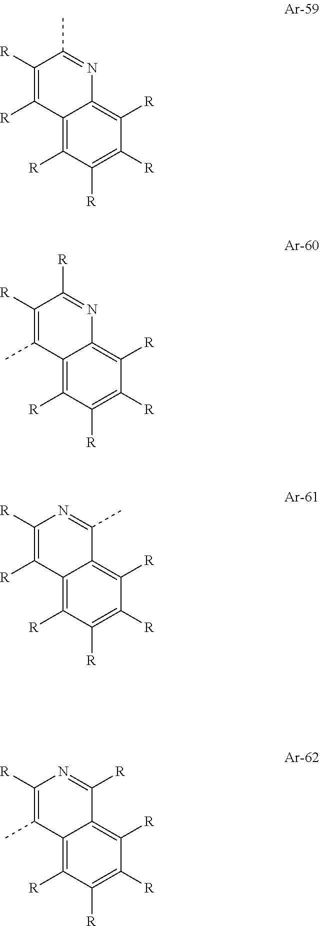

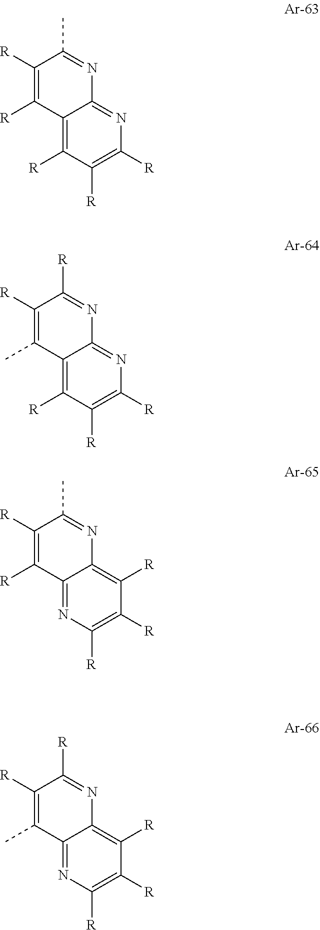

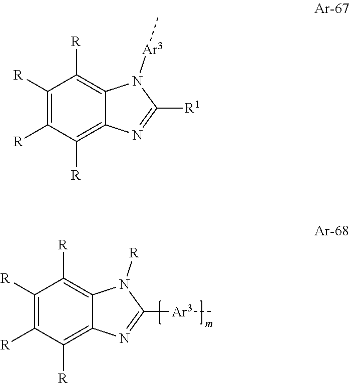

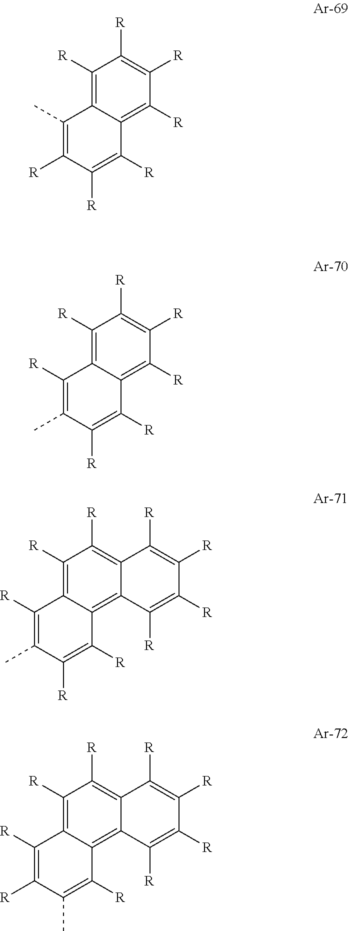

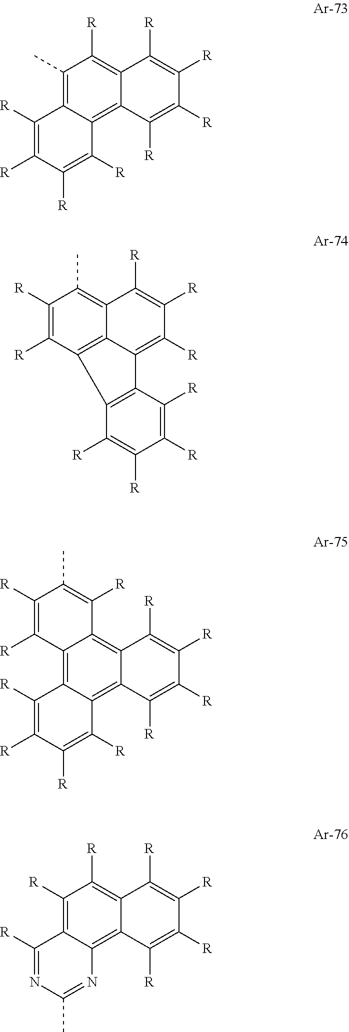

where the dotted line represents the bond to the nitrogen atom in the case of Ar, and in addition:
Ar³ is the same or different at each instance and is a bivalent aromatic or heteroaromatic ring system which has 6 to 18 aromatic ring atoms and may be substituted in each case by one or more R radicals;
Ar³ is an aromatic or heteroaromatic ring system which has 5 to 40 aromatic ring atoms and may be substituted by one or more R radicals;
A¹ is the same or different at each instance and is NAr², O, S or C(R)₂;
n is 0 or 1, where n=0 means that no A¹ group is bonded at this position and R radicals are bonded to the corresponding carbon atoms in its place;
m is 0 or 1, where m=0 means that the Ar³ group is absent and that the corresponding aromatic or heteroaromatic group is bonded directly to the nitrogen atom.

In a preferred embodiment of the invention, R is the same or different at each instance and is selected from the group consisting of H, D, F, N(R¹)₂, CN, OR¹, a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may each be substituted by one or more R¹ radicals, but is preferably unsubstituted, and where one or more nonadjacent CH₂ groups may be replaced by O, or an aromatic or heteroaromatic ring system which has 6 to 30 aromatic ring atoms and may be substituted in each case by one or more R¹ radicals; at the same time, two R radicals together may also form an aliphatic, aromatic or heteroaromatic ring system. More preferably, R is the same or different at each instance and is selected from the group consisting of H, N(R¹)₂, a straight-chain alkyl group having 1 to 6 carbon atoms, especially having 1, 2, 3 or 4 carbon atoms, or a branched or cyclic alkyl group having 3 to 6 carbon atoms, where the alkyl group in each case may be substituted by one or more R¹ radicals, but is preferably unsubstituted, or an aromatic or heteroaromatic ring system which has 6 to 24 aromatic ring atoms and may be substituted in each case by one or more R¹ radicals, preferably nonaromatic R¹ radicals. Most preferably, R is the same or different at each instance and is selected from the group consisting of H or an aromatic or heteroaromatic ring system which has 6 to 24 aromatic ring atoms and may be substituted in each case by one or more R² radicals, preferably nonaromatic R¹ radicals. It may additionally be preferable when R is a triaryl- or -heteroarylamine group which may be substituted by one or more R¹ radicals. This group is one embodiment of an aromatic or heteroaromatic ring system, in which case two or more aryl or heteroaryl groups are joined to one another by a nitrogen atom. When R is a triaryl- or -heteroarylamine group, this group preferably has 18 to 30 aromatic ring atoms and may be substituted by one or more R¹ radicals, preferably nonaromatic R¹ radicals.

Suitable aromatic or heteroaromatic ring systems R are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene which may be joined via the 1 or 2 position, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, quinazoline, benzimidazole, phenanthrene, triphenylene or a combination of two or three of these groups, each of which may be substituted by one or more R¹ radicals. When R is a heteroaryl group, especially triazine, pyrimidine or quinazoline, preference may also be given to aromatic or heteroaromatic R¹ radicals on this heteroaryl group.

The R groups here, when they are an aromatic or heteroaromatic ring system, are preferably selected from the groups of the following formulae R-1 to R-76:

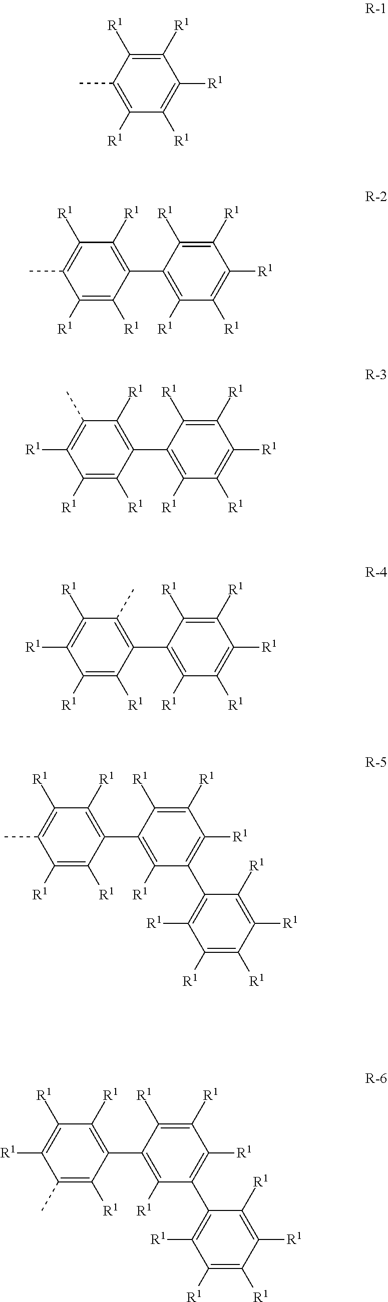

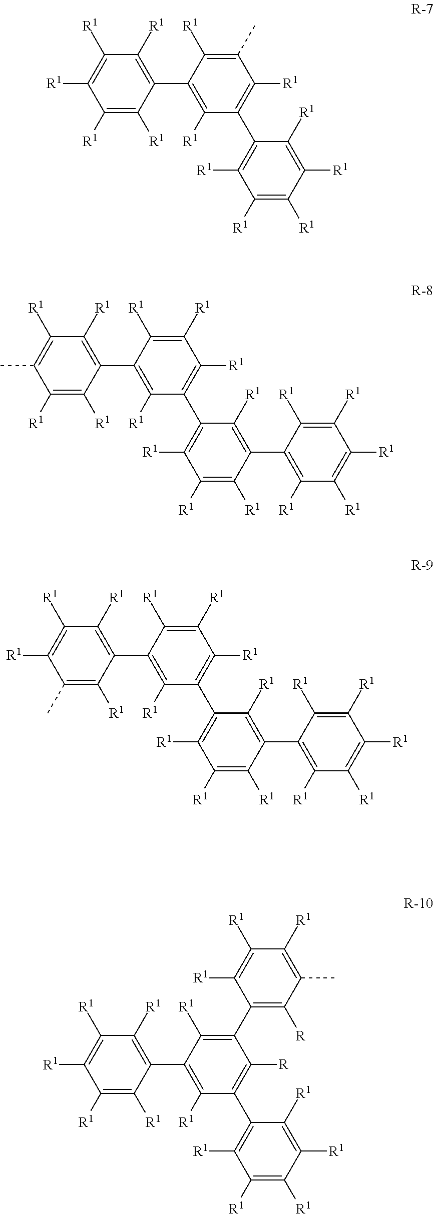

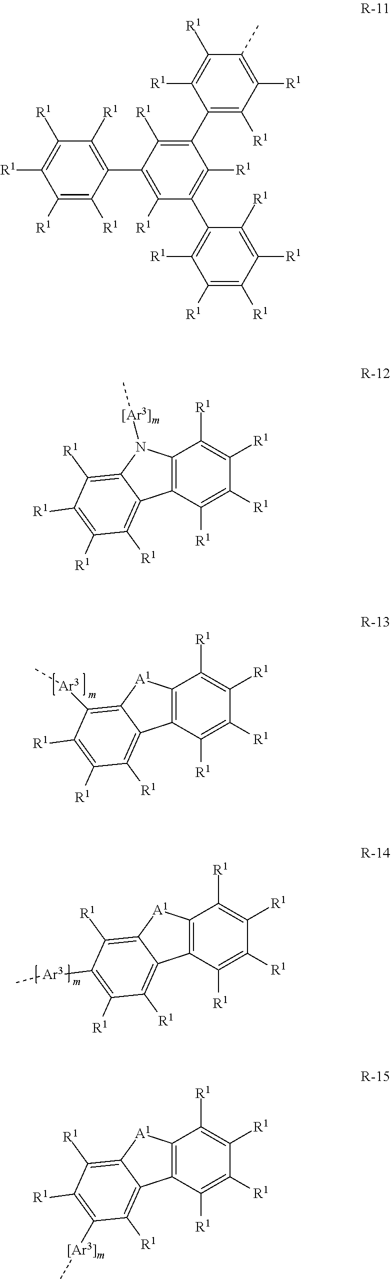

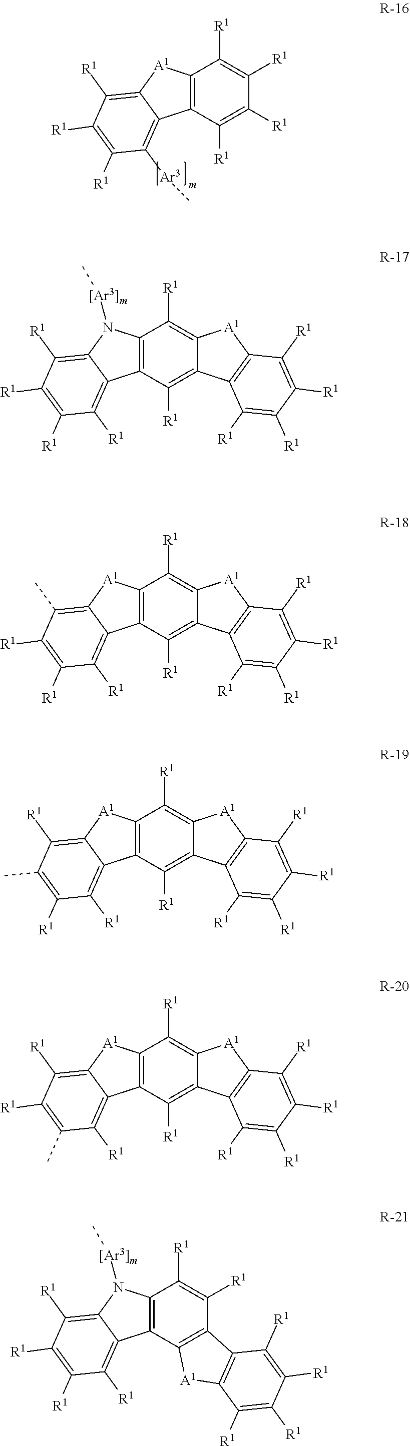

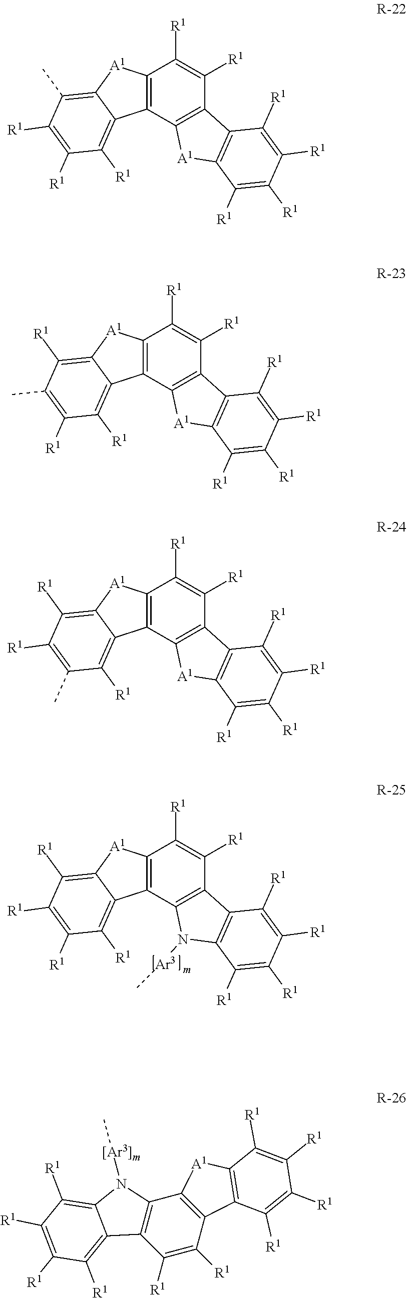

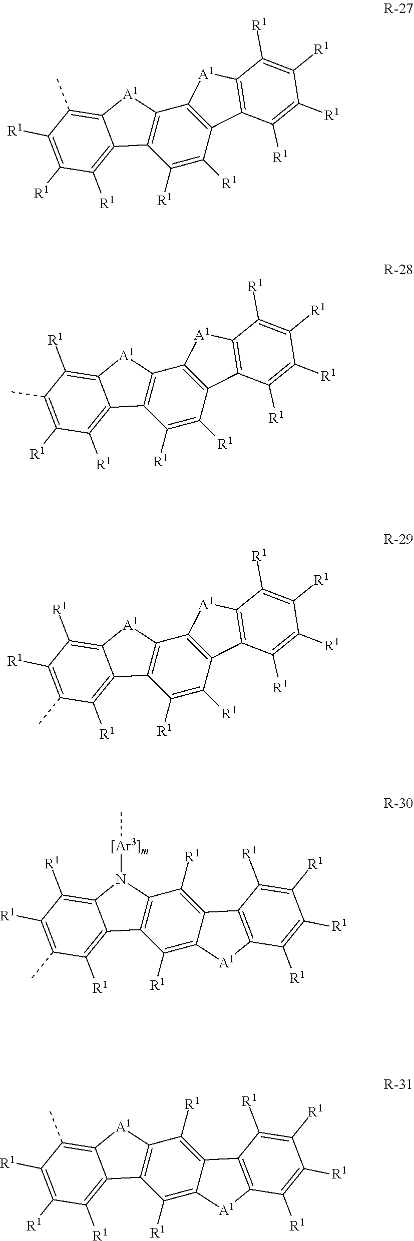

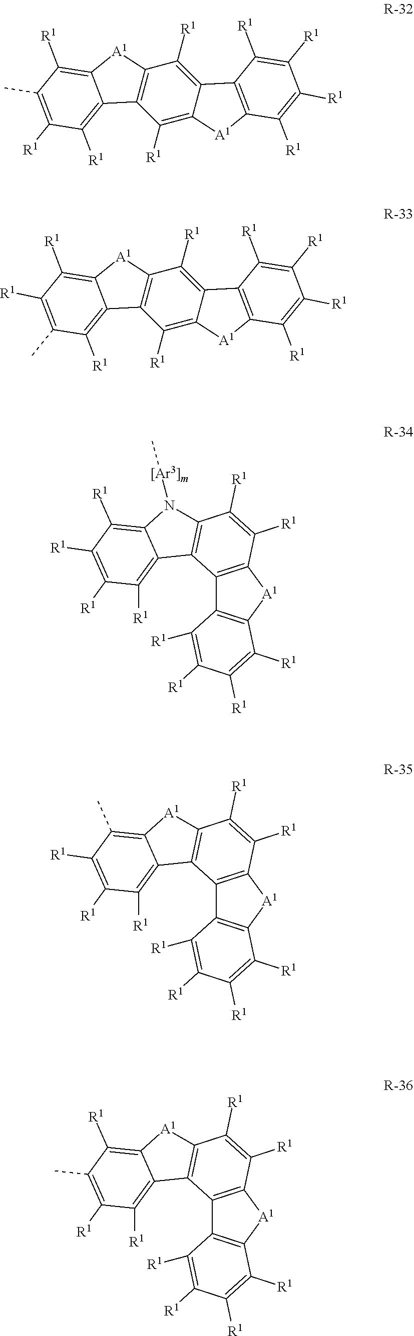

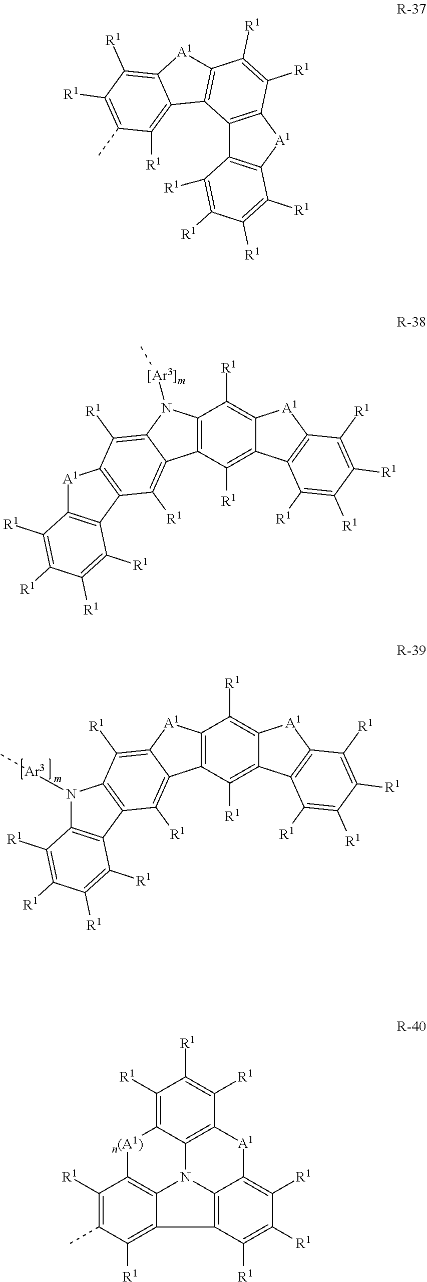

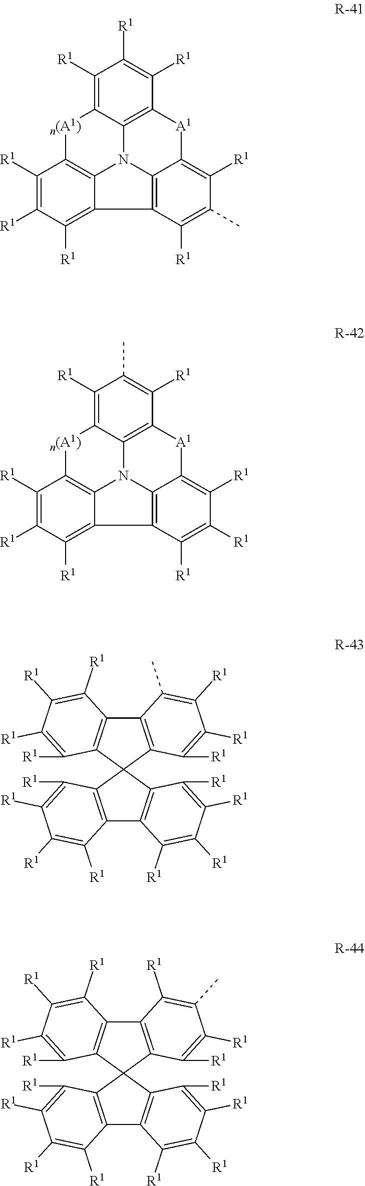

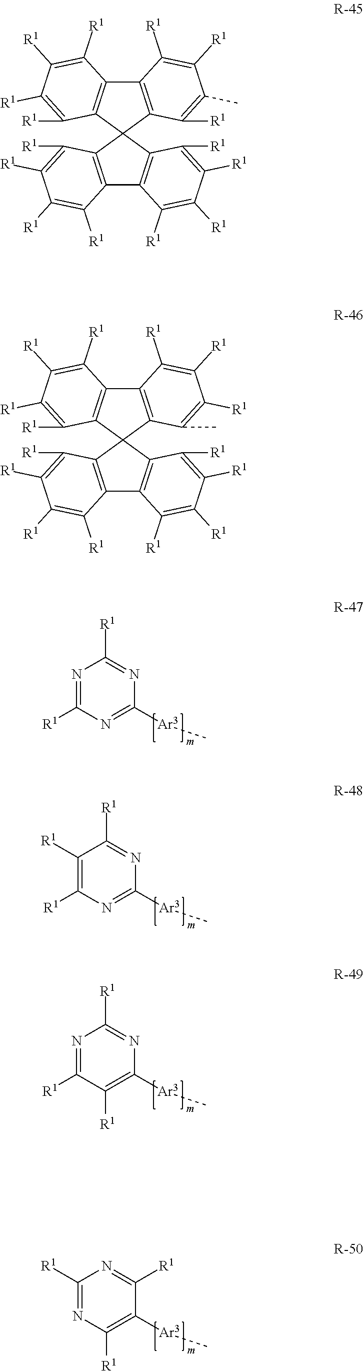

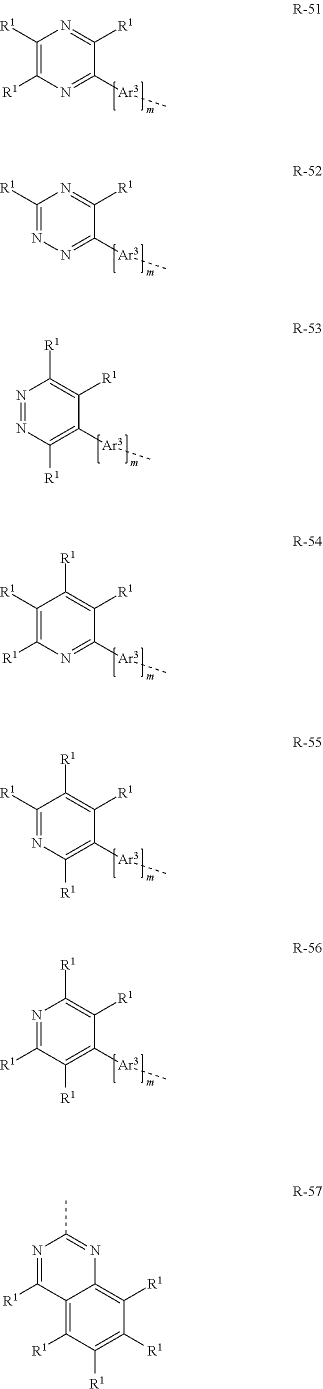

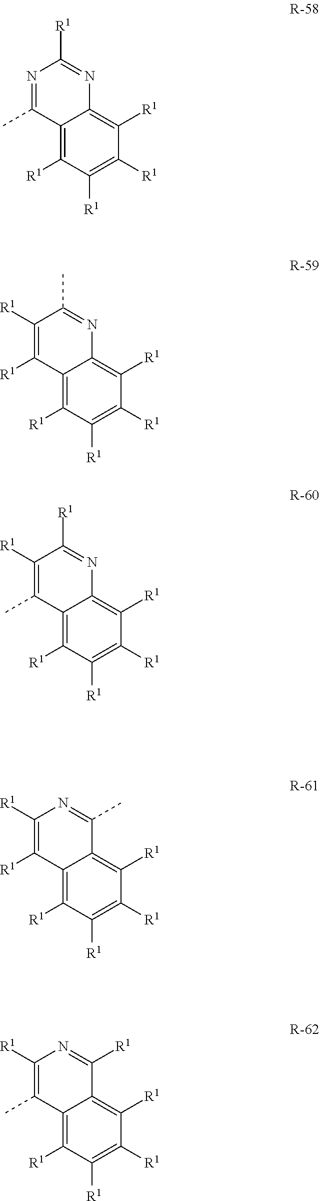

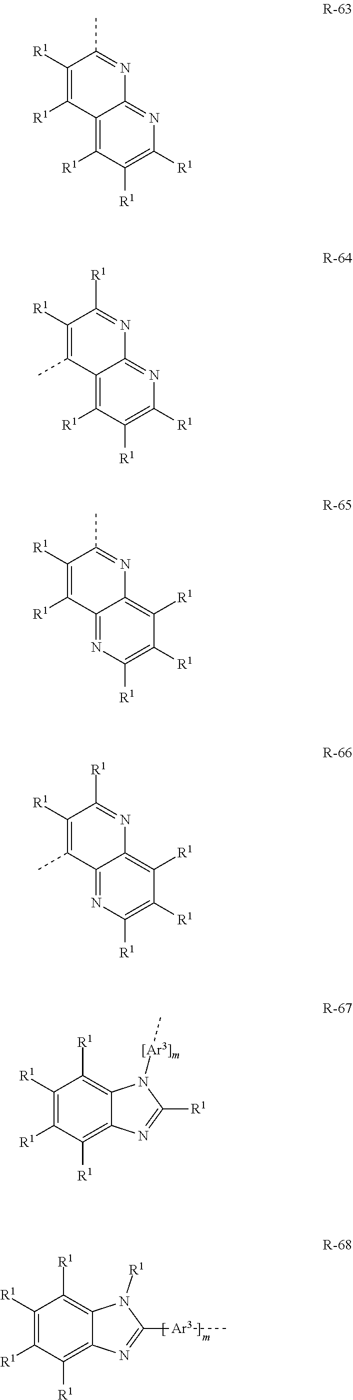

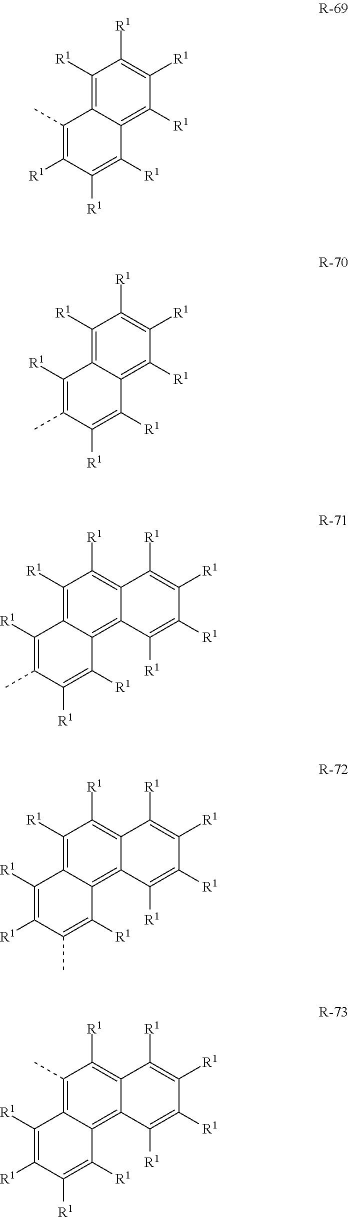

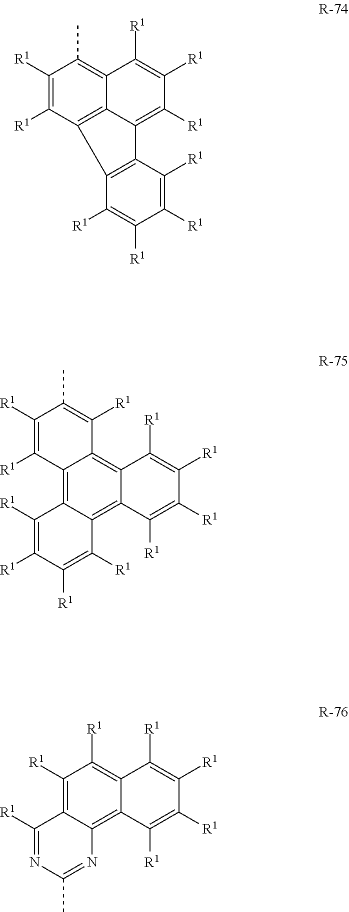

where R¹ has the definitions given above, the dotted bond represents the bond to a carbon atom in the base skeleton in formula (1) or (2) or in the preferred embodiments, and in addition:
Ar³ is the same or different at each instance and is a bivalent aromatic or heteroaromatic ring system which has 6 to 18 aromatic ring atoms and may be substituted in each case by one or more R¹ radicals;
A¹ is the same or different at each instance and is C(R¹)₂, NR¹, O or S;
n is 0 or 1, where n=0 means that no A group is bonded at this position and R¹ radicals are bonded to the corresponding carbon atoms instead;
m is 0 or 1, where m=0 means that the Ar³ group is absent and that the corresponding aromatic or heteroaromatic group is bonded directly to a carbon atom in the base skeleton in formula (1) or formula (2) or in the preferred embodiments;

When the abovementioned Ar-1 to Ar-76 groups for Ar and R-1 to R-76 groups for R have two or more A¹ groups, possible options for these include all combinations from the definition of A¹. Preferred embodiments in that case are those in which one A¹ group is NR or NR¹ and the other A group is C(R)₂ or C(R¹)₂ or in which both A¹ groups are NR or NR¹ or in which both A¹ groups are O. In a particularly preferred embodiment of the invention, in Ar¹, Ar² or R groups having two or more A¹ groups, at least one A¹ group is C(R¹)₂ or is NR¹.

When A¹ is NR¹, the substituent R¹ bonded to the nitrogen atom is preferably an aromatic or heteroaromatic ring system which has 5 to 24 aromatic ring atoms and may also be substituted by one or more R² radicals. In a particularly preferred embodiment, this R¹ substituent is the same or different at each instance and is an aromatic or heteroaromatic ring system which has 6 to 24 aromatic ring atoms, preferably 6 to 12 aromatic ring atoms, and which does not have any fused aryl groups or heteroaryl groups in which two or more aromatic or heteroaromatic 6-membered ring groups are fused directly to one another, and which may also be substituted in each case by one or more R² radicals. Particular preference is given to phenyl, biphenyl, terphenyl and quaterphenyl having bonding patterns as listed above for Ar-1 to Ar-11 or R-1 to R-11, where these structures may be substituted by one or more R² radicals, but are preferably unsubstituted.

When A¹ is C(R¹)₂, the substituents R¹ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R² radicals. Most preferably, R¹ is a methyl group or a phenyl group. In this case, the R¹ radicals together may also form a ring system, which leads to a spiro system.

When Y or Y¹ is CR₂, the substituents R bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R¹ radicals. Most preferably, these substituents R are a methyl group or a phenyl group. In this case, the R radicals together may also form a ring system, which leads to a spiro system.

In one embodiment of the invention, at least one R radical is an electron-rich heteroaromatic ring system. This electron-rich heteroaromatic ring system is preferably selected from the above-depicted R-13 to R-42 groups, where, in the R-13 to R-16, R-18 to R-20, R-22 to R-24, R-27 to R-29, R-31 to R-33 and R-35 to R-37 groups, at least one A¹ group is NR¹ where R¹ is preferably an aromatic or heteroaromatic ring system, especially an aromatic ring system. Particular preference is given to the R-15 group with m=0 and A¹=NR¹.

In a further embodiment of the invention, at least one R radical is an electron-deficient heteroaromatic ring system. This electron-deficient heteroaromatic ring system is preferably selected from the above-depicted R-47 to R-50, R-57, R-58 and R-76 groups.

In one embodiment of the invention, at least one Ar radical is an electron-rich heteroaromatic ring system. This electron-rich heteroaromatic ring system is preferably selected from the above-depicted groups Ar-13 to Ar-42, where, in groups Ar-13 to Ar-16, Ar-18 to Ar-20, Ar-22 to Ar-24, Ar-27 to Ar-29, Ar-31 to Ar-33 and Ar-35 to Ar-37, preferably at least one A¹ group is NAr² where Ar² is preferably an aromatic ring system.

In a further embodiment of the invention, at least one Ar radical is an electron-deficient heteroaromatic ring system. This electron-deficient heteroaromatic ring system is preferably selected from the above-depicted Ar-47 to Ar-50, Ar-57, Ar-58 and Ar-76 groups.

In a further preferred embodiment of the invention, R¹ is the same or different at each instance and is selected from the group consisting of H, D, F, CN, OR², a straight-chain alkyl group having 1 to 10 carbon atoms or an alkenyl group having 2 to 10 carbon atoms or a branched or cyclic alkyl group having 3 to 10 carbon atoms, where the alkyl or alkenyl group may in each case be substituted by one or more R² radicals, and where one or more nonadjacent CH₂ groups may be replaced by O, or an aromatic or heteroaromatic ring system which has 6 to 30 aromatic ring atoms and may be substituted in each case by one or more R² radicals; at the same time, two or more R¹ radicals together may form an aliphatic ring system. In a particularly preferred embodiment of the invention, R¹ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R² radicals, but is preferably unsubstituted, or an aromatic or heteroaromatic ring system which has 6 to 24 aromatic ring atoms and may be substituted in each case by one or more R² radicals, but is preferably unsubstituted.

In a further preferred embodiment of the invention, R² is the same or different at each instance and is H, F, an alkyl group having 1 to 4 carbon atoms or an aryl group having 6 to 10 carbon atoms, which may be substituted by an alkyl group having 1 to 4 carbon atoms, but is preferably unsubstituted.

At the same time, the alkyl groups in compounds of the invention which are processed by vacuum evaporation preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. It is especially preferable when the Ar, R, R¹ and R² radicals do not contain any fused aryl or heteroaryl groups in which two or more six-membered rings are fused directly to one another. An exception to this is formed by phenanthrene, triphenylene and quinazoline, which, because of their high triplet energy, may be preferable in spite of the presence of fused aromatic six-membered rings.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

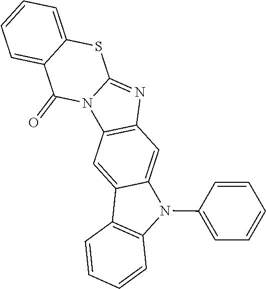 [SMILES: O=c1c2ccccc2sc2nc3cc4c(cc3n12)c1ccccc1n4-c1ccccc1]

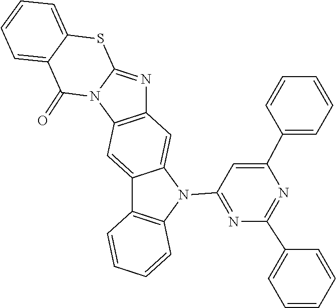 [SMILES: O=c1c2ccccc2sc2nc3cc4c(cc3n12)c1ccccc1n4-c1cc(-c2ccccc2)nc(-c2ccccc2)n1]

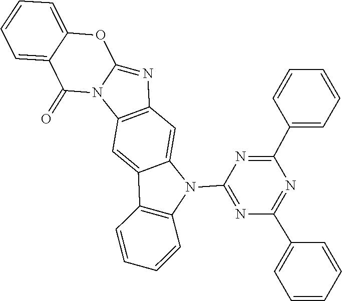 [SMILES: O=c1c2ccccc2oc2nc3cc4c(cc3n12)c1ccccc1n4-c1nc(-c2ccccc2)nc(-c2ccccc2)n1]

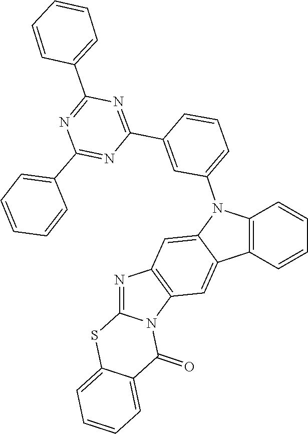 [SMILES: O=c1c2ccccc2sc2nc3cc4c(cc3n12)c1ccccc1n4-c1cccc(-c2nc(-c3ccccc3)nc(-c3ccccc3)n2)c1]

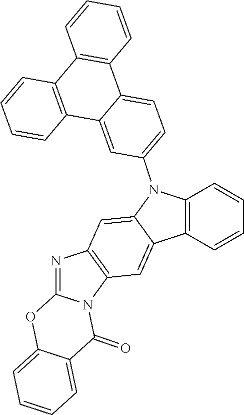 [SMILES: O=c1c2ccccc2oc2nc3cc4c(cc3n12)c1ccccc1n4-c1ccc2c3ccccc3c3ccccc3c2c1]

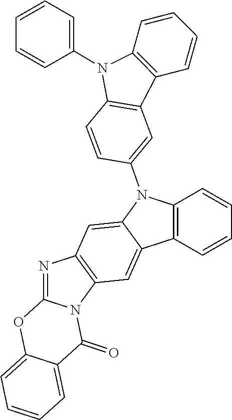 [SMILES: O=c1c2ccccc2oc2nc3cc4c(cc3n12)c1ccccc1n4-c1ccc2c(c1)c1ccccc1n2-c1ccccc1]

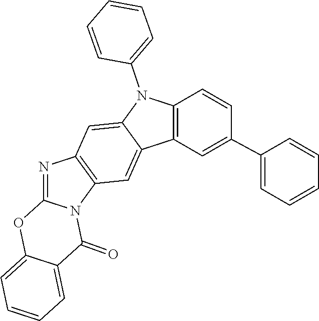 [SMILES: O=c1c2ccccc2oc2nc3cc4c(cc3n12)c1cc(-c2ccccc2)ccc1n4-c1ccccc1]

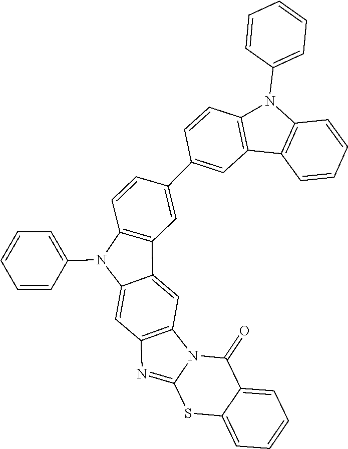 [SMILES: O=c1c2ccccc2sc2nc3cc4c(cc3n12)c1cc(-c2ccc3c(c2)c2ccccc2n3-c2ccccc2)ccc1n4-c1ccccc1]

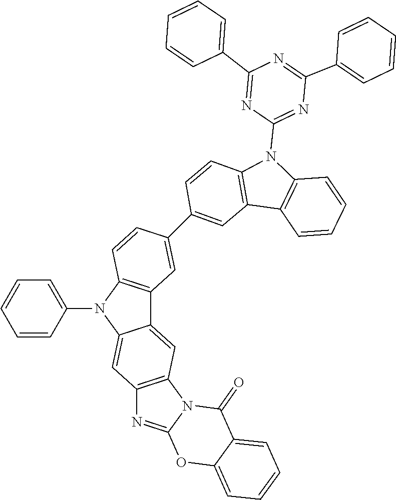 [SMILES: O=c1c2ccccc2oc2nc3cc4c(cc3n12)c1cc(-c2ccc3c(c2)c2ccccc2n3-c2nc(-c3ccccc3)nc(-c3ccccc3)n2)ccc1n4-c1ccccc1]

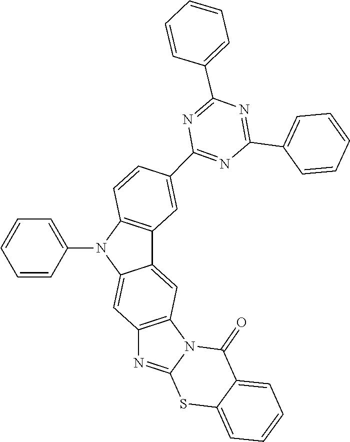 [SMILES: O=c1c2ccccc2sc2nc3cc4c(cc3n12)c1cc(-c2nc(-c3ccccc3)nc(-c3ccccc3)n2)ccc1n4-c1ccccc1]

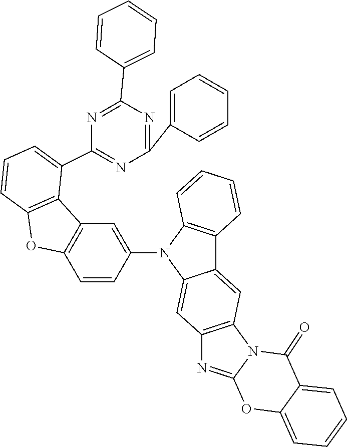 [SMILES: O=c1c2ccccc2oc2nc3cc4c(cc3n12)c1ccccc1n4-c1ccc2oc3cccc(-c4nc(-c5ccccc5)nc(-c5ccccc5)n4)c3c2c1]

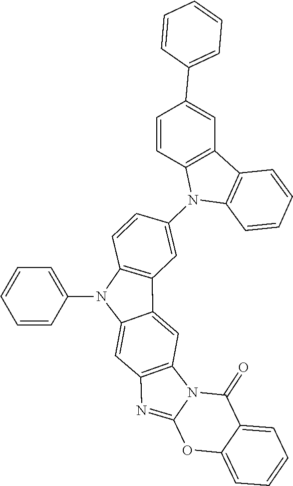 [SMILES: O=c1c2ccccc2oc2nc3cc4c(cc3n12)c1cc(-n2c3ccccc3c3cc(-c5ccccc5)ccc32)ccc1n4-c1ccccc1]

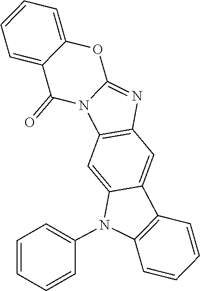 [SMILES: O=c1c2ccccc2oc2nc3cc4c5ccccc5n(-c5ccccc5)c4cc3n12]

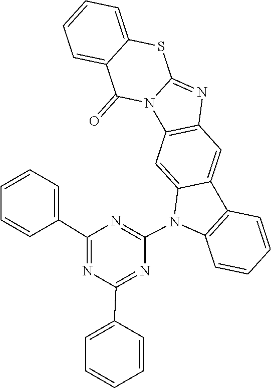 [SMILES: O=c1c2ccccc2sc2nc3cc4c5ccccc5n(-c5nc(-c6ccccc6)nc(-c6ccccc6)n5)c4cc3n12]

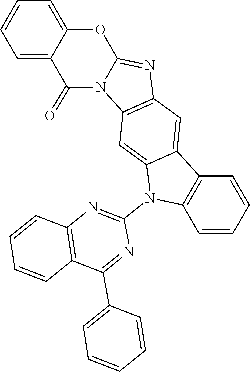 [SMILES: O=c1c2ccccc2oc2nc3cc4c5ccccc5n(-c5nc(-c6ccccc6)c6ccccc6n5)c4cc3n12]

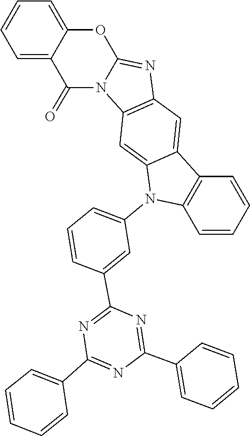 [SMILES: O=c1c2ccccc2oc2nc3cc4c5ccccc5n(-c5cccc(-c6nc(-c7ccccc7)nc(-c7ccccc7)n6)c5)c4cc3n12]

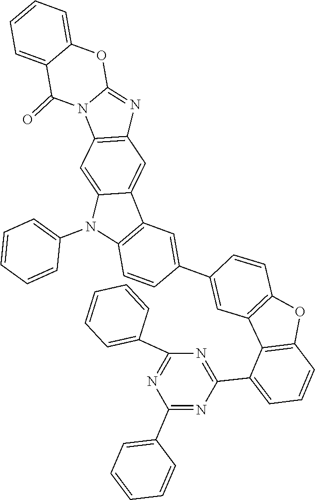 [SMILES: O=c1c2ccccc2oc2nc3cc4c5cc(-c6ccc7oc8cccc(-c9nc(-c%10ccccc%10)nc(-c%10ccccc%10)n9)c8c7c6)ccc5n(-c5ccccc5)c4cc3n12]

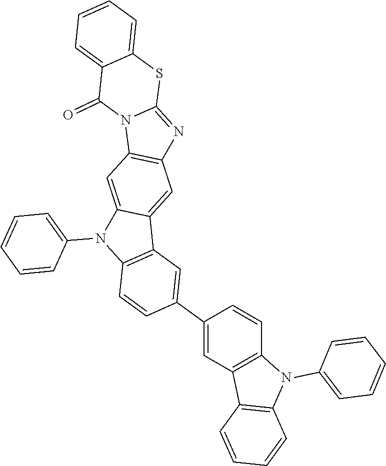 [SMILES: O=c1c2ccccc2sc2nc3cc4c5cc(-c6ccc7c(c6)c6ccccc6n7-c6ccccc6)ccc5n(-c5ccccc5)c4cc3n12]

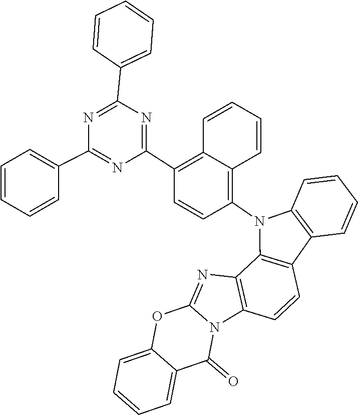 [SMILES: O=c1c2ccccc2oc2nc3c4c(ccc3n12)c1ccccc1n4-c1ccc(-c2nc(-c3ccccc3)nc(-c3ccccc3)n2)c2ccccc12]

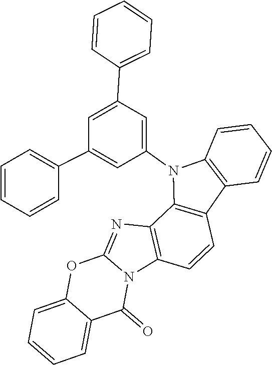 [SMILES: O=c1c2ccccc2oc2nc3c4c(ccc3n12)c1ccccc1n4-c1cc(-c2ccccc2)cc(-c2ccccc2)c1]

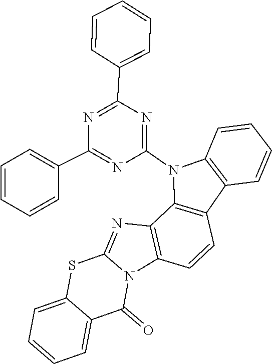 [SMILES: O=c1c2ccccc2sc2nc3c4c(ccc3n12)c1ccccc1n4-c1nc(-c2ccccc2)nc(-c2ccccc2)n1]

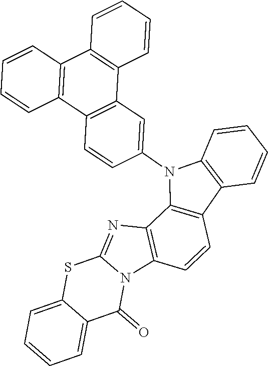 [SMILES: O=c1c2ccccc2sc2nc3c4c(ccc3n12)c1ccccc1n4-c1ccc2c3ccccc3c3ccccc3c2c1]

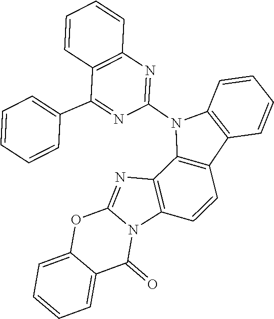 [SMILES: O=c1c2ccccc2oc2nc3c4c(ccc3n12)c1ccccc1n4-c1nc(-c2ccccc2)c2ccccc2n1]

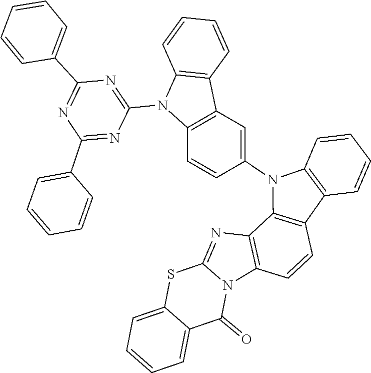 [SMILES: O=c1c2ccccc2sc2nc3c4c(ccc3n12)c1ccccc1n4-c1ccc2c(c1)c1ccccc1n2-c1nc(-c2ccccc2)nc(-c2ccccc2)n1]

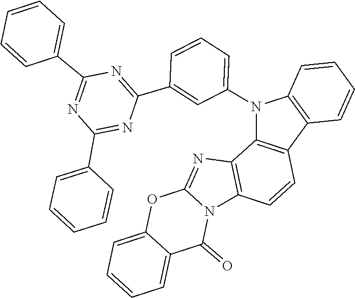 [SMILES: O=c1c2ccccc2oc2nc3c4c(ccc3n12)c1ccccc1n4-c1cccc(-c2nc(-c3ccccc3)nc(-c3ccccc3)n2)c1]

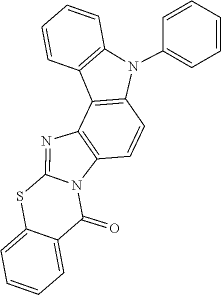 [SMILES: O=c1c2ccccc2sc2nc3c4c5ccccc5n(-c5ccccc5)c4ccc3n12]

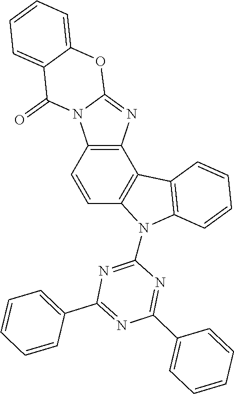 [SMILES: O=c1c2ccccc2oc2nc3c4c5ccccc5n(-c5nc(-c6ccccc6)nc(-c6ccccc6)n5)c4ccc3n12]

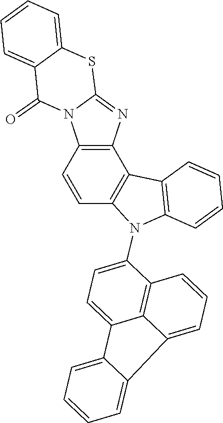 [SMILES: O=c1c2ccccc2sc2nc3c4c5ccccc5n(-c5ccc6c7c(cccc57)-c5ccccc5-6)c4ccc3n12]

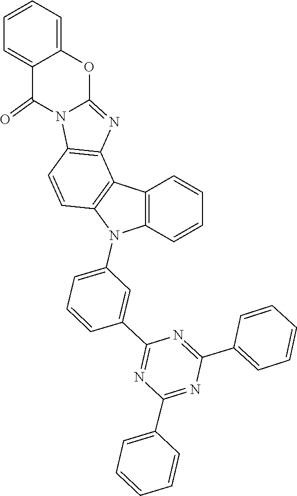 [SMILES: O=c1c2ccccc2oc2nc3c4c5ccccc5n(-c5cccc(-c6nc(-c7ccccc7)nc(-c7ccccc7)n6)c5)c4ccc3n12]

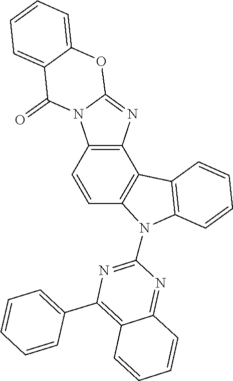 [SMILES: O=c1c2ccccc2oc2nc3c4c5ccccc5n(-c5nc(-c6ccccc6)c6ccccc6n5)c4ccc3n12]

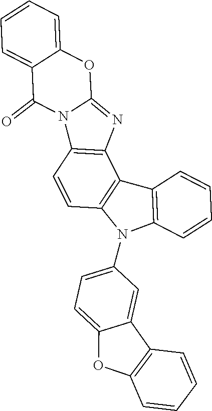 [SMILES: O=c1c2ccccc2oc2nc3c4c5ccccc5n(-c5ccc6oc7ccccc7c6c5)c4ccc3n12]

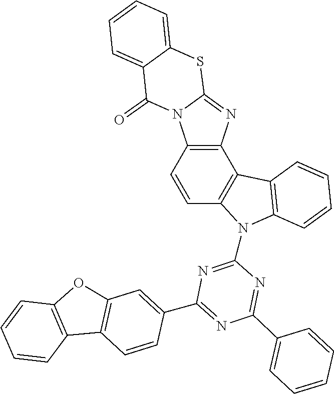 [SMILES: O=c1c2ccccc2sc2nc3c4c5ccccc5n(-c5nc(-c6ccccc6)nc(-c6ccc7c(c6)oc6ccccc67)n5)c4ccc3n12]

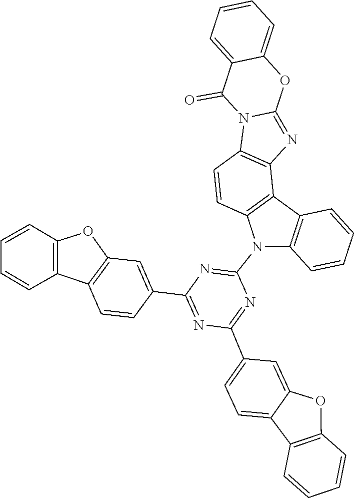 [SMILES: O=c1c2ccccc2oc2nc3c4c5ccccc5n(-c5nc(-c6ccc7c(c6)oc6ccccc67)nc(-c6ccc7c(c6)oc6ccccc67)n5)c4ccc3n12]

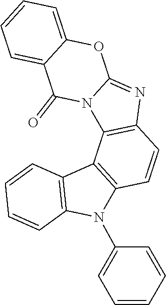 [SMILES: O=c1c2ccccc2oc2nc3ccc4c(c5ccccc5n4-c4ccccc4)c3n12]

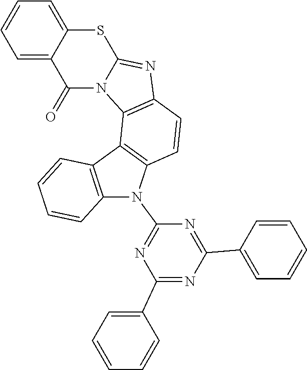 [SMILES: O=c1c2ccccc2sc2nc3ccc4c(c5ccccc5n4-c4nc(-c5ccccc5)nc(-c5ccccc5)n4)c3n12]

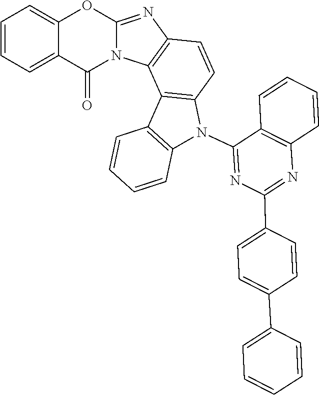 [SMILES: O=c1c2ccccc2oc2nc3ccc4c(c5ccccc5n4-c4nc(-c5ccc(-c6ccccc6)cc5)nc5ccccc45)c3n12]

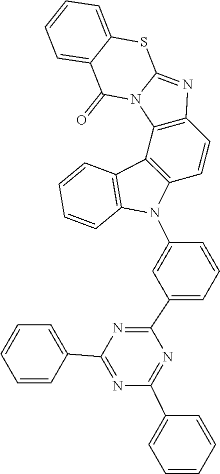 [SMILES: O=c1c2ccccc2sc2nc3ccc4c(c5ccccc5n4-c4cccc(-c5nc(-c6ccccc6)nc(-c6ccccc6)n5)c4)c3n12]

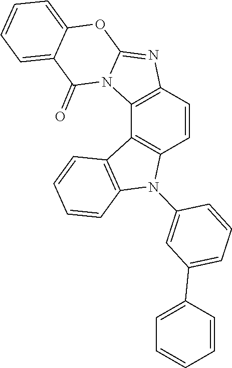 [SMILES: O=c1c2ccccc2oc2nc3ccc4c(c5ccccc5n4-c4cccc(-c5ccccc5)c4)c3n12]

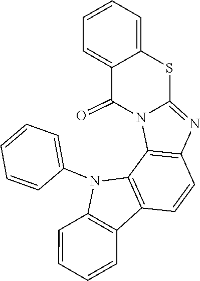 [SMILES: O=c1c2ccccc2sc2nc3ccc4c5ccccc5n(-c5ccccc5)c4c3n12]

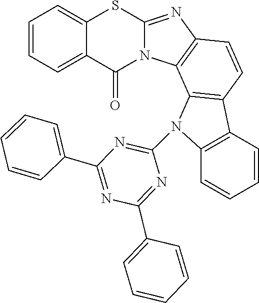 [SMILES: O=c1c2ccccc2sc2nc3ccc4c5ccccc5n(-c5nc(-c6ccccc6)nc(-c6ccccc6)n5)c4c3n12]

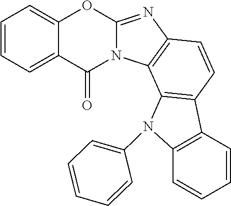 [SMILES: O=c1c2ccccc2oc2nc3ccc4c5ccccc5n(-c5ccccc5)c4c3n12]

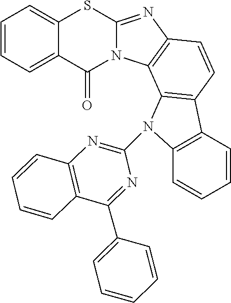 [SMILES: O=c1c2ccccc2sc2nc3ccc4c5ccccc5n(-c5nc(-c6ccccc6)c6ccccc6n5)c4c3n12]

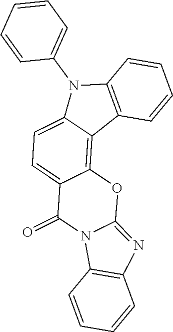 [SMILES: O=c1c2ccc3c(c4ccccc4n3-c3ccccc3)c2oc2nc3ccccc3n12]

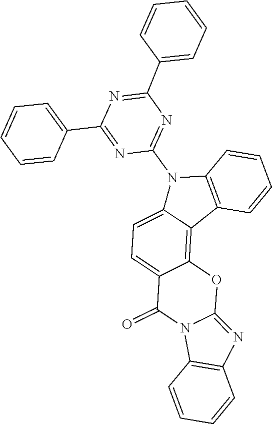 [SMILES: O=c1c2ccc3c(c4ccccc4n3-c3nc(-c4ccccc4)nc(-c4ccccc4)n3)c2oc2nc3ccccc3n12]

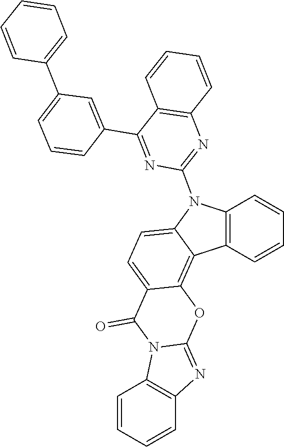 [SMILES: O=c1c2ccc3c(c4ccccc4n3-c3nc(-c4cccc(-c5ccccc5)c4)c4ccccc4n3)c2oc2nc3ccccc3n12]

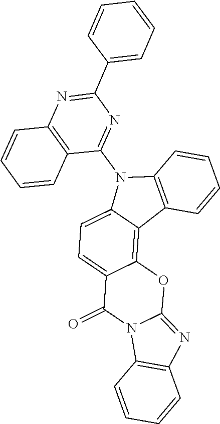 [SMILES: O=c1c2ccc3c(c4ccccc4n3-c3nc(-c4ccccc4)nc4ccccc34)c2oc2nc3ccccc3n12]

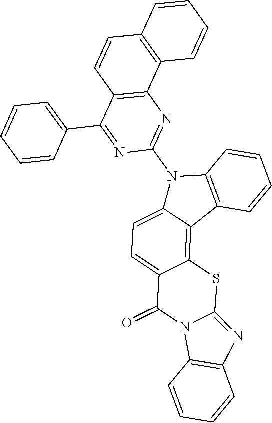 [SMILES: O=c1c2ccc3c(c4ccccc4n3-c3nc(-c4ccccc4)c4ccc5ccccc5c4n3)c2sc2nc3ccccc3n12]

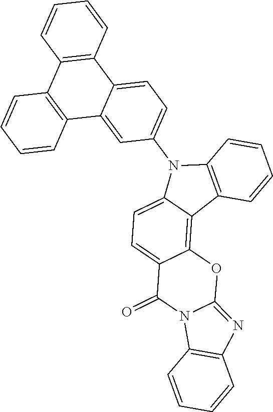 [SMILES: O=c1c2ccc3c(c4ccccc4n3-c3ccc4c5ccccc5c5ccccc5c4c3)c2oc2nc3ccccc3n12]

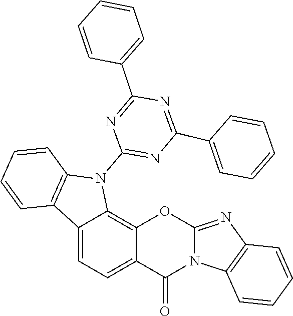 [SMILES: O=c1c2ccc3c4ccccc4n(-c4nc(-c5ccccc5)nc(-c5ccccc5)n4)c3c2oc2nc3ccccc3n12]

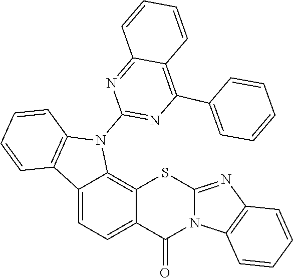 [SMILES: O=c1c2ccc3c4ccccc4n(-c4nc(-c5ccccc5)c5ccccc5n4)c3c2sc2nc3ccccc3n12]

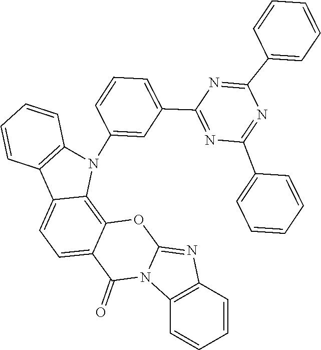 [SMILES: O=c1c2ccc3c4ccccc4n(-c4cccc(-c5nc(-c6ccccc6)nc(-c6ccccc6)n5)c4)c3c2oc2nc3ccccc3n12]

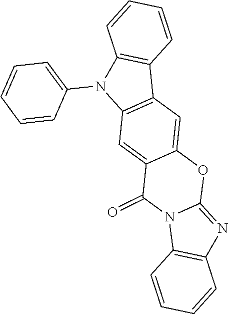 [SMILES: O=c1c2cc3c(cc2oc2nc4ccccc4n12)c1ccccc1n3-c1ccccc1]

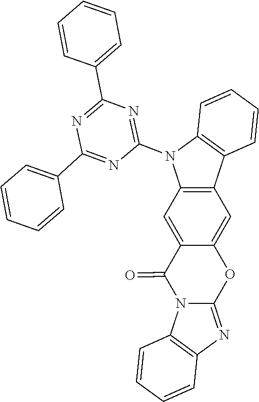 [SMILES: O=c1c2cc3c(cc2oc2nc4ccccc4n12)c1ccccc1n3-c1nc(-c2ccccc2)nc(-c2ccccc2)n1]

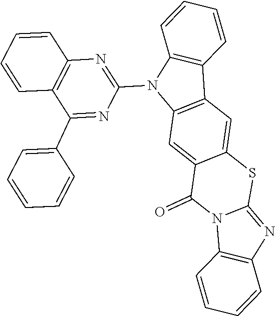 [SMILES: O=c1c2cc3c(cc2sc2nc4ccccc4n12)c1ccccc1n3-c1nc(-c2ccccc2)c2ccccc2n1]

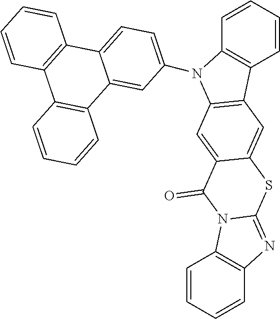 [SMILES: O=c1c2cc3c(cc2sc2nc4ccccc4n12)c1ccccc1n3-c1ccc2c3ccccc3c3ccccc3c2c1]

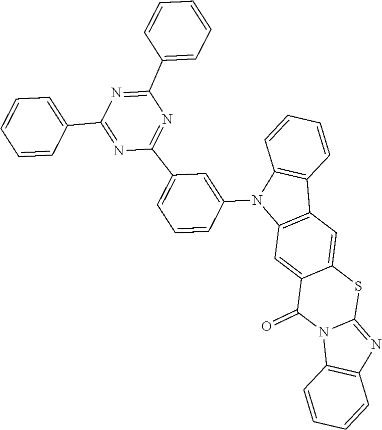 [SMILES: O=c1c2cc3c(cc2sc2nc4ccccc4n12)c1ccccc1n3-c1cccc(-c2nc(-c3ccccc3)nc(-c3ccccc3)n2)c1]

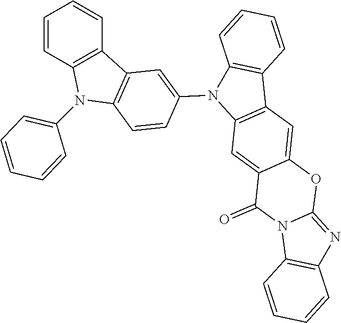 [SMILES: O=c1c2cc3c(cc2oc2nc4ccccc4n12)c1ccccc1n3-c1ccc2c(c1)c1ccccc1n2-c1ccccc1]

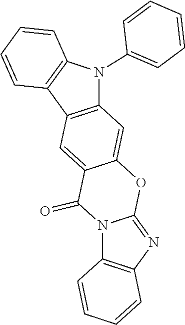 [SMILES: O=c1c2cc3c4ccccc4n(-c4ccccc4)c3cc2oc2nc3ccccc3n12]

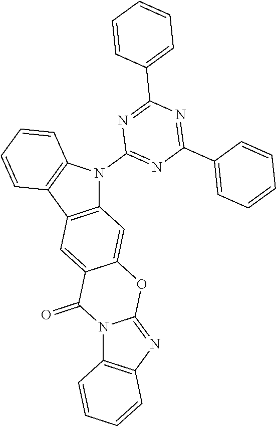 [SMILES: O=c1c2cc3c4ccccc4n(-c4nc(-c5ccccc5)nc(-c5ccccc5)n4)c3cc2oc2nc3ccccc3n12]

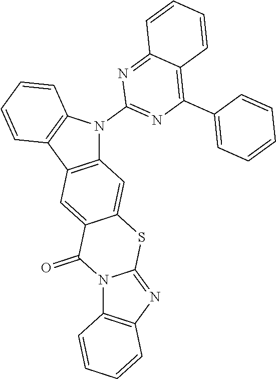 [SMILES: O=c1c2cc3c4ccccc4n(-c4nc(-c5ccccc5)c5ccccc5n4)c3cc2sc2nc3ccccc3n12]

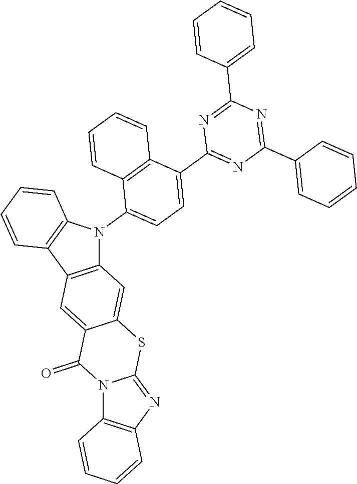 [SMILES: O=c1c2cc3c4ccccc4n(-c4ccc(-c5nc(-c6ccccc6)nc(-c6ccccc6)n5)c5ccccc45)c3cc2sc2nc3ccccc3n12]

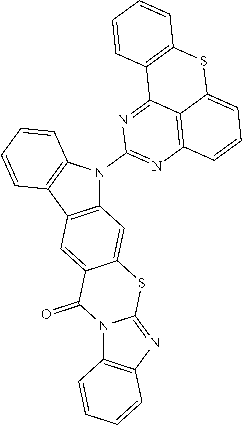 [SMILES: O=c1c2cc3c4ccccc4n(-c4nc5c6c(cccc6n4)Sc4ccccc4-5)c3cc2sc2nc3ccccc3n12]

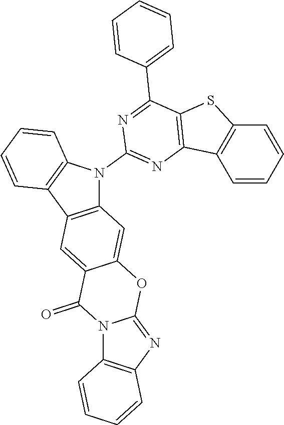 [SMILES: O=c1c2cc3c4ccccc4n(-c4nc(-c5ccccc5)c5sc6ccccc6c5n4)c3cc2oc2nc3ccccc3n12]

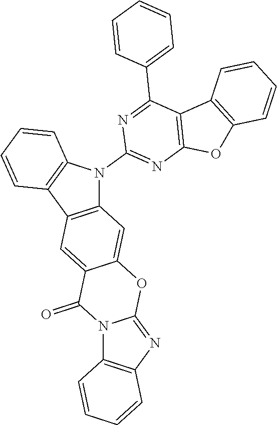 [SMILES: O=c1c2cc3c4ccccc4n(-c4nc(-c5ccccc5)c5c(n4)oc4ccccc45)c3cc2oc2nc3ccccc3n12]

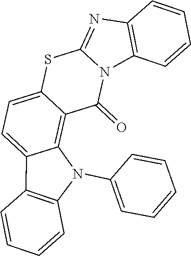 [SMILES: O=c1c2c(ccc3c4ccccc4n(-c4ccccc4)c32)sc2nc3ccccc3n12]

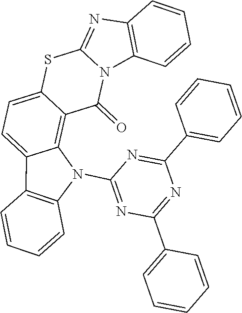 [SMILES: O=c1c2c(ccc3c4ccccc4n(-c4nc(-c5ccccc5)nc(-c5ccccc5)n4)c32)sc2nc3ccccc3n12]

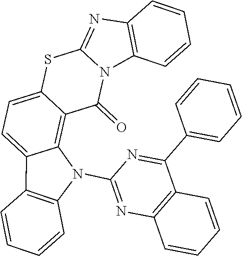 [SMILES: O=c1c2c(ccc3c4ccccc4n(-c4nc(-c5ccccc5)c5ccccc5n4)c32)sc2nc3ccccc3n12]

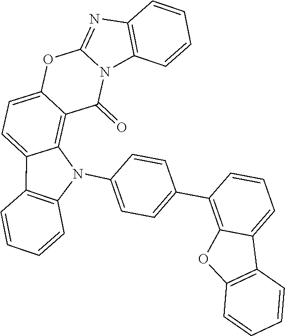 [SMILES: O=c1c2c(ccc3c4ccccc4n(-c4ccc(-c5cccc6c5oc5ccccc56)cc4)c32)oc2nc3ccccc3n12]

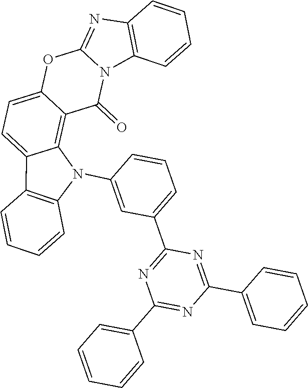 [SMILES: O=c1c2c(ccc3c4ccccc4n(-c4cccc(-c5nc(-c6ccccc6)nc(-c6ccccc6)n5)c4)c32)oc2nc3ccccc3n12]

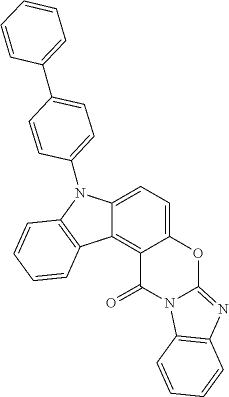 [SMILES: O=c1c2c(ccc3c2c2ccccc2n3-c2ccc(-c3ccccc3)cc2)oc2nc3ccccc3n12]

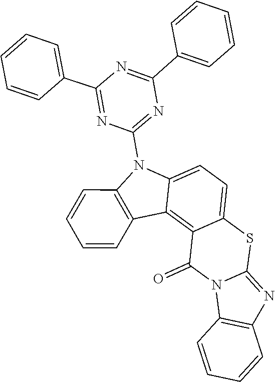 [SMILES: O=c1c2c(ccc3c2c2ccccc2n3-c2nc(-c3ccccc3)nc(-c3ccccc3)n2)sc2nc3ccccc3n12]

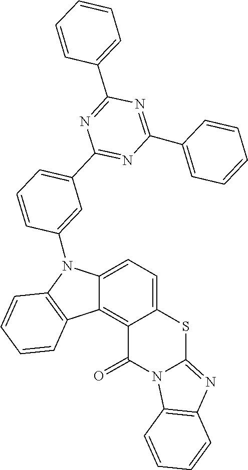 [SMILES: O=c1c2c(ccc3c2c2ccccc2n3-c2cccc(-c3nc(-c4ccccc4)nc(-c4ccccc4)n3)c2)sc2nc3ccccc3n12]

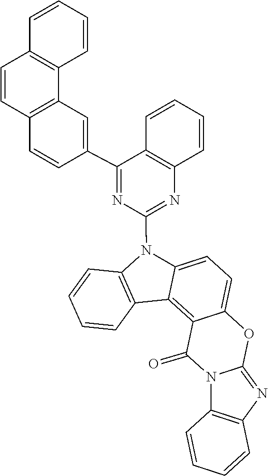 [SMILES: O=c1c2c(ccc3c2c2ccccc2n3-c2nc(-c3ccc4ccc5ccccc5c4c3)c3ccccc3n2)oc2nc3ccccc3n12]

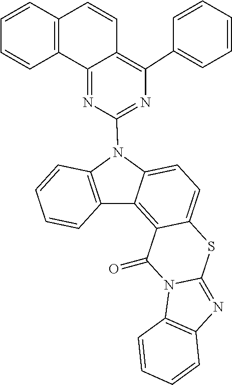 [SMILES: O=c1c2c(ccc3c2c2ccccc2n3-c2nc(-c3ccccc3)c3ccc4ccccc4c3n2)sc2nc3ccccc3n12]

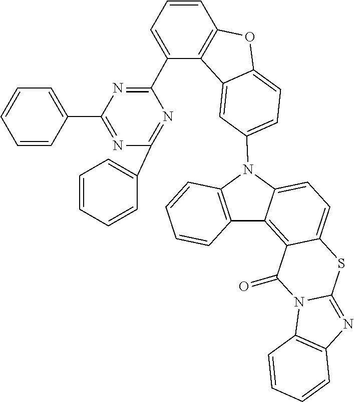 [SMILES: O=c1c2c(ccc3c2c2ccccc2n3-c2ccc3oc4cccc(-c5nc(-c6ccccc6)nc(-c6ccccc6)n5)c4c3c2)sc2nc3ccccc3n12]

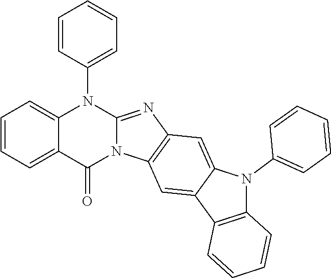 [SMILES: O=c1c2ccccc2n(-c2ccccc2)c2nc3cc4c(cc3n12)c1ccccc1n4-c1ccccc1]

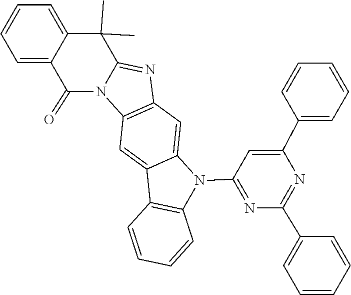 [SMILES: CC1(C)c2ccccc2C(=O)n2c1nc1cc3c(cc12)c1ccccc1n3-c1cc(-c2ccccc2)nc(-c2ccccc2)n1]

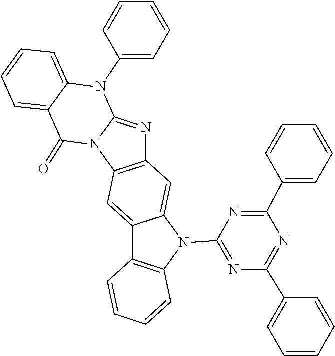 [SMILES: O=c1c2ccccc2n(-c2ccccc2)c2nc3cc4c(cc3n12)c1ccccc1n4-c1nc(-c2ccccc2)nc(-c2ccccc2)n1]

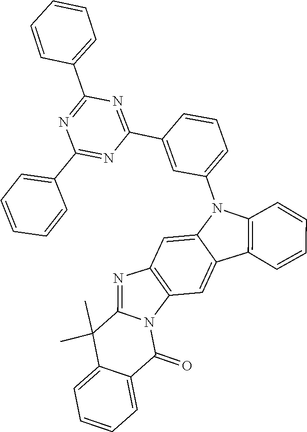 [SMILES: CC1(C)c2ccccc2C(=O)n2c1nc1cc3c(cc12)c1ccccc1n3-c1cccc(-c2nc(-c3ccccc3)nc(-c3ccccc3)n2)c1]

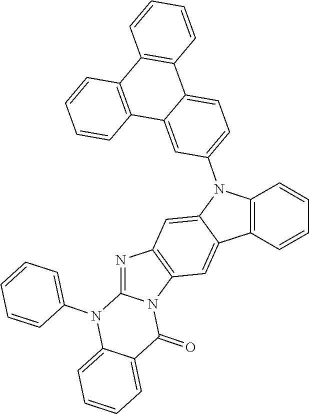 [SMILES: O=c1c2ccccc2n(-c2ccccc2)c2nc3cc4c(cc3n12)c1ccccc1n4-c1ccc2c3ccccc3c3ccccc3c2c1]

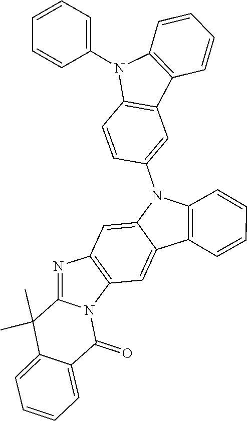 [SMILES: CC1(C)c2ccccc2C(=O)n2c1nc1cc3c(cc12)c1ccccc1n3-c1ccc2c(c1)c1ccccc1n2-c1ccccc1]

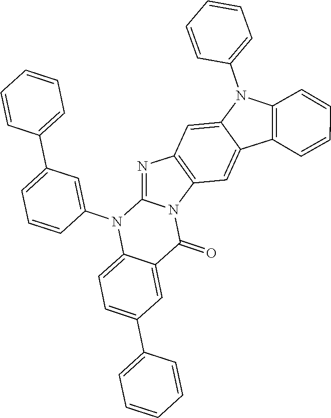 [SMILES: O=c1c2cc(-c3ccccc3)ccc2n(-c2cccc(-c3ccccc3)c2)c2nc3cc4c(cc3n12)c1ccccc1n4-c1ccccc1]

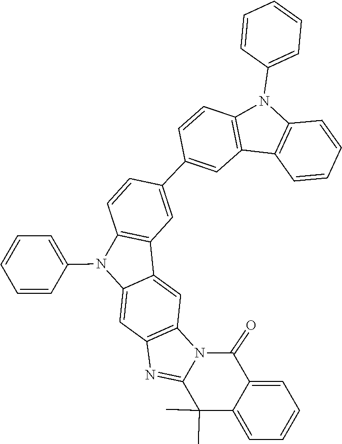 [SMILES: CC1(C)c2ccccc2C(=O)n2c1nc1cc3c(cc12)c1cc(-c2ccc4c(c2)c2ccccc2n4-c2ccccc2)ccc1n3-c1ccccc1]

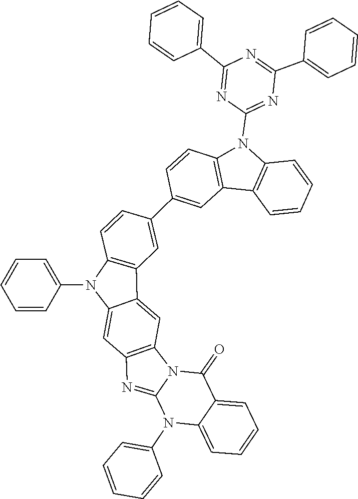 [SMILES: O=c1c2ccccc2n(-c2ccccc2)c2nc3cc4c(cc3n12)c1cc(-c2ccc3c(c2)c2ccccc2n3-c2nc(-c3ccccc3)nc(-c3ccccc3)n2)ccc1n4-c1ccccc1]

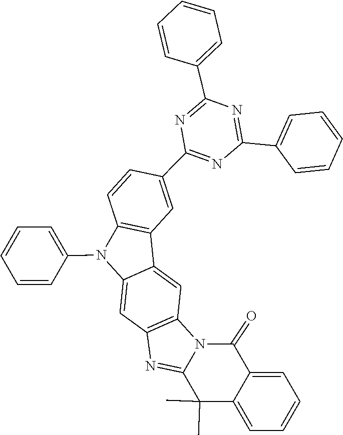 [SMILES: CC1(C)c2ccccc2C(=O)n2c1nc1cc3c(cc12)c1cc(-c2nc(-c4ccccc4)nc(-c4ccccc4)n2)ccc1n3-c1ccccc1]

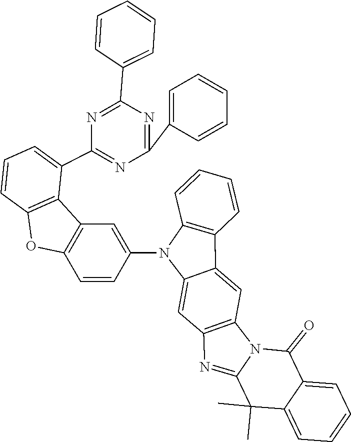 [SMILES: CC1(C)c2ccccc2C(=O)n2c1nc1cc3c(cc12)c1ccccc1n3-c1ccc2oc3cccc(-c4nc(-c5ccccc5)nc(-c5ccccc5)n4)c3c2c1]

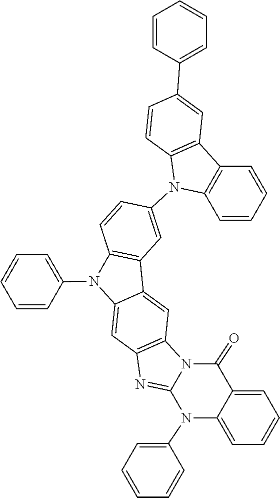 [SMILES: O=c1c2ccccc2n(-c2ccccc2)c2nc3cc4c(cc3n12)c1cc(-n2c3ccccc3c3cc(-c5ccccc5)ccc32)ccc1n4-c1ccccc1]

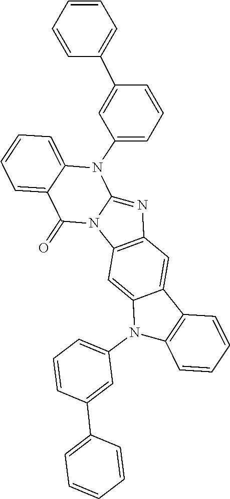 [SMILES: O=c1c2ccccc2n(-c2cccc(-c3ccccc3)c2)c2nc3cc4c5ccccc5n(-c5cccc(-c6ccccc6)c5)c4cc3n12]

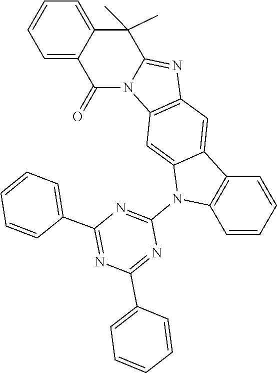 [SMILES: CC1(C)c2ccccc2C(=O)n2c1nc1cc3c4ccccc4n(-c4nc(-c5ccccc5)nc(-c5ccccc5)n4)c3cc12]

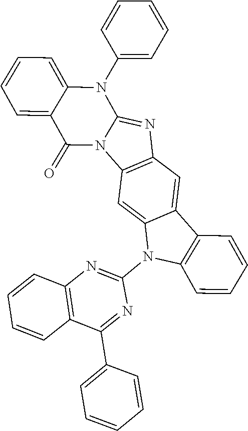 [SMILES: O=c1c2ccccc2n(-c2ccccc2)c2nc3cc4c5ccccc5n(-c5nc(-c6ccccc6)c6ccccc6n5)c4cc3n12]

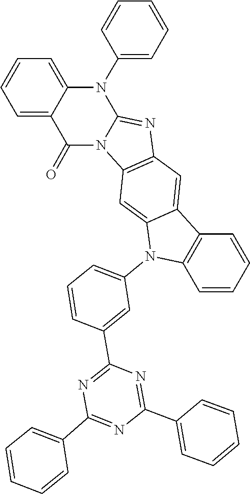 [SMILES: O=c1c2ccccc2n(-c2ccccc2)c2nc3cc4c5ccccc5n(-c5cccc(-c6nc(-c7ccccc7)nc(-c7ccccc7)n6)c5)c4cc3n12]

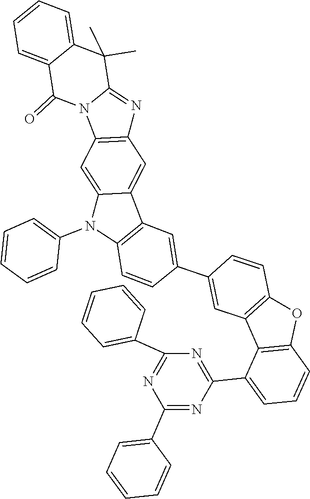 [SMILES: CC1(C)c2ccccc2C(=O)n2c1nc1cc3c4cc(-c5ccc6oc7cccc(-c8nc(-c9ccccc9)nc(-c9ccccc9)n8)c7c6c5)ccc4n(-c4ccccc4)c3cc12]

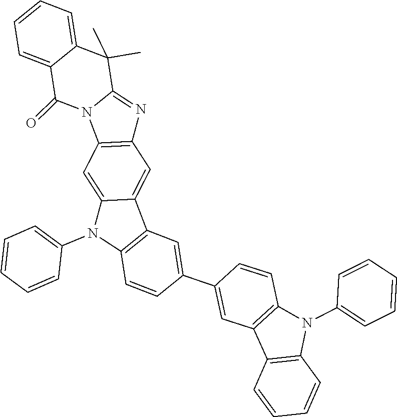 [SMILES: CC1(C)c2ccccc2C(=O)n2c1nc1cc3c4cc(-c5ccc6c(c5)c5ccccc5n6-c5ccccc5)ccc4n(-c4ccccc4)c3cc12]

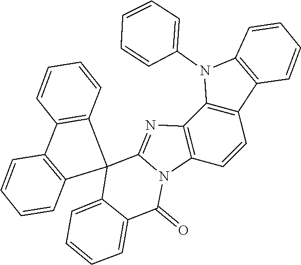 [SMILES: O=C1c2ccccc2C2(c3ccccc3-c3ccccc32)c2nc3c(ccc4c5ccccc5n(-c5ccccc5)c43)n21]

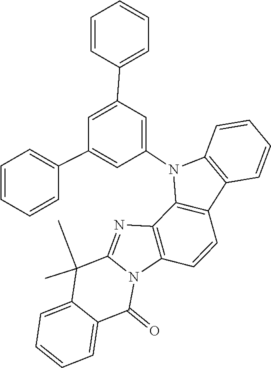 [SMILES: CC1(C)c2ccccc2C(=O)n2c1nc1c2ccc2c3ccccc3n(-c3cc(-c4ccccc4)cc(-c4ccccc4)c3)c21]

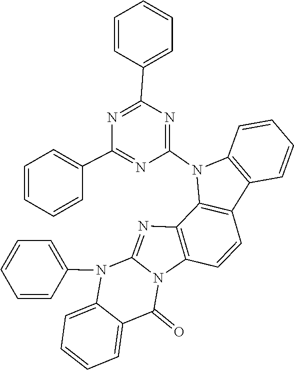 [SMILES: O=c1c2ccccc2n(-c2ccccc2)c2nc3c4c(ccc3n12)c1ccccc1n4-c1nc(-c2ccccc2)nc(-c2ccccc2)n1]

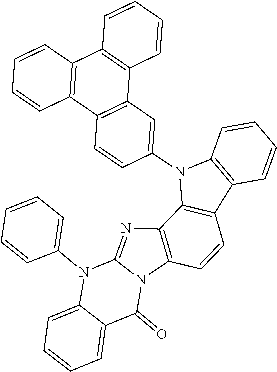 [SMILES: O=c1c2ccccc2n(-c2ccccc2)c2nc3c4c(ccc3n12)c1ccccc1n4-c1ccc2c3ccccc3c3ccccc3c2c1]

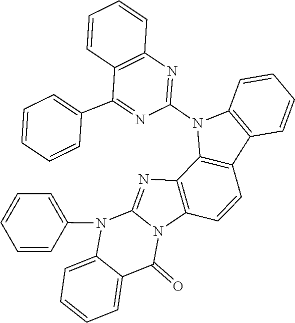 [SMILES: O=c1c2ccccc2n(-c2ccccc2)c2nc3c4c(ccc3n12)c1ccccc1n4-c1nc(-c2ccccc2)c2ccccc2n1]

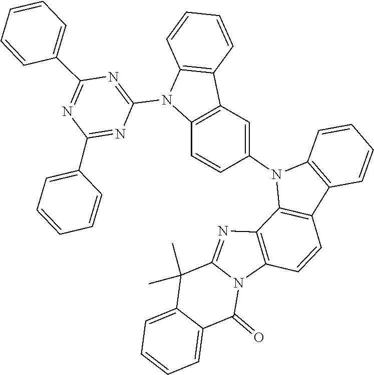 [SMILES: CC1(C)c2ccccc2C(=O)n2c1nc1c2ccc2c3ccccc3n(-c3ccc4c(c3)c3ccccc3n4-c3nc(-c4ccccc4)nc(-c4ccccc4)n3)c21]

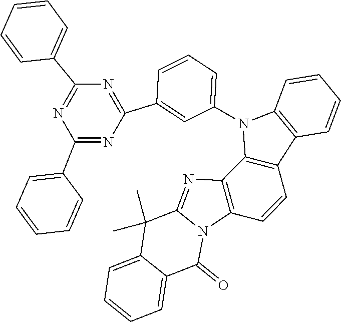 [SMILES: CC1(C)c2ccccc2C(=O)n2c1nc1c2ccc2c3ccccc3n(-c3cccc(-c4nc(-c5ccccc5)nc(-c5ccccc5)n4)c3)c21]

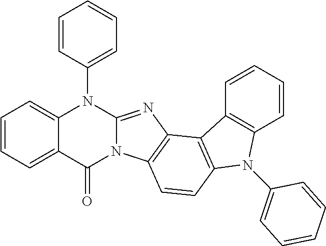 [SMILES: O=c1c2ccccc2n(-c2ccccc2)c2nc3c4c5ccccc5n(-c5ccccc5)c4ccc3n12]

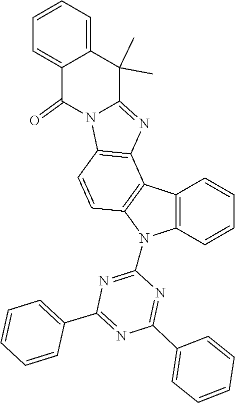 [SMILES: CC1(C)c2ccccc2C(=O)n2c1nc1c3c4ccccc4n(-c4nc(-c5ccccc5)nc(-c5ccccc5)n4)c3ccc12]

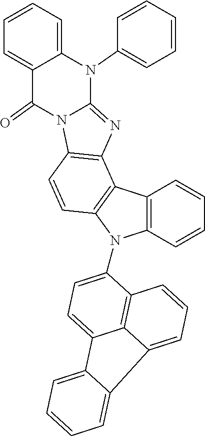 [SMILES: O=c1c2ccccc2n(-c2ccccc2)c2nc3c4c5ccccc5n(-c5ccc6c7c(cccc57)-c5ccccc5-6)c4ccc3n12]

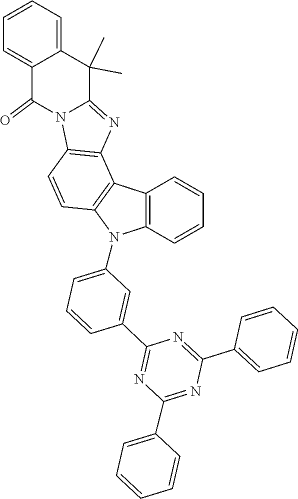 [SMILES: CC1(C)c2ccccc2C(=O)n2c1nc1c3c4ccccc4n(-c4cccc(-c5nc(-c6ccccc6)nc(-c6ccccc6)n5)c4)c3ccc12]

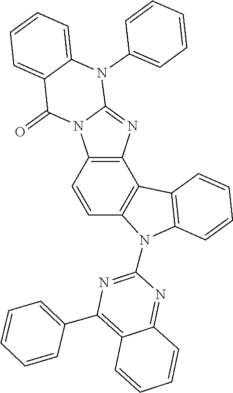 [SMILES: O=c1c2ccccc2n(-c2ccccc2)c2nc3c4c5ccccc5n(-c5nc(-c6ccccc6)c6ccccc6n5)c4ccc3n12]

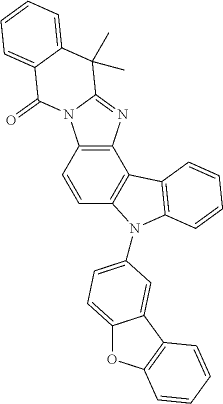 [SMILES: CC1(C)c2ccccc2C(=O)n2c1nc1c3c4ccccc4n(-c4ccc5oc6ccccc6c5c4)c3ccc12]

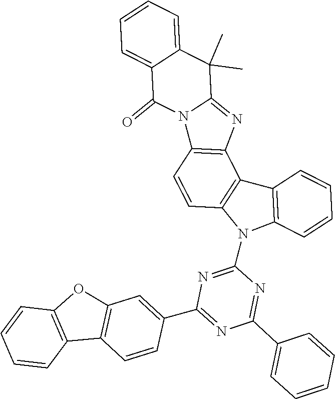 [SMILES: CC1(C)c2ccccc2C(=O)n2c1nc1c3c4ccccc4n(-c4nc(-c5ccccc5)nc(-c5ccc6c(c5)oc5ccccc56)n4)c3ccc12]

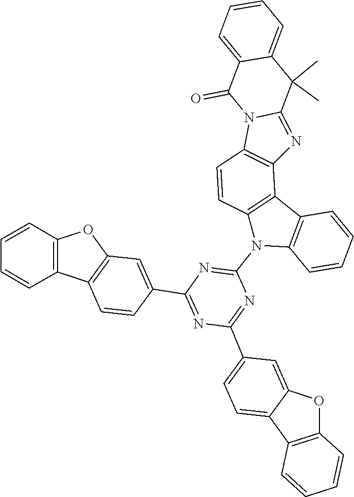 [SMILES: CC1(C)c2ccccc2C(=O)n2c1nc1c3c4ccccc4n(-c4nc(-c5ccc6c(c5)oc5ccccc56)nc(-c5ccc6c(c5)oc5ccccc56)n4)c3ccc12]

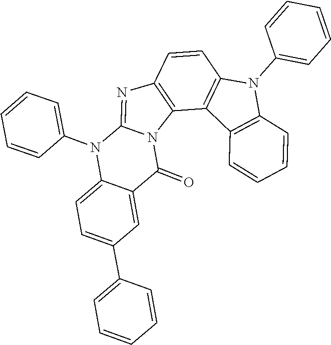 [SMILES: O=c1c2cc(-c3ccccc3)ccc2n(-c2ccccc2)c2nc3ccc4c(c5ccccc5n4-c4ccccc4)c3n12]

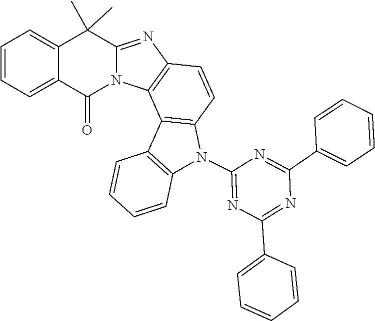 [SMILES: CC1(C)c2ccccc2C(=O)n2c1nc1ccc3c(c4ccccc4n3-c3nc(-c4ccccc4)nc(-c4ccccc4)n3)c12]

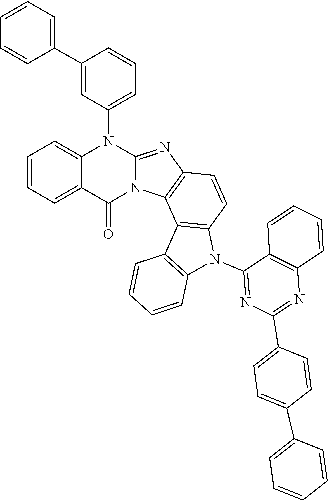 [SMILES: O=c1c2ccccc2n(-c2cccc(-c3ccccc3)c2)c2nc3ccc4c(c5ccccc5n4-c4nc(-c5ccc(-c6ccccc6)cc5)nc5ccccc45)c3n12]

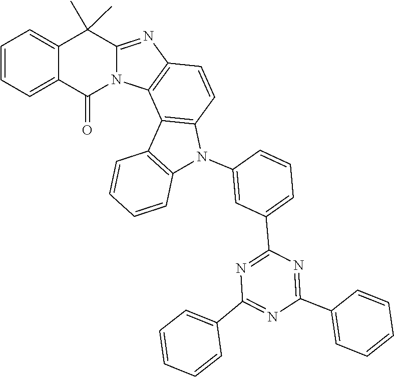 [SMILES: CC1(C)c2ccccc2C(=O)n2c1nc1ccc3c(c4ccccc4n3-c3cccc(-c4nc(-c5ccccc5)nc(-c5ccccc5)n4)c3)c12]

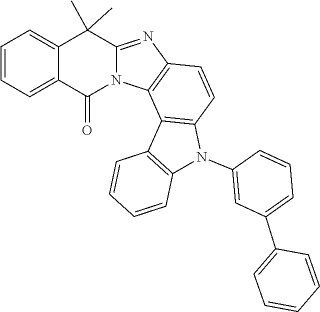 [SMILES: CC1(C)c2ccccc2C(=O)n2c1nc1ccc3c(c4ccccc4n3-c3cccc(-c4ccccc4)c3)c12]

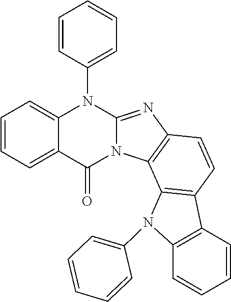 [SMILES: O=c1c2ccccc2n(-c2ccccc2)c2nc3ccc4c5ccccc5n(-c5ccccc5)c4c3n12]

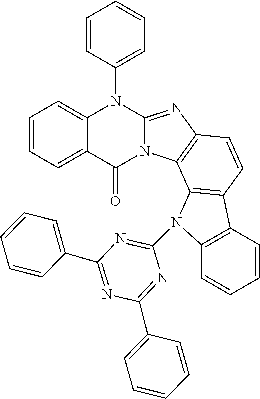 [SMILES: O=c1c2ccccc2n(-c2ccccc2)c2nc3ccc4c5ccccc5n(-c5nc(-c6ccccc6)nc(-c6ccccc6)n5)c4c3n12]

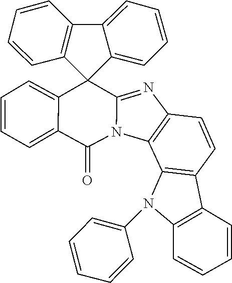 [SMILES: O=C1c2ccccc2C2(c3ccccc3-c3ccccc32)c2nc3ccc4c5ccccc5n(-c5ccccc5)c4c3n21]

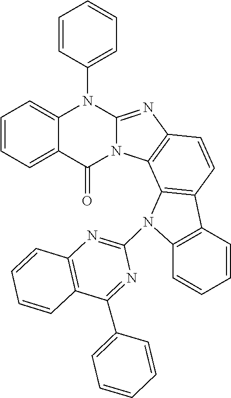 [SMILES: O=c1c2ccccc2n(-c2ccccc2)c2nc3ccc4c5ccccc5n(-c5nc(-c6ccccc6)c6ccccc6n5)c4c3n12]

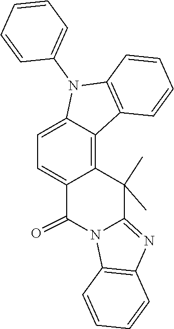 [SMILES: CC1(C)c2c(ccc3c2c2ccccc2n3-c2ccccc2)C(=O)n2c1nc1ccccc12]

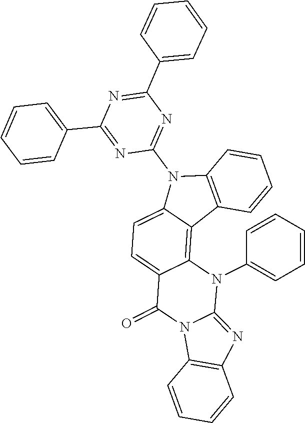 [SMILES: O=c1c2ccc3c(c4ccccc4n3-c3nc(-c4ccccc4)nc(-c4ccccc4)n3)c2n(-c2ccccc2)c2nc3ccccc3n12]

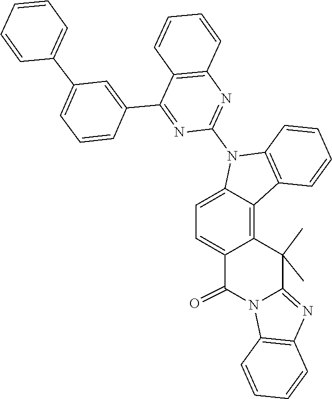 [SMILES: CC1(C)c2c(ccc3c2c2ccccc2n3-c2nc(-c3cccc(-c4ccccc4)c3)c3ccccc3n2)C(=O)n2c1nc1ccccc12]

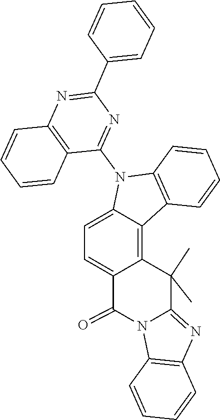 [SMILES: CC1(C)c2c(ccc3c2c2ccccc2n3-c2nc(-c3ccccc3)nc3ccccc23)C(=O)n2c1nc1ccccc12]

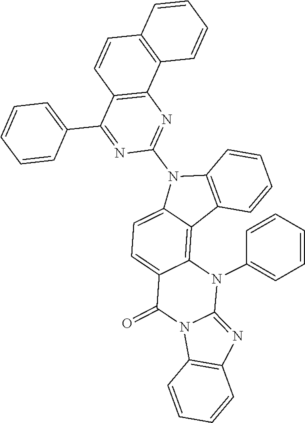 [SMILES: O=c1c2ccc3c(c4ccccc4n3-c3nc(-c4ccccc4)c4ccc5ccccc5c4n3)c2n(-c2ccccc2)c2nc3ccccc3n12]

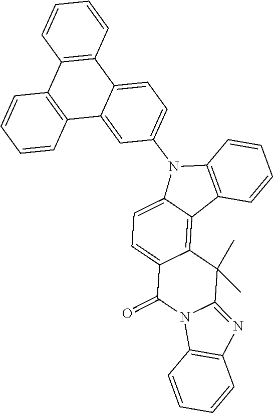 [SMILES: CC1(C)c2c(ccc3c2c2ccccc2n3-c2ccc3c4ccccc4c4ccccc4c3c2)C(=O)n2c1nc1ccccc12]

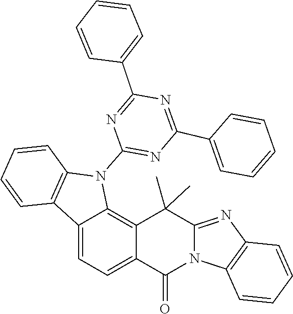 [SMILES: CC1(C)c2c(ccc3c4ccccc4n(-c4nc(-c5ccccc5)nc(-c5ccccc5)n4)c23)C(=O)n2c1nc1ccccc12]

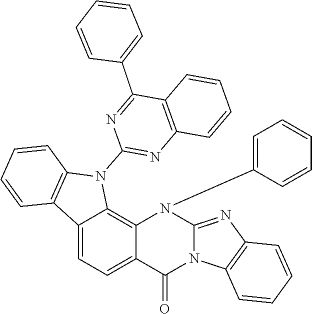 [SMILES: O=c1c2ccc3c4ccccc4n(-c4nc(-c5ccccc5)c5ccccc5n4)c3c2n(-c2ccccc2)c2nc3ccccc3n12]

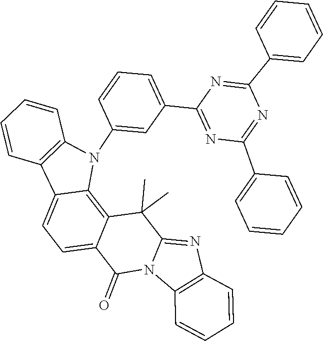 [SMILES: CC1(C)c2c(ccc3c4ccccc4n(-c4cccc(-c5nc(-c6ccccc6)nc(-c6ccccc6)n5)c4)c23)C(=O)n2c1nc1ccccc12]

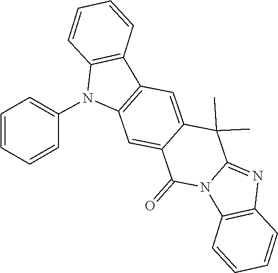 [SMILES: CC1(C)c2cc3c4ccccc4n(-c4ccccc4)c3cc2C(=O)n2c1nc1ccccc12]

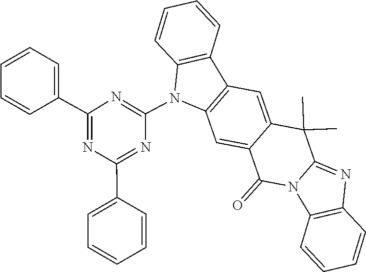 [SMILES: CC1(C)c2cc3c4ccccc4n(-c4nc(-c5ccccc5)nc(-c5ccccc5)n4)c3cc2C(=O)n2c1nc1ccccc12]

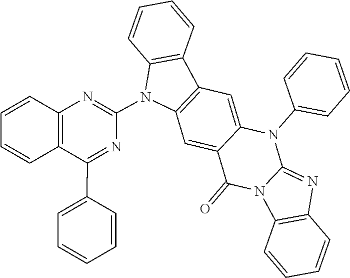 [SMILES: O=c1c2cc3c(cc2n(-c2ccccc2)c2nc4ccccc4n12)c1ccccc1n3-c1nc(-c2ccccc2)c2ccccc2n1]

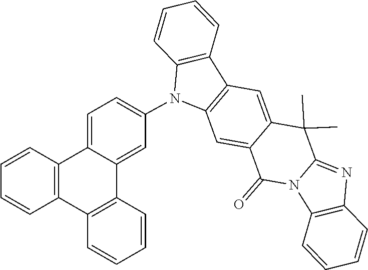 [SMILES: CC1(C)c2cc3c4ccccc4n(-c4ccc5c6ccccc6c6ccccc6c5c4)c3cc2C(=O)n2c1nc1ccccc12]

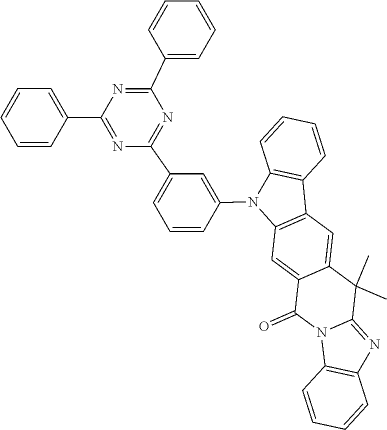 [SMILES: CC1(C)c2cc3c4ccccc4n(-c4cccc(-c5nc(-c6ccccc6)nc(-c6ccccc6)n5)c4)c3cc2C(=O)n2c1nc1ccccc12]

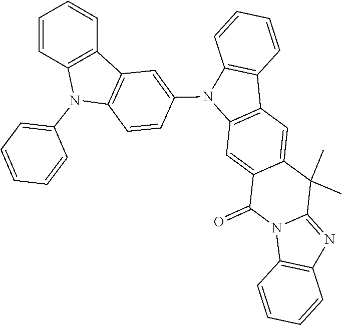 [SMILES: CC1(C)c2cc3c4ccccc4n(-c4ccc5c(c4)c4ccccc4n5-c4ccccc4)c3cc2C(=O)n2c1nc1ccccc12]

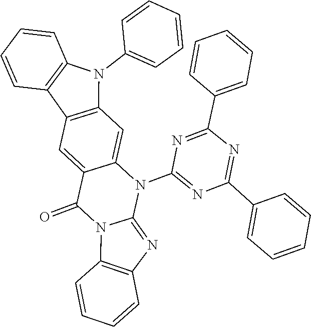 [SMILES: O=c1c2cc3c4ccccc4n(-c4ccccc4)c3cc2n(-c2nc(-c3ccccc3)nc(-c3ccccc3)n2)c2nc3ccccc3n12]

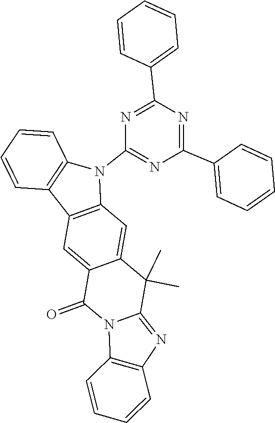 [SMILES: CC1(C)c2cc3c(cc2C(=O)n2c1nc1ccccc12)c1ccccc1n3-c1nc(-c2ccccc2)nc(-c2ccccc2)n1]

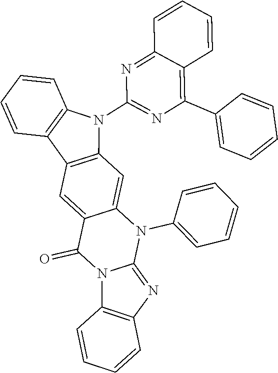 [SMILES: O=c1c2cc3c4ccccc4n(-c4nc(-c5ccccc5)c5ccccc5n4)c3cc2n(-c2ccccc2)c2nc3ccccc3n12]

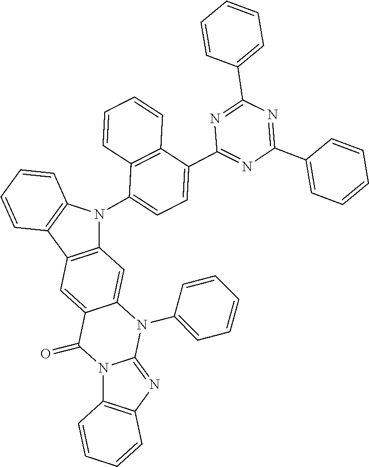 [SMILES: O=c1c2cc3c4ccccc4n(-c4ccc(-c5nc(-c6ccccc6)nc(-c6ccccc6)n5)c5ccccc45)c3cc2n(-c2ccccc2)c2nc3ccccc3n12]

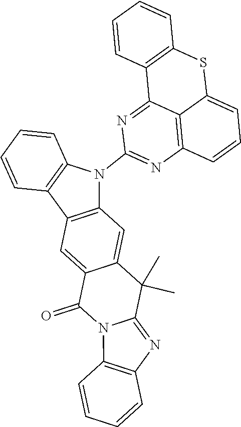 [SMILES: CC1(C)c2cc3c(cc2C(=O)n2c1nc1ccccc12)c1ccccc1n3-c1nc2c3c(cccc3n1)Sc1ccccc1-2]

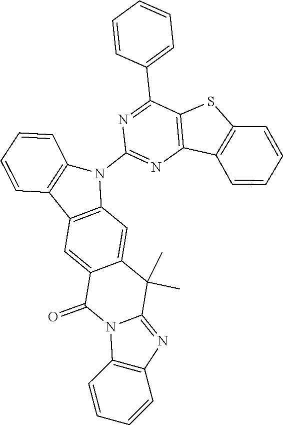 [SMILES: CC1(C)c2cc3c(cc2C(=O)n2c1nc1ccccc12)c1ccccc1n3-c1nc(-c2ccccc2)c2sc3ccccc3c2n1]

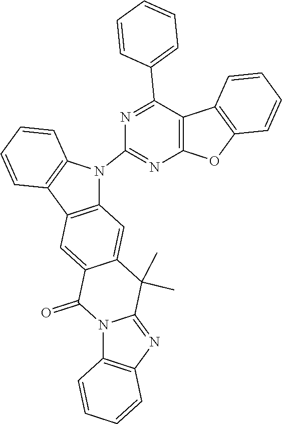 [SMILES: CC1(C)c2cc3c(cc2C(=O)n2c1nc1ccccc12)c1ccccc1n3-c1nc(-c2ccccc2)c2c(n1)oc1ccccc12]

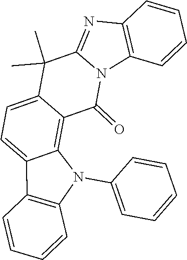 [SMILES: CC1(C)c2ccc3c4ccccc4n(-c4ccccc4)c3c2C(=O)n2c1nc1ccccc12]

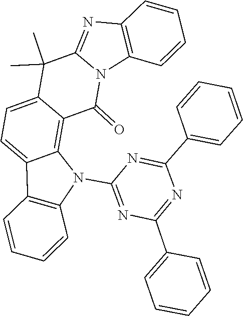 [SMILES: CC1(C)c2ccc3c4ccccc4n(-c4nc(-c5ccccc5)nc(-c5ccccc5)n4)c3c2C(=O)n2c1nc1ccccc12]

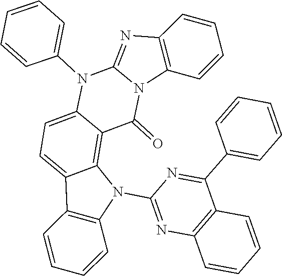 [SMILES: O=c1c2c(ccc3c4ccccc4n(-c4nc(-c5ccccc5)c5ccccc5n4)c32)n(-c2ccccc2)c2nc3ccccc3n12]

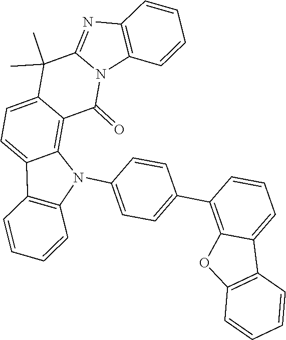 [SMILES: CC1(C)c2ccc3c4ccccc4n(-c4ccc(-c5cccc6c5oc5ccccc56)cc4)c3c2C(=O)n2c1nc1ccccc12]

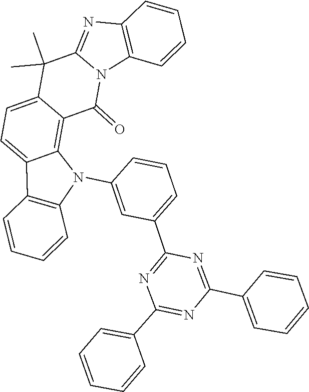 [SMILES: CC1(C)c2ccc3c4ccccc4n(-c4cccc(-c5nc(-c6ccccc6)nc(-c6ccccc6)n5)c4)c3c2C(=O)n2c1nc1ccccc12]

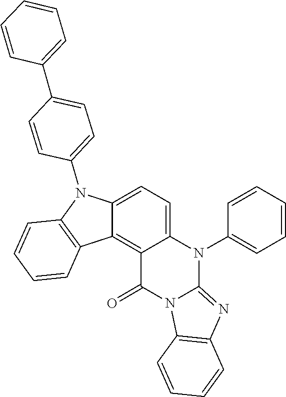 [SMILES: O=c1c2c3c4ccccc4n(-c4ccc(-c5ccccc5)cc4)c3ccc2n(-c2ccccc2)c2nc3ccccc3n12]

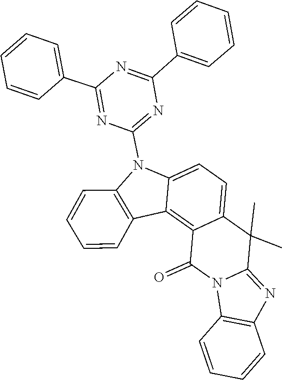 [SMILES: CC1(C)c2ccc3c(c2C(=O)n2c1nc1ccccc12)c1ccccc1n3-c1nc(-c2ccccc2)nc(-c2ccccc2)n1]

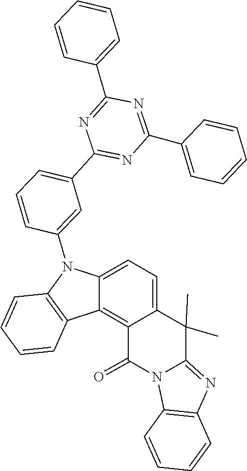 [SMILES: CC1(C)c2ccc3c(c2C(=O)n2c1nc1ccccc12)c1ccccc1n3-c1cccc(-c2nc(-c3ccccc3)nc(-c3ccccc3)n2)c1]

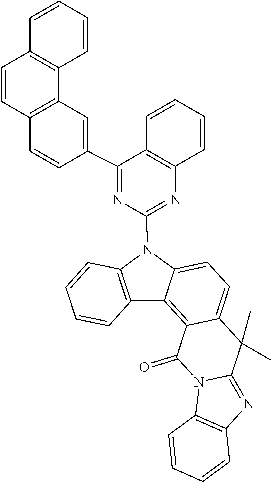 [SMILES: CC1(C)c2ccc3c(c2C(=O)n2c1nc1ccccc12)c1ccccc1n3-c1nc(-c2ccc3ccc4ccccc4c3c2)c2ccccc2n1]

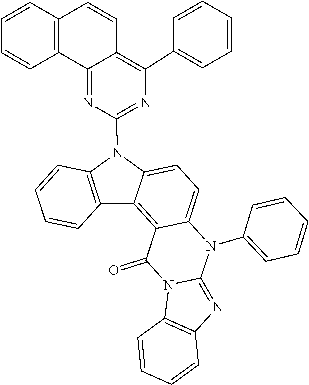 [SMILES: O=c1c2c3c4ccccc4n(-c4nc(-c5ccccc5)c5ccc6ccccc6c5n4)c3ccc2n(-c2ccccc2)c2nc3ccccc3n12]

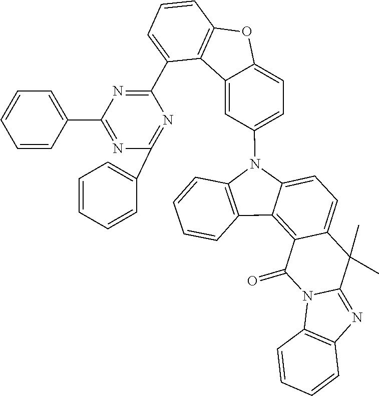 [SMILES: CC1(C)c2ccc3c(c2C(=O)n2c1nc1ccccc12)c1ccccc1n3-c1ccc2oc3cccc(-c4nc(-c5ccccc5)nc(-c5ccccc5)n4)c3c2c1]

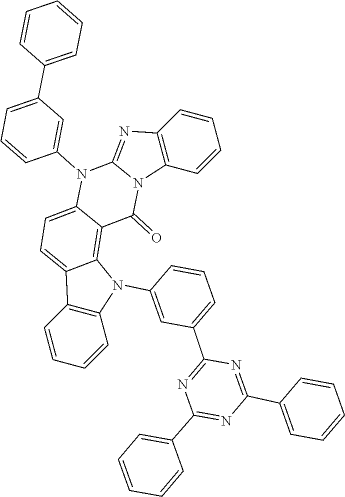 [SMILES: O=c1c2c(ccc3c4ccccc4n(-c4cccc(-c5nc(-c6ccccc6)nc(-c6ccccc6)n5)c4)c32)n(-c2cccc(-c3ccccc3)c2)c2nc3ccccc3n12]

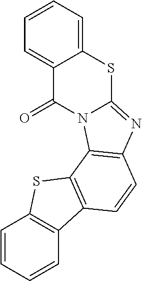 [SMILES: O=c1c2ccccc2sc2nc3ccc4c5ccccc5sc4c3n12]

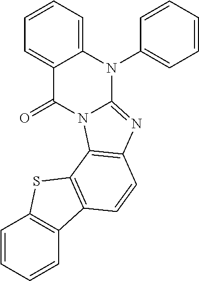 [SMILES: O=c1c2ccccc2n(-c2ccccc2)c2nc3ccc4c5ccccc5sc4c3n12]

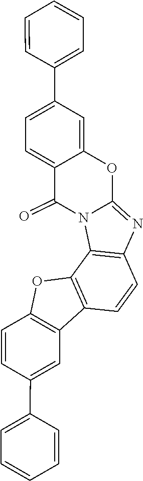 [SMILES: O=c1c2ccc(-c3ccccc3)cc2oc2nc3ccc4c5cc(-c6ccccc6)ccc5oc4c3n12]

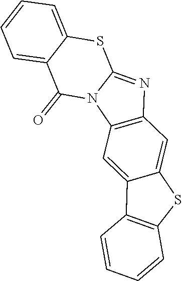 [SMILES: O=c1c2ccccc2sc2nc3cc4sc5ccccc5c4cc3n12]

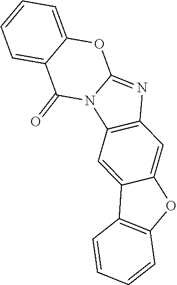 [SMILES: O=c1c2ccccc2oc2nc3cc4oc5ccccc5c4cc3n12]

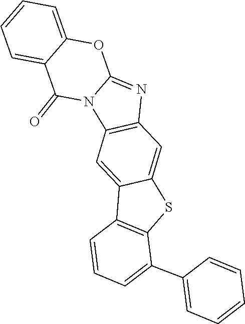 [SMILES: O=c1c2ccccc2oc2nc3cc4sc5c(-c6ccccc6)cccc5c4cc3n12]

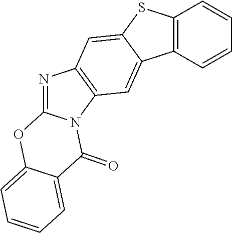 [SMILES: O=c1c2ccccc2oc2nc3cc4sc5ccccc5c4cc3n12]

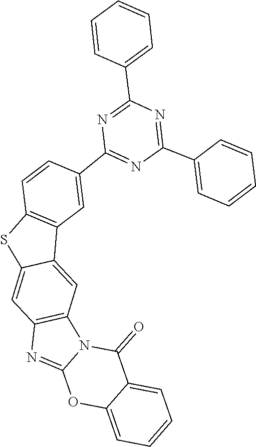 [SMILES: O=c1c2ccccc2oc2nc3cc4sc5ccc(-c6nc(-c7ccccc7)nc(-c7ccccc7)n6)cc5c4cc3n12]

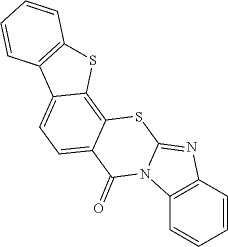 [SMILES: O=c1c2ccc3c4ccccc4sc3c2sc2nc3ccccc3n12]

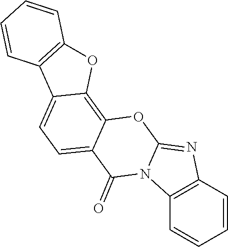 [SMILES: O=c1c2ccc3c4ccccc4oc3c2oc2nc3ccccc3n12]

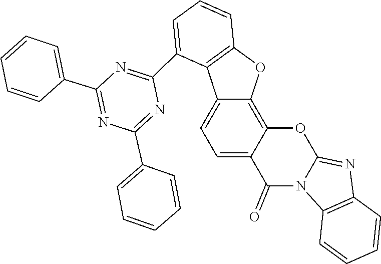 [SMILES: O=c1c2ccc3c(oc4cccc(-c5nc(-c6ccccc6)nc(-c6ccccc6)n5)c43)c2oc2nc3ccccc3n12]

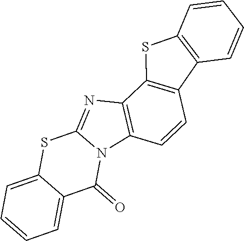 [SMILES: O=c1c2ccccc2sc2nc3c4sc5ccccc5c4ccc3n12]

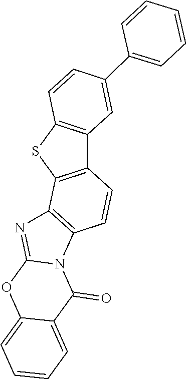 [SMILES: O=c1c2ccccc2oc2nc3c4sc5ccc(-c6ccccc6)cc5c4ccc3n12]

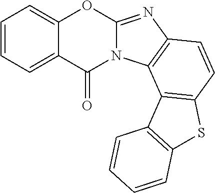 [SMILES: O=c1c2ccccc2oc2nc3ccc4sc5ccccc5c4c3n12]

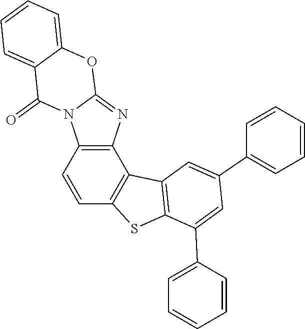 [SMILES: O=c1c2ccccc2oc2nc3c4c(ccc3n12)sc1c(-c2ccccc2)cc(-c2ccccc2)cc14]

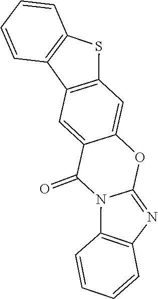 [SMILES: O=c1c2cc3c(cc2oc2nc4ccccc4n12)sc1ccccc13]

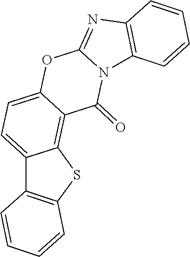 [SMILES: O=c1c2c(ccc3c4ccccc4sc32)oc2nc3ccccc3n12]

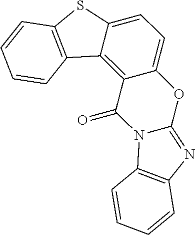 [SMILES: O=c1c2c(ccc3sc4ccccc4c32)oc2nc3ccccc3n12]

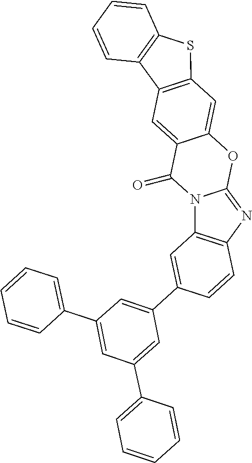 [SMILES: O=c1c2cc3c(cc2oc2nc4ccc(-c5cc(-c6ccccc6)cc(-c6ccccc6)c5)cc4n12)sc1ccccc13]

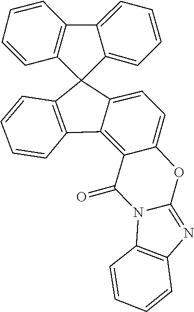 [SMILES: O=c1c2c3c(ccc2oc2nc4ccccc4n12)C1(c2ccccc2-c2ccccc21)c1ccccc1-3]

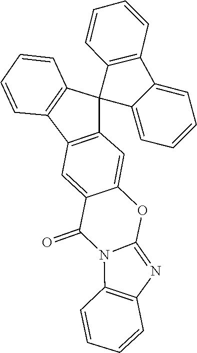 [SMILES: O=c1c2cc3c(cc2oc2nc4ccccc4n12)C1(c2ccccc2-c2ccccc21)c1ccccc1-3]

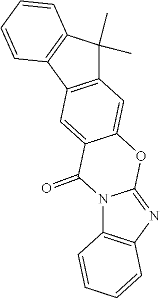 [SMILES: CC1(C)c2ccccc2-c2cc3c(=O)n4c(nc5ccccc54)oc3cc21]

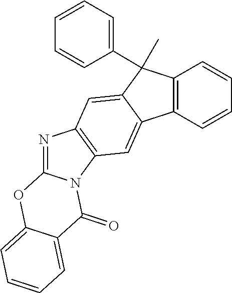 [SMILES: CC1(c2ccccc2)c2ccccc2-c2cc3c(cc21)nc1oc2ccccc2c(=O)n13]

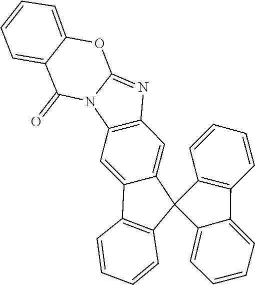 [SMILES: O=c1c2ccccc2oc2nc3cc4c(cc3n12)-c1ccccc1C41c2ccccc2-c2ccccc21]

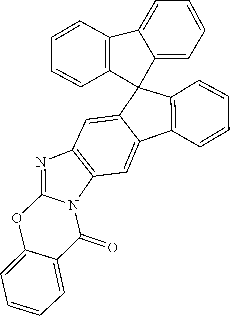 [SMILES: O=c1c2ccccc2oc2nc3cc4c(cc3n12)-c1ccccc1C41c2ccccc2-c2ccccc21]

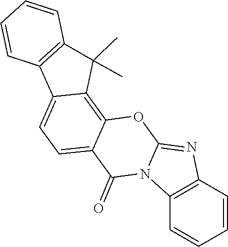 [SMILES: CC1(C)c2ccccc2-c2ccc3c(=O)n4c(nc5ccccc54)oc3c21]

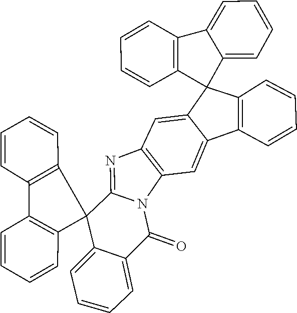 [SMILES: O=C1c2ccccc2C2(c3ccccc3-c3ccccc32)c2nc3cc4c(cc3n21)-c1ccccc1C41c2ccccc2-c2ccccc21]

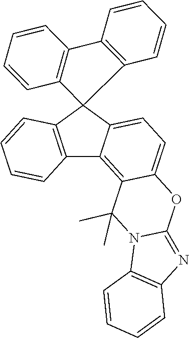 [SMILES: CC1(C)c2c(ccc3c2-c2ccccc2C32c3ccccc3-c3ccccc32)Oc2nc3ccccc3n21]

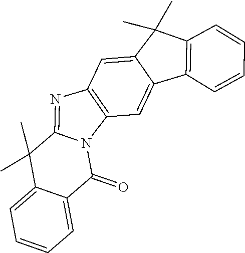 [SMILES: CC1(C)c2ccccc2-c2cc3c(cc21)nc1n3C(=O)c2ccccc2C1(C)C]

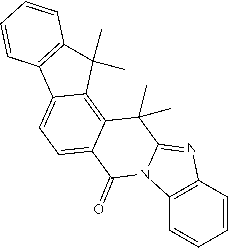 [SMILES: CC1(C)c2ccccc2-c2ccc3c(c21)C(C)(C)c1nc2ccccc2n1C3=O]

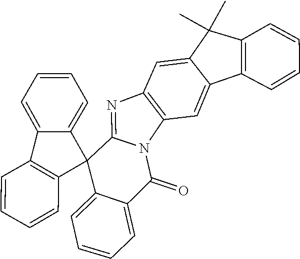 [SMILES: CC1(C)c2ccccc2-c2cc3c(cc21)nc1n3C(=O)c2ccccc2C12c1ccccc1-c1ccccc12]

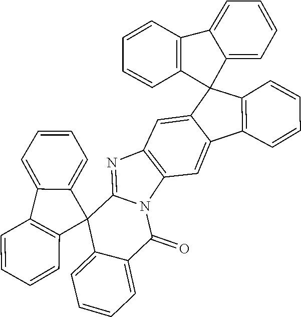 [SMILES: O=C1c2ccccc2C2(c3ccccc3-c3ccccc32)c2nc3cc4c(cc3n21)-c1ccccc1C41c2ccccc2-c2ccccc21]

The base structure of the compounds of the invention can be prepared by the routes outlined in schemes 1 to 6. Schemes 1 and 2 show the synthesis of the compounds with Y¹═NAr by two alternative routes.

Scheme 3 shows the synthesis of the compounds with Y¹ ═S. Schemes 4 and 5 show the synthesis of the compounds with Y¹ ═O by two alternative routes. Scheme 6 shows the synthesis with Y¹ ═CR₂.

This involves first constructing the base structure of the formula (1) that still does not bear a group of formula (2). The synthesis of the base skeleton is known in the literature. A precursor of the group of the formula (2) can then be introduced in two steps by first conducting a first coupling reaction, for example a Suzuki or Hartwig-Buchwald coupling. Thereafter, the group of the formula (2) is introduced by a cyclization. Both these reactions may be coupling reactions, but also substitution reactions. When the base structure is substituted by a reactive leaving group, for example chlorine or bromine, this may be replaced by other substituents in a further reaction, for example by aromatic or heteroaromatic substituents R in a Suzuki coupling reaction.

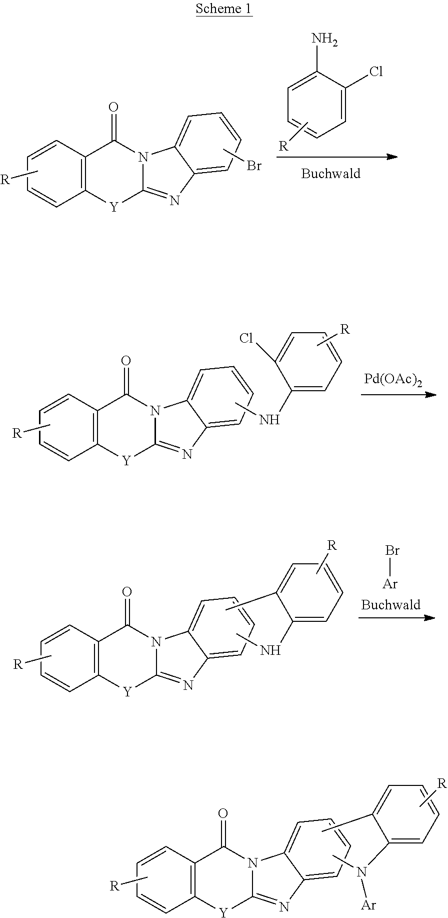

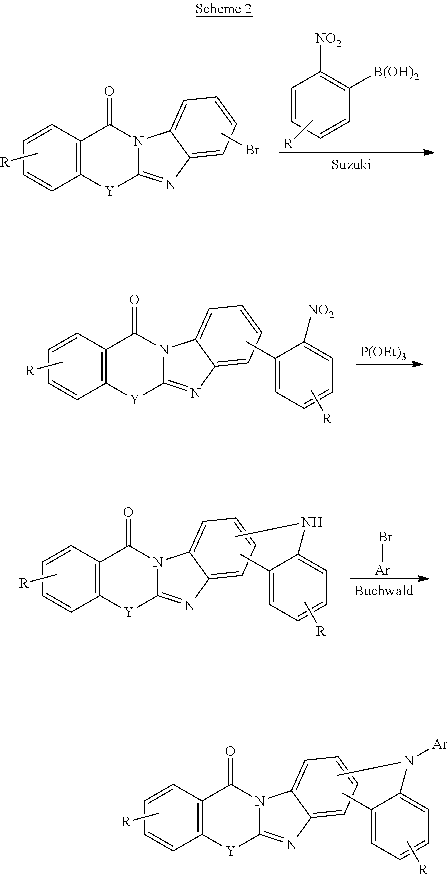

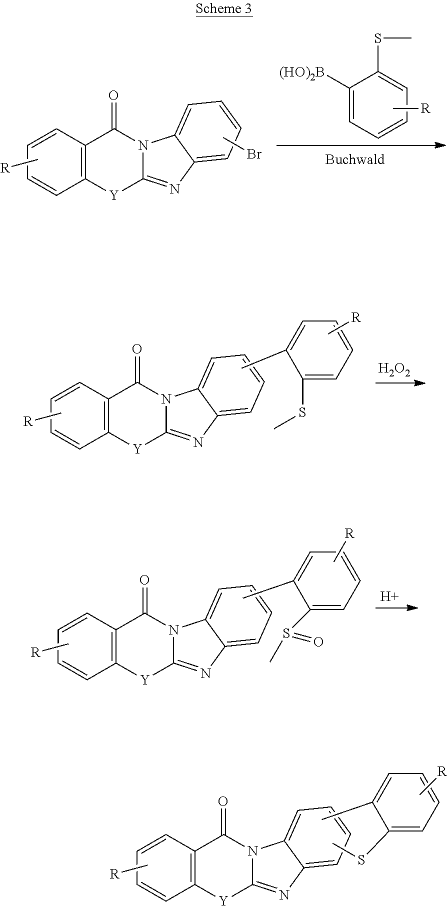

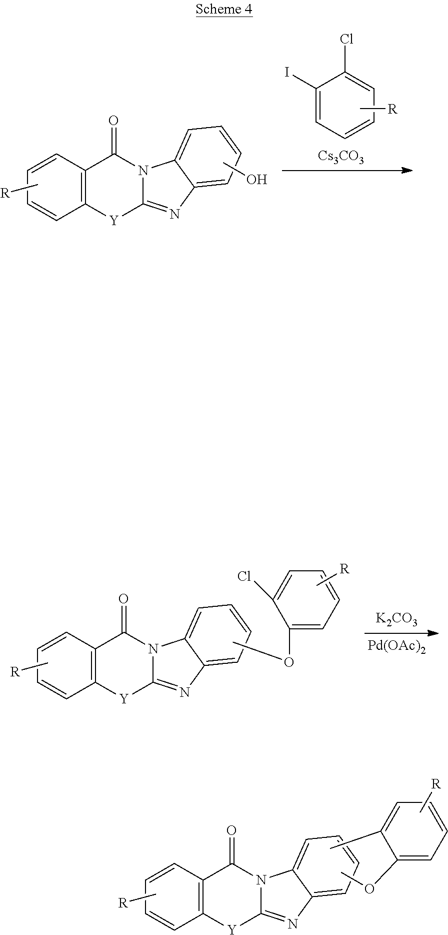

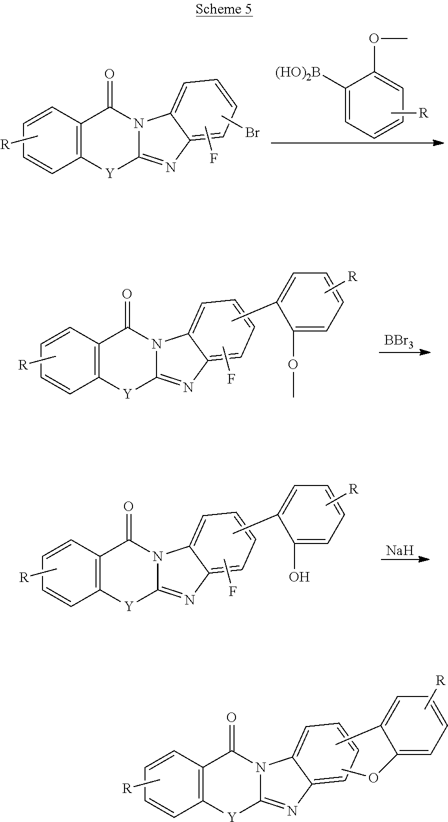

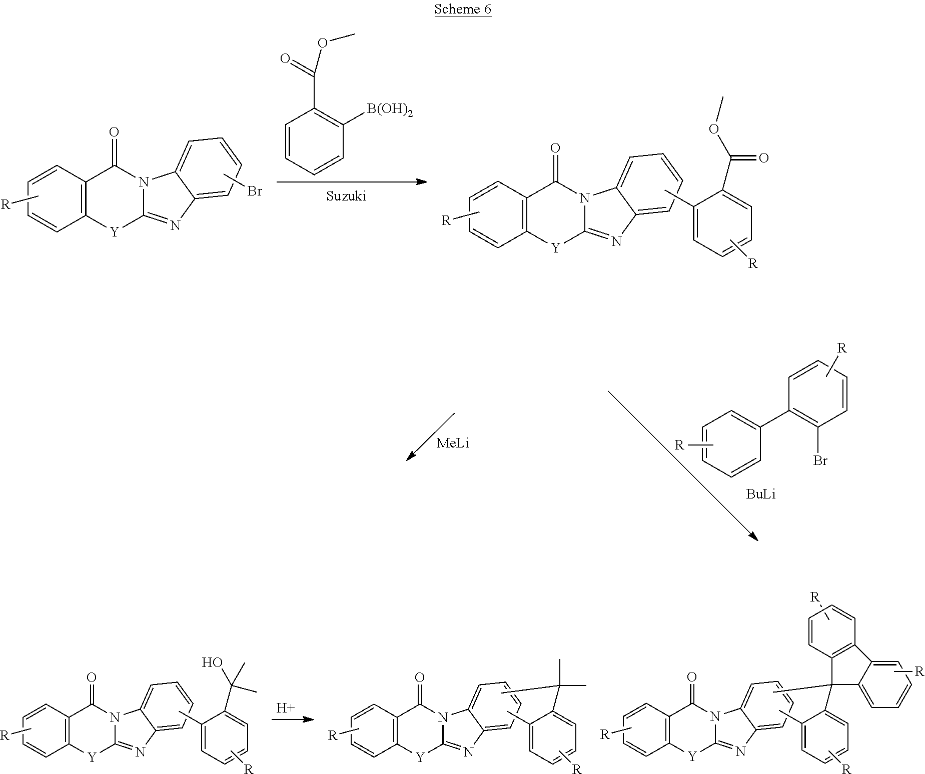

The present invention therefore further provides a process for preparing the compounds of the invention, characterized by the following steps:
(A) synthesizing the base skeleton of formula (1) that does not yet bear a group of the formula (2); and
(B) introducing the group of the formula (2) by at least one coupling reaction.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation comprising at least one compound of the invention and at least one further compound. The further compound may, for example, be a solvent, especially one of the abovementioned solvents or a mixture of these solvents. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. This further compound may also be polymeric.

The compounds of the invention are suitable for use in an electronic device, especially in an organic electroluminescent device.

The present invention therefore further provides for the use of a compound of the invention in an electronic device, especially in an organic electroluminescent device.

The present invention still further provides an electronic device comprising at least one compound of the invention.

An electronic device in the context of the present invention is a device comprising at least one layer comprising at least one organic compound. This component may also comprise inorganic materials or else layers formed entirely from inorganic materials.

The electronic device is preferably selected from the group consisting of organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), dye-sensitized organic solar cells (DSSCs), organic optical detectors, organic photoreceptors, organic field-quench devices (O-FQDs), light-emitting electrochemical cells (LECs), organic laser diodes (O-lasers) and organic plasmon emitting devices, but preferably organic electroluminescent devices (OLEDs), more preferably phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem OLED, especially for white-emitting OLEDs.

The compound of the invention according to the above-detailed embodiments may be used in different layers, according to the exact structure. Preference is given to an organic electroluminescent device comprising a compound of formula (1) or the above-recited preferred embodiments in an emitting layer as matrix material for phosphorescent emitters or for emitters that exhibit TADF (thermally activated delayed fluorescence), especially for phosphorescent emitters. In this case, the organic electroluminescent device may contain an emitting layer, or it may contain a plurality of emitting layers, where at least one emitting layer contains at least one compound of the invention as matrix material. In addition, the compound of the invention can also be used in an electron transport layer and/or in a hole blocker layer and/or in a hole transport layer and/or in an exciton blocker layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material.

Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

A further preferred embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, or dibenzofuran derivatives, for example according to WO 2015/169412, WO 2016/015810, WO 2016/023608, WO 2017/148564 or WO 2017/148565. It is likewise possible for a further phosphorescent emitter having shorter-wavelength emission than the actual emitter to be present as co-host in the mixture, or a compound not involved in charge transport to a significant extent, if at all, as described, for example, in WO 2010/108579.

In a preferred embodiment of the invention, the materials are used in combination with a further matrix material. Preferred co-matrix materials, especially when the compound of the invention is substituted by an electron-deficient heteroaromatic ring system, are selected from the group of the biscarbazoles, the bridged carbazoles, the triarylamines, the dibenzofuranyl-carbazole derivatives or dibenzofuranyl-amine derivatives and the carbazoleamines.

Preferred biscarbazoles are the structures of the following formulae (19) and (20):

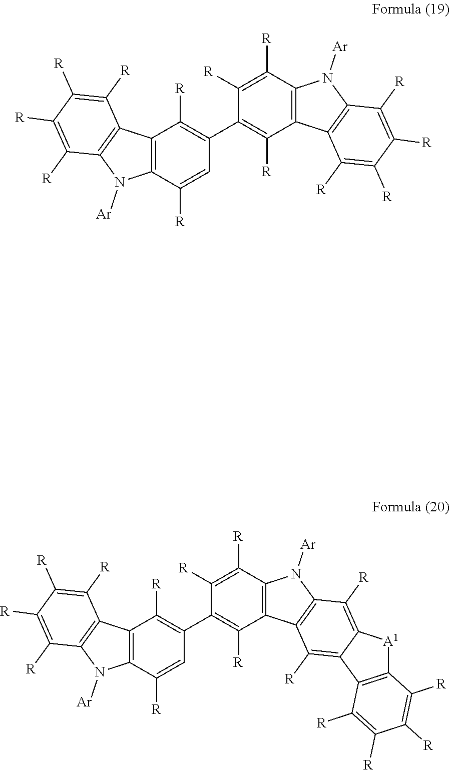

where Ar and A¹ have the definitions given above in the case of Ar, and R has the definition given above. In a preferred embodiment of the invention, A¹ is CR₂.

Preferred embodiments of the compounds of the formulae (19) and (20) are the compounds of the following formulae (19a) and (20a):

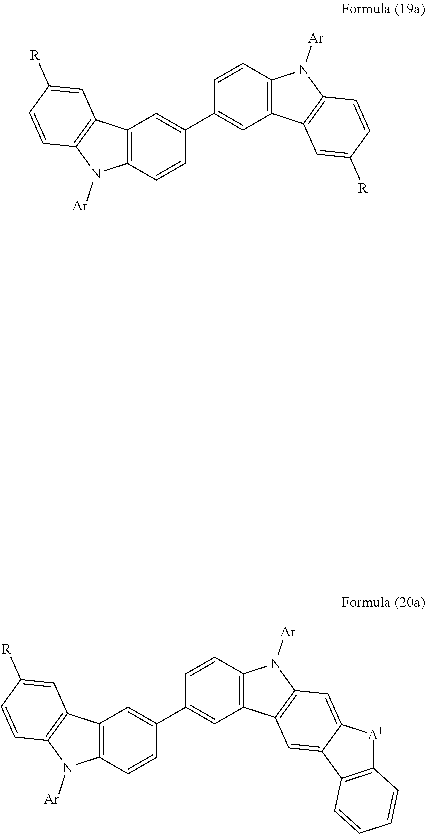

where the symbols used have the definitions given above.

Examples of suitable compounds of formulae (19) and (20) are the compounds depicted below:

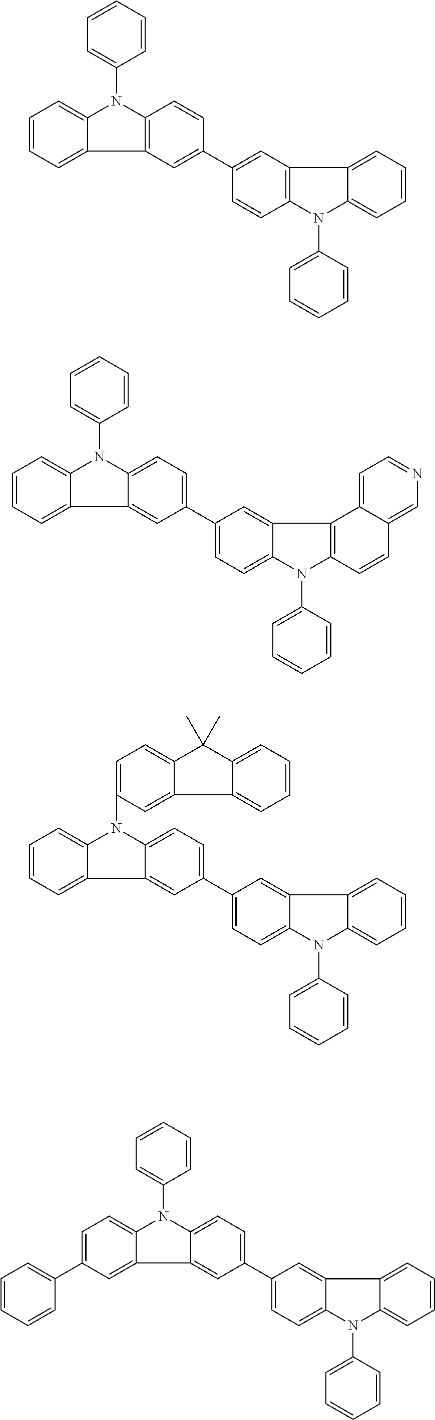 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4c6ccncc6ccc4n5-c4ccccc4)ccc32)cc1 ; CC1(C)c2ccccc2-c2cc(-n3c4ccccc4c4cc(-c5ccc6c(c5)c5ccccc5n6-c5ccccc5)ccc43)ccc21 ; c1ccc(-c2ccc3c(c2)c2cc(-c4ccc5c(c4)c4ccccc4n5-c4ccccc4)ccc2n3-c2ccccc2)cc1]

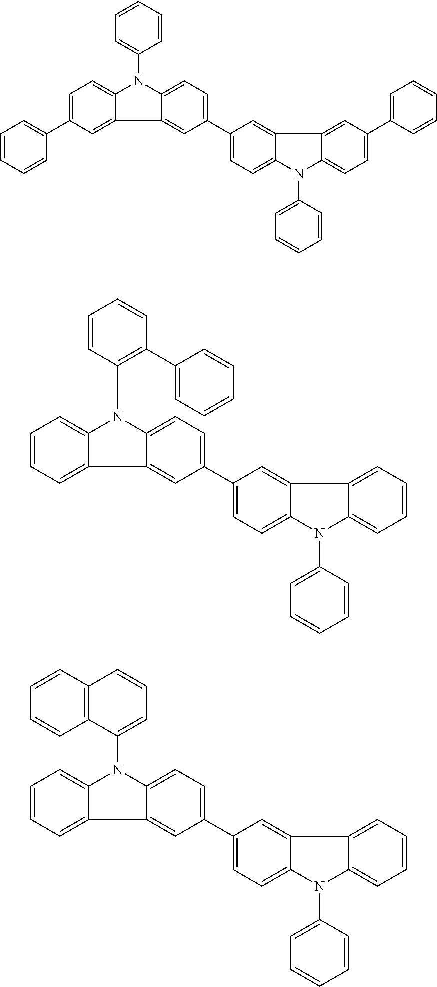 [SMILES: c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1]

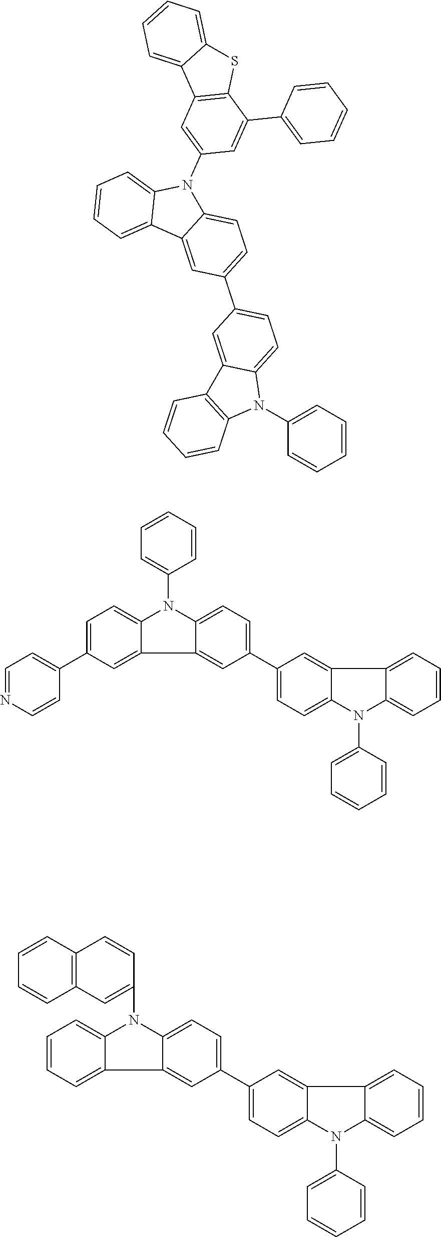 [SMILES: c1ccc(-c2cc(-n3c4ccccc4c4cc(-c5ccc6c(c5)c5ccccc5n6-c5ccccc5)ccc43)cc3c2sc2ccccc23)cc1 ; c1ccc(-n2c3ccccc3c3cc(-c4ccc5c(c4)c4cc(-c6ccncc6)ccc4n5-c4ccccc4)ccc32)cc1 ; c1ccc(-n2c3ccccc3c3cc(-c4ccc5c(c4)c4ccccc4n5-c4ccc5ccccc5c4)ccc32)cc1]

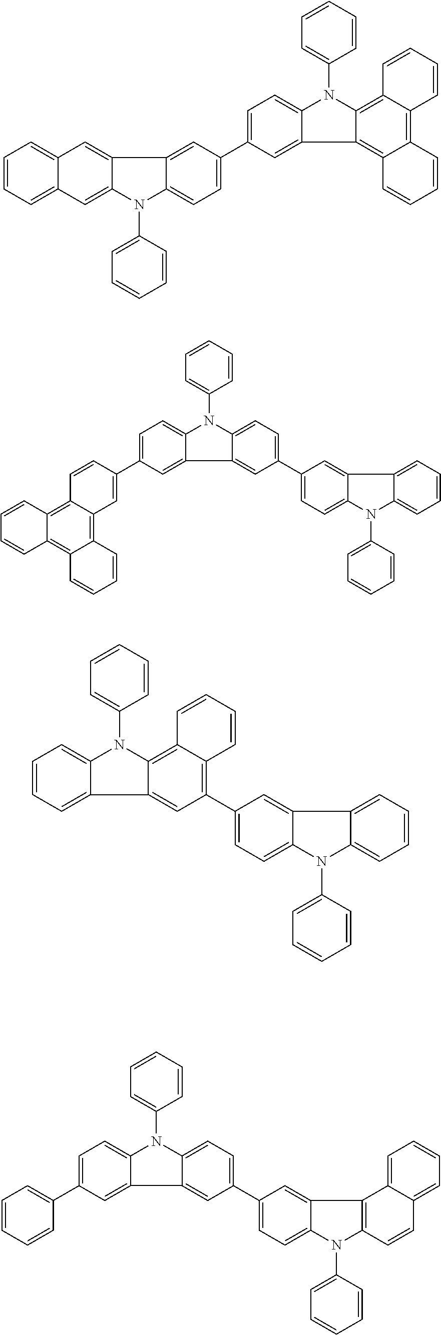 [SMILES: c1ccc(-n2c3ccc(-c4ccc5c(c4)c4c6ccccc6c6ccccc6c4n5-c4ccccc4)cc3c3cc4ccccc4cc32)cc1 ; c1ccc(-n2c3ccccc3c3cc(-c4ccc5c(c4)c4cc(-c6ccc7c8ccccc8c8ccccc8c7c6)ccc4n5-c4ccccc4)ccc32)cc1 ; c1ccc(-n2c3ccccc3c3cc(-c4cc5c6ccccc6n(-c6ccccc6)c5c5ccccc45)ccc32)cc1 ; c1ccc(-c2ccc3c(c2)c2cc(-c4ccc5c(c4)c4c6ccccc6ccc4n5-c4ccccc4)ccc2n3-c2ccccc2)cc1]

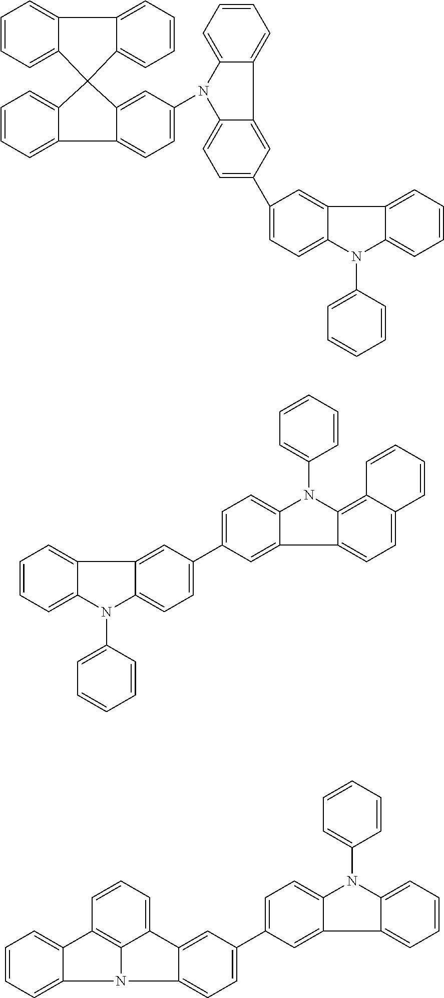 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccc6ccccc6c4n5-c4ccccc4)ccc32)cc1 ; c1ccc(-n2c3ccccc3c3cc(-c4ccc5c(c4)c4cccc6c7ccccc7n5c64)ccc32)cc1]

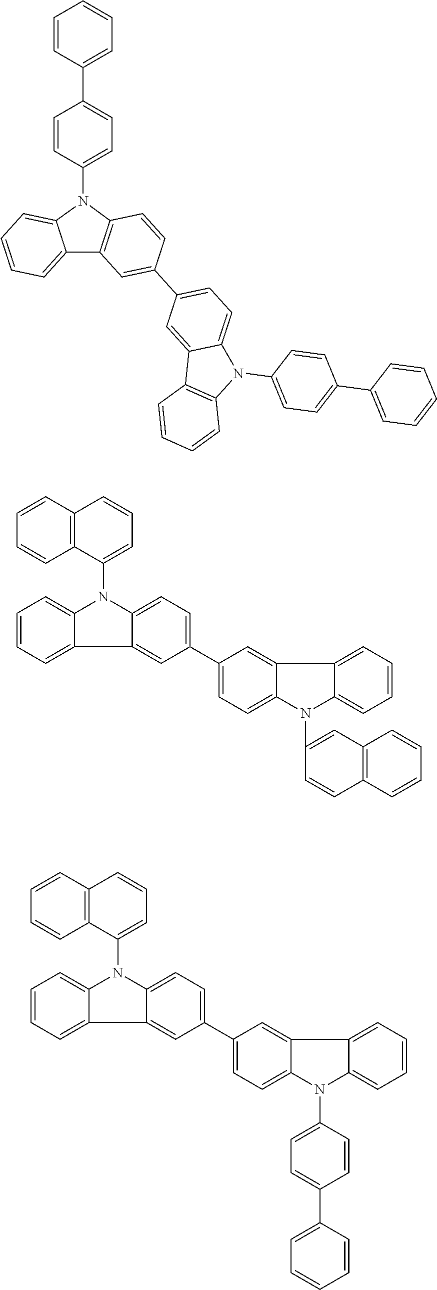 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1 ; c1ccc(-c2ccc(-n3c4ccccc4c4cc(-c5ccc6c(c5)c5ccccc5n6-c5cccc6ccccc56)ccc43)cc2)cc1]

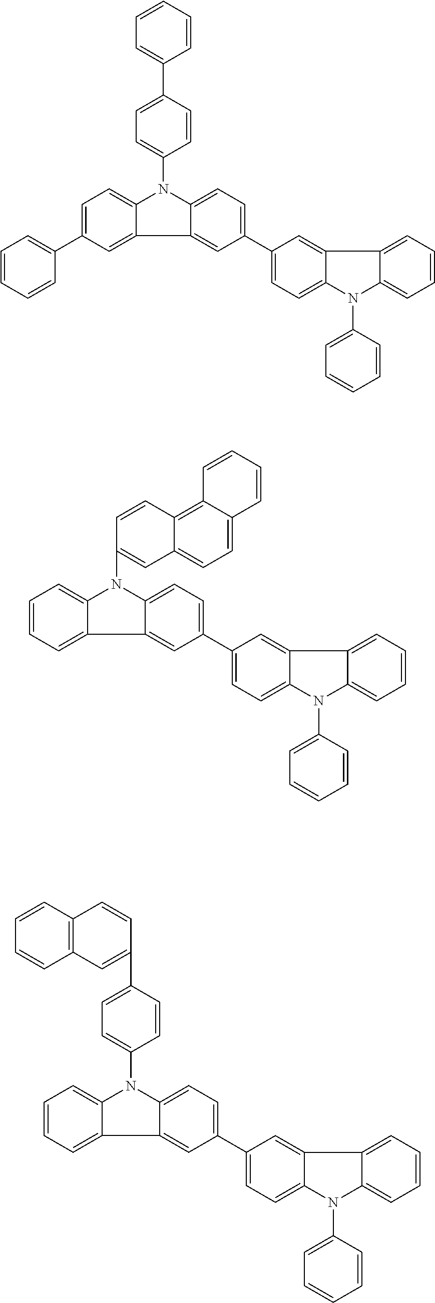 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc(-c5ccc6ccccc6c5)cc4)ccc32)cc1]

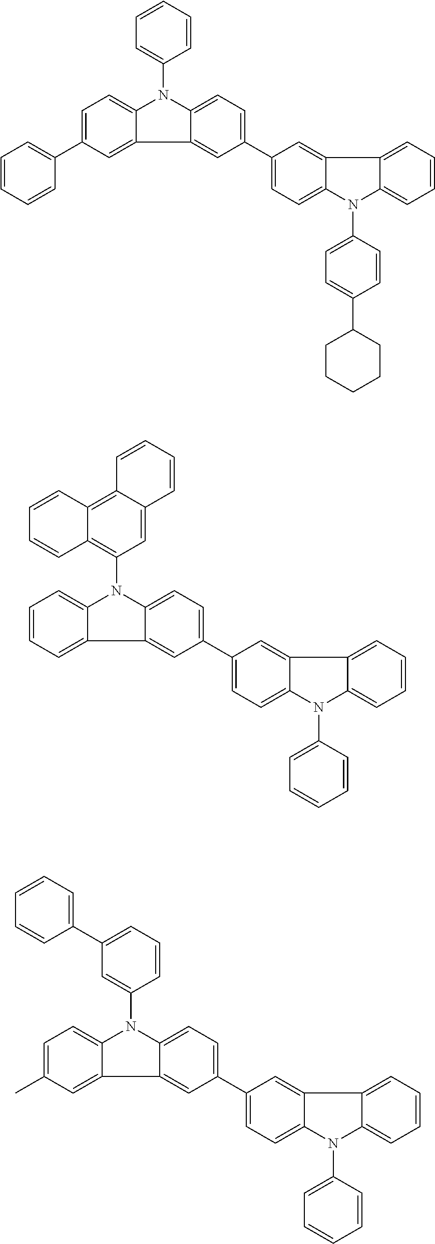 [SMILES: c1ccc(-c2ccc3c(c2)c2cc(-c4ccc5c(c4)c4ccccc4n5-c4ccc(C5CCCCC5)cc4)ccc2n3-c2ccccc2)cc1 ; c1ccc(-n2c3ccccc3c3cc(-c4ccc5c(c4)c4ccccc4n5-c4cc5ccccc5c5ccccc45)ccc32)cc1 ; Cc1ccc2c(c1)c1cc(-c3ccc4c(c3)c3ccccc3n4-c3ccccc3)ccc1n2-c1cccc(-c2ccccc2)c1]

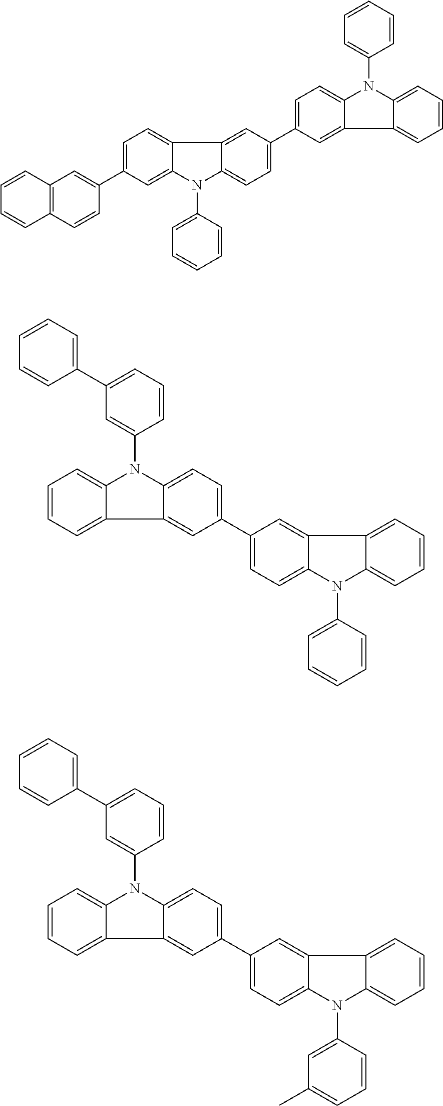 [SMILES: c1ccc(-n2c3ccccc3c3cc(-c4ccc5c(c4)c4ccc(-c6ccc7ccccc7c6)cc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; Cc1cccc(-n2c3ccccc3c3cc(-c4ccc5c(c4)c4ccccc4n5-c4cccc(-c5ccccc5)c4)ccc32)c1]

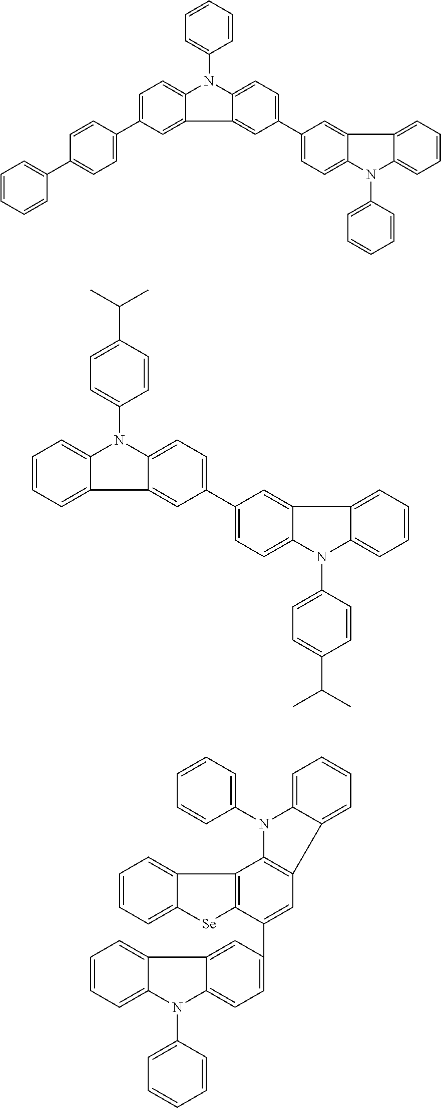 [SMILES: c1ccc(-c2ccc(-c3ccc4c(c3)c3cc(-c5ccc6c(c5)c5ccccc5n6-c5ccccc5)ccc3n4-c3ccccc3)cc2)cc1 ; CC(C)c1ccc(-n2c3ccccc3c3cc(-c4ccc5c(c4)c4ccccc4n5-c4ccc(C(C)C)cc4)ccc32)cc1 ; c1ccc(-n2c3ccccc3c3cc(-c4cc5c6ccccc6n(-c6ccccc6)c5c5c4[se]c4ccccc45)ccc32)cc1]

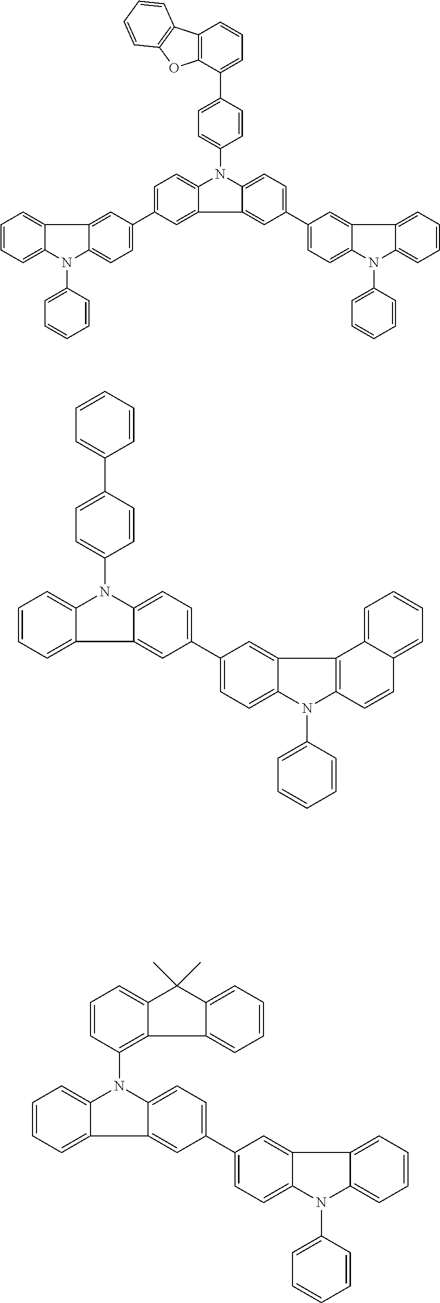 [SMILES: c1ccc(-n2c3ccccc3c3cc(-c4ccc5c(c4)c4cc(-c6ccc7c(c6)c6ccccc6n7-c6ccccc6)ccc4n5-c4ccc(-c5cccc6c5oc5ccccc56)cc4)ccc32)cc1 ; c1ccc(-c2ccc(-n3c4ccccc4c4cc(-c5ccc6c(c5)c5c7ccccc7ccc5n6-c5ccccc5)ccc43)cc2)cc1 ; CC1(C)c2ccccc2-c2c(-n3c4ccccc4c4cc(-c5ccc6c(c5)c5ccccc5n6-c5ccccc5)ccc43)cccc21]

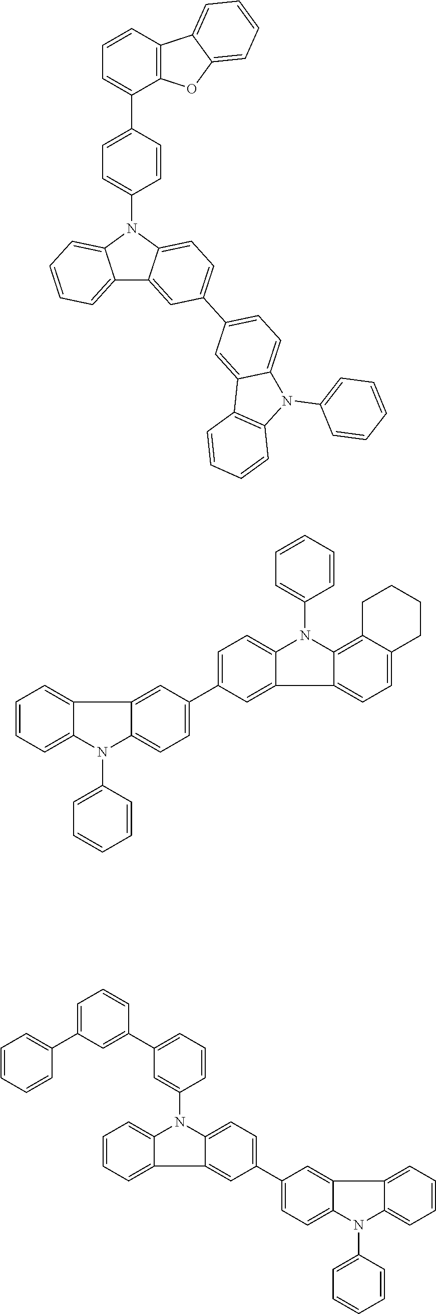 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccc6c(c4n5-c4ccccc4)CCCC6)ccc32)cc1 ; c1ccc(-c2cccc(-c3cccc(-n4c5ccccc5c5cc(-c6ccc7c(c6)c6ccccc6n7-c6ccccc6)ccc54)c3)c2)cc1]

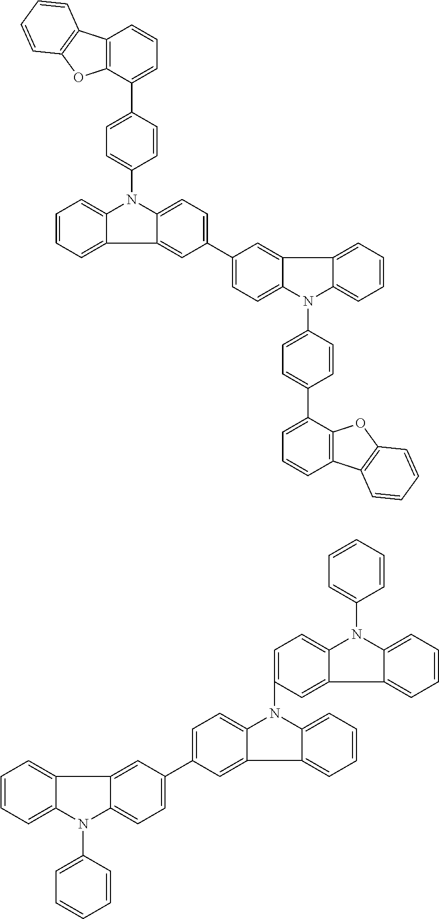 [SMILES: c1ccc2c(c1)oc1c(-c3ccc(-n4c5ccccc5c5cc(-c6ccc7c(c6)c6ccccc6n7-c6ccc(-c7cccc8c7oc7ccccc78)cc6)ccc54)cc3)cccc12 ; c1ccc(-n2c3ccccc3c3cc(-c4ccc5c(c4)c4ccccc4n5-c4ccc5c(c4)c4ccccc4n5-c4ccccc4)ccc32)cc1]

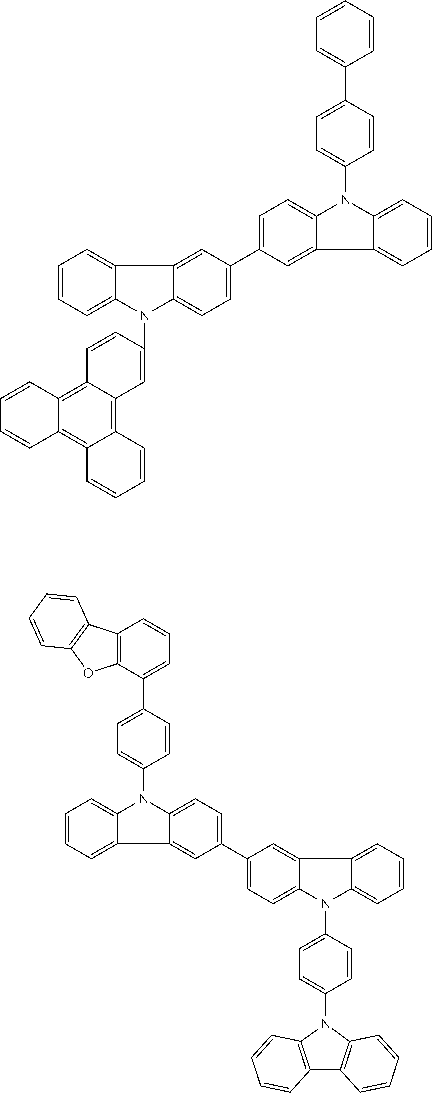 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; c1ccc2c(c1)oc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

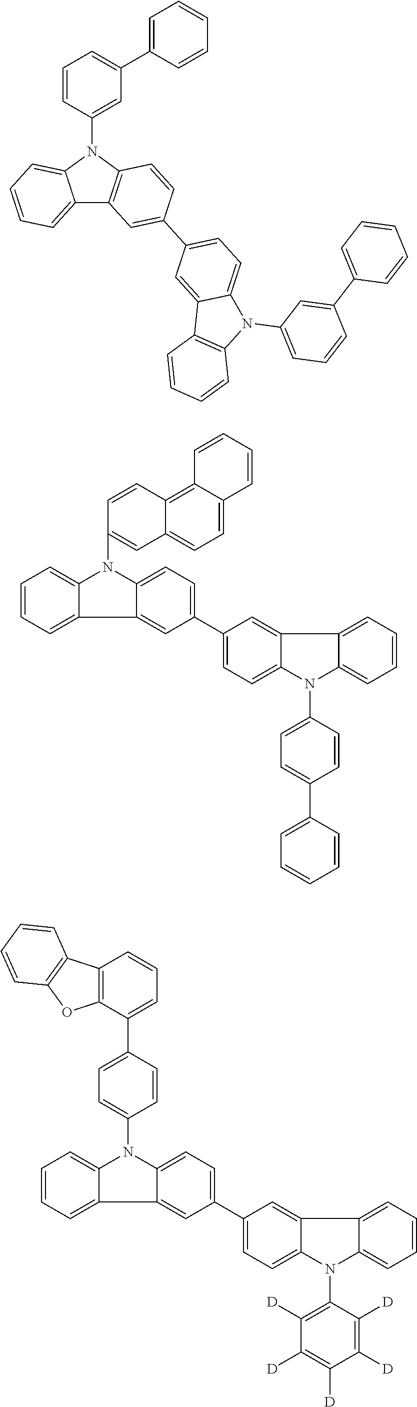

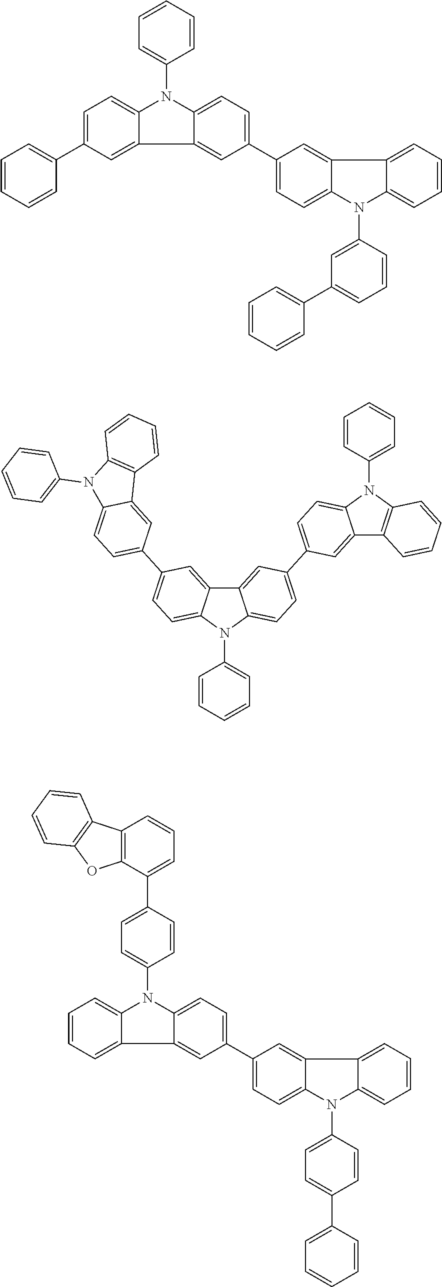 [SMILES: c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(-n3c4ccccc4c4cc(-c5ccc6c(c5)c5ccccc5n6-c5ccc(-c6cccc7c6oc6ccccc67)cc5)ccc43)cc2)cc1]

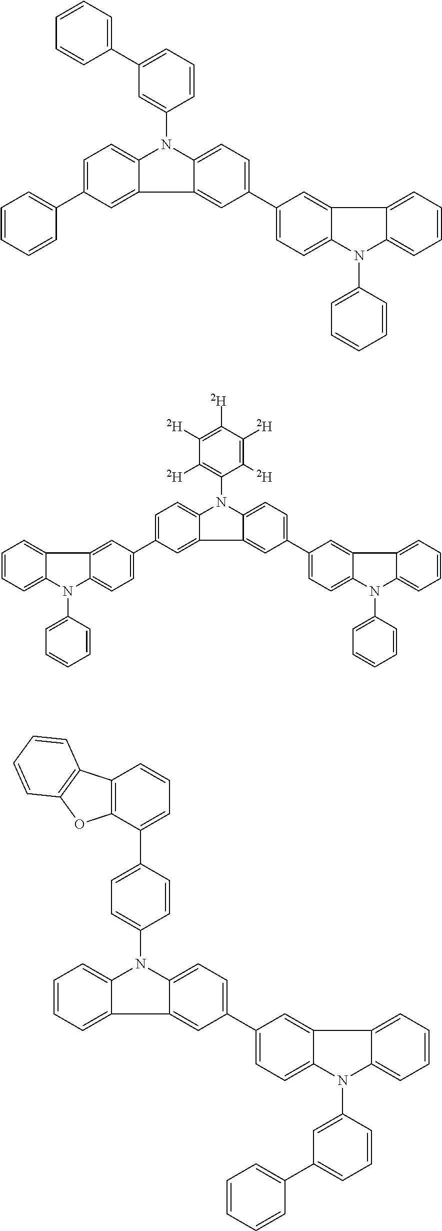 [SMILES: c1ccc(-c2cccc(-n3c4ccc(-c5ccccc5)cc4c4cc(-c5ccc6c(c5)c5ccccc5n6-c5ccccc5)ccc43)c2)cc1 ; [2H]c1c([2H])c([2H])c(-n2c3ccc(-c4ccc5c(c4)c4ccccc4n5-c4ccccc4)cc3c3cc(-c4ccc5c(c4)c4ccccc4n5-c4ccccc4)ccc32)c([2H])c1[2H] ; c1ccc(-c2cccc(-n3c4ccccc4c4cc(-c5ccc6c(c5)c5ccccc5n6-c5ccc(-c6cccc7c6oc6ccccc67)cc5)ccc43)c2)cc1]

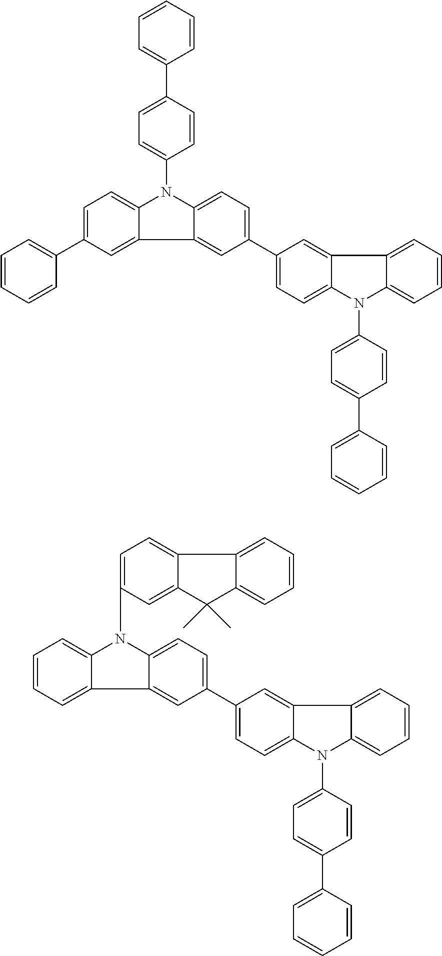 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

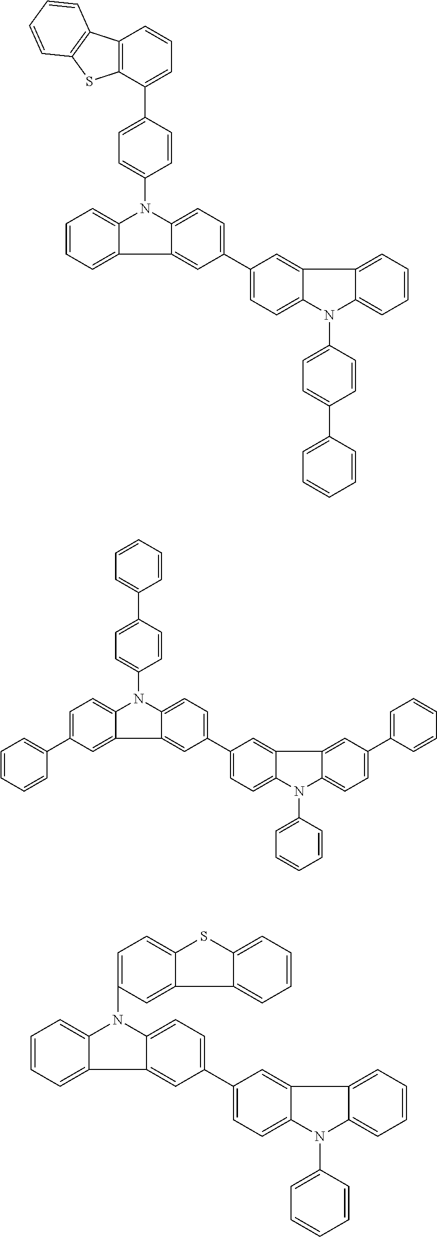 [SMILES: c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1 ; c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4ccc5sc6ccccc6c5c4)ccc32)cc1]

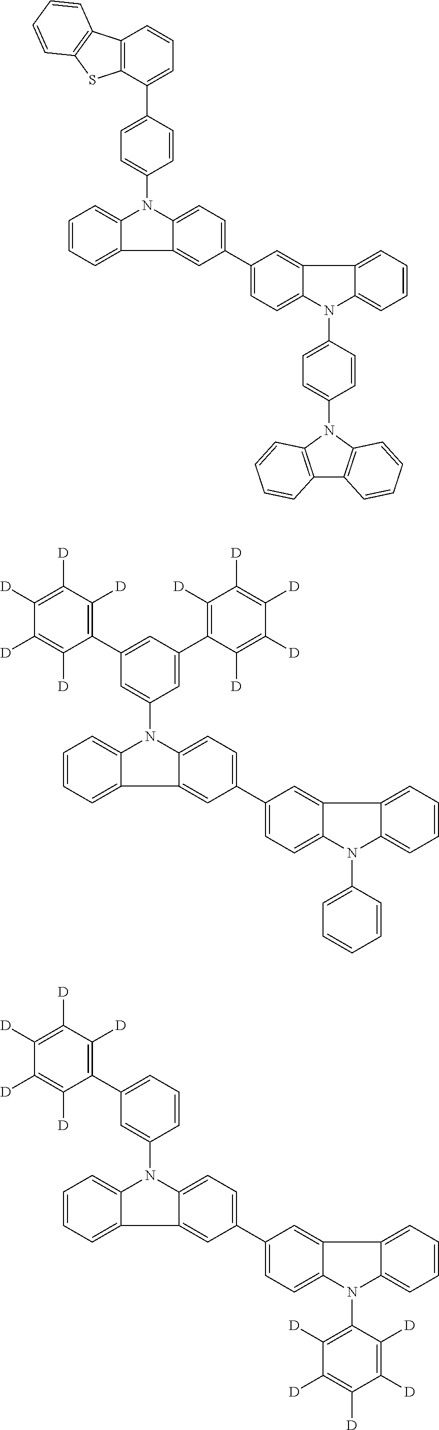 [SMILES: c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12 ; [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H] ; [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H]]

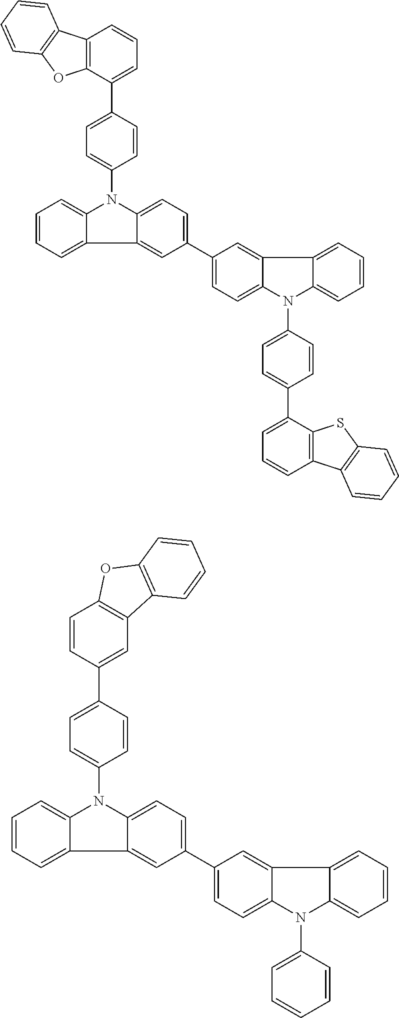 [SMILES: c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12 ; c1ccc(-n2c3ccccc3c3cc(-c4ccc5c(c4)c4ccccc4n5-c4ccc(-c5ccc6oc7ccccc7c6c5)cc4)ccc32)cc1]

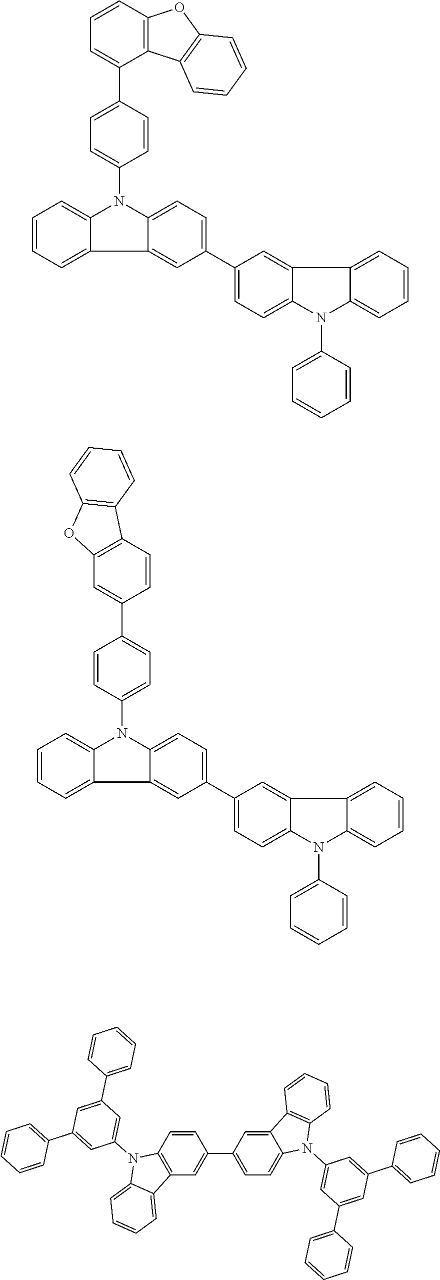 [SMILES: c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4ccc(-c5ccc6c(c5)oc5ccccc56)cc4)ccc32)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1]

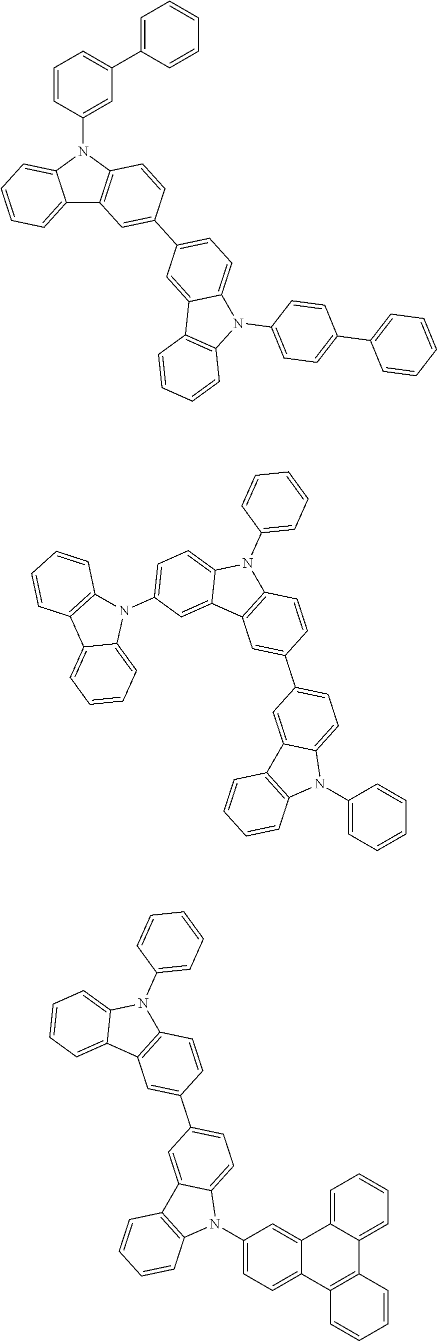 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-n2c3ccccc3c3cc(-c4ccc5c(c4)c4cc(-n6c7ccccc7c7ccccc76)ccc4n5-c4ccccc4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

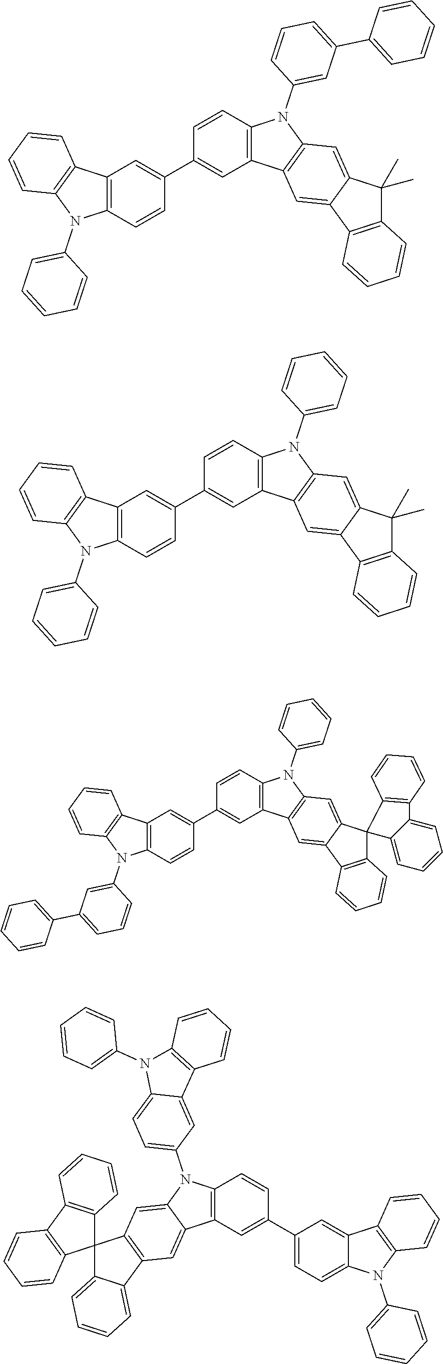 [SMILES: CC1(C)c2ccccc2-c2cc3c4cc(-c5ccc6c(c5)c5ccccc5n6-c5ccccc5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4ccccc4)c3cc21 ; c1ccc(-c2cccc(-n3c4ccccc4c4cc(-c5ccc6c(c5)c5cc7c(cc5n6-c5ccccc5)C5(c6ccccc6-c6ccccc65)c5ccccc5-7)ccc43)c2)cc1 ; c1ccc(-n2c3ccccc3c3cc(-c4ccc5c(c4)c4cc6c(cc4n5-c4ccc5c(c4)c4ccccc4n5-c4ccccc4)C4(c5ccccc5-c5ccccc54)c4ccccc4-6)ccc32)cc1]

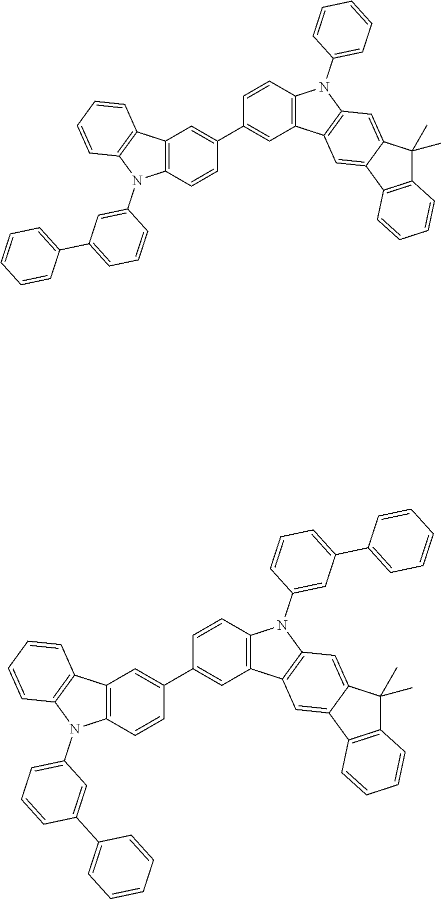 [SMILES: CC1(C)c2ccccc2-c2cc3c4cc(-c5ccc6c(c5)c5ccccc5n6-c5cccc(-c6ccccc6)c5)ccc4n(-c4ccccc4)c3cc21 ; CC1(C)c2ccccc2-c2cc3c4cc(-c5ccc6c(c5)c5ccccc5n6-c5cccc(-c6ccccc6)c5)ccc4n(-c4cccc(-c5ccccc5)c4)c3cc21]

Preferred bridged carbazoles are the structures of the following formula (21):

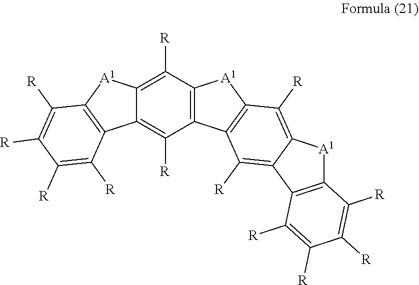

where A¹ and R have the definitions given above and A¹ is preferably the same or different at each instance and is selected from the group consisting of NAr and CR₂.

Preferred dibenzofuran derivatives are the compounds of the following formula (22):

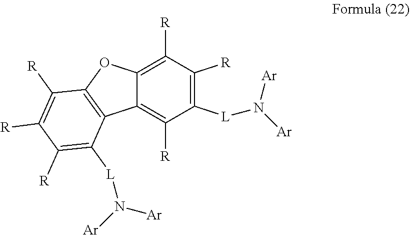

where the oxygen may also be replaced by sulfur so as to form a dibenzothiophene, L is a single bond or an aromatic or heteroaromatic ring system which has 5 to 30 aromatic ring atoms and may also be substituted by one or more R radicals, and R and Ar have the definitions given above. It is also possible here for the two Ar groups that bind to the same nitrogen atom, or for one Ar group and one L group that bind to the same nitrogen atom, to be bonded to one another, for example to give a carbazole.

Examples of suitable dibenzofuran derivatives are the compounds depicted below.

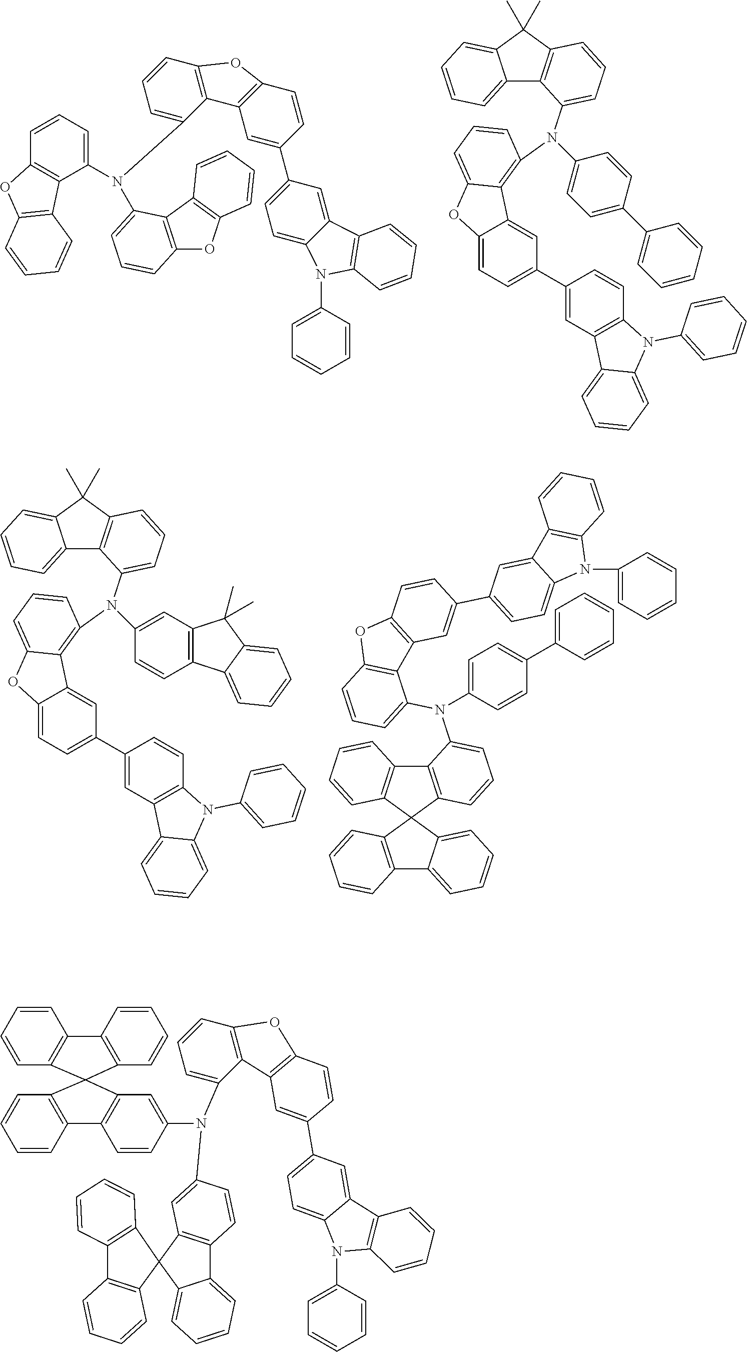

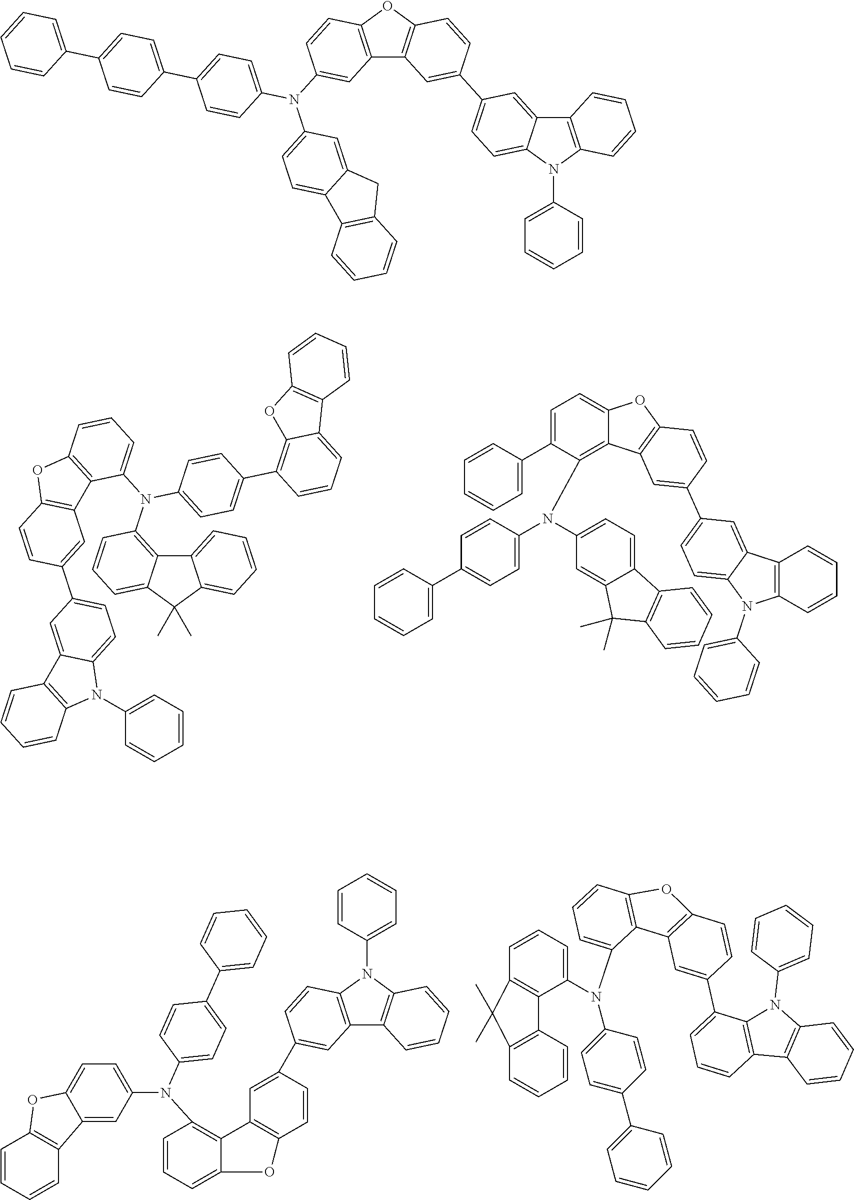

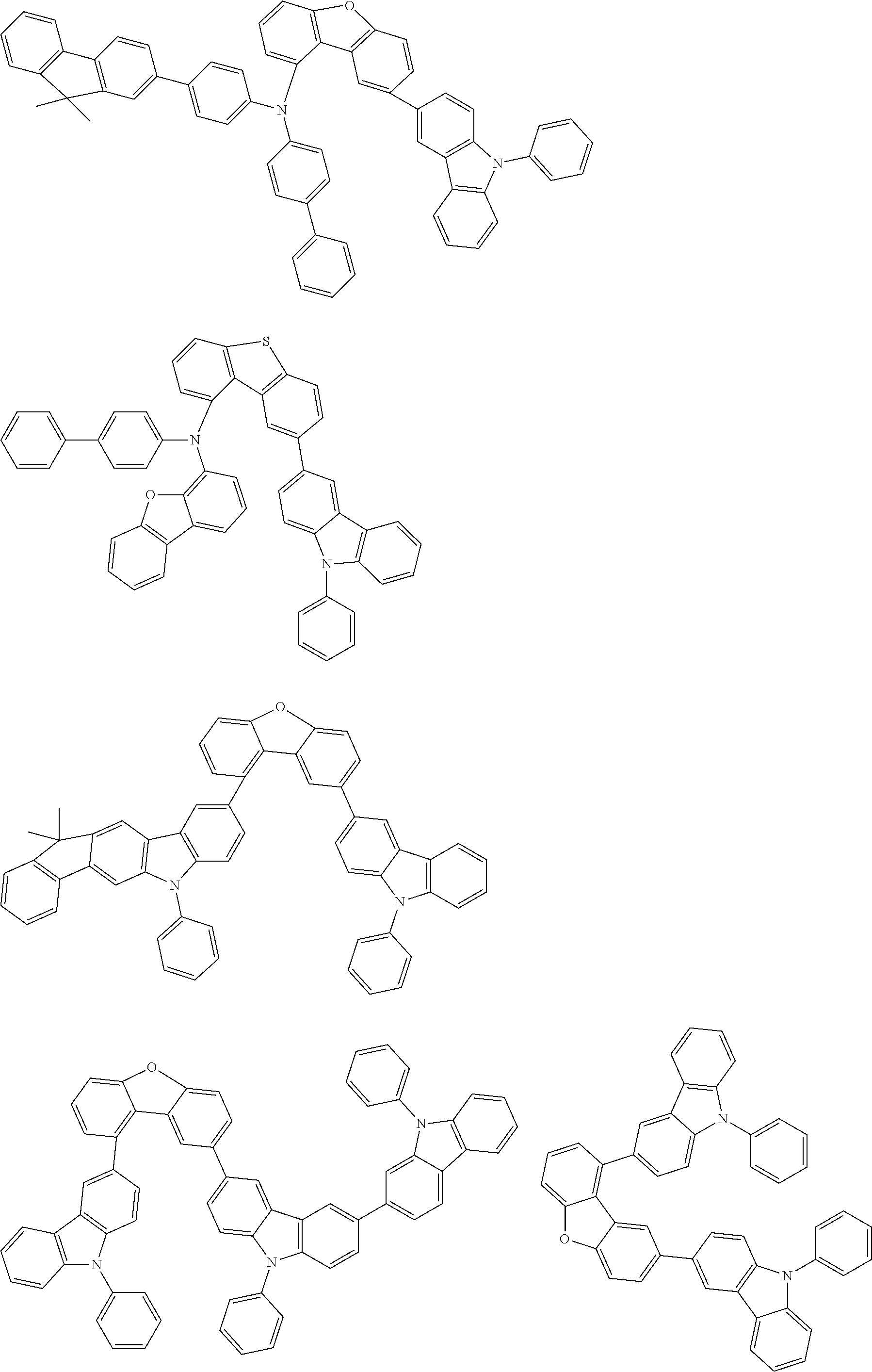

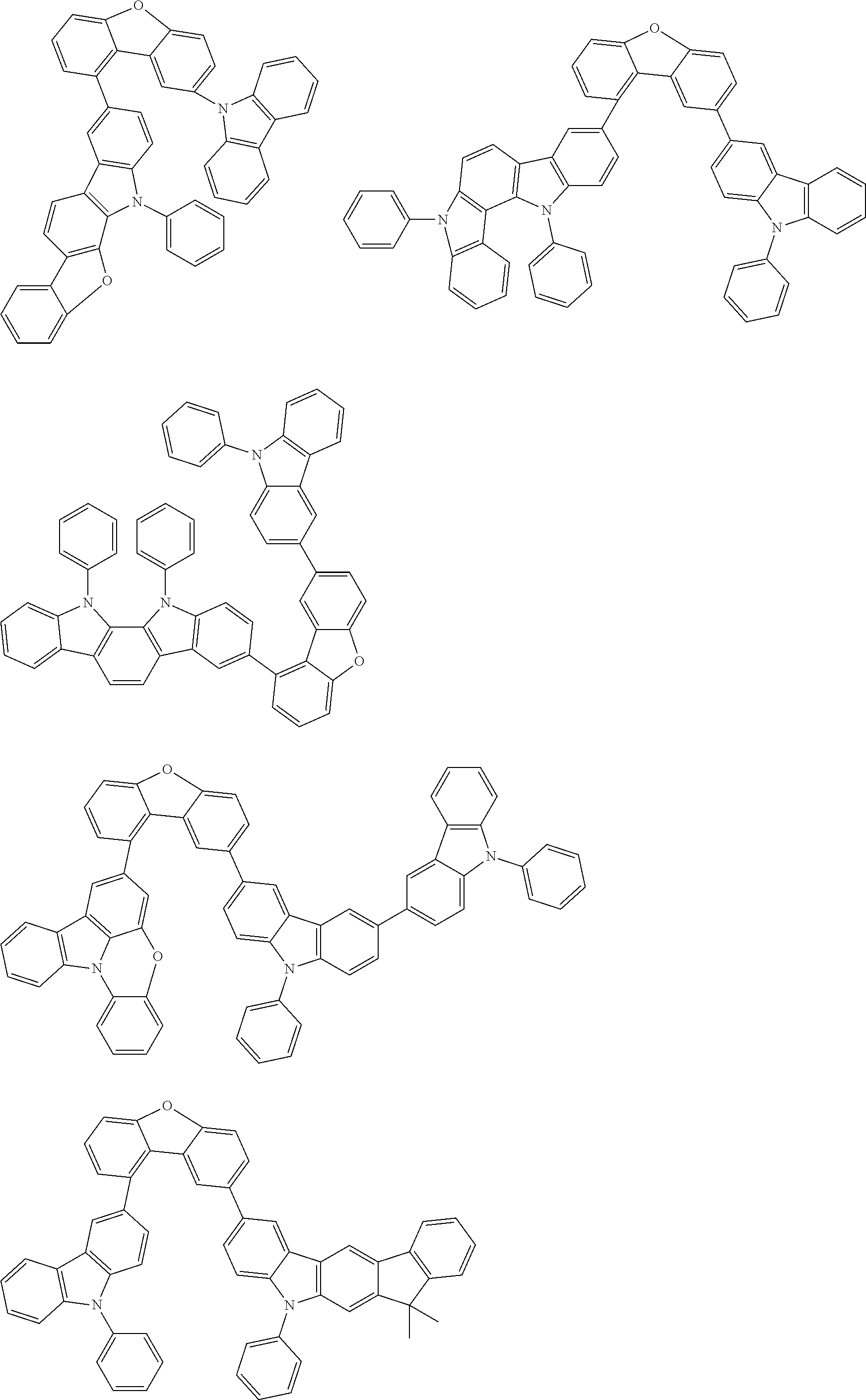

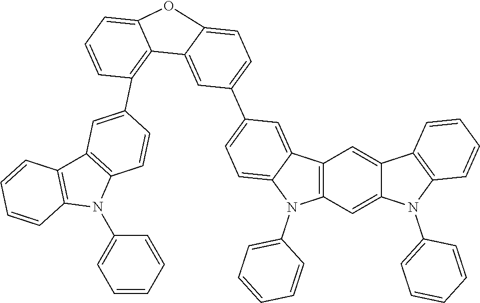 [SMILES: c1ccc(-n2c3ccccc3c3cc(-c4cccc5oc6ccc(-c7ccc8c(c7)c7cc9c%10ccccc%10n(-c%10ccccc%10)c9cc7n8-c7ccccc7)cc6c45)ccc32)cc1]

Preferred carbazoleamines are the structures of the following formulae (23), (24) and (25):

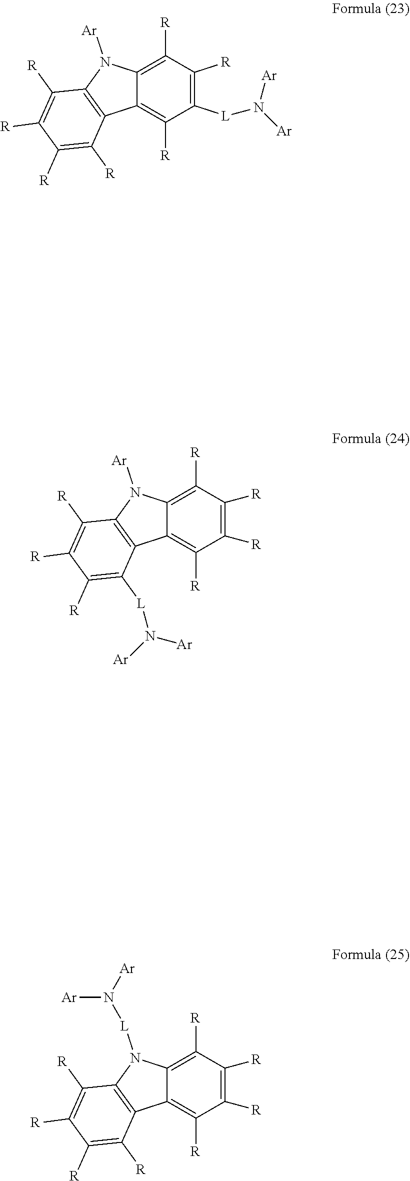

where L is an aromatic or heteroaromatic ring system which has 5 to 30 aromatic ring atoms and may be substituted by one or more R radicals, and R and Ar have the definitions given above.

Examples of suitable carbazolamine derivatives are the compounds depicted below.

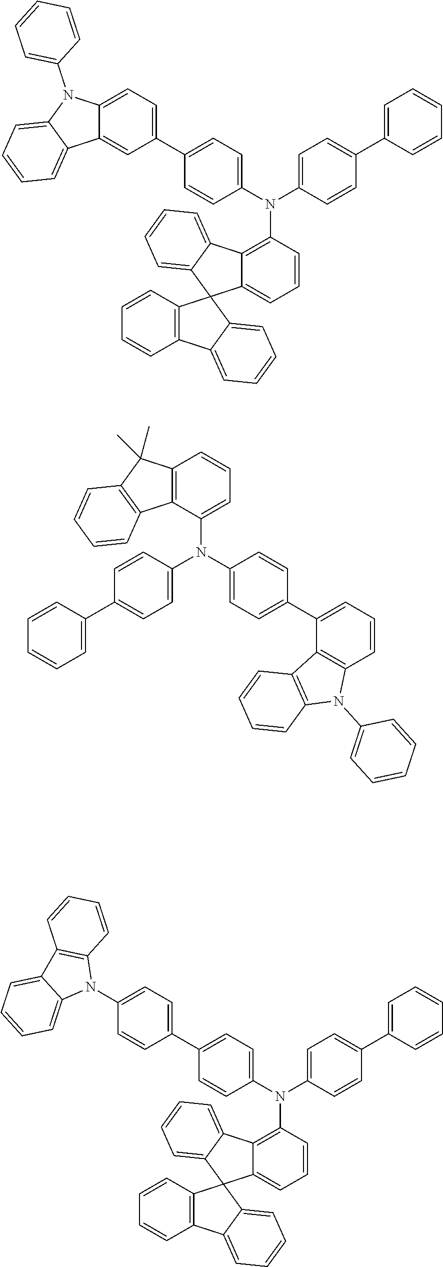 [SMILES: c1ccc(-c2ccc(N(c3ccc(-c4ccc5c(c4)c4ccccc4n5-c4ccccc4)cc3)c3cccc4c3-c3ccccc3C43c4ccccc4-c4ccccc43)cc2)cc1 ; CC1(C)c2ccccc2-c2c(N(c3ccc(-c4ccccc4)cc3)c3ccc(-c4cccc5c4c4ccccc4n5-c4ccccc4)cc3)cccc21 ; c1ccc(-c2ccc(N(c3ccc(-c4ccc(-n5c6ccccc6c6ccccc65)cc4)cc3)c3cccc4c3-c3ccccc3C43c4ccccc4-c4ccccc43)cc2)cc1]

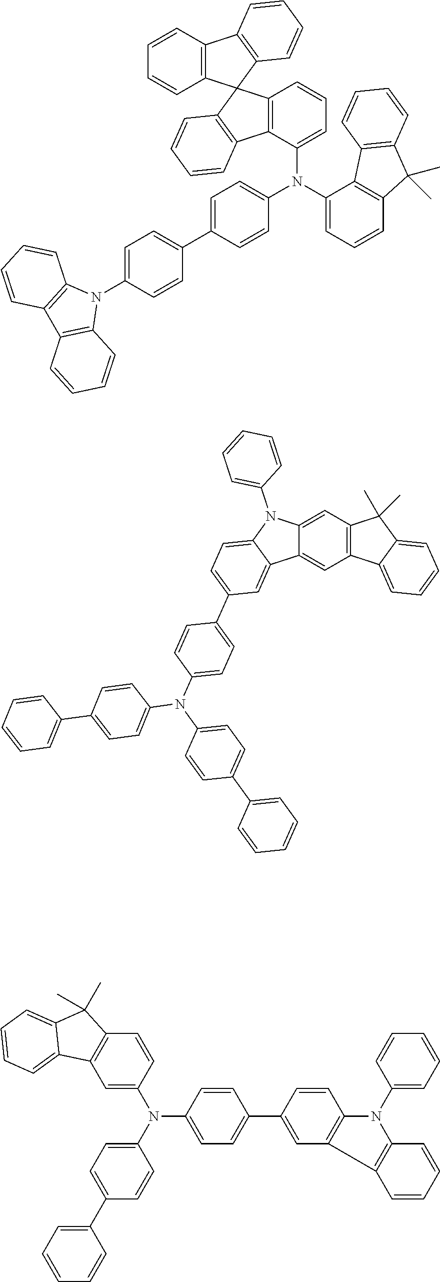 [SMILES: CC1(C)c2ccccc2-c2c(N(c3ccc(-c4ccc(-n5c6ccccc6c6ccccc65)cc4)cc3)c3cccc4c3-c3ccccc3C43c4ccccc4-c4ccccc43)cccc21 ; CC1(C)c2ccccc2-c2cc3c4cc(-c5ccc(N(c6ccc(-c7ccccc7)cc6)c6ccc(-c7ccccc7)cc6)cc5)ccc4n(-c4ccccc4)c3cc21 ; CC1(C)c2ccccc2-c2cc(N(c3ccc(-c4ccccc4)cc3)c3ccc(-c4ccc5c(c4)c4ccccc4n5-c4ccccc4)cc3)ccc21]

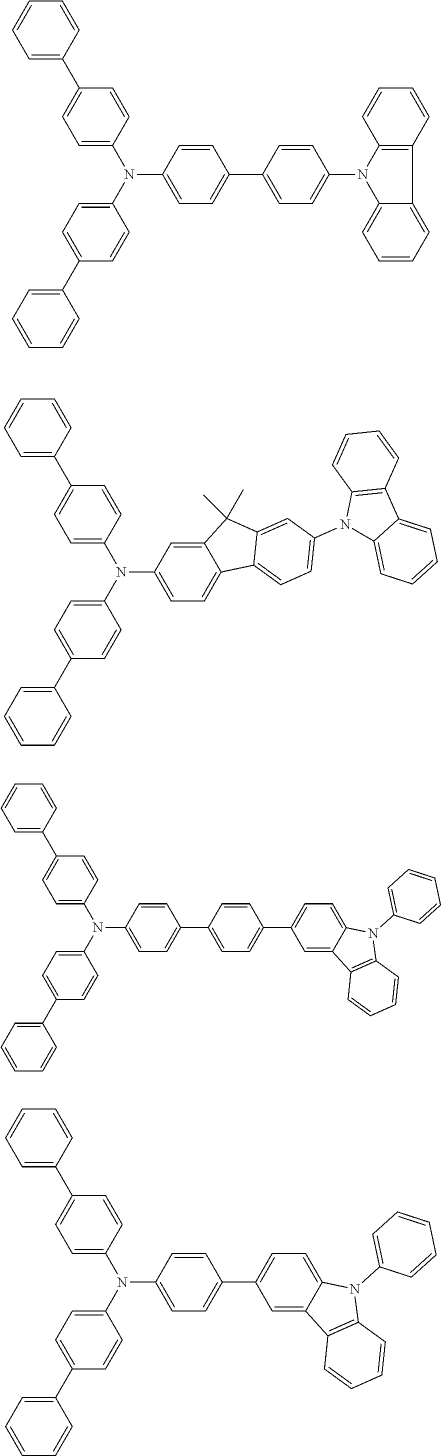

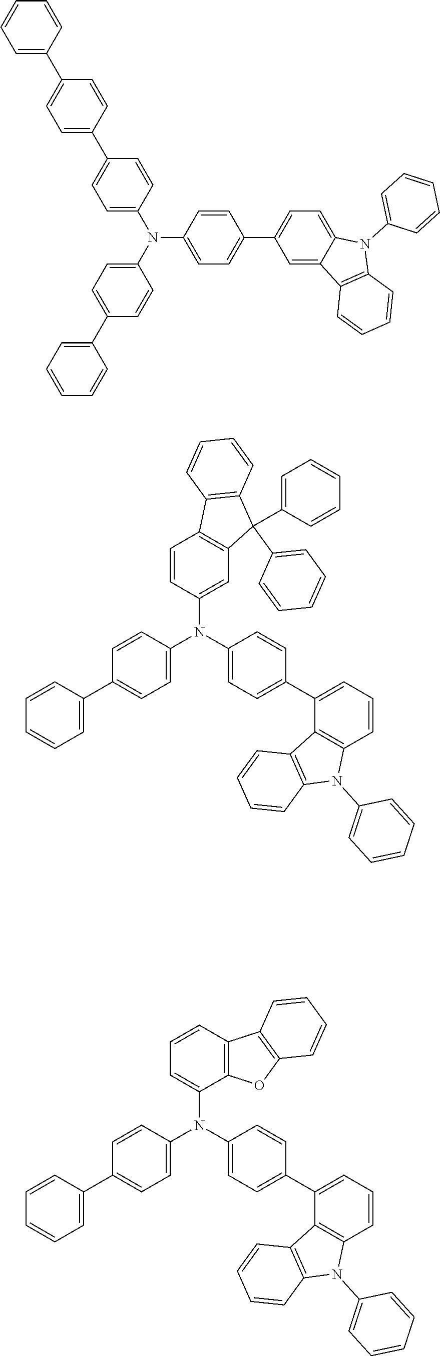 [SMILES: c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccc(N(c3ccc(-c4cccc5c4c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4cccc5c4c4ccccc4n5-c4ccccc4)cc3)c3cccc4c3oc3ccccc34)cc2)cc1]

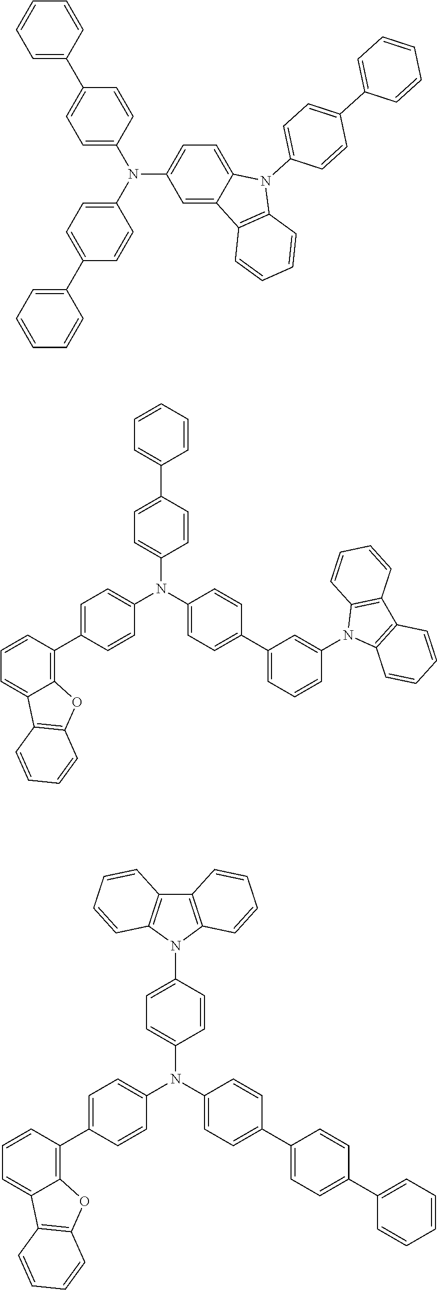 [SMILES: c1ccc(-c2ccc(N(c3ccc(-c4ccccc4)cc3)c3ccc4c(c3)c3ccccc3n4-c3ccc(-c4ccccc4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

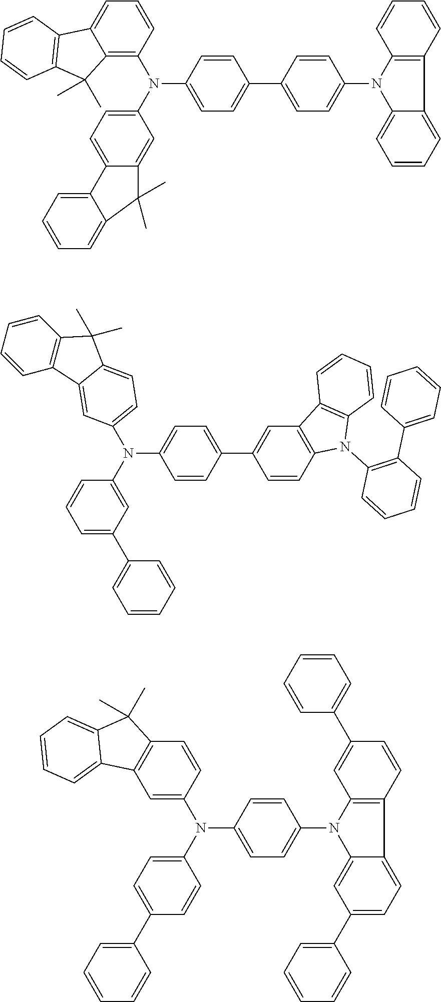 [SMILES: CC1(C)c2ccccc2-c2ccc(N(c3ccc(-c4ccc(-n5c6ccccc6c6ccccc65)cc4)cc3)c3cccc4c3C(C)(C)c3ccccc3-4)cc21 ; CC1(C)c2ccccc2-c2cc(N(c3ccc(-c4ccc5c(c4)c4ccccc4n5-c4ccccc4-c4ccccc4)cc3)c3cccc(-c4ccccc4)c3)ccc21 ; CC1(C)c2ccccc2-c2cc(N(c3ccc(-c4ccccc4)cc3)c3ccc(-n4c5cc(-c6ccccc6)ccc5c5ccc(-c6ccccc6)cc54)cc3)ccc21]

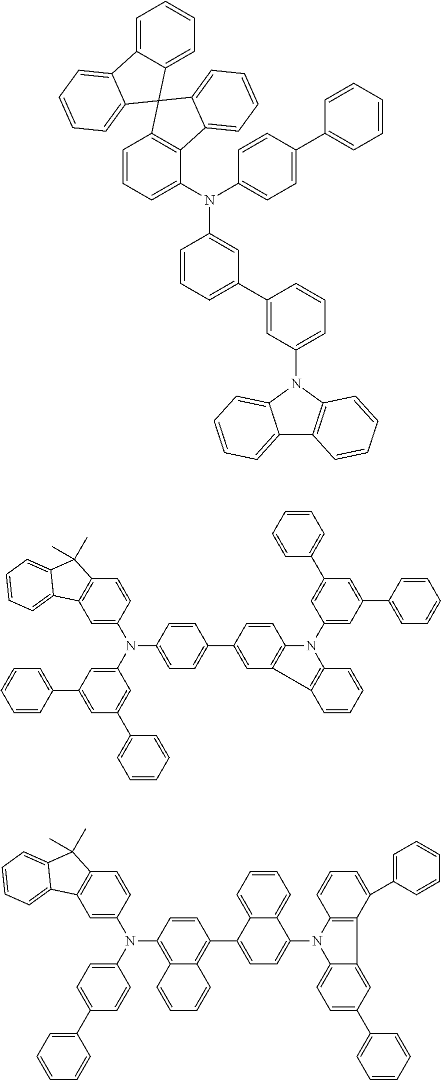 [SMILES: c1ccc(-c2ccc(N(c3cccc(-c4cccc(-n5c6ccccc6c6ccccc65)c4)c3)c3cccc4c3-c3ccccc3C43c4ccccc4-c4ccccc43)cc2)cc1 ; CC1(C)c2ccccc2-c2cc(N(c3ccc(-c4ccc5c(c4)c4ccccc4n5-c4cc(-c5ccccc5)cc(-c5ccccc5)c4)cc3)c3cc(-c4ccccc4)cc(-c4ccccc4)c3)ccc21 ; CC1(C)c2ccccc2-c2cc(N(c3ccc(-c4ccccc4)cc3)c3ccc(-c4ccc(-n5c6ccc(-c7ccccc7)cc6c6c(-c7ccccc7)cccc65)c5ccccc45)c4ccccc34)ccc21]

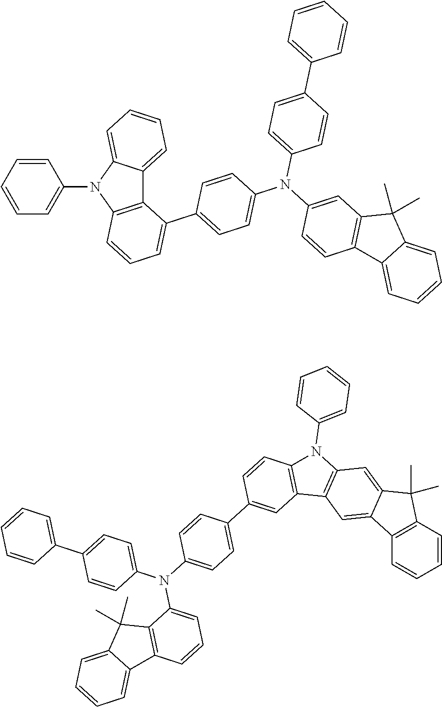 [SMILES: CC1(C)c2ccccc2-c2ccc(N(c3ccc(-c4ccccc4)cc3)c3ccc(-c4cccc5c4c4ccccc4n5-c4ccccc4)cc3)cc21 ; CC1(C)c2ccccc2-c2cc3c4cc(-c5ccc(N(c6ccc(-c7ccccc7)cc6)c6cccc7c6C(C)(C)c6ccccc6-7)cc5)ccc4n(-c4ccccc4)c3cc21]

Preferred co-matrix materials, especially when the compound of the invention is substituted by an electron-rich heteroaromatic ring system, for example a carbazole group, are also selected from the group consisting of triazine derivatives, pyrimidine derivatives, quinazoline derivatives and quinoxaline derivatives. Preferred triazine, quinazoline, quinoxaline or pyrimidine derivatives that can be used as a mixture together with the compounds of the invention are the compounds of the following formulae (26), (27), (28) and (29):

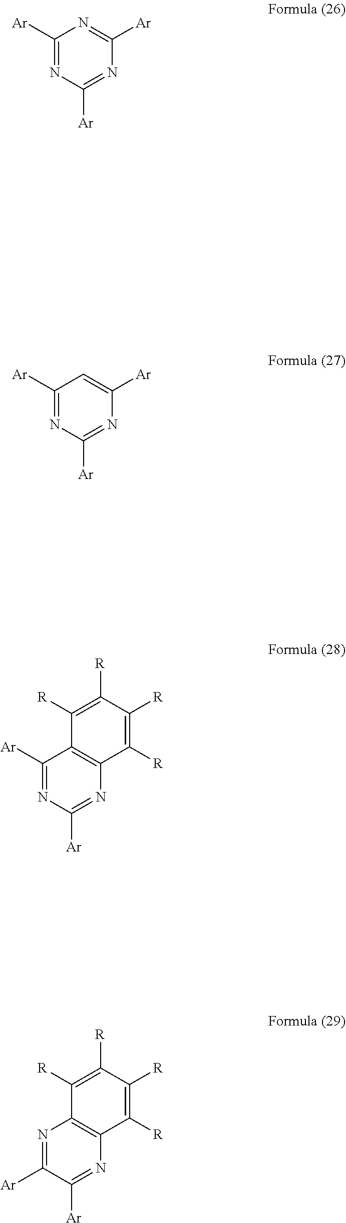

where Ar and R have the definitions given above.

Particular preference is given to the triazine derivatives of the formula (26) and the quinoxaline derivatives of the formula (29), especially the triazine derivatives of the formula (26).

In a preferred embodiment of the invention, Ar in the formulae (26), (27), (28) and (29) is the same or different at each instance and is an aromatic or heteroaromatic ring system which has 6 to 30 aromatic ring atoms, especially 6 to 24 aromatic ring atoms, and may be substituted by one or more R radicals. Suitable aromatic or heteroaromatic ring systems Ar here are the same as set out above as embodiments for Ar, especially the structures Ar-1 to Ar-76.

Examples of suitable triazine compounds that may be used as matrix materials together with the compounds of the invention are the compounds depicted in the following table:

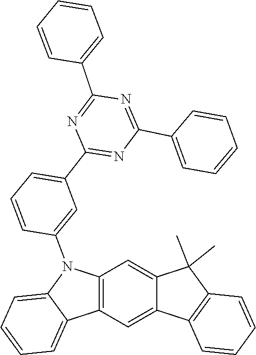 [SMILES: CC1(C)c2ccccc2-c2cc3c4ccccc4n(-c4cccc(-c5nc(-c6ccccc6)nc(-c6ccccc6)n5)c4)c3cc21]

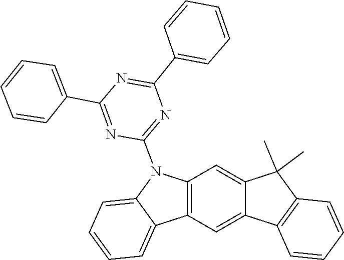 [SMILES: CC1(C)c2ccccc2-c2cc3c4ccccc4n(-c4nc(-c5ccccc5)nc(-c5ccccc5)n4)c3cc21]

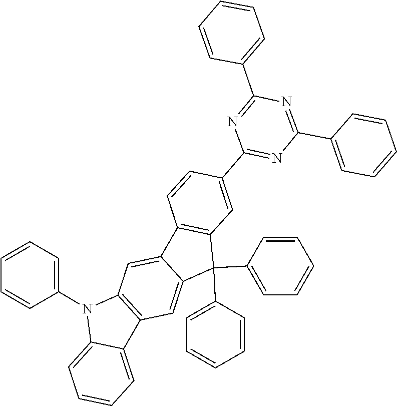 [SMILES: c1ccc(-c2nc(-c3ccccc3)nc(-c3ccc4c(c3)C(c3ccccc3)(c3ccccc3)c3cc5c6ccccc6n(-c6ccccc6)c5cc3-4)n2)cc1]

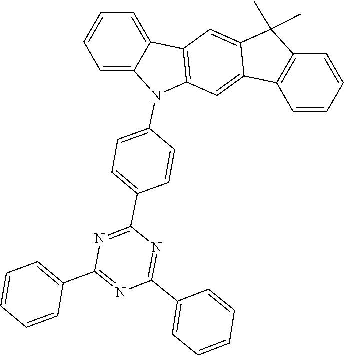 [SMILES: CC1(C)c2ccccc2-c2cc3c(cc21)c1ccccc1n3-c1ccc(-c2nc(-c3ccccc3)nc(-c3ccccc3)n2)cc1]

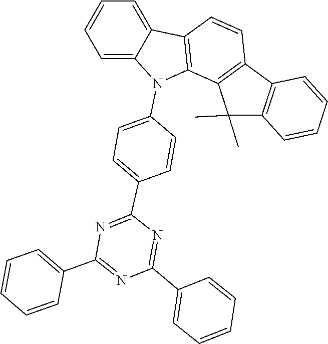 [SMILES: CC1(C)c2ccccc2-c2ccc3c4ccccc4n(-c4ccc(-c5nc(-c6ccccc6)nc(-c6ccccc6)n5)cc4)c3c21]

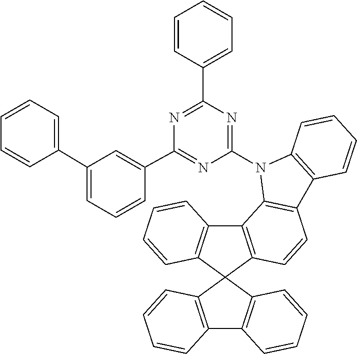 [SMILES: c1ccc(-c2cccc(-c3nc(-c4ccccc4)nc(-n4c5ccccc5c5ccc6c(c54)-c4ccccc4C64c5ccccc5-c5ccccc54)n3)c2)cc1]

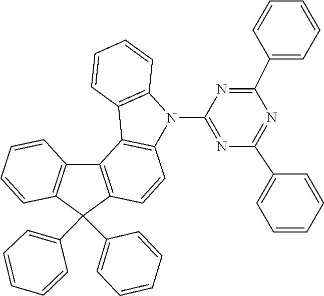 [SMILES: c1ccc(-c2nc(-c3ccccc3)nc(-n3c4ccccc4c4c5c(ccc43)C(c3ccccc3)(c3ccccc3)c3ccccc3-5)n2)cc1]

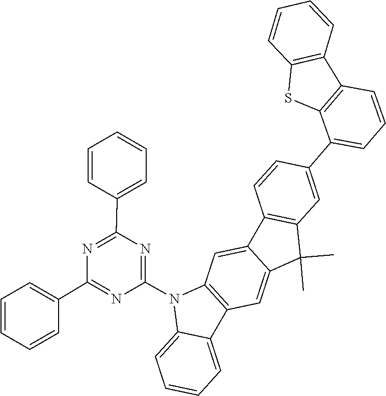 [SMILES: CC1(C)c2cc(-c3cccc4c3sc3ccccc34)ccc2-c2cc3c(cc21)c1ccccc1n3-c1nc(-c2ccccc2)nc(-c2ccccc2)n1]

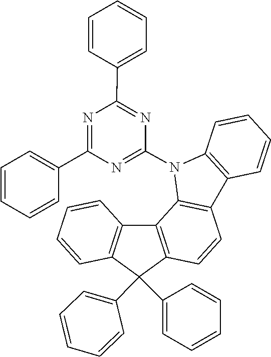 [SMILES: c1ccc(-c2nc(-c3ccccc3)nc(-n3c4ccccc4c4ccc5c(c43)-c3ccccc3C5(c3ccccc3)c3ccccc3)n2)cc1]

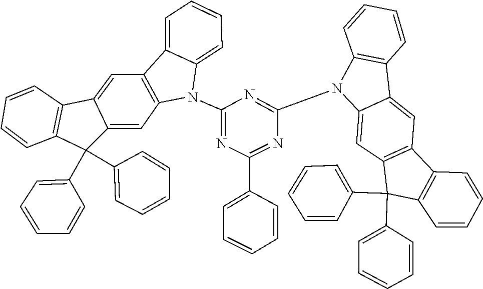 [SMILES: c1ccc(-c2nc(-n3c4ccccc4c4cc5c(cc43)C(c3ccccc3)(c3ccccc3)c3ccccc3-5)nc(-n3c4ccccc4c4cc5c(cc43)C(c3ccccc3)(c3ccccc3)c3ccccc3-5)n2)cc1]

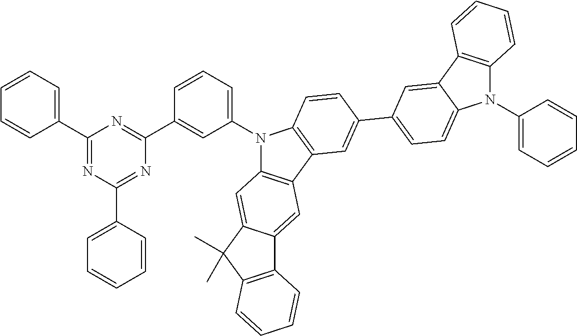 [SMILES: CC1(C)c2ccccc2-c2cc3c4cc(-c5ccc6c(c5)c5ccccc5n6-c5ccccc5)ccc4n(-c4cccc(-c5nc(-c6ccccc6)nc(-c6ccccc6)n5)c4)c3cc21]

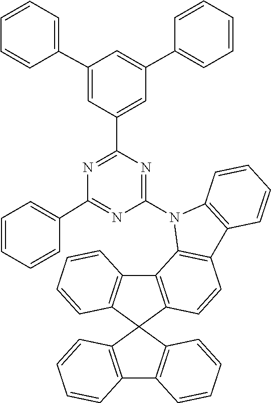 [SMILES: c1ccc(-c2cc(-c3ccccc3)cc(-c3nc(-c4ccccc4)nc(-n4c5ccccc5c5ccc6c(c54)-c4ccccc4C64c5ccccc5-c5ccccc54)n3)c2)cc1]

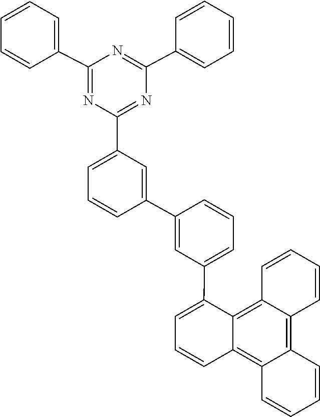 [SMILES: c1ccc(-c2nc(-c3ccccc3)nc(-c3cccc(-c4cccc(-c5cccc6c7ccccc7c7ccccc7c56)c4)c3)n2)cc1]

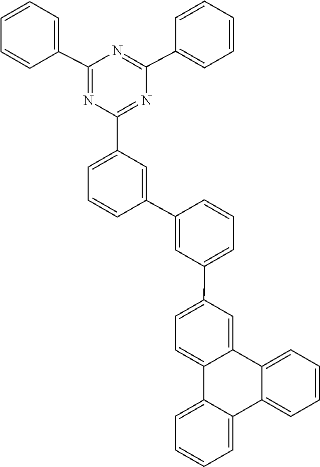 [SMILES: c1ccc(-c2nc(-c3ccccc3)nc(-c3cccc(-c4cccc(-c5ccc6c7ccccc7c7ccccc7c6c5)c4)c3)n2)cc1]

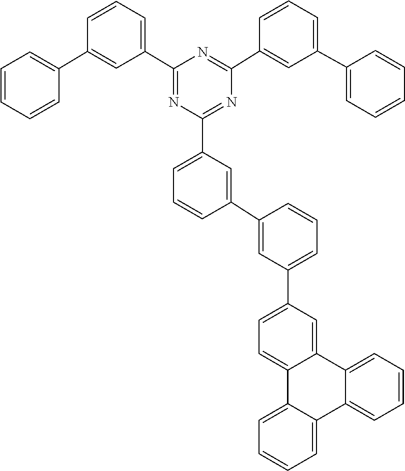 [SMILES: c1ccc(-c2cccc(-c3nc(-c4cccc(-c5ccccc5)c4)nc(-c4cccc(-c5cccc(-c6ccc7c8ccccc8c8ccccc8c7c6)c5)c4)n3)c2)cc1]

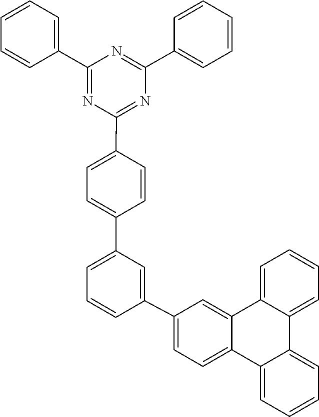 [SMILES: c1ccc(-c2nc(-c3ccccc3)nc(-c3ccc(-c4cccc(-c5ccc6c7ccccc7c7ccccc7c6c5)c4)cc3)n2)cc1]

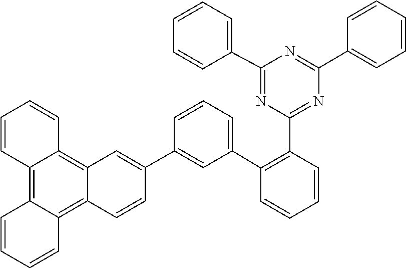 [SMILES: c1ccc(-c2nc(-c3ccccc3)nc(-c3ccccc3-c3cccc(-c4ccc5c6ccccc6c6ccccc6c5c4)c3)n2)cc1]

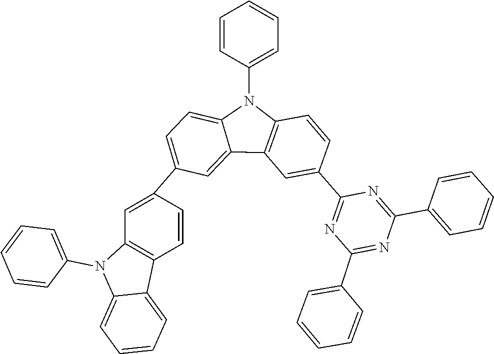 [SMILES: c1ccc(-c2nc(-c3ccccc3)nc(-c3ccc4c(c3)c3cc(-c5ccc6c7ccccc7n(-c7ccccc7)c6c5)ccc3n4-c3ccccc3)n2)cc1]

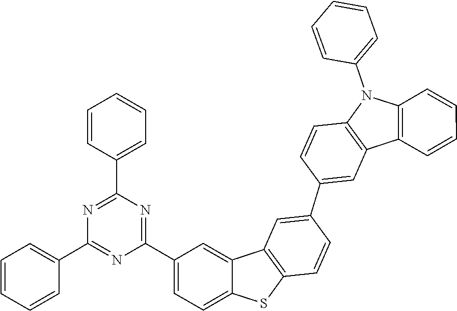 [SMILES: c1ccc(-c2nc(-c3ccccc3)nc(-c3ccc4sc5ccc(-c6ccc7c(c6)c6ccccc6n7-c6ccccc6)cc5c4c3)n2)cc1]

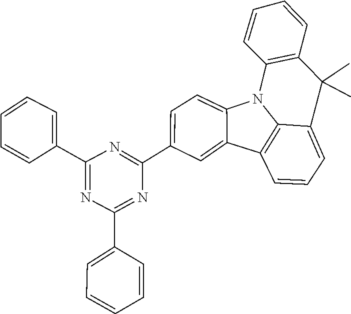 [SMILES: CC1(C)c2ccccc2-n2c3ccc(-c4nc(-c5ccccc5)nc(-c5ccccc5)n4)cc3c3cccc1c32]

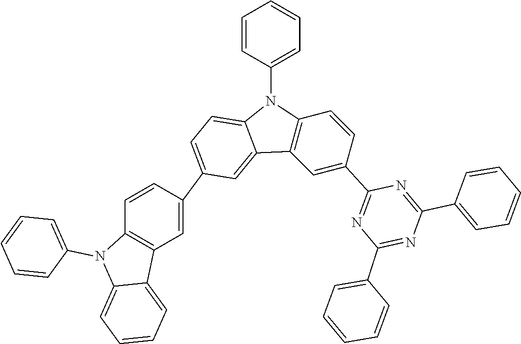 [SMILES: c1ccc(-c2nc(-c3ccccc3)nc(-c3ccc4c(c3)c3cc(-c5ccc6c(c5)c5ccccc5n6-c5ccccc5)ccc3n4-c3ccccc3)n2)cc1]

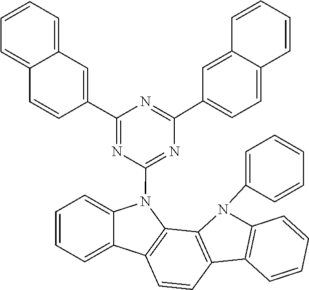 [SMILES: c1ccc(-n2c3ccccc3c3ccc4c5ccccc5n(-c5nc(-c6ccc7ccccc7c6)nc(-c6ccc7ccccc7c6)n5)c4c32)cc1]

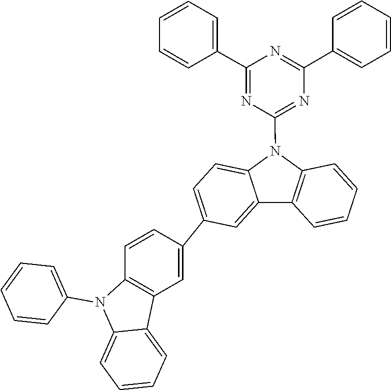 [SMILES: c1ccc(-c2nc(-c3ccccc3)nc(-n3c4ccccc4c4cc(-c5ccc6c(c5)c5ccccc5n6-c5ccccc5)ccc43)n2)cc1]

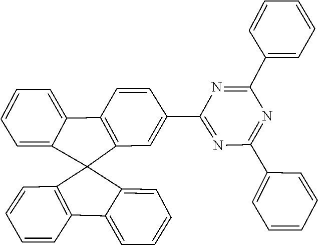 [SMILES: c1ccc(-c2nc(-c3ccccc3)nc(-c3ccc4c(c3)C3(c5ccccc5-c5ccccc53)c3ccccc3-4)n2)cc1]

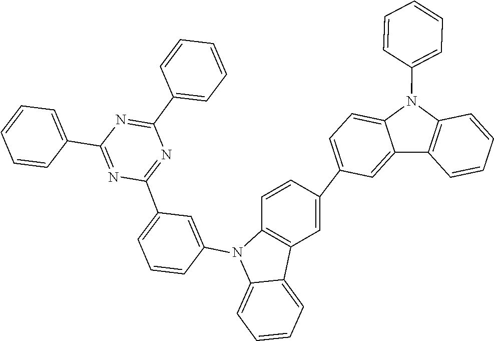 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

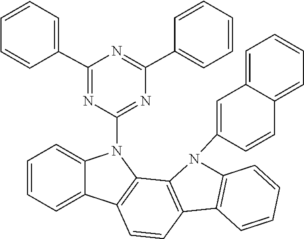 [SMILES: c1ccc(-c2nc(-c3ccccc3)nc(-n3c4ccccc4c4ccc5c6ccccc6n(-c6ccc7ccccc7c6)c5c43)n2)cc1]

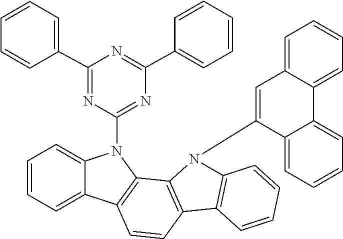 [SMILES: c1ccc(-c2nc(-c3ccccc3)nc(-n3c4ccccc4c4ccc5c6ccccc6n(-c6cc7ccccc7c7ccccc67)c5c43)n2)cc1]

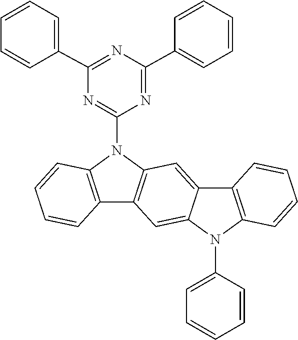 [SMILES: c1ccc(-c2nc(-c3ccccc3)nc(-n3c4ccccc4c4cc5c(cc43)c3ccccc3n5-c3ccccc3)n2)cc1]

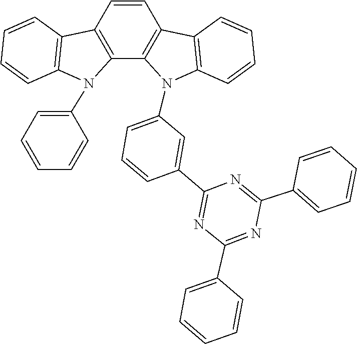 [SMILES: c1ccc(-c2nc(-c3ccccc3)nc(-c3cccc(-n4c5ccccc5c5ccc6c7ccccc7n(-c7ccccc7)c6c54)c3)n2)cc1]

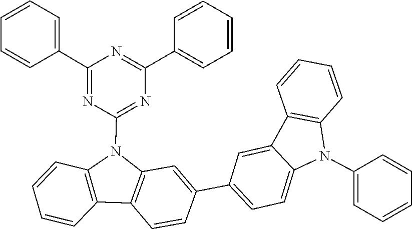 [SMILES: c1ccc(-c2nc(-c3ccccc3)nc(-n3c4ccccc4c4ccc(-c5ccc6c(c5)c5ccccc5n6-c5ccccc5)cc43)n2)cc1]

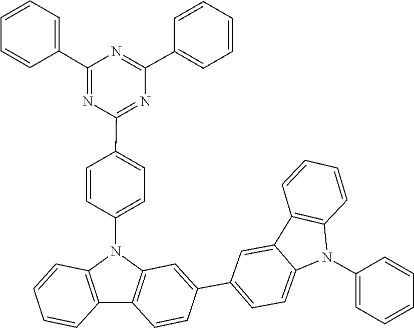 [SMILES: c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1]

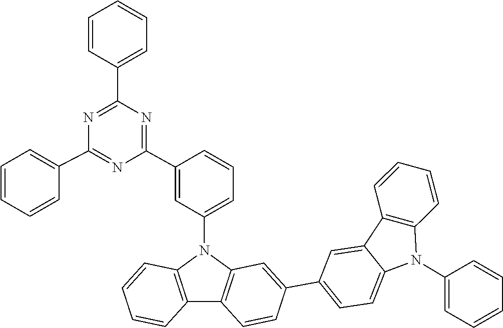 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

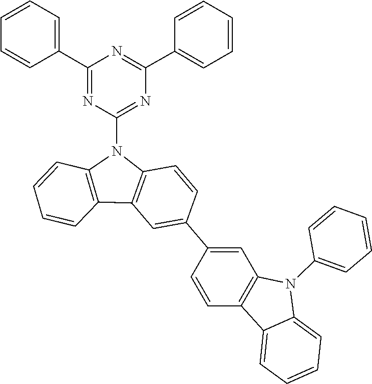 [SMILES: c1ccc(-c2nc(-c3ccccc3)nc(-n3c4ccccc4c4cc(-c5ccc6c7ccccc7n(-c7ccccc7)c6c5)ccc43)n2)cc1]

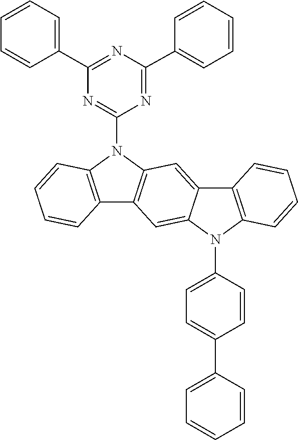 [SMILES: c1ccc(-c2ccc(-n3c4ccccc4c4cc5c(cc43)c3ccccc3n5-c3nc(-c4ccccc4)nc(-c4ccccc4)n3)cc2)cc1]

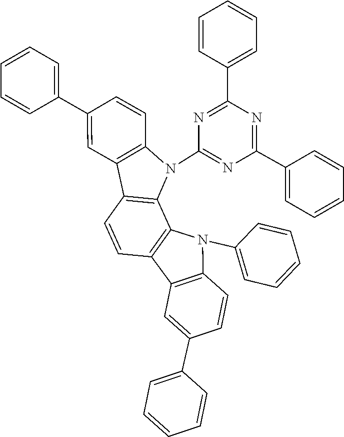 [SMILES: c1ccc(-c2ccc3c(c2)c2ccc4c5cc(-c6ccccc6)ccc5n(-c5nc(-c6ccccc6)nc(-c6ccccc6)n5)c4c2n3-c2ccccc2)cc1]

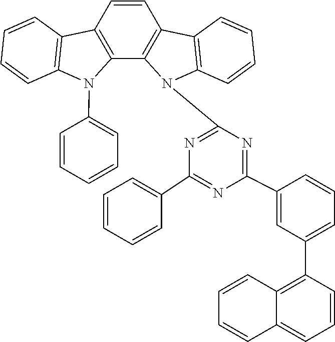 [SMILES: c1ccc(-c2nc(-c3cccc(-c4cccc5ccccc45)c3)nc(-n3c4ccccc4c4ccc5c6ccccc6n(-c6ccccc6)c5c43)n2)cc1]

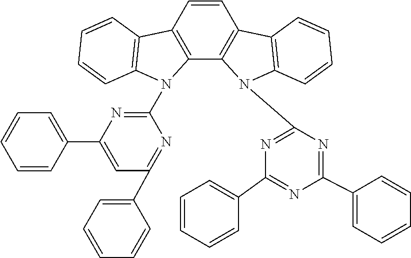 [SMILES: c1ccc(-c2cc(-c3ccccc3)nc(-n3c4ccccc4c4ccc5c6ccccc6n(-c6nc(-c7ccccc7)nc(-c7ccccc7)n6)c5c43)n2)cc1]

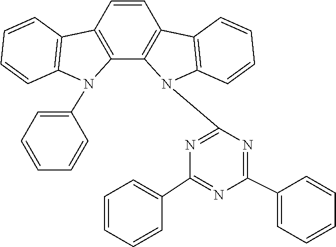 [SMILES: c1ccc(-c2nc(-c3ccccc3)nc(-n3c4ccccc4c4ccc5c6ccccc6n(-c6ccccc6)c5c43)n2)cc1]

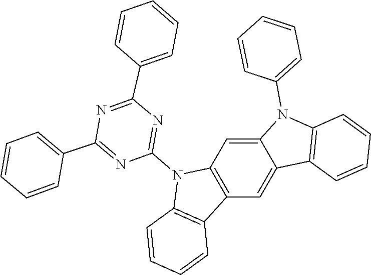 [SMILES: c1ccc(-c2nc(-c3ccccc3)nc(-n3c4ccccc4c4cc5c6ccccc6n(-c6ccccc6)c5cc43)n2)cc1]

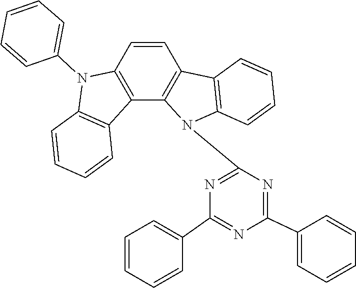 [SMILES: c1ccc(-c2nc(-c3ccccc3)nc(-n3c4ccccc4c4ccc5c(c6ccccc6n5-c5ccccc5)c43)n2)cc1]

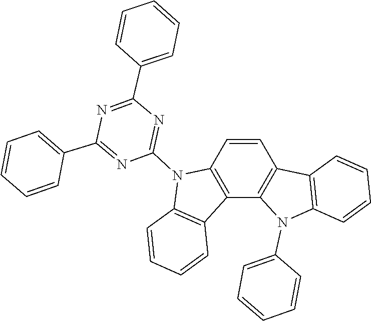 [SMILES: c1ccc(-c2nc(-c3ccccc3)nc(-n3c4ccccc4c4c3ccc3c5ccccc5n(-c5ccccc5)c34)n2)cc1]

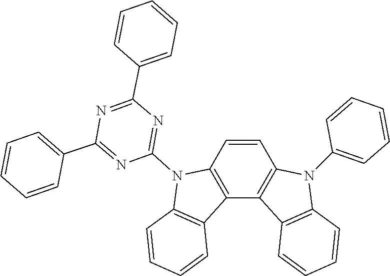 [SMILES: c1ccc(-c2nc(-c3ccccc3)nc(-n3c4ccccc4c4c5c6ccccc6n(-c6ccccc6)c5ccc43)n2)cc1]

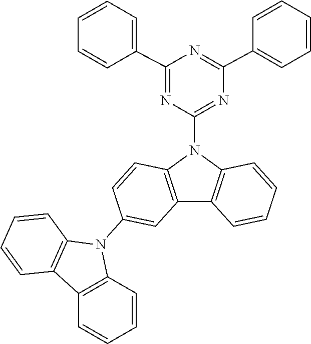 [SMILES: c1ccc(-c2nc(-c3ccccc3)nc(-n3c4ccccc4c4cc(-n5c6ccccc6c6ccccc65)ccc43)n2)cc1]

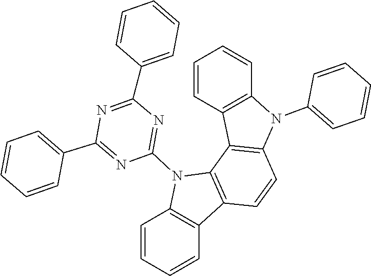 [SMILES: c1ccc(-c2nc(-c3ccccc3)nc(-n3c4ccccc4c4ccc5c(c6ccccc6n5-c5ccccc5)c43)n2)cc1]

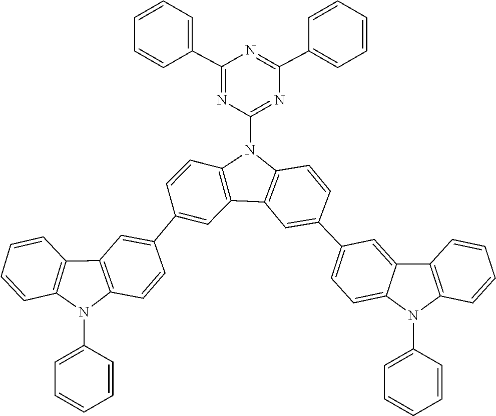 [SMILES: c1ccc(-c2nc(-c3ccccc3)nc(-n3c4ccc(-c5ccc6c(c5)c5ccccc5n6-c5ccccc5)cc4c4cc(-c5ccc6c(c5)c5ccccc5n6-c5ccccc5)ccc43)n2)cc1]

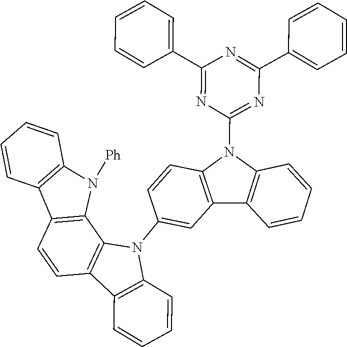 [SMILES: c1ccc(-c2nc(-c3ccccc3)nc(-n3c4ccccc4c4cc(-n5c6ccccc6c6ccc7c8ccccc8n(-c8ccccc8)c7c65)ccc43)n2)cc1]

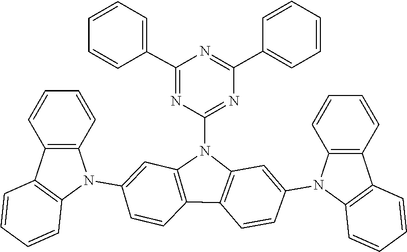 [SMILES: c1ccc(-c2nc(-c3ccccc3)nc(-n3c4cc(-n5c6ccccc6c6ccccc65)ccc4c4ccc(-n5c6ccccc6c6ccccc65)cc43)n2)cc1]

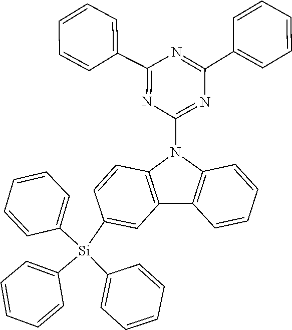 [SMILES: c1ccc(-c2nc(-c3ccccc3)nc(-n3c4ccccc4c4cc([Si](c5ccccc5)(c5ccccc5)c5ccccc5)ccc43)n2)cc1]

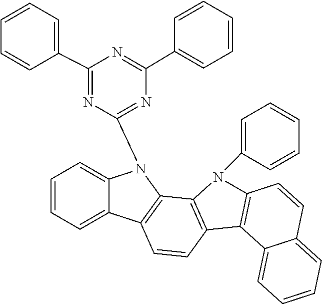 [SMILES: c1ccc(-c2nc(-c3ccccc3)nc(-n3c4ccccc4c4ccc5c6c7ccccc7ccc6n(-c6ccccc6)c5c43)n2)cc1]

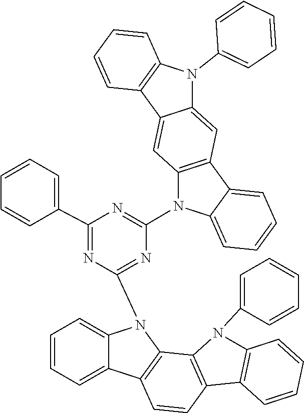 [SMILES: c1ccc(-c2nc(-n3c4ccccc4c4cc5c(cc43)c3ccccc3n5-c3ccccc3)nc(-n3c4ccccc4c4ccc5c6ccccc6n(-c6ccccc6)c5c43)n2)cc1]

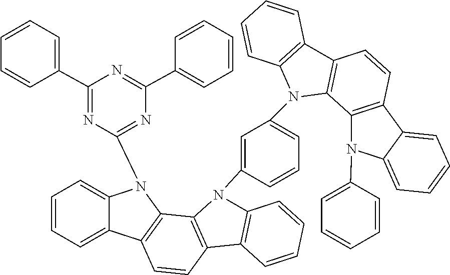 [SMILES: c1ccc(-c2nc(-c3ccccc3)nc(-n3c4ccccc4c4ccc5c6ccccc6n(-c6cccc(-n7c8ccccc8c8ccc9c%10ccccc%10n(-c%10ccccc%10)c9c87)c6)c5c43)n2)cc1]

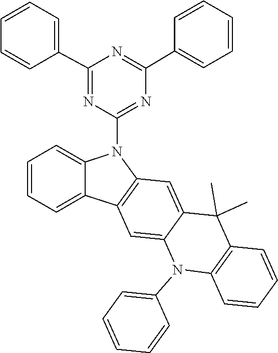 [SMILES: CC1(C)c2ccccc2N(c2ccccc2)c2cc3c4ccccc4n(-c4nc(-c5ccccc5)nc(-c5ccccc5)n4)c3cc21]

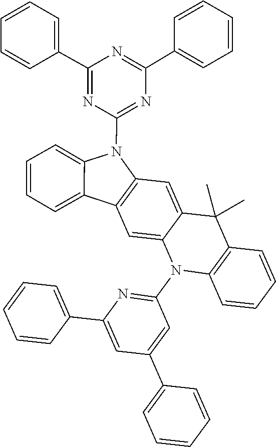 [SMILES: CC1(C)c2ccccc2N(c2cc(-c3ccccc3)cc(-c3ccccc3)n2)c2cc3c4ccccc4n(-c4nc(-c5ccccc5)nc(-c5ccccc5)n4)c3cc21]

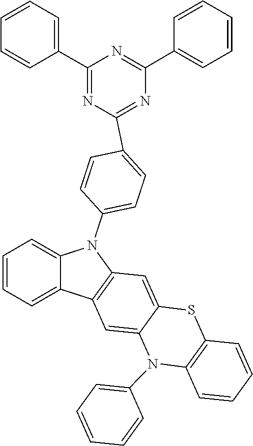 [SMILES: c1ccc(-c2nc(-c3ccccc3)nc(-c3ccc(-n4c5ccccc5c5cc6c(cc54)Sc4ccccc4N6c4ccccc4)cc3)n2)cc1]

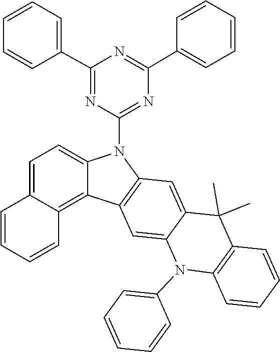 [SMILES: CC1(C)c2ccccc2N(c2ccccc2)c2cc3c4c5ccccc5ccc4n(-c4nc(-c5ccccc5)nc(-c5ccccc5)n4)c3cc21]

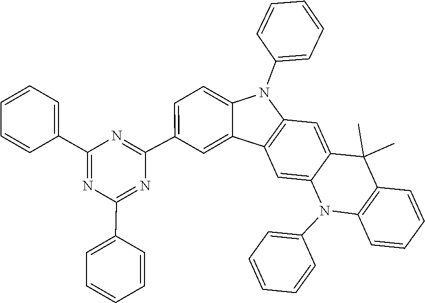 [SMILES: CC1(C)c2ccccc2N(c2ccccc2)c2cc3c4cc(-c5nc(-c6ccccc6)nc(-c6ccccc6)n5)ccc4n(-c4ccccc4)c3cc21]

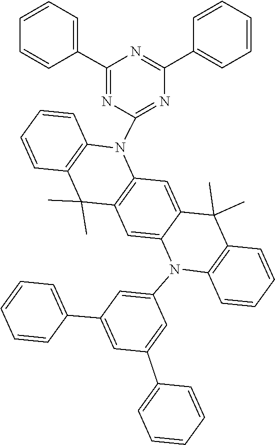 [SMILES: CC1(C)c2ccccc2N(c2cc(-c3ccccc3)cc(-c3ccccc3)c2)c2cc3c(cc21)N(c1nc(-c2ccccc2)nc(-c2ccccc2)n1)c1ccccc1C3(C)C]

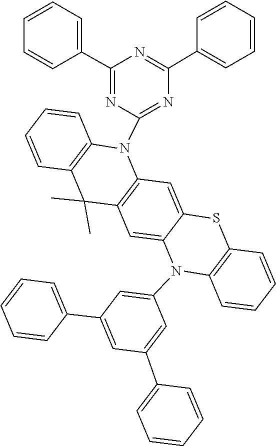 [SMILES: CC1(C)c2ccccc2N(c2nc(-c3ccccc3)nc(-c3ccccc3)n2)c2cc3c(cc21)N(c1cc(-c2ccccc2)cc(-c2ccccc2)c1)c1ccccc1S3]

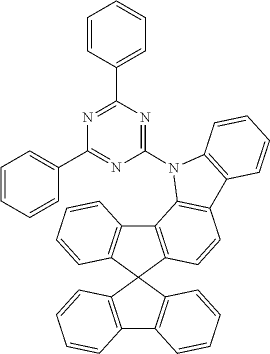 [SMILES: c1ccc(-c2nc(-c3ccccc3)nc(-n3c4ccccc4c4ccc5c(c43)-c3ccccc3C53c4ccccc4-c4ccccc43)n2)cc1]

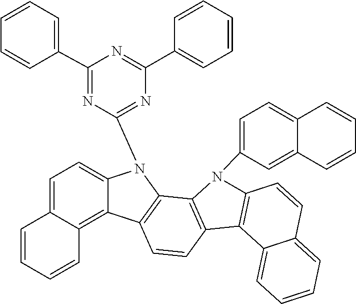 [SMILES: c1ccc(-c2nc(-c3ccccc3)nc(-n3c4ccc5ccccc5c4c4ccc5c6c7ccccc7ccc6n(-c6ccc7ccccc7c6)c5c43)n2)cc1]

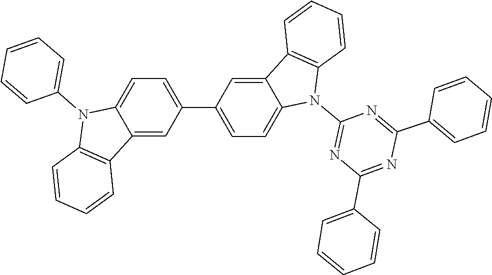 [SMILES: c1ccc(-c2nc(-c3ccccc3)nc(-n3c4ccccc4c4cc(-c5ccc6c(c5)c5ccccc5n6-c5ccccc5)ccc43)n2)cc1]

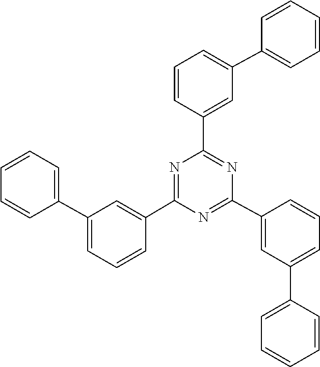 [SMILES: c1ccc(-c2cccc(-c3nc(-c4cccc(-c5ccccc5)c4)nc(-c4cccc(-c5ccccc5)c4)n3)c2)cc1]

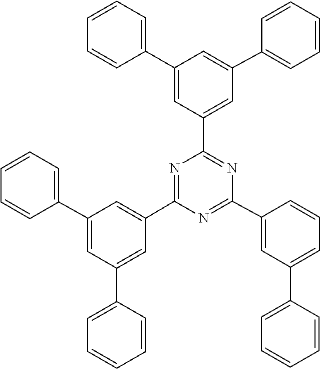 [SMILES: c1ccc(-c2cccc(-c3nc(-c4cc(-c5ccccc5)cc(-c5ccccc5)c4)nc(-c4cc(-c5ccccc5)cc(-c5ccccc5)c4)n3)c2)cc1]

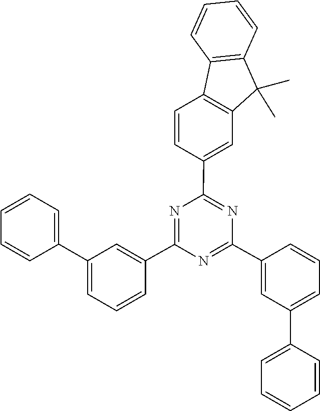 [SMILES: CC1(C)c2ccccc2-c2ccc(-c3nc(-c4cccc(-c5ccccc5)c4)nc(-c4cccc(-c5ccccc5)c4)n3)cc21]

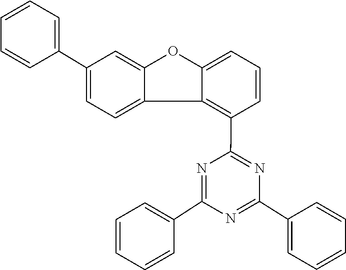 [SMILES: c1ccc(-c2ccc3c(c2)oc2cccc(-c4nc(-c5ccccc5)nc(-c5ccccc5)n4)c23)cc1]

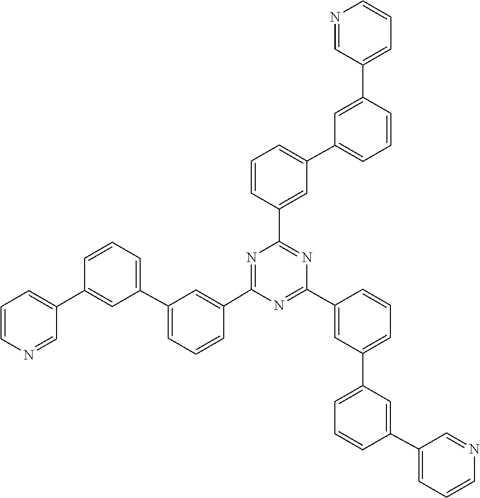 [SMILES: c1cncc(-c2cccc(-c3cccc(-c4nc(-c5cccc(-c6cccc(-c7cccnc7)c6)c5)nc(-c5cccc(-c6cccc(-c7cccnc7)c6)c5)n4)c3)c2)c1]

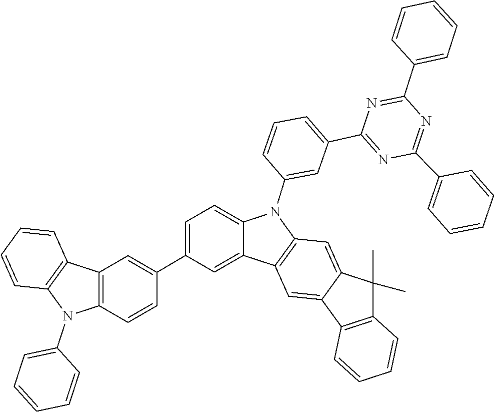 [SMILES: CC1(C)c2ccccc2-c2cc3c4cc(-c5ccc6c(c5)c5ccccc5n6-c5ccccc5)ccc4n(-c4cccc(-c5nc(-c6ccccc6)nc(-c6ccccc6)n5)c4)c3cc21]

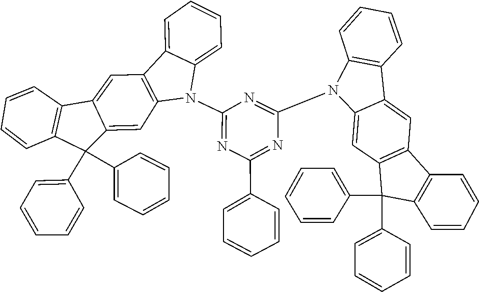 [SMILES: c1ccc(-c2nc(-n3c4ccccc4c4cc5c(cc43)C(c3ccccc3)(c3ccccc3)c3ccccc3-5)nc(-n3c4ccccc4c4cc5c(cc43)C(c3ccccc3)(c3ccccc3)c3ccccc3-5)n2)cc1]

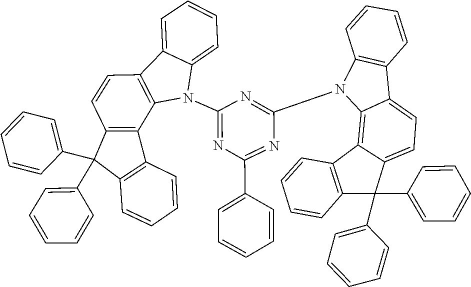 [SMILES: c1ccc(-c2nc(-n3c4ccccc4c4ccc5c(c43)-c3ccccc3C5(c3ccccc3)c3ccccc3)nc(-n3c4ccccc4c4ccc5c(c43)-c3ccccc3C5(c3ccccc3)c3ccccc3)n2)cc1]

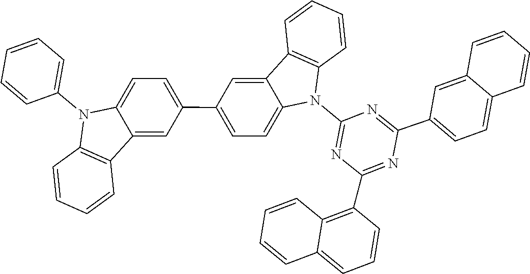 [SMILES: c1ccc(-n2c3ccccc3c3cc(-c4ccc5c(c4)c4ccccc4n5-c4nc(-c5ccc6ccccc6c5)nc(-c5cccc6ccccc56)n4)ccc32)cc1]

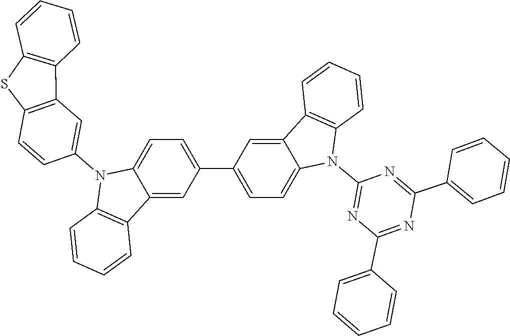 [SMILES: c1ccc(-c2nc(-c3ccccc3)nc(-n3c4ccccc4c4cc(-c5ccc6c(c5)c5ccccc5n6-c5ccc6sc7ccccc7c6c5)ccc43)n2)cc1]

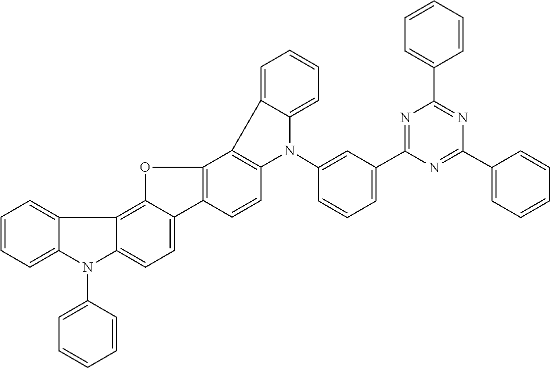 [SMILES: c1ccc(-c2nc(-c3ccccc3)nc(-c3cccc(-n4c5ccccc5c5c6oc7c(ccc8c7c7ccccc7n8-c7ccccc7)c6ccc54)c3)n2)cc1]

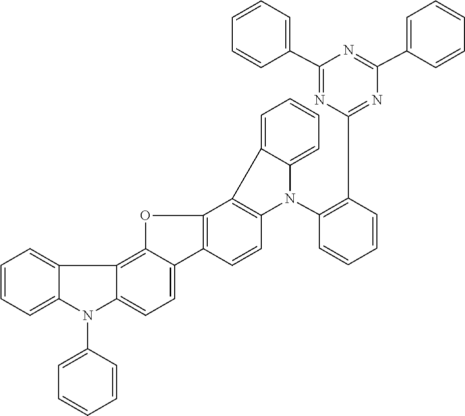 [SMILES: c1ccc(-c2nc(-c3ccccc3)nc(-c3ccccc3-n3c4ccccc4c4c5oc6c(ccc7c6c6ccccc6n7-c6ccccc6)c5ccc43)n2)cc1]

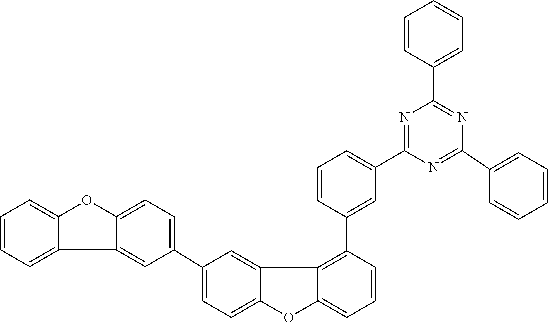 [SMILES: c1ccc(-c2nc(-c3ccccc3)nc(-c3cccc(-c4cccc5oc6ccc(-c7ccc8oc9ccccc9c8c7)cc6c45)c3)n2)cc1]

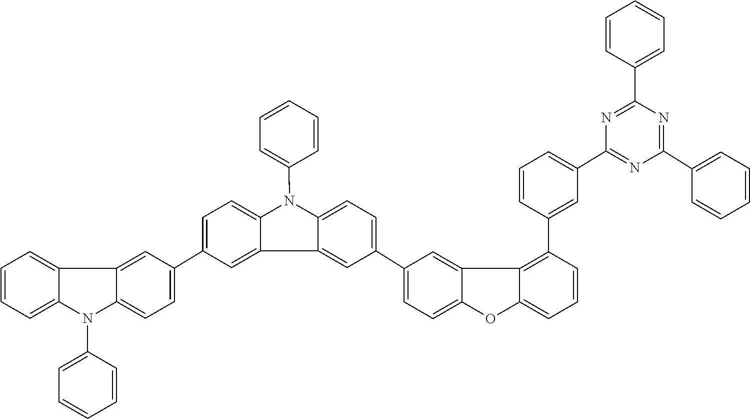 [SMILES: c1ccc(-c2nc(-c3ccccc3)nc(-c3cccc(-c4cccc5oc6ccc(-c7ccc8c(c7)c7cc(-c9ccc%10c(c9)c9ccccc9n%10-c9ccccc9)ccc7n8-c7ccccc7)cc6c45)c3)n2)cc1]

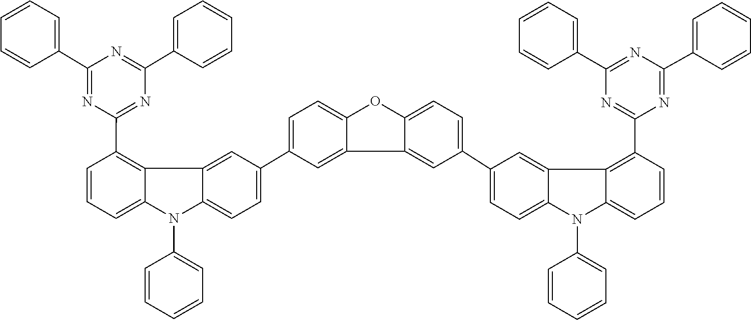 [SMILES: c1ccc(-c2nc(-c3ccccc3)nc(-c3cccc4c3c3cc(-c5ccc6oc7ccc(-c8ccc9c(c8)c8c(-c%10nc(-c%11ccccc%11)nc(-c%11ccccc%11)n%10)cccc8n9-c8ccccc8)cc7c6c5)ccc3n4-c3ccccc3)n2)cc1]

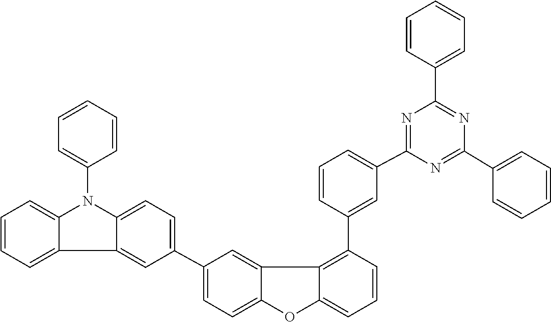 [SMILES: c1ccc(-c2nc(-c3ccccc3)nc(-c3cccc(-c4cccc5oc6ccc(-c7ccc8c(c7)c7ccccc7n8-c7ccccc7)cc6c45)c3)n2)cc1]

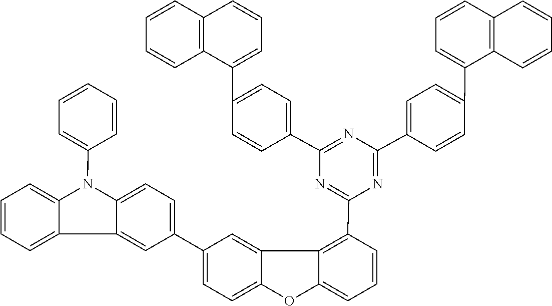 [SMILES: c1ccc(-n2c3ccccc3c3cc(-c4ccc5oc6cccc(-c7nc(-c8ccc(-c9cccc%10ccccc9%10)cc8)nc(-c8ccc(-c9cccc%10ccccc9%10)cc8)n7)c6c5c4)ccc32)cc1]

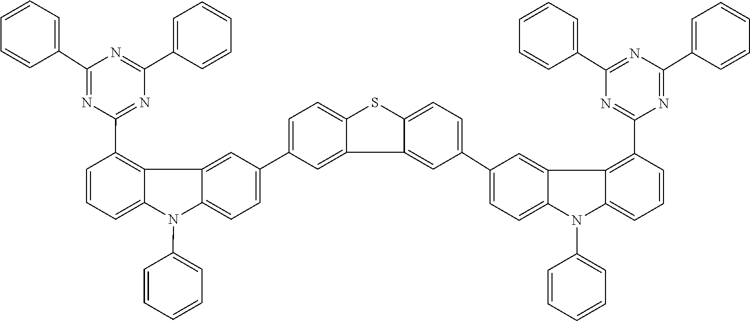 [SMILES: c1ccc(-c2nc(-c3ccccc3)nc(-c3cccc4c3c3cc(-c5ccc6sc7ccc(-c8ccc9c(c8)c8c(-c%10nc(-c%11ccccc%11)nc(-c%11ccccc%11)n%10)cccc8n9-c8ccccc8)cc7c6c5)ccc3n4-c3ccccc3)n2)cc1]

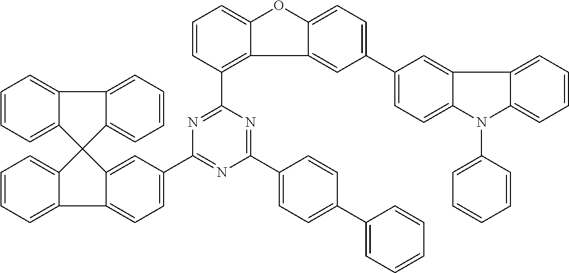 [SMILES: c1ccc(-c2ccc(-c3nc(-c4ccc5c(c4)C4(c6ccccc6-c6ccccc64)c4ccccc4-5)nc(-c4cccc5oc6ccc(-c7ccc8c(c7)c7ccccc7n8-c7ccccc7)cc6c45)n3)cc2)cc1]

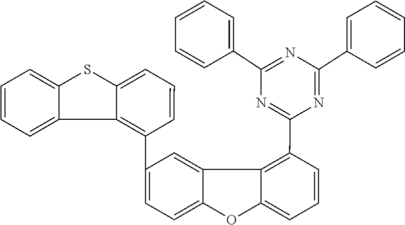 [SMILES: c1ccc(-c2nc(-c3ccccc3)nc(-c3cccc4oc5ccc(-c6cccc7sc8ccccc8c67)cc5c34)n2)cc1]

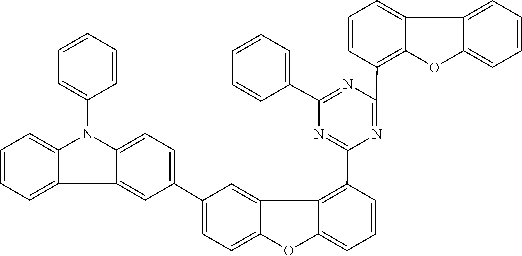 [SMILES: c1ccc(-c2nc(-c3cccc4c3oc3ccccc34)nc(-c3cccc4oc5ccc(-c6ccc7c(c6)c6ccccc6n7-c6ccccc6)cc5c34)n2)cc1]

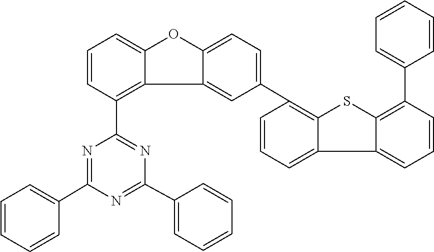 [SMILES: c1ccc(-c2nc(-c3ccccc3)nc(-c3cccc4oc5ccc(-c6cccc7c6sc6c(-c8ccccc8)cccc67)cc5c34)n2)cc1]

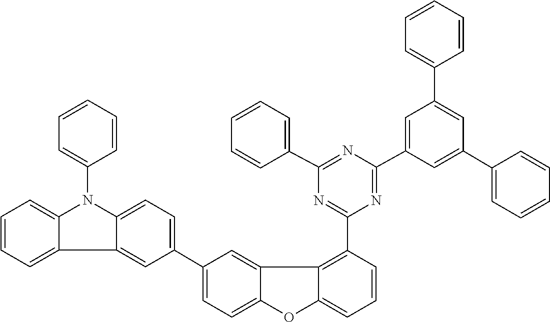 [SMILES: c1ccc(-c2cc(-c3ccccc3)cc(-c3nc(-c4ccccc4)nc(-c4cccc5oc6ccc(-c7ccc8c(c7)c7ccccc7n8-c7ccccc7)cc6c45)n3)c2)cc1]

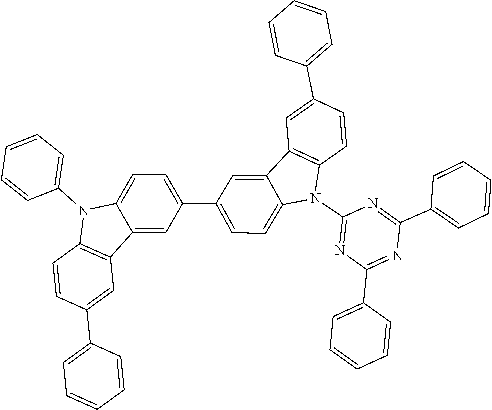 [SMILES: c1ccc(-c2ccc3c(c2)c2cc(-c4ccc5c(c4)c4cc(-c6ccccc6)ccc4n5-c4nc(-c5ccccc5)nc(-c5ccccc5)n4)ccc2n3-c2ccccc2)cc1]

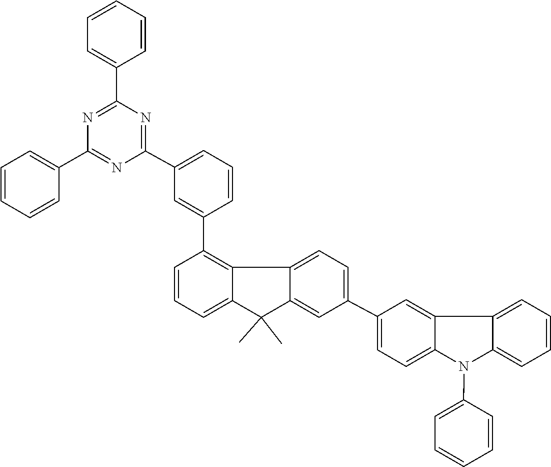 [SMILES: CC1(C)c2cc(-c3ccc4c(c3)c3ccccc3n4-c3ccccc3)ccc2-c2c(-c3cccc(-c4nc(-c5ccccc5)nc(-c5ccccc5)n4)c3)cccc21]

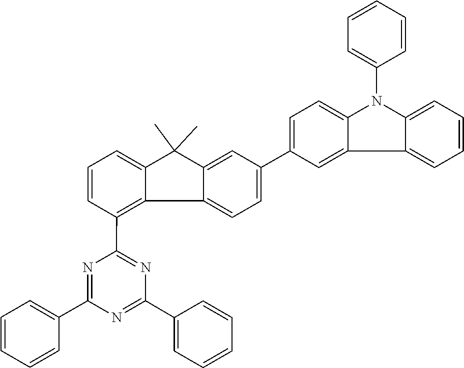 [SMILES: CC1(C)c2cc(-c3ccc4c(c3)c3ccccc3n4-c3ccccc3)ccc2-c2c(-c3nc(-c4ccccc4)nc(-c4ccccc4)n3)cccc21]

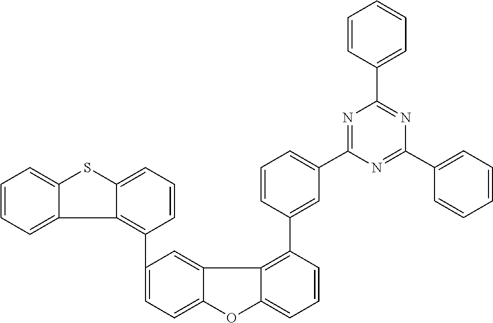 [SMILES: c1ccc(-c2nc(-c3ccccc3)nc(-c3cccc(-c4cccc5oc6ccc(-c7cccc8sc9ccccc9c78)cc6c45)c3)n2)cc1]

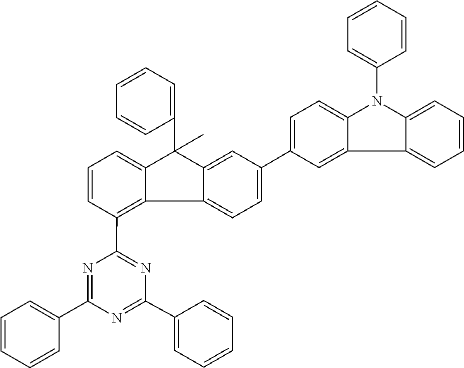 [SMILES: CC1(c2ccccc2)c2cc(-c3ccc4c(c3)c3ccccc3n4-c3ccccc3)ccc2-c2c(-c3nc(-c4ccccc4)nc(-c4ccccc4)n3)cccc21]

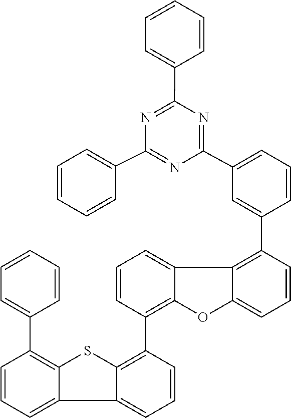 [SMILES: c1ccc(-c2nc(-c3ccccc3)nc(-c3cccc(-c4cccc5oc6c(-c7cccc8c7sc7c(-c9ccccc9)cccc78)cccc6c45)c3)n2)cc1]

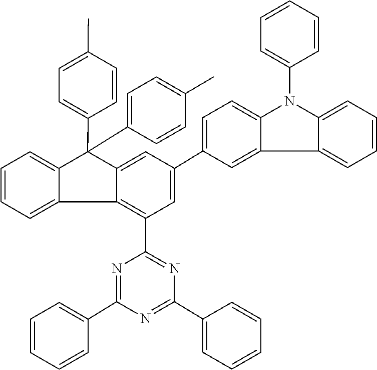 [SMILES: Cc1ccc(C2(c3ccc(C)cc3)c3ccccc3-c3c(-c4nc(-c5ccccc5)nc(-c5ccccc5)n4)cc(-c4ccc5c(c4)c4ccccc4n5-c4ccccc4)cc32)cc1]

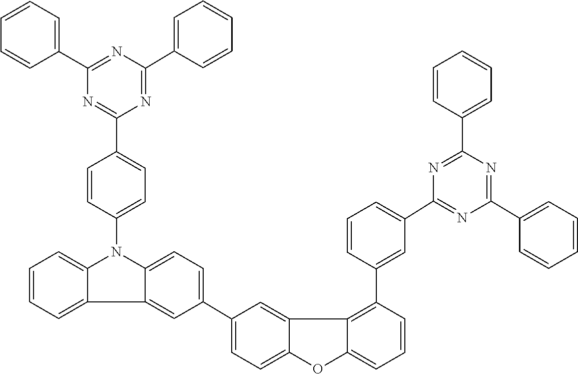 [SMILES: c1ccc(-c2nc(-c3ccccc3)nc(-c3ccc(-n4c5ccccc5c5cc(-c6ccc7oc8cccc(-c9cccc(-c%10nc(-c%11ccccc%11)nc(-c%11ccccc%11)n%10)c9)c8c7c6)ccc54)cc3)n2)cc1]

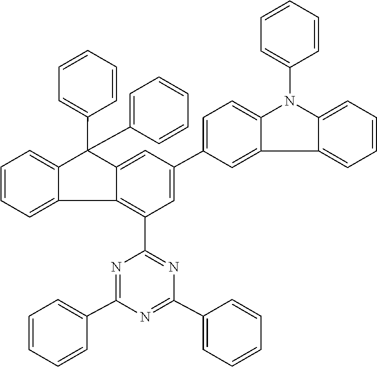 [SMILES: c1ccc(-c2nc(-c3ccccc3)nc(-c3cc(-c4ccc5c(c4)c4ccccc4n5-c4ccccc4)cc4c3-c3ccccc3C4(c3ccccc3)c3ccccc3)n2)cc1]

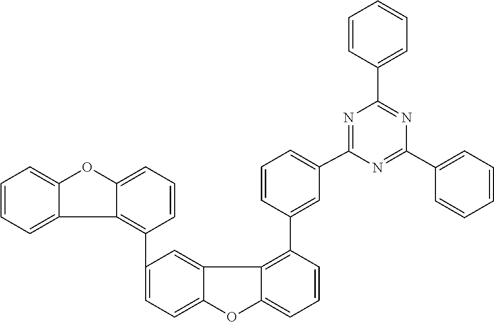 [SMILES: c1ccc(-c2nc(-c3ccccc3)nc(-c3cccc(-c4cccc5oc6ccc(-c7cccc8oc9ccccc9c78)cc6c45)c3)n2)cc1]

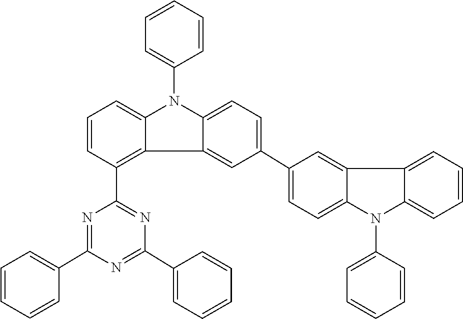 [SMILES: c1ccc(-c2nc(-c3ccccc3)nc(-c3cccc4c3c3cc(-c5ccc6c(c5)c5ccccc5n6-c5ccccc5)ccc3n4-c3ccccc3)n2)cc1]

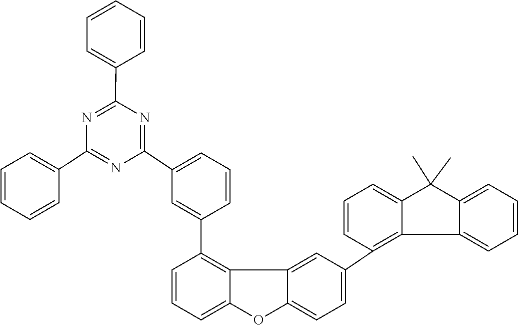 [SMILES: CC1(C)c2ccccc2-c2c(-c3ccc4oc5cccc(-c6cccc(-c7nc(-c8ccccc8)nc(-c8ccccc8)n7)c6)c5c4c3)cccc21]

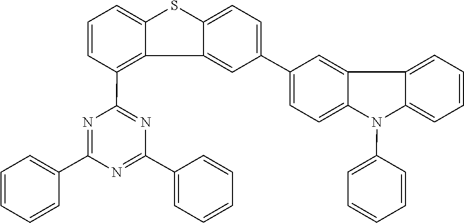 [SMILES: c1ccc(-c2nc(-c3ccccc3)nc(-c3cccc4sc5ccc(-c6ccc7c(c6)c6ccccc6n7-c6ccccc6)cc5c34)n2)cc1]

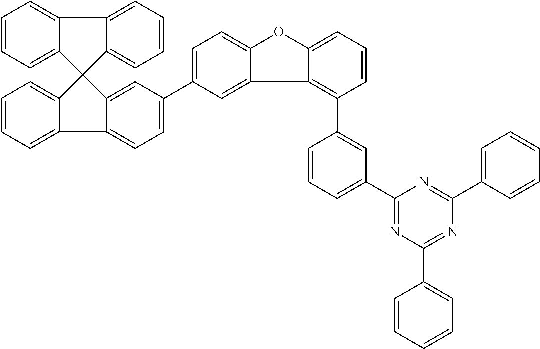 [SMILES: c1ccc(-c2nc(-c3ccccc3)nc(-c3cccc(-c4cccc5oc6ccc(-c7ccc8c(c7)C7(c9ccccc9-c9ccccc97)c7ccccc7-8)cc6c45)c3)n2)cc1]

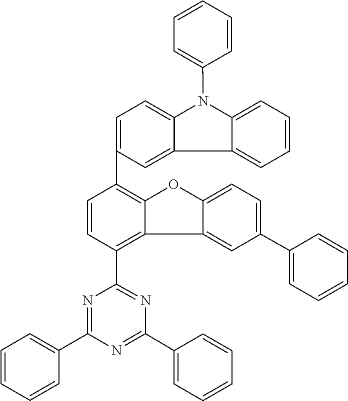 [SMILES: c1ccc(-c2ccc3oc4c(-c5ccc6c(c5)c5ccccc5n6-c5ccccc5)ccc(-c5nc(-c6ccccc6)nc(-c6ccccc6)n5)c4c3c2)cc1]

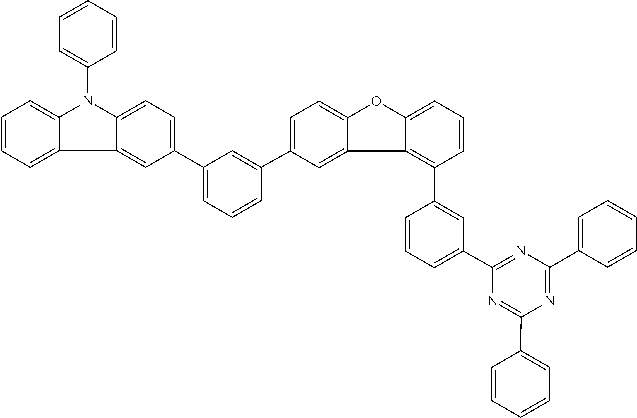 [SMILES: c1ccc(-c2nc(-c3ccccc3)nc(-c3cccc(-c4cccc5oc6ccc(-c7cccc(-c8ccc9c(c8)c8ccccc8n9-c8ccccc8)c7)cc6c45)c3)n2)cc1]

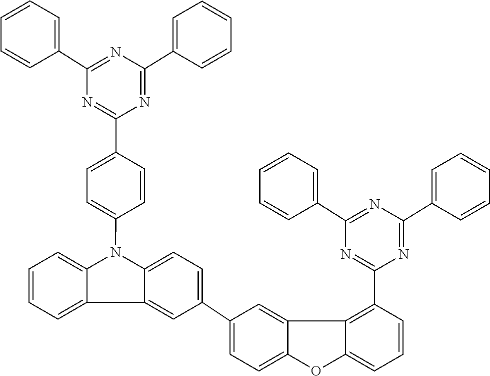 [SMILES: c1ccc(-c2nc(-c3ccccc3)nc(-c3ccc(-n4c5ccccc5c5cc(-c6ccc7oc8cccc(-c9nc(-c%10ccccc%10)nc(-c%10ccccc%10)n9)c8c7c6)ccc54)cc3)n2)cc1]

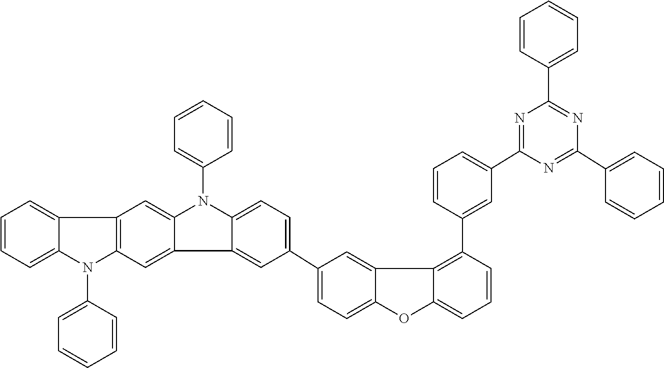 [SMILES: c1ccc(-c2nc(-c3ccccc3)nc(-c3cccc(-c4cccc5oc6ccc(-c7ccc8c(c7)c7cc9c(cc7n8-c7ccccc7)c7ccccc7n9-c7ccccc7)cc6c45)c3)n2)cc1]

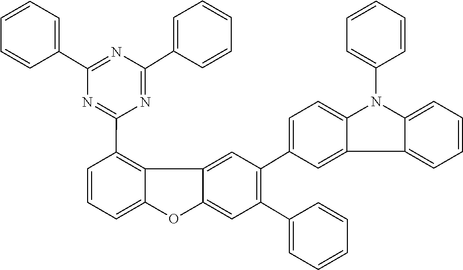 [SMILES: c1ccc(-c2nc(-c3ccccc3)nc(-c3cccc4oc5cc(-c6ccccc6)c(-c6ccc7c(c6)c6ccccc6n7-c6ccccc6)cc5c34)n2)cc1]

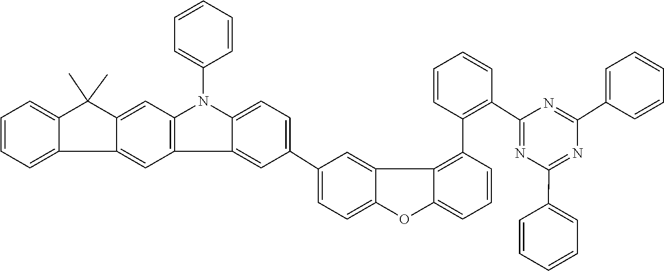 [SMILES: CC1(C)c2ccccc2-c2cc3c4cc(-c5ccc6oc7cccc(-c8ccccc8-c8nc(-c9ccccc9)nc(-c9ccccc9)n8)c7c6c5)ccc4n(-c4ccccc4)c3cc21]

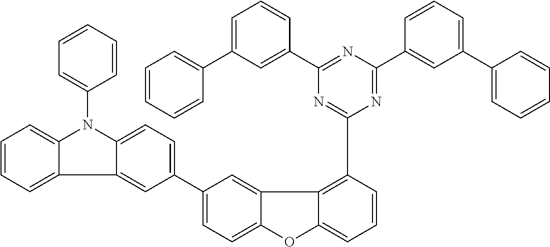 [SMILES: c1ccc(-c2cccc(-c3nc(-c4cccc(-c5ccccc5)c4)nc(-c4cccc5oc6ccc(-c7ccc8c(c7)c7ccccc7n8-c7ccccc7)cc6c45)n3)c2)cc1]

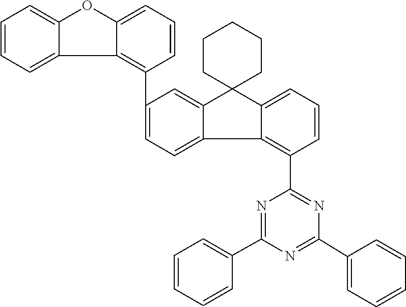 [SMILES: c1ccc(-c2nc(-c3ccccc3)nc(-c3cccc4c3-c3ccc(-c5cccc6oc7ccccc7c56)cc3C43CCCCC3)n2)cc1]

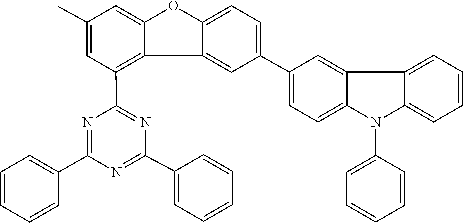 [SMILES: Cc1cc(-c2nc(-c3ccccc3)nc(-c3ccccc3)n2)c2c(c1)oc1ccc(-c3ccc4c(c3)c3ccccc3n4-c3ccccc3)cc12]

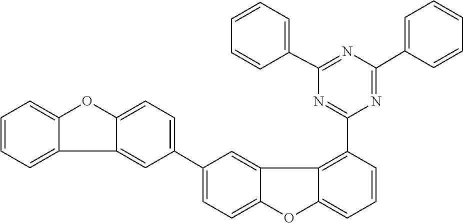 [SMILES: c1ccc(-c2nc(-c3ccccc3)nc(-c3cccc4oc5ccc(-c6ccc7oc8ccccc8c7c6)cc5c34)n2)cc1]

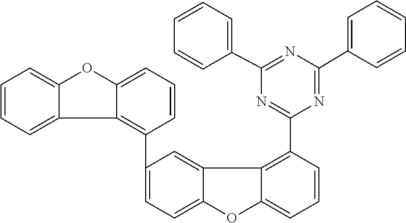 [SMILES: c1ccc(-c2nc(-c3ccccc3)nc(-c3cccc4oc5ccc(-c6cccc7oc8ccccc8c67)cc5c34)n2)cc1]

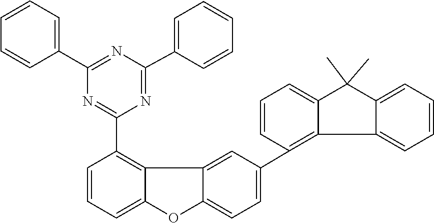 [SMILES: CC1(C)c2ccccc2-c2c(-c3ccc4oc5cccc(-c6nc(-c7ccccc7)nc(-c7ccccc7)n6)c5c4c3)cccc21]

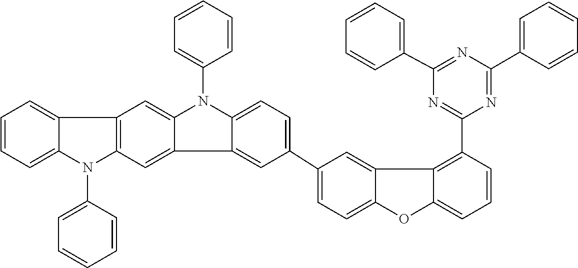 [SMILES: c1ccc(-c2nc(-c3ccccc3)nc(-c3cccc4oc5ccc(-c6ccc7c(c6)c6cc8c(cc6n7-c6ccccc6)c6ccccc6n8-c6ccccc6)cc5c34)n2)cc1]

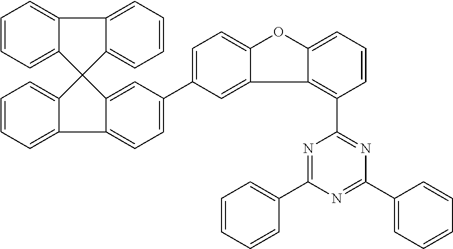 [SMILES: c1ccc(-c2nc(-c3ccccc3)nc(-c3cccc4oc5ccc(-c6ccc7c(c6)C6(c8ccccc8-c8ccccc86)c6ccccc6-7)cc5c34)n2)cc1]

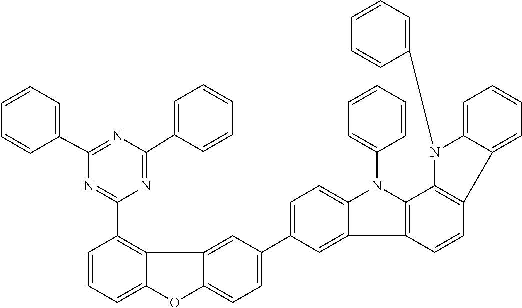 [SMILES: c1ccc(-c2nc(-c3ccccc3)nc(-c3cccc4oc5ccc(-c6ccc7c(c6)c6ccc8c9ccccc9n(-c9ccccc9)c8c6n7-c6ccccc6)cc5c34)n2)cc1]

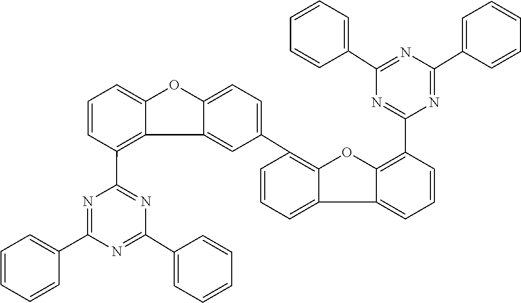 [SMILES: c1ccc(-c2nc(-c3ccccc3)nc(-c3cccc4c3oc3c(-c5ccc6oc7cccc(-c8nc(-c9ccccc9)nc(-c9ccccc9)n8)c7c6c5)cccc34)n2)cc1]

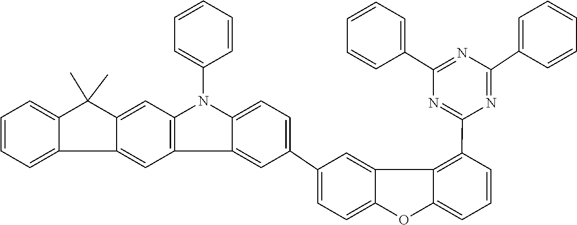 [SMILES: CC1(C)c2ccccc2-c2cc3c4cc(-c5ccc6oc7cccc(-c8nc(-c9ccccc9)nc(-c9ccccc9)n8)c7c6c5)ccc4n(-c4ccccc4)c3cc21]

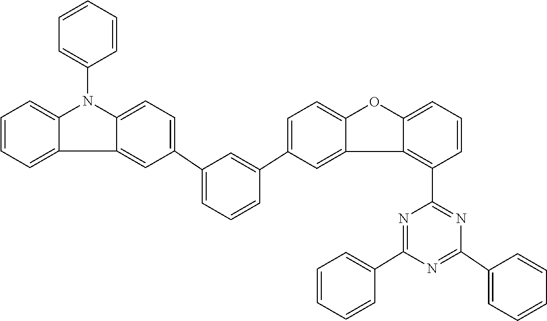 [SMILES: c1ccc(-c2nc(-c3ccccc3)nc(-c3cccc4oc5ccc(-c6cccc(-c7ccc8c(c7)c7ccccc7n8-c7ccccc7)c6)cc5c34)n2)cc1]

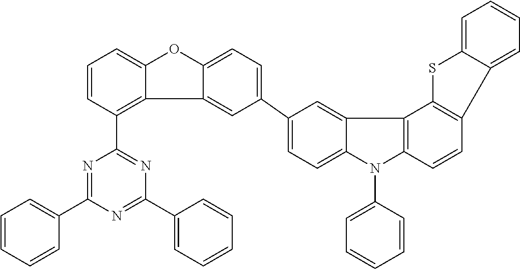 [SMILES: c1ccc(-c2nc(-c3ccccc3)nc(-c3cccc4oc5ccc(-c6ccc7c(c6)c6c8sc9ccccc9c8ccc6n7-c6ccccc6)cc5c34)n2)cc1]

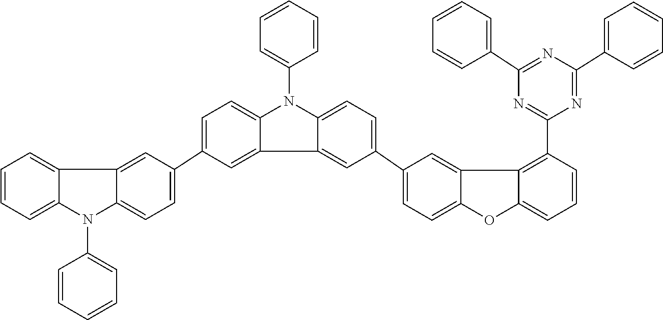 [SMILES: c1ccc(-c2nc(-c3ccccc3)nc(-c3cccc4oc5ccc(-c6ccc7c(c6)c6cc(-c8ccc9c(c8)c8ccccc8n9-c8ccccc8)ccc6n7-c6ccccc6)cc5c34)n2)cc1]

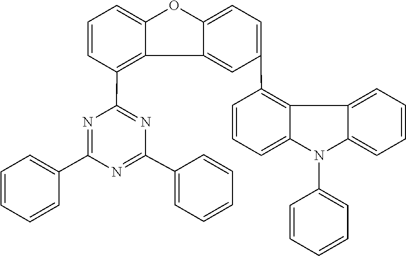 [SMILES: c1ccc(-c2nc(-c3ccccc3)nc(-c3cccc4oc5ccc(-c6cccc7c6c6ccccc6n7-c6ccccc6)cc5c34)n2)cc1]

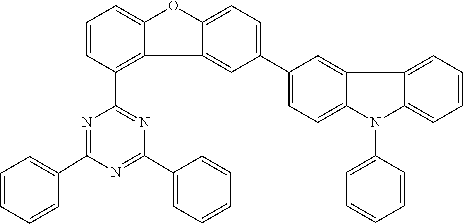 [SMILES: c1ccc(-c2nc(-c3ccccc3)nc(-c3cccc4oc5ccc(-c6ccc7c(c6)c6ccccc6n7-c6ccccc6)cc5c34)n2)cc1]

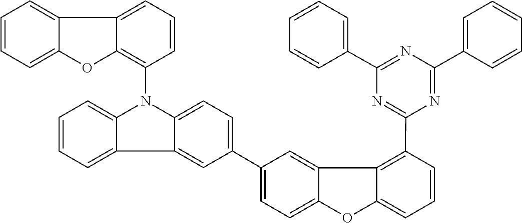 [SMILES: c1ccc(-c2nc(-c3ccccc3)nc(-c3cccc4oc5ccc(-c6ccc7c(c6)c6ccccc6n7-c6cccc7c6oc6ccccc67)cc5c34)n2)cc1]

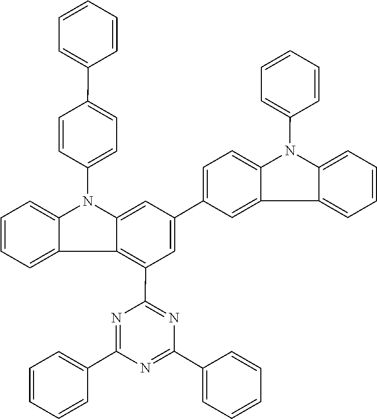 [SMILES: c1ccc(-c2ccc(-n3c4ccccc4c4c(-c5nc(-c6ccccc6)nc(-c6ccccc6)n5)cc(-c5ccc6c(c5)c5ccccc5n6-c5ccccc5)cc43)cc2)cc1]

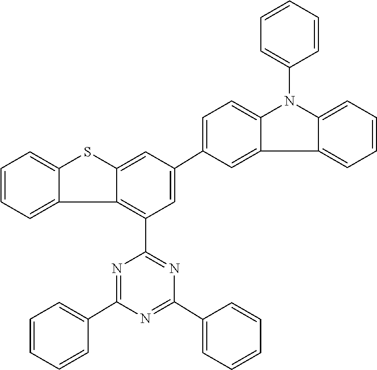 [SMILES: c1ccc(-c2nc(-c3ccccc3)nc(-c3cc(-c4ccc5c(c4)c4ccccc4n5-c4ccccc4)cc4sc5ccccc5c34)n2)cc1]

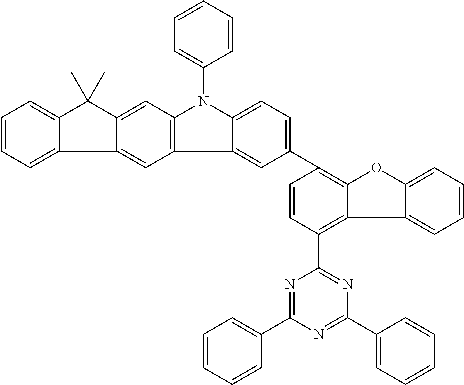 [SMILES: CC1(C)c2ccccc2-c2cc3c4cc(-c5ccc(-c6nc(-c7ccccc7)nc(-c7ccccc7)n6)c6c5oc5ccccc56)ccc4n(-c4ccccc4)c3cc21]

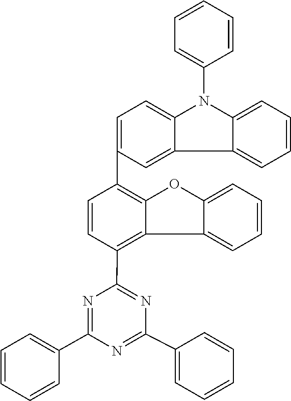 [SMILES: c1ccc(-c2nc(-c3ccccc3)nc(-c3ccc(-c4ccc5c(c4)c4ccccc4n5-c4ccccc4)c4oc5ccccc5c34)n2)cc1]

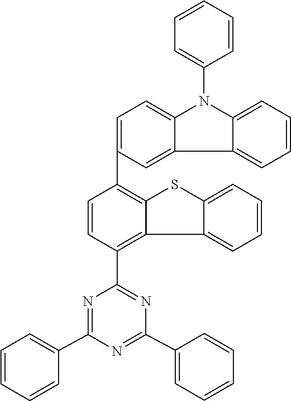 [SMILES: c1ccc(-c2nc(-c3ccccc3)nc(-c3ccc(-c4ccc5c(c4)c4ccccc4n5-c4ccccc4)c4sc5ccccc5c34)n2)cc1]

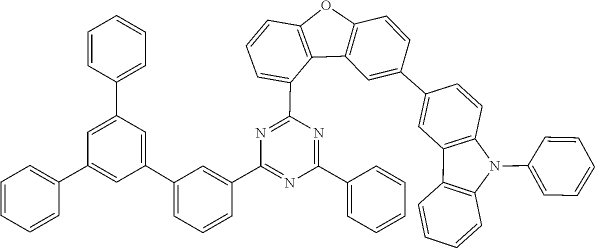 [SMILES: c1ccc(-c2cc(-c3ccccc3)cc(-c3cccc(-c4nc(-c5ccccc5)nc(-c5cccc6oc7ccc(-c8ccc9c(c8)c8ccccc8n9-c8ccccc8)cc7c56)n4)c3)c2)cc1]

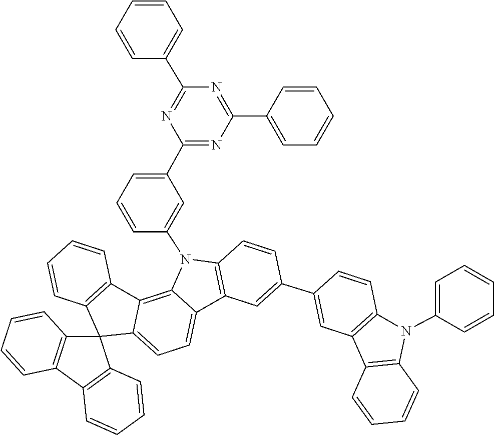 [SMILES: c1ccc(-c2nc(-c3ccccc3)nc(-c3cccc(-n4c5ccc(-c6ccc7c(c6)c6ccccc6n7-c6ccccc6)cc5c5ccc6c(c54)-c4ccccc4C64c5ccccc5-c5ccccc54)c3)n2)cc1]

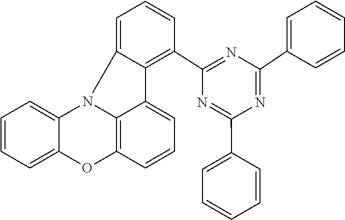 [SMILES: c1ccc(-c2nc(-c3ccccc3)nc(-c3cccc4c3c3cccc5c3n4-c3ccccc3O5)n2)cc1]

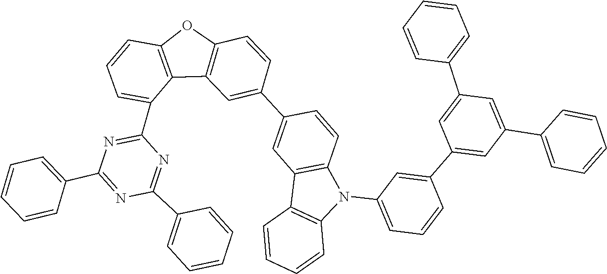 [SMILES: c1ccc(-c2cc(-c3ccccc3)cc(-c3cccc(-n4c5ccccc5c5cc(-c6ccc7oc8cccc(-c9nc(-c%10ccccc%10)nc(-c%10ccccc%10)n9)c8c7c6)ccc54)c3)c2)cc1]

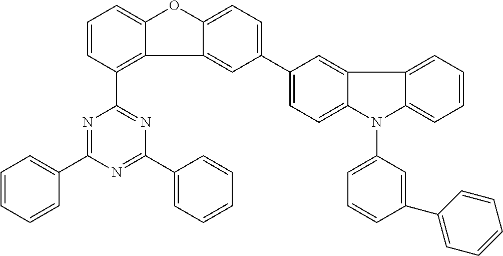 [SMILES: c1ccc(-c2cccc(-n3c4ccccc4c4cc(-c5ccc6oc7cccc(-c8nc(-c9ccccc9)nc(-c9ccccc9)n8)c7c6c5)ccc43)c2)cc1]

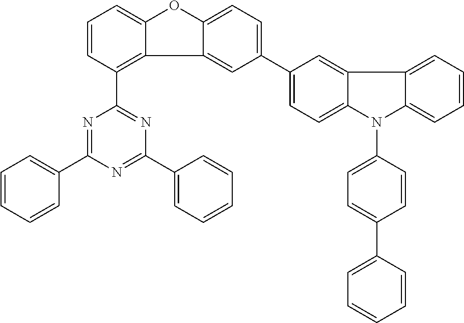 [SMILES: c1ccc(-c2ccc(-n3c4ccccc4c4cc(-c5ccc6oc7cccc(-c8nc(-c9ccccc9)nc(-c9ccccc9)n8)c7c6c5)ccc43)cc2)cc1]

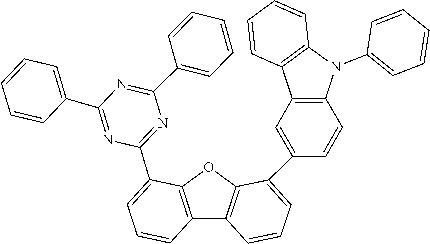 [SMILES: c1ccc(-c2nc(-c3ccccc3)nc(-c3cccc4c3oc3c(-c5ccc6c(c5)c5ccccc5n6-c5ccccc5)cccc34)n2)cc1]

Examples of suitable quinazoline compounds are the compounds depicted in the following table:

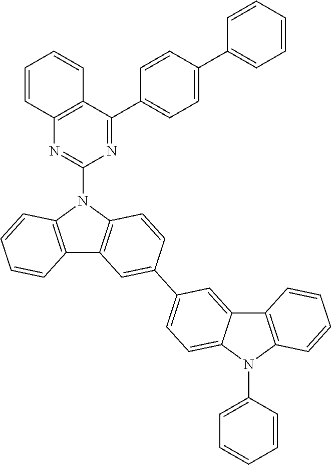 [SMILES: c1ccc(-c2ccc(-c3nc(-n4c5ccccc5c5cc(-c6ccc7c(c6)c6ccccc6n7-c6ccccc6)ccc54)nc4ccccc34)cc2)cc1]

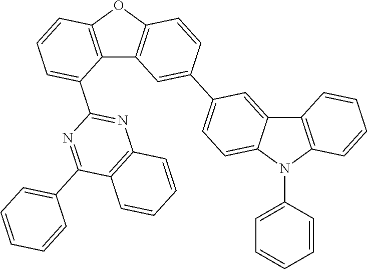 [SMILES: c1ccc(-c2nc(-c3cccc4oc5ccc(-c6ccc7c(c6)c6ccccc6n7-c6ccccc6)cc5c34)nc3ccccc23)cc1]

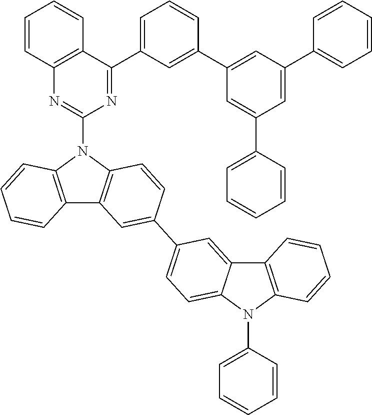 [SMILES: c1ccc(-c2cc(-c3ccccc3)cc(-c3cccc(-c4nc(-n5c6ccccc6c6cc(-c7ccc8c(c7)c7ccccc7n8-c7ccccc7)ccc65)nc5ccccc45)c3)c2)cc1]

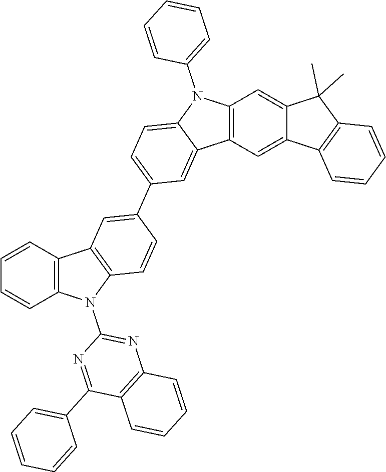 [SMILES: CC1(C)c2ccccc2-c2cc3c4cc(-c5ccc6c(c5)c5ccccc5n6-c5nc(-c6ccccc6)c6ccccc6n5)ccc4n(-c4ccccc4)c3cc21]

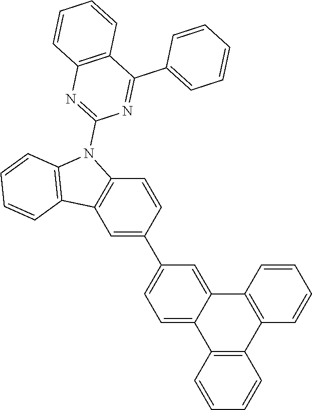 [SMILES: c1ccc(-c2nc(-n3c4ccccc4c4cc(-c5ccc6c7ccccc7c7ccccc7c6c5)ccc43)nc3ccccc23)cc1]

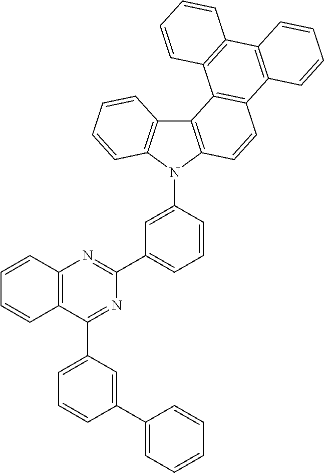 [SMILES: c1ccc(-c2cccc(-c3nc(-c4cccc(-n5c6ccccc6c6c7c8ccccc8c8ccccc8c7ccc65)c4)nc4ccccc34)c2)cc1]

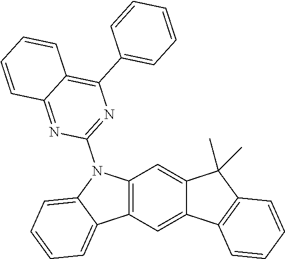 [SMILES: CC1(C)c2ccccc2-c2cc3c4ccccc4n(-c4nc(-c5ccccc5)c5ccccc5n4)c3cc21]

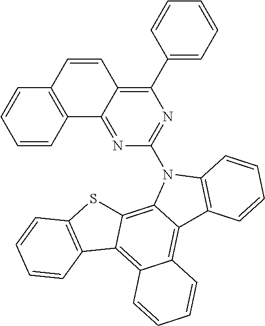 [SMILES: c1ccc(-c2nc(-n3c4ccccc4c4c5ccccc5c5c6ccccc6sc5c43)nc3c2ccc2ccccc23)cc1]

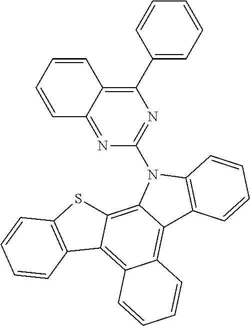 [SMILES: c1ccc(-c2nc(-n3c4ccccc4c4c5ccccc5c5c6ccccc6sc5c43)nc3ccccc23)cc1]

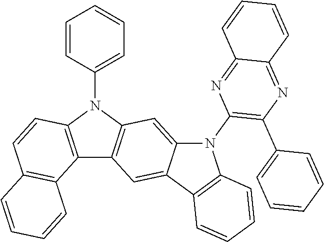 [SMILES: c1ccc(-c2nc3ccccc3nc2-n2c3ccccc3c3cc4c5c6ccccc6ccc5n(-c5ccccc5)c4cc32)cc1]

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the emitters described above can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439, WO 2018/011186, WO 2018/041769, WO 2019/020538, WO 2018/178001, WO 2019/115423 and WO 2019/158453. In general, all phosphorescent complexes as used for phosphorescent OLEDs according to the prior art and as known to those skilled in the art in the field of organic electroluminescence are suitable, and the person skilled in the art will be able to use further phosphorescent complexes without exercising inventive skill.

Examples of phosphorescent dopants are adduced below.

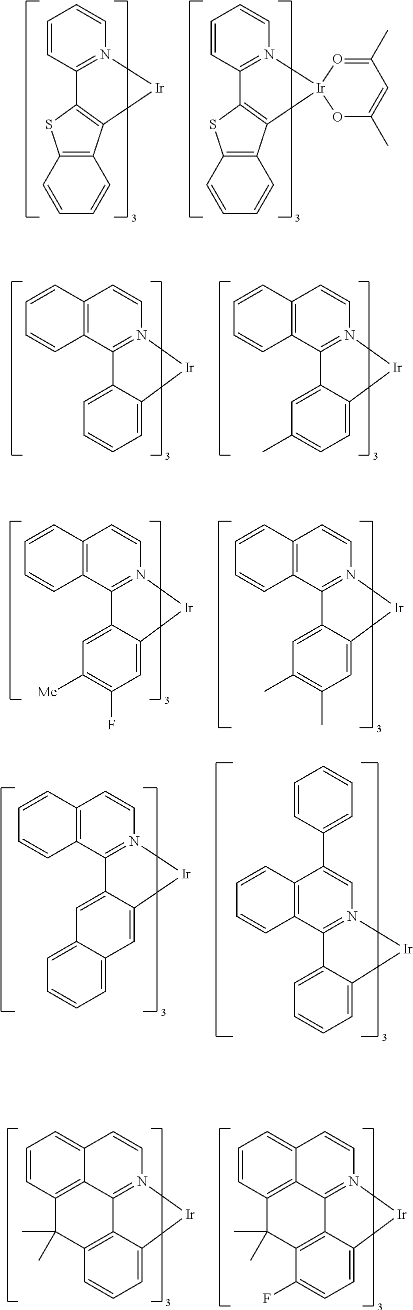

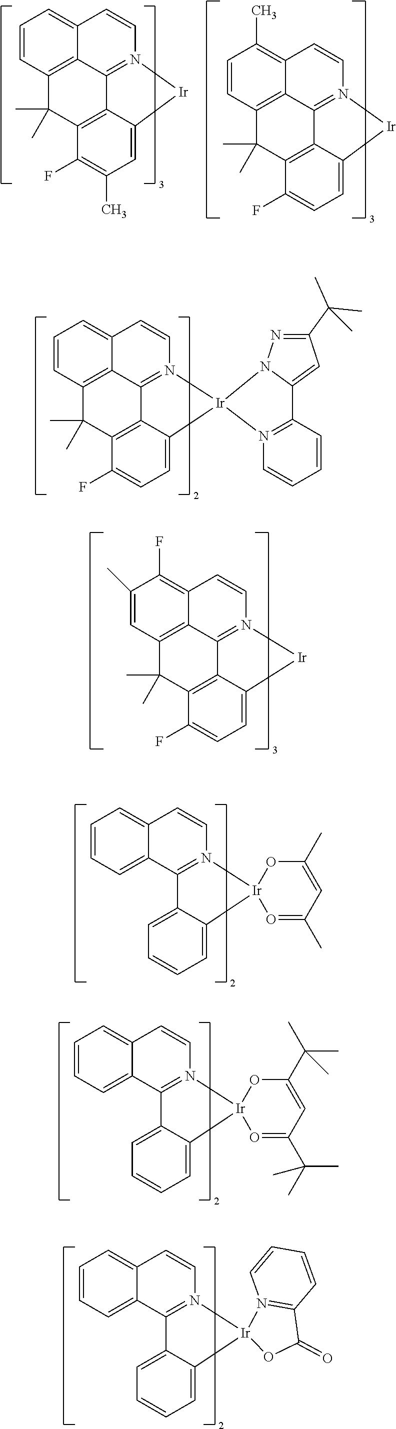

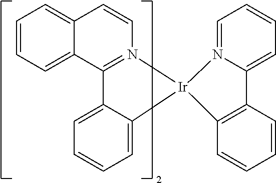

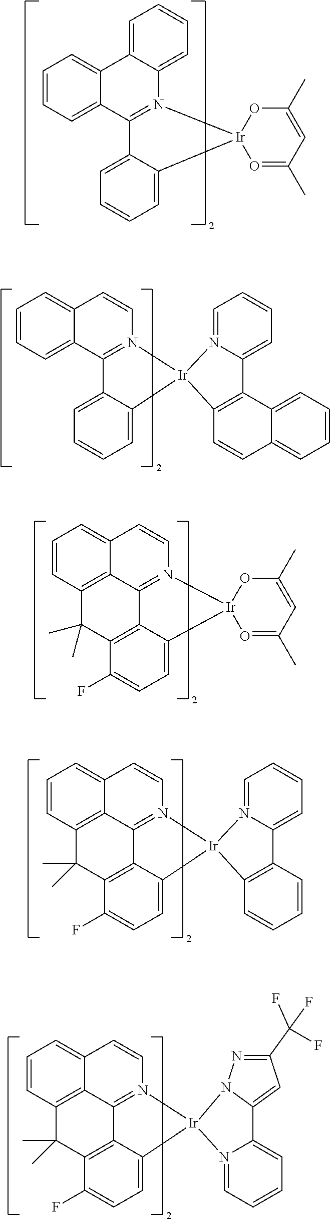

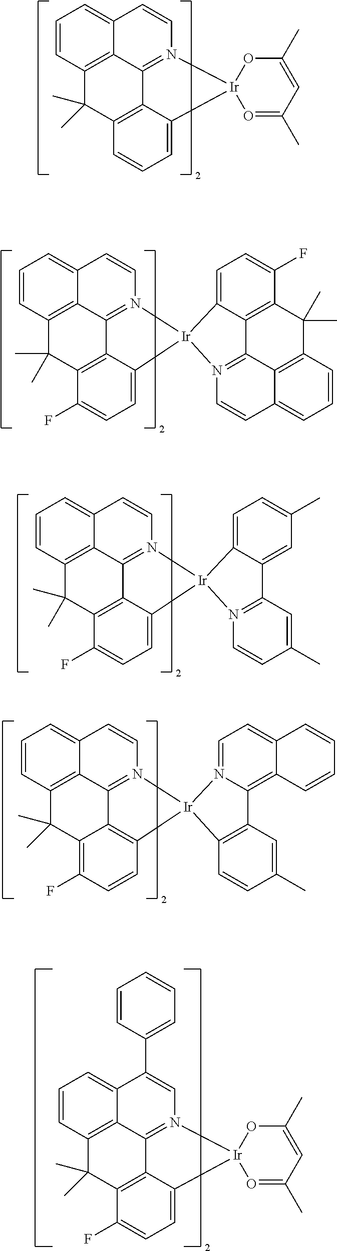

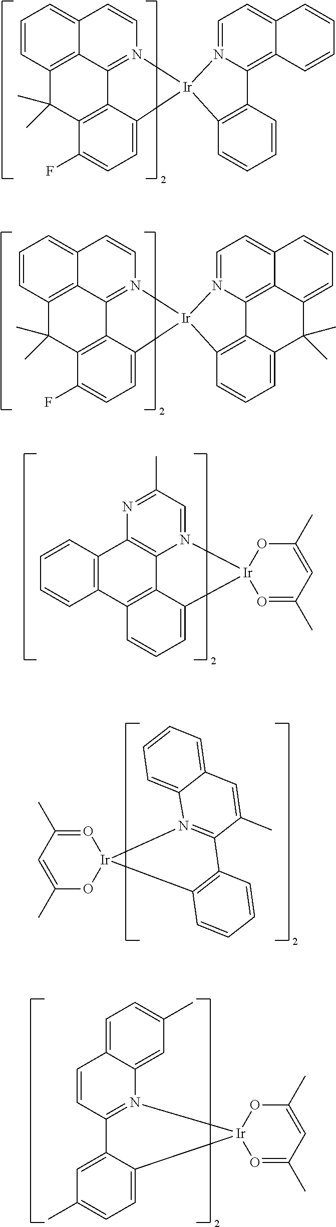

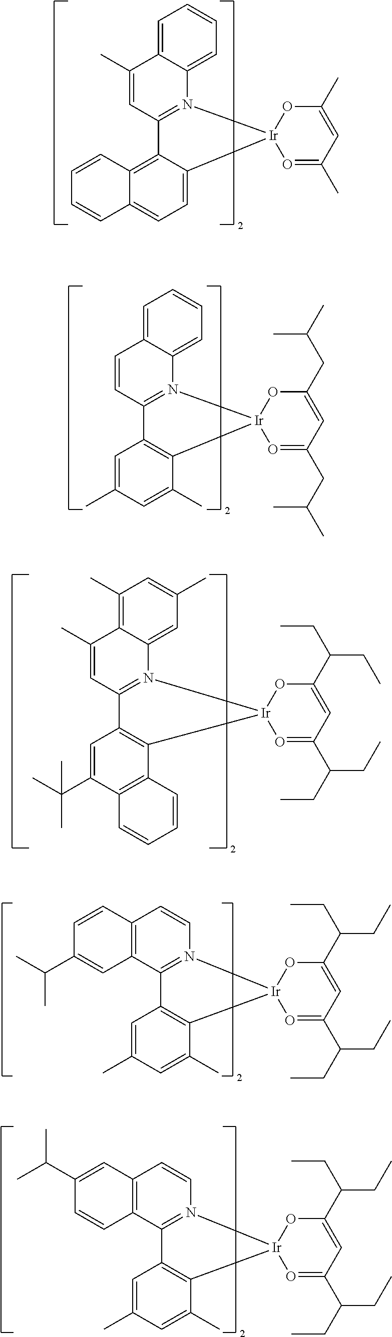

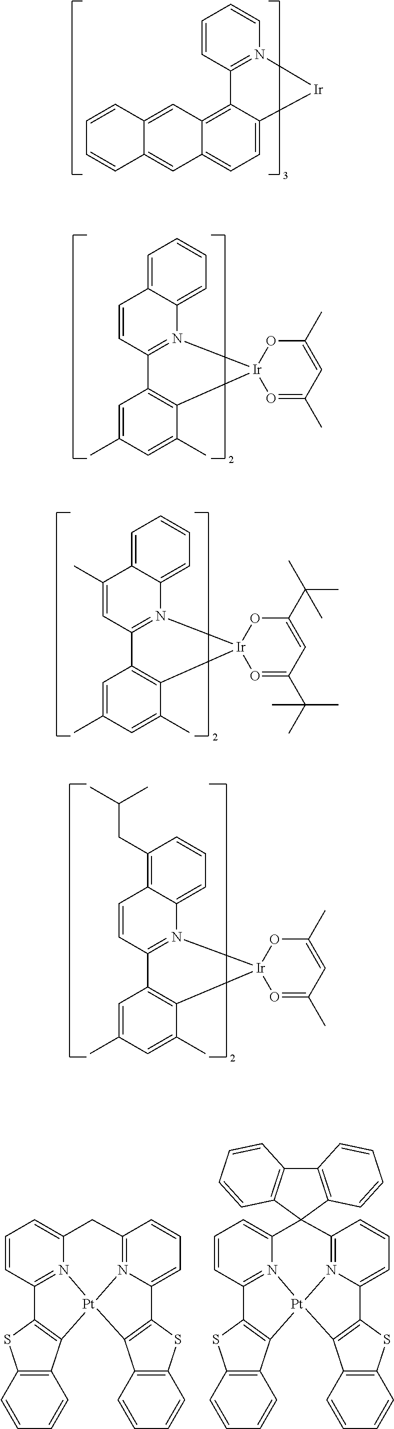

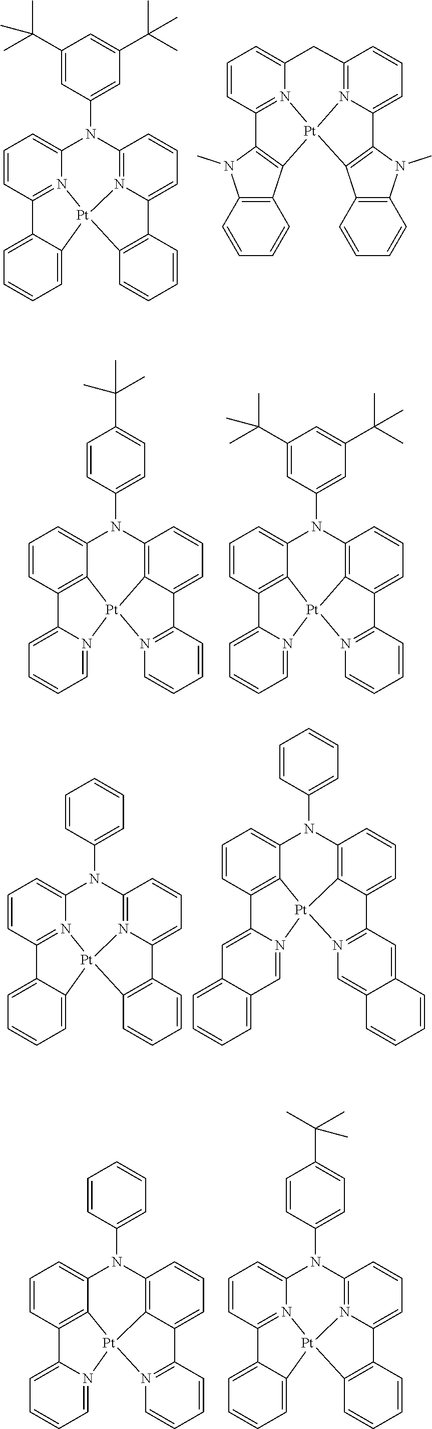

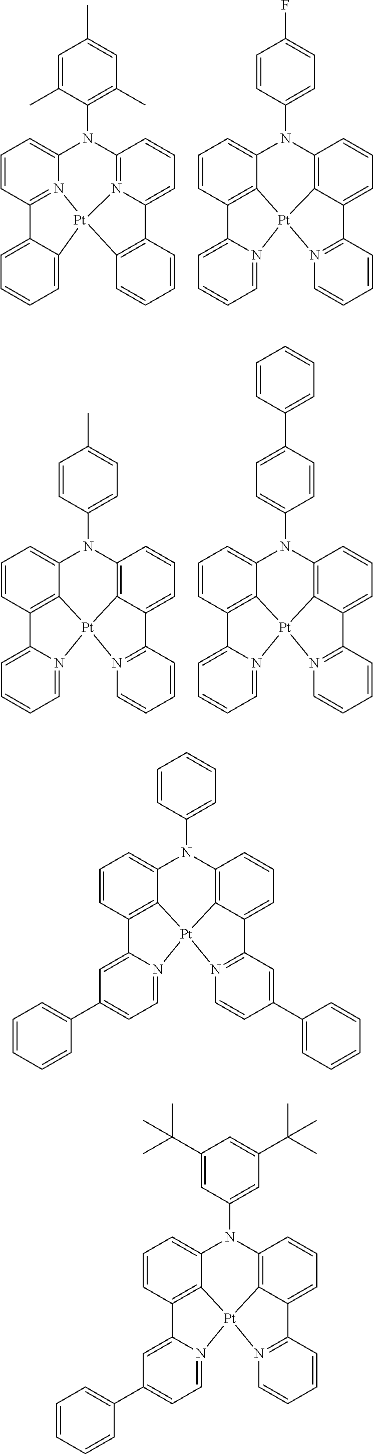

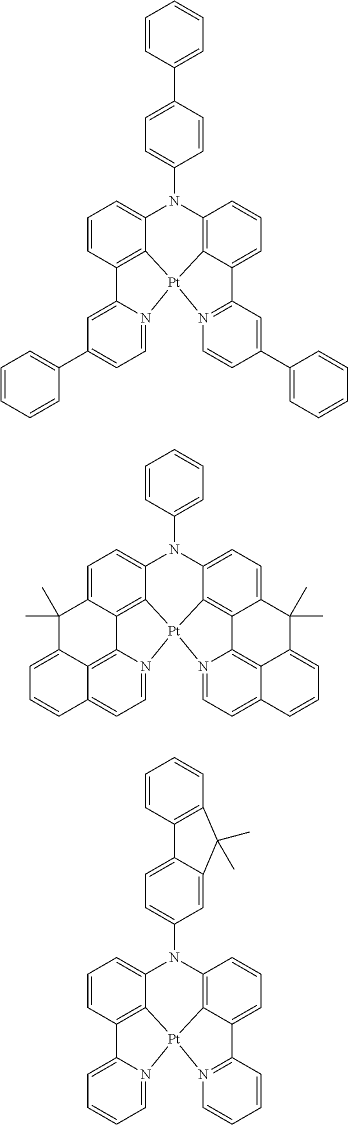

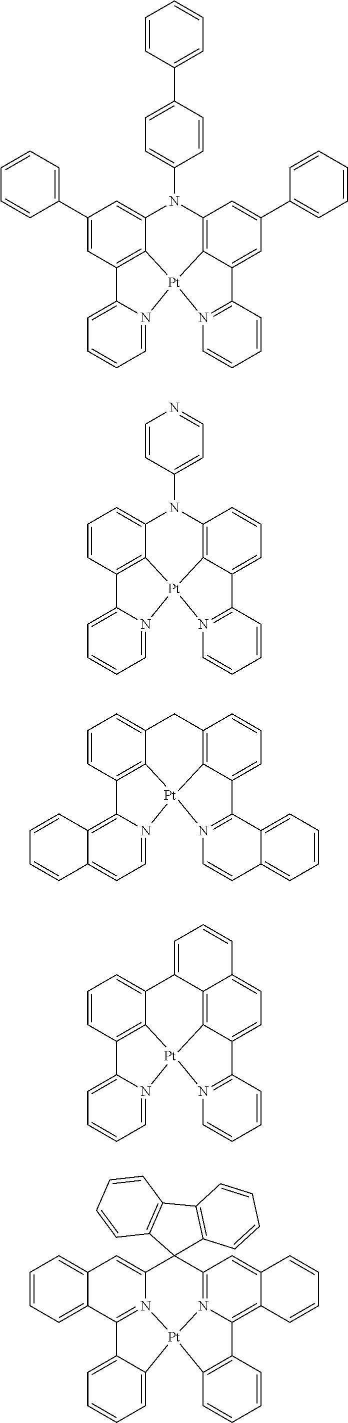

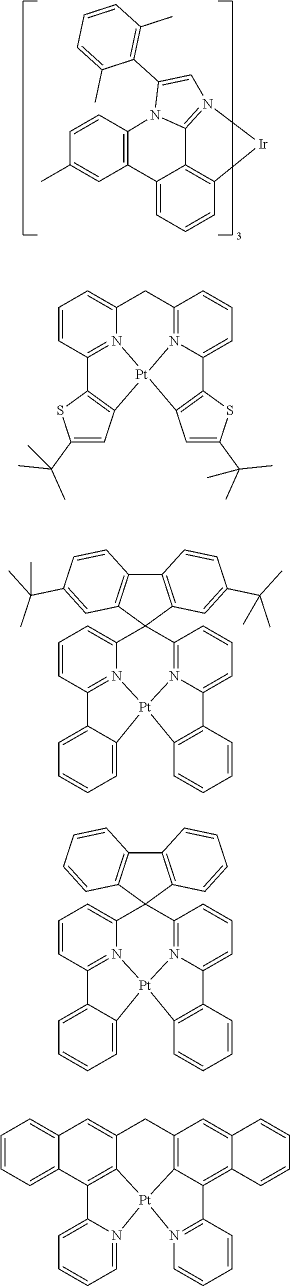

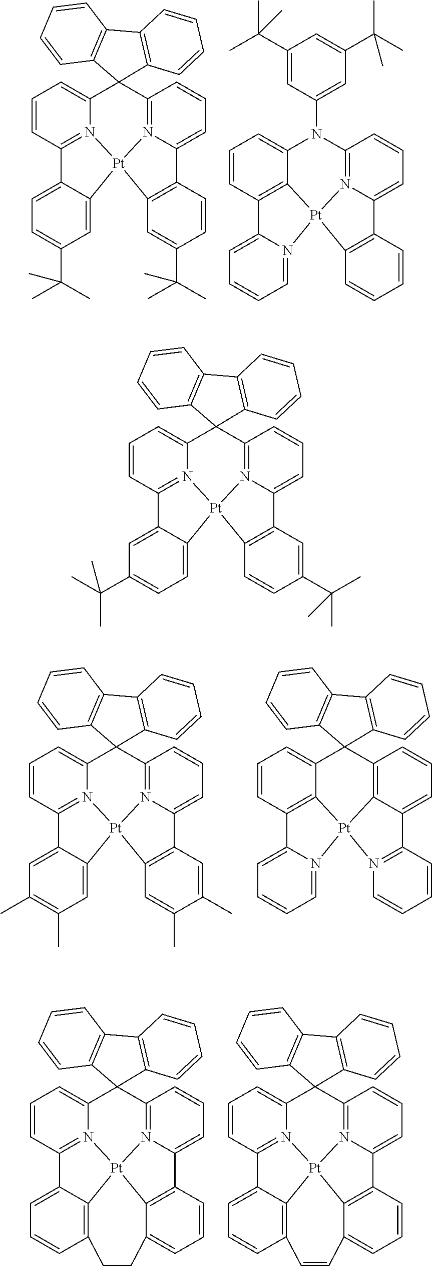

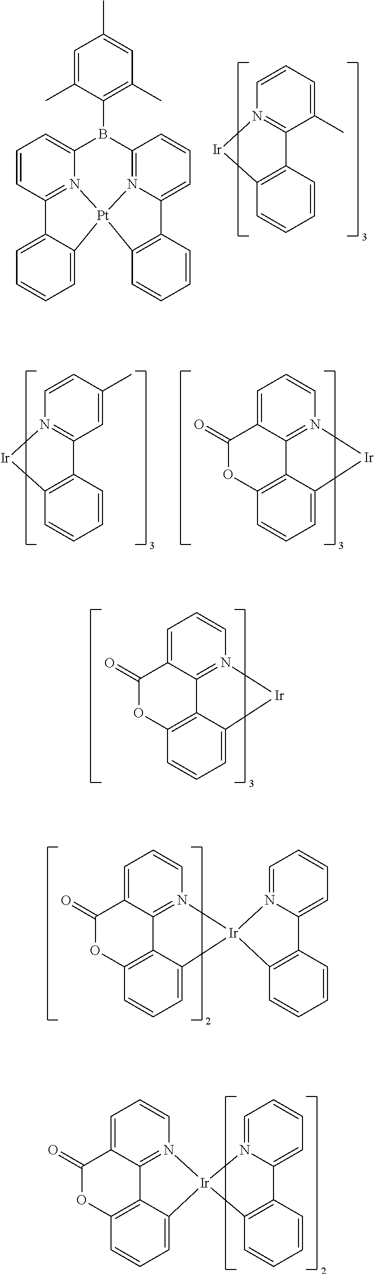

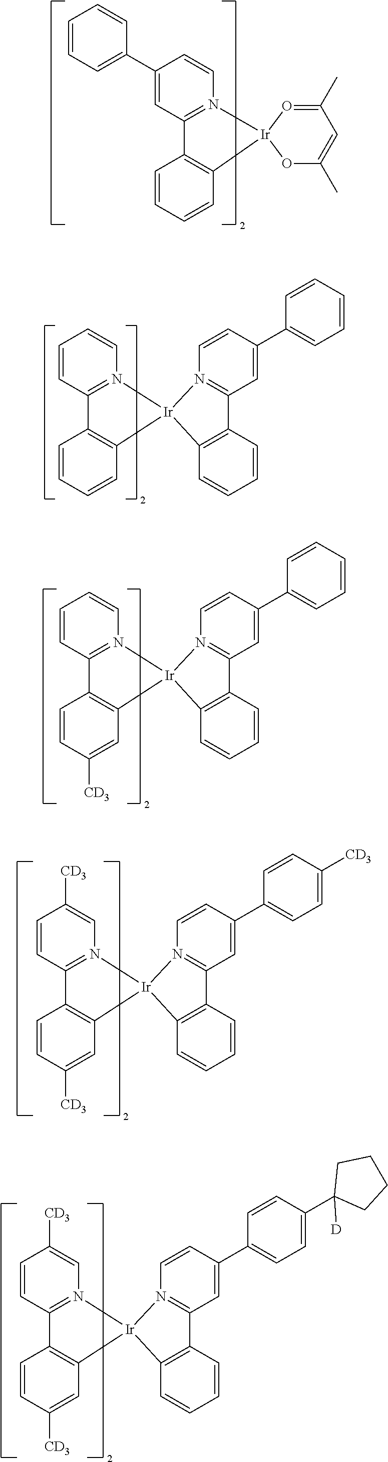

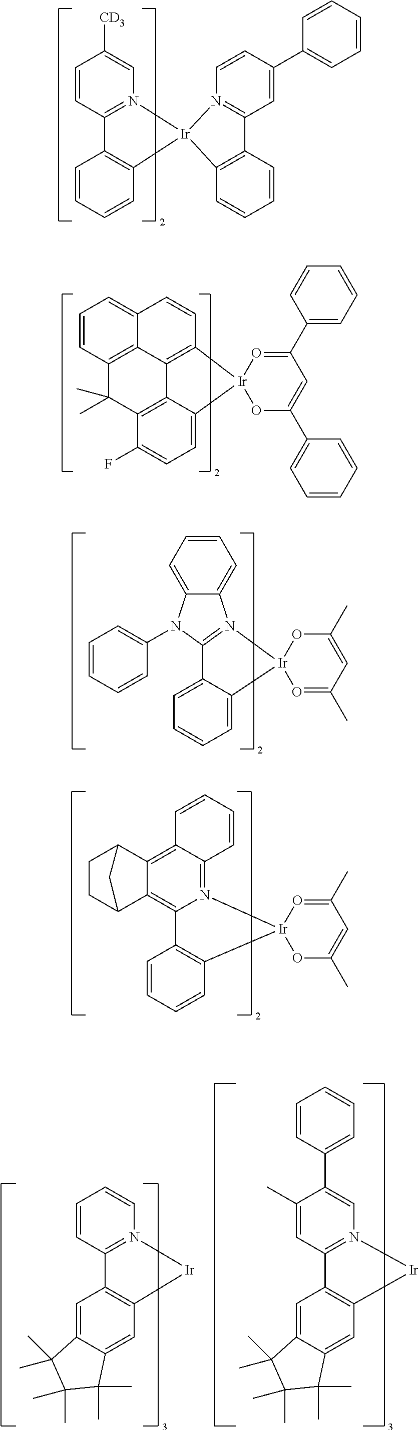

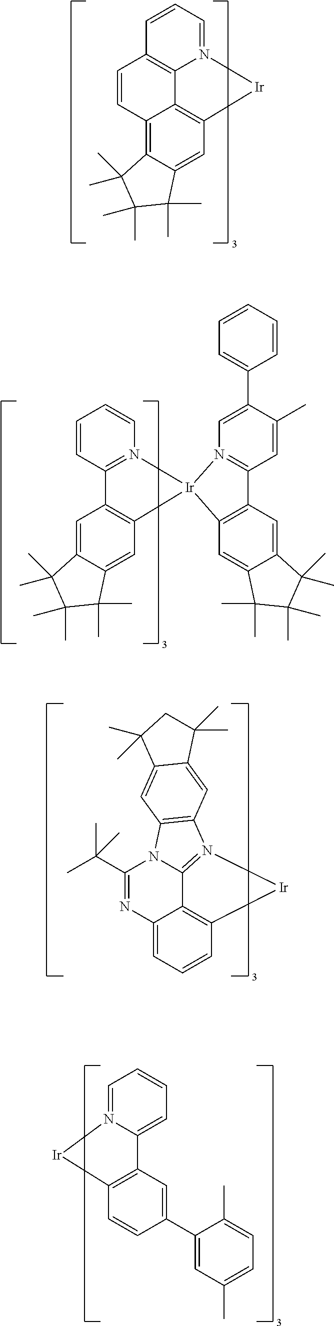

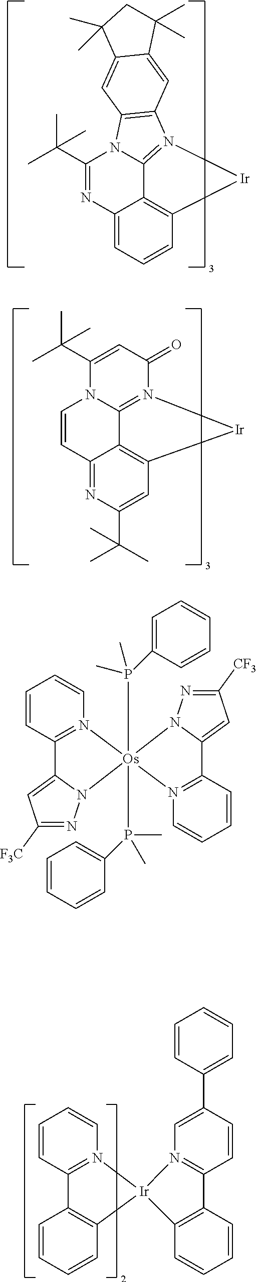

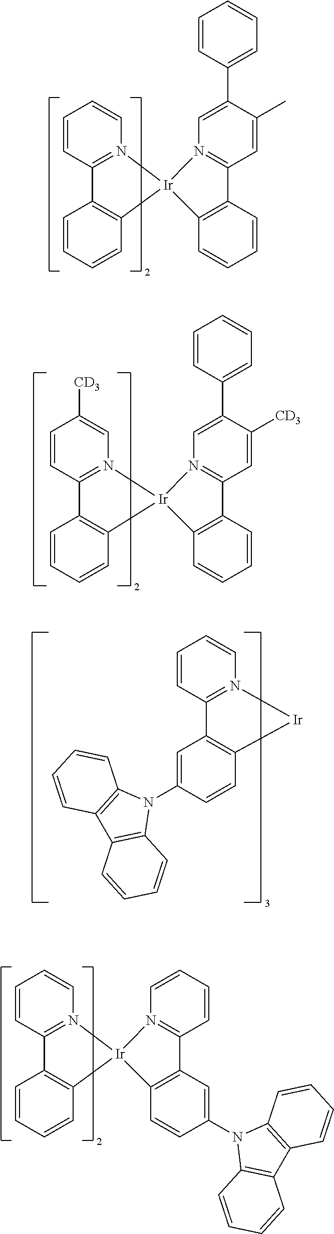

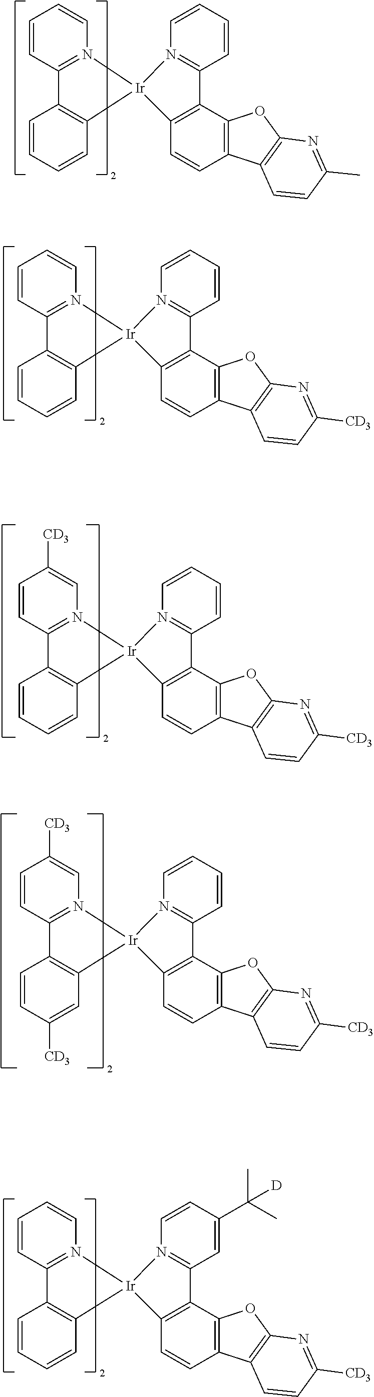

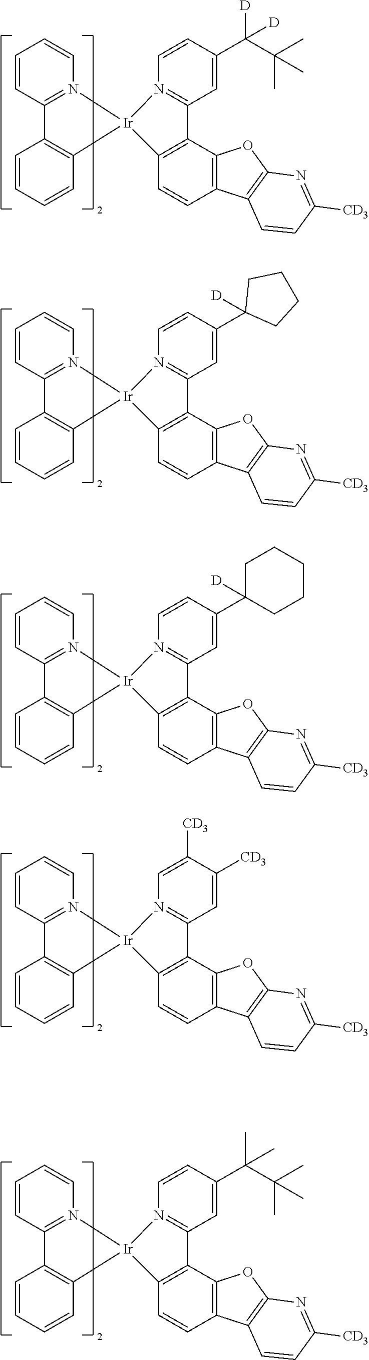

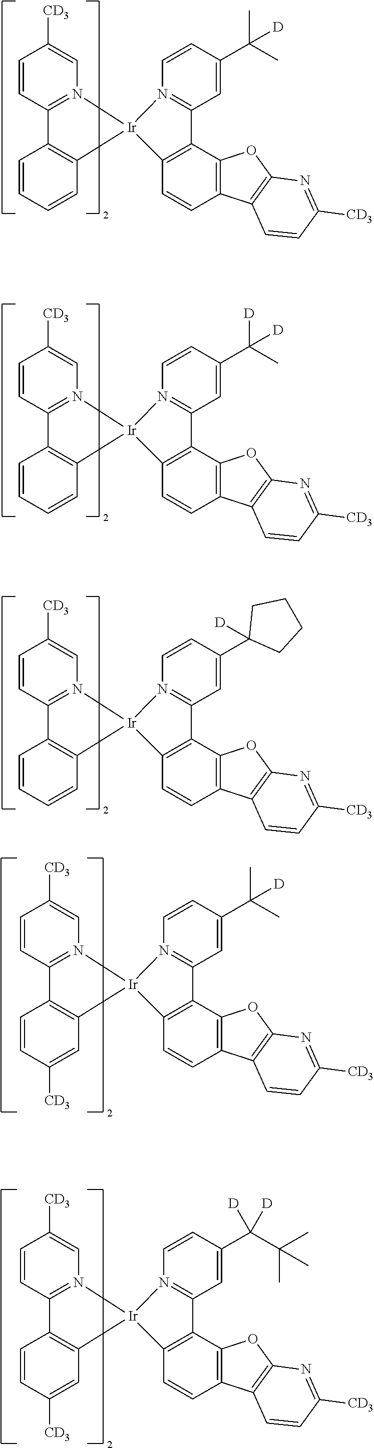

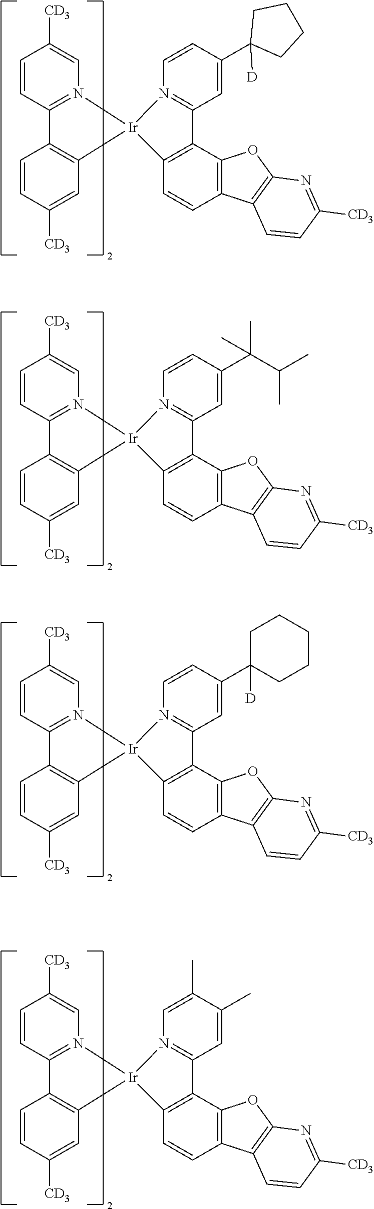

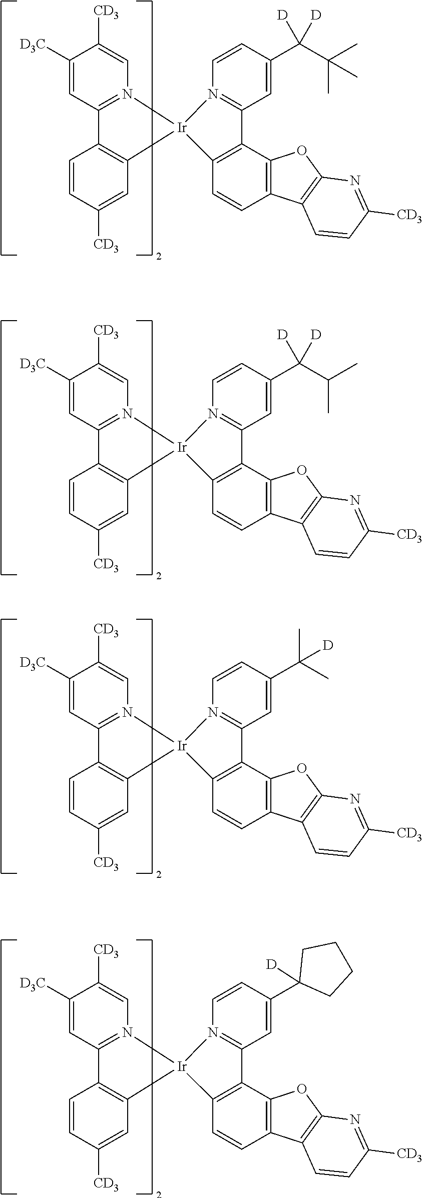

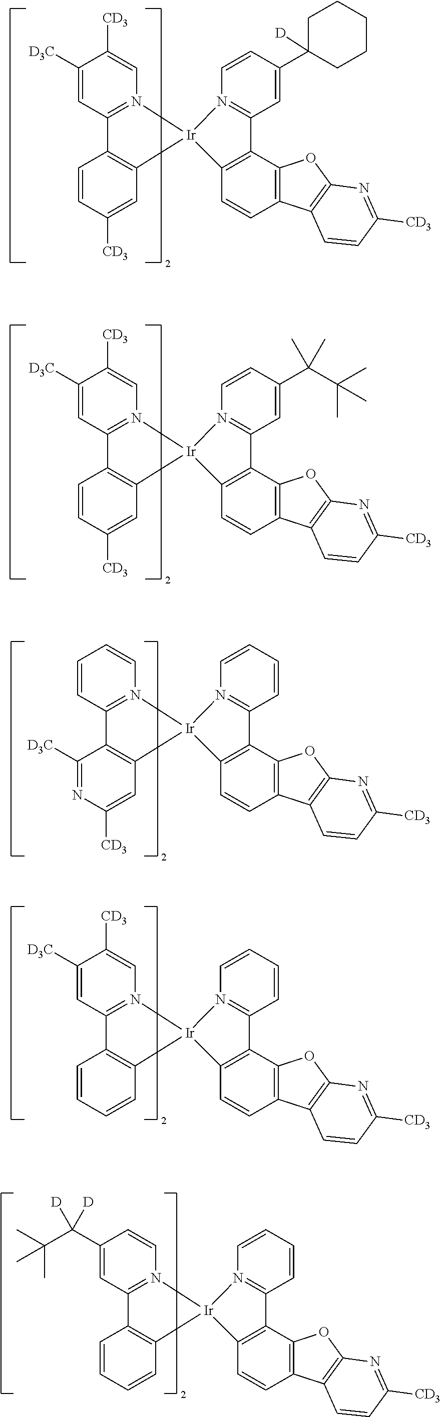

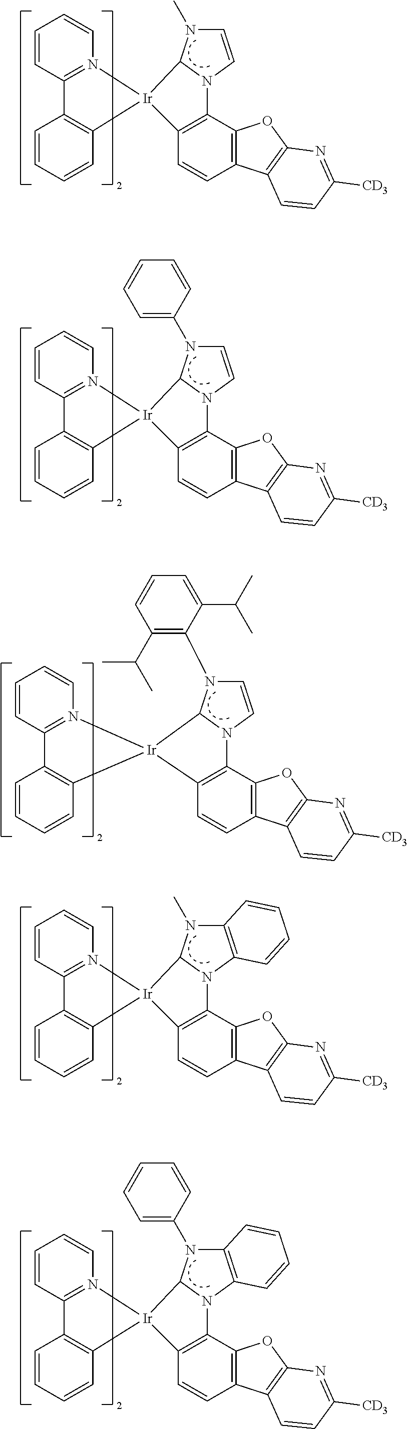

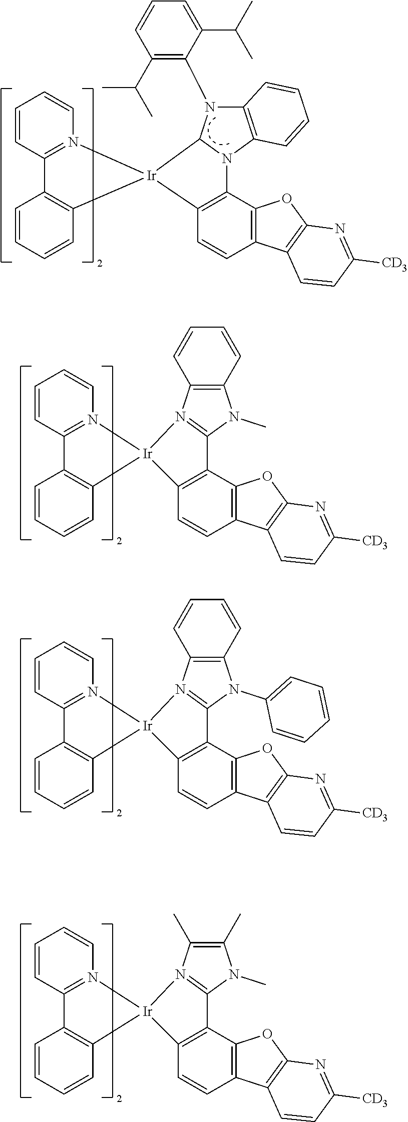

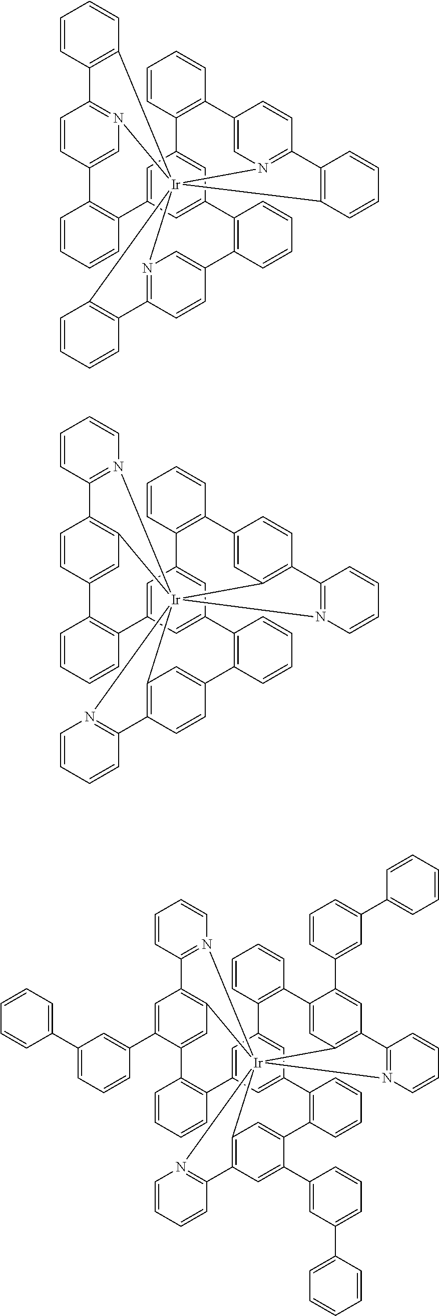

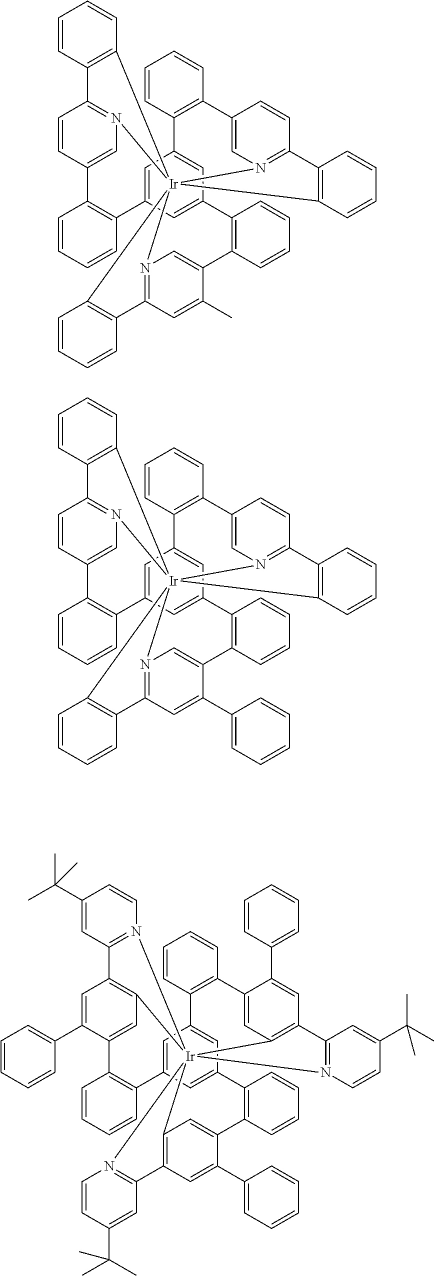

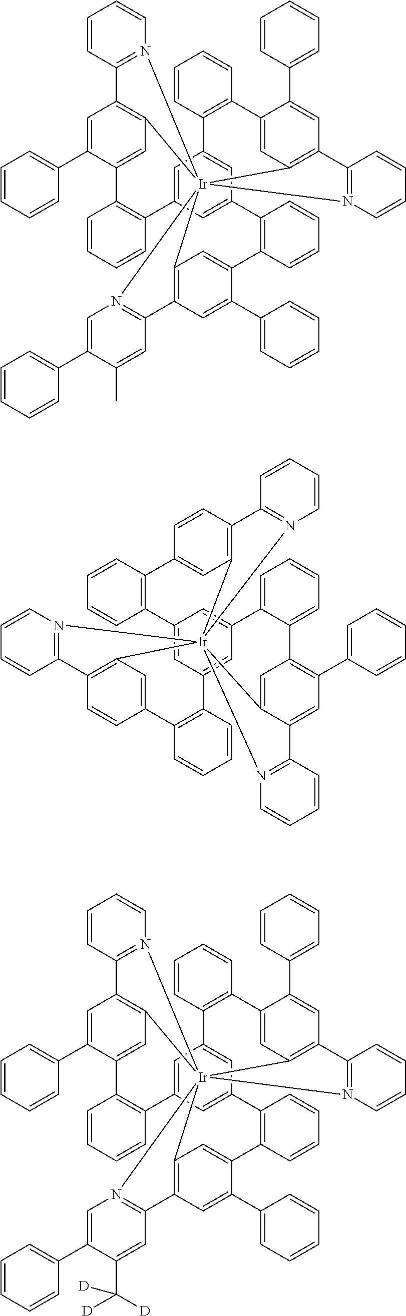

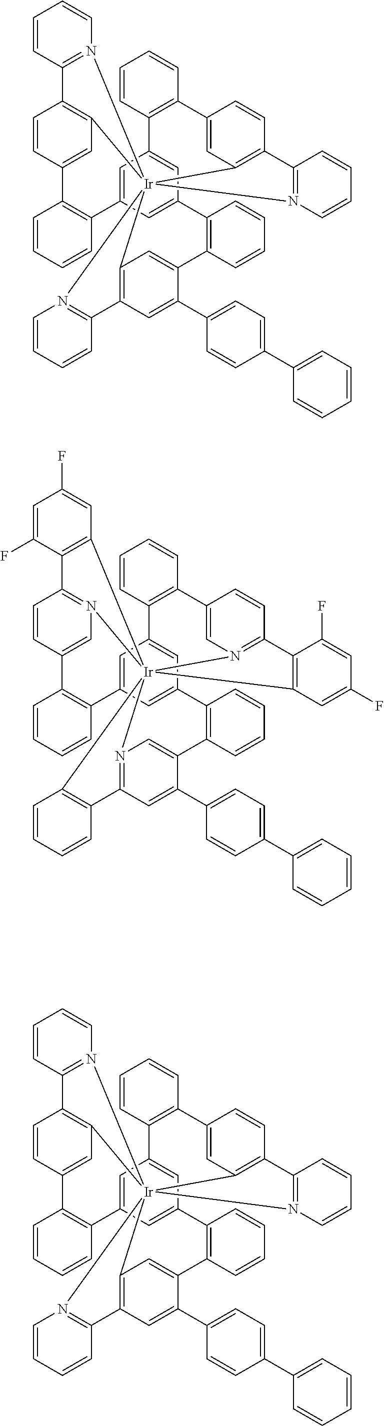

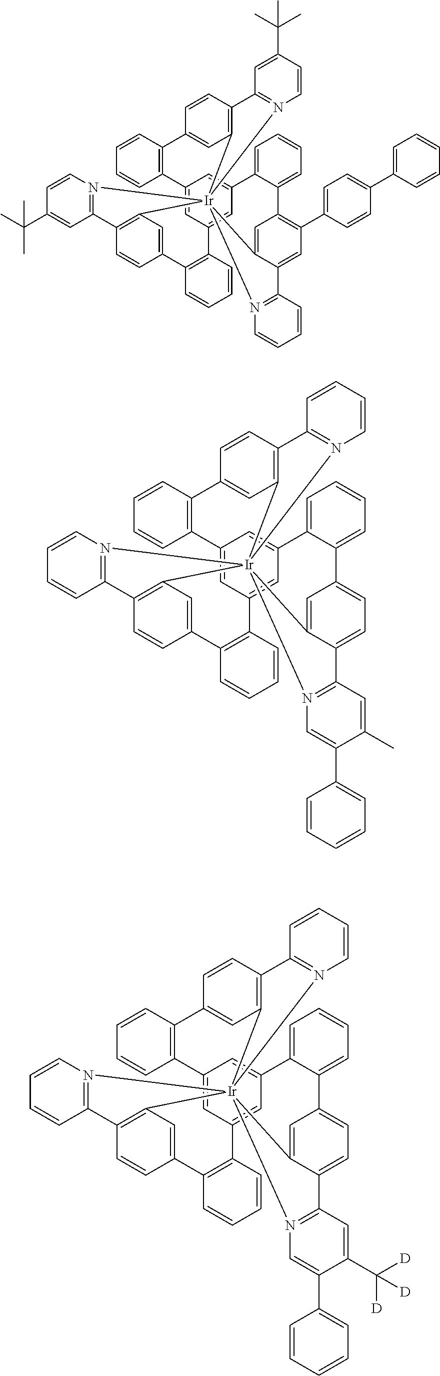

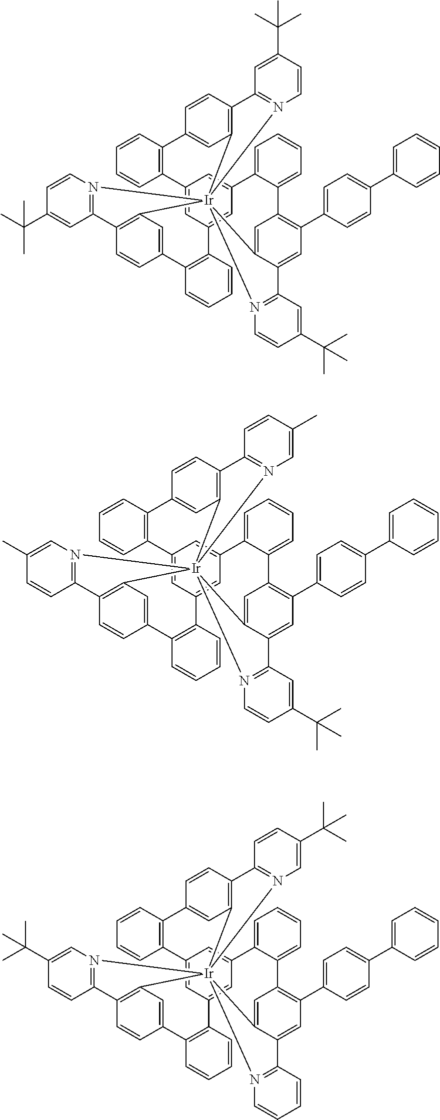

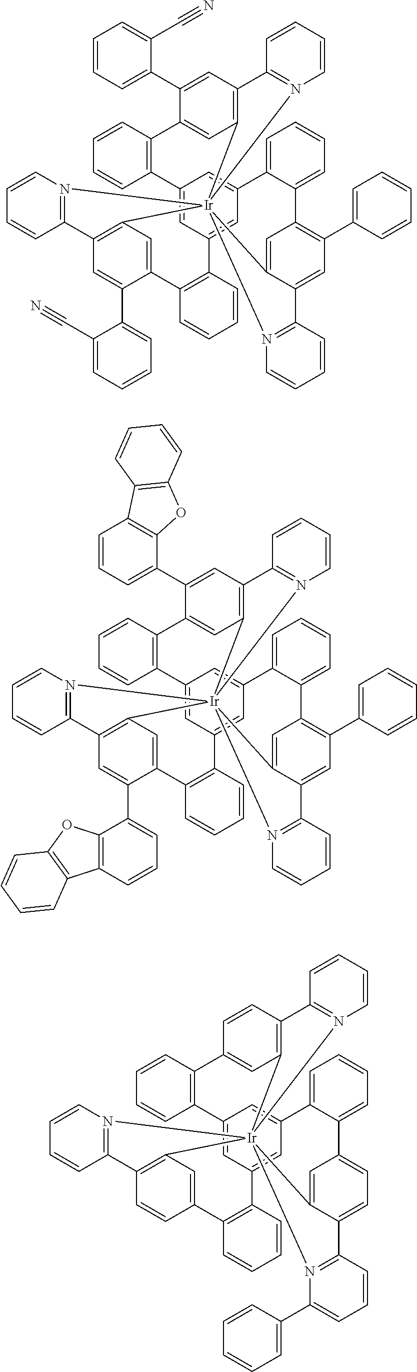

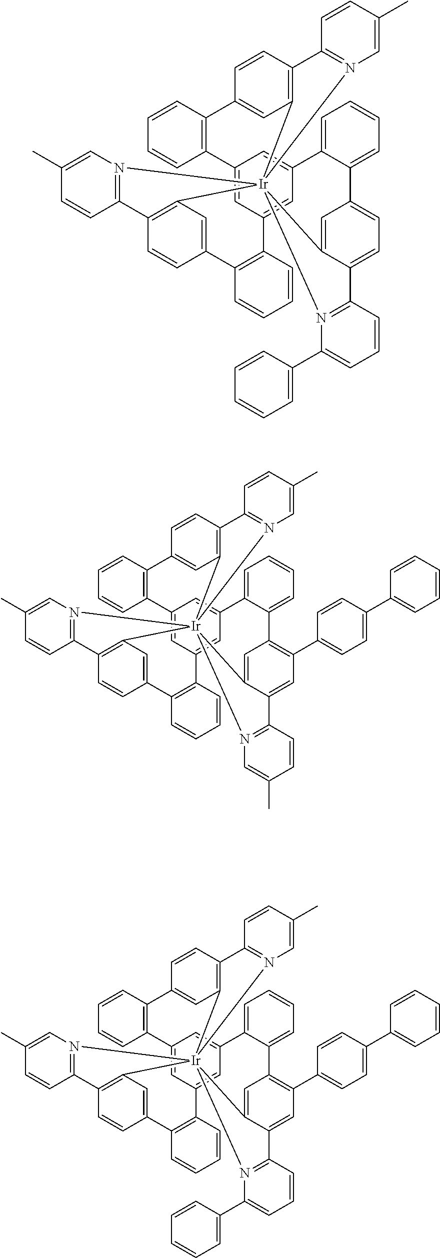

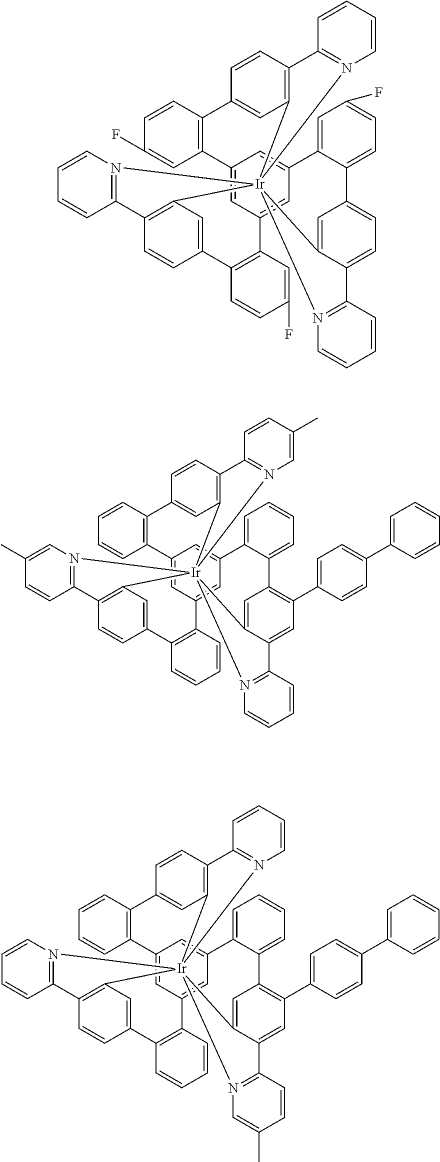

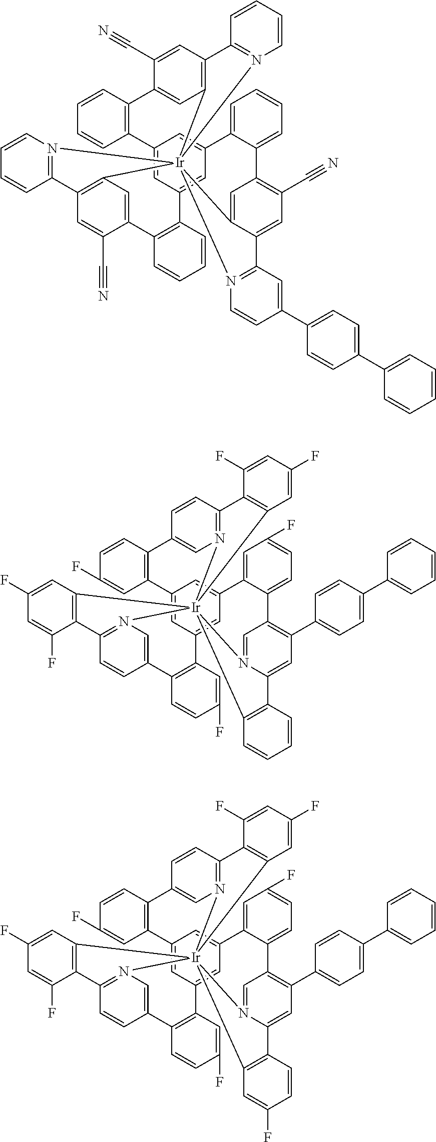

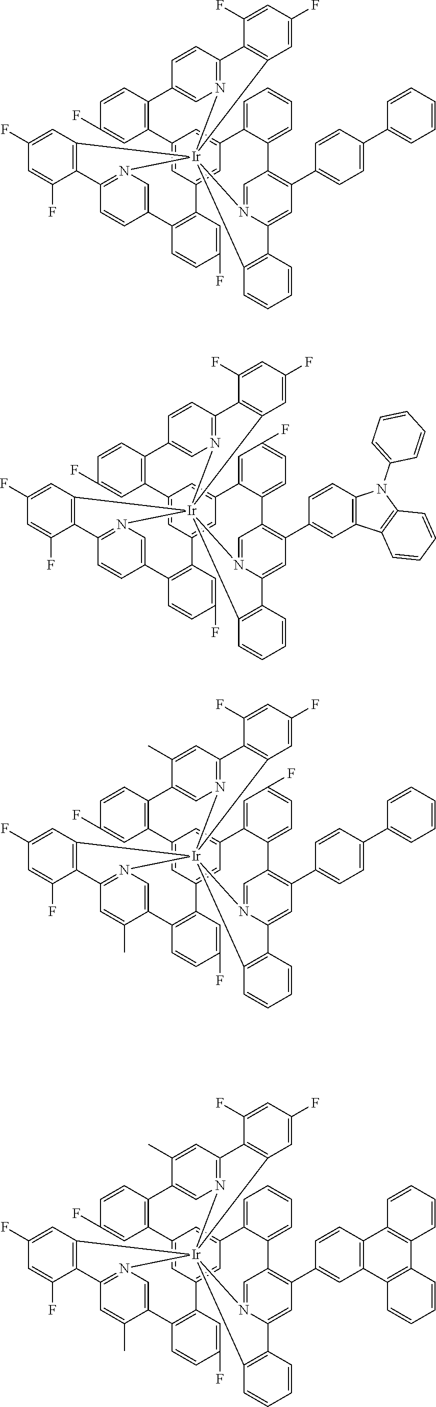

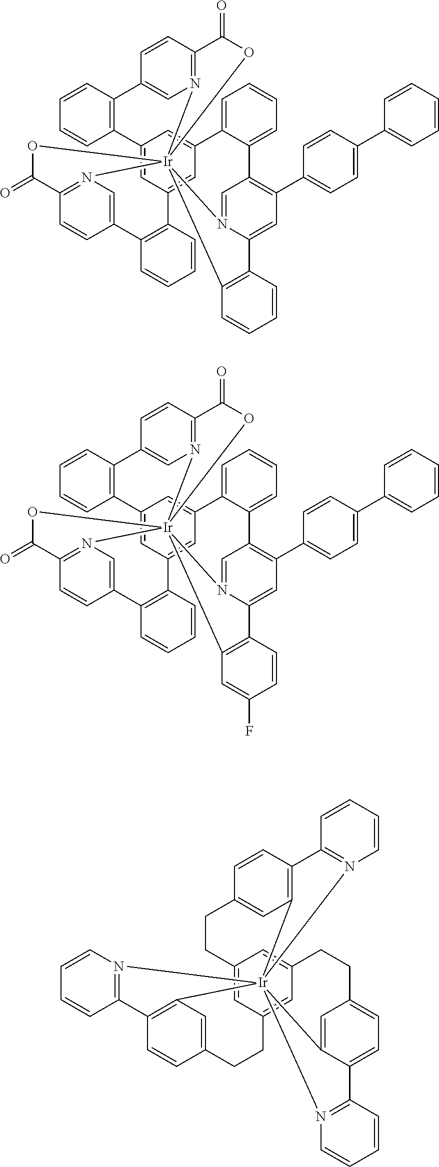

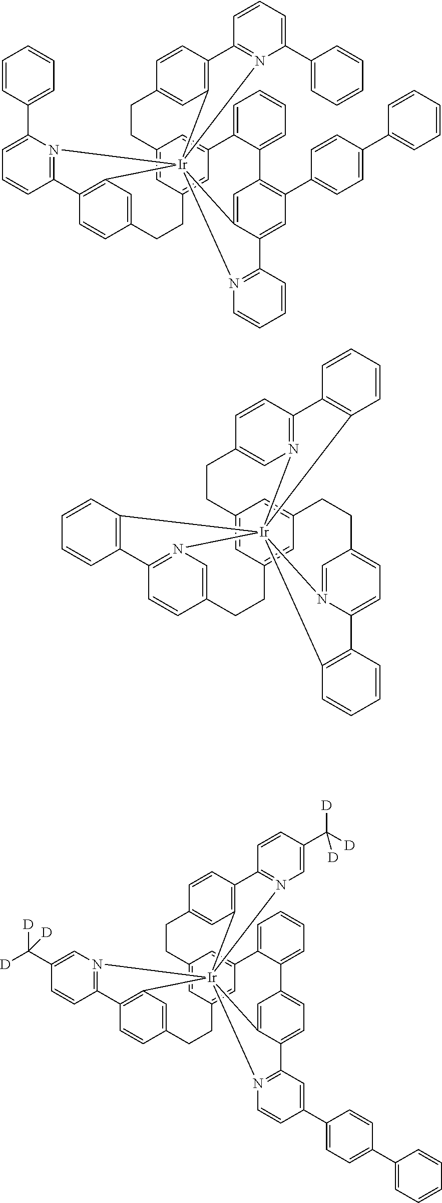

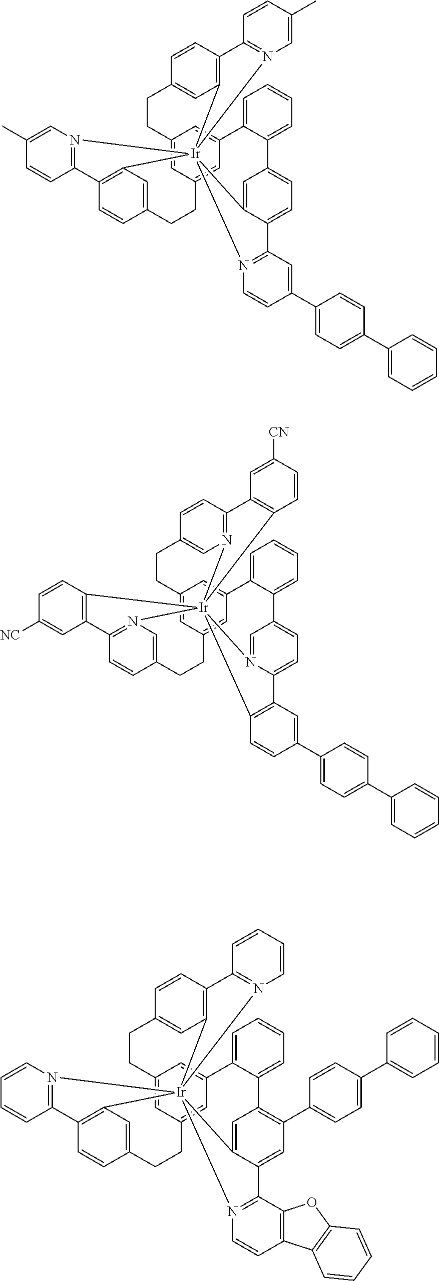

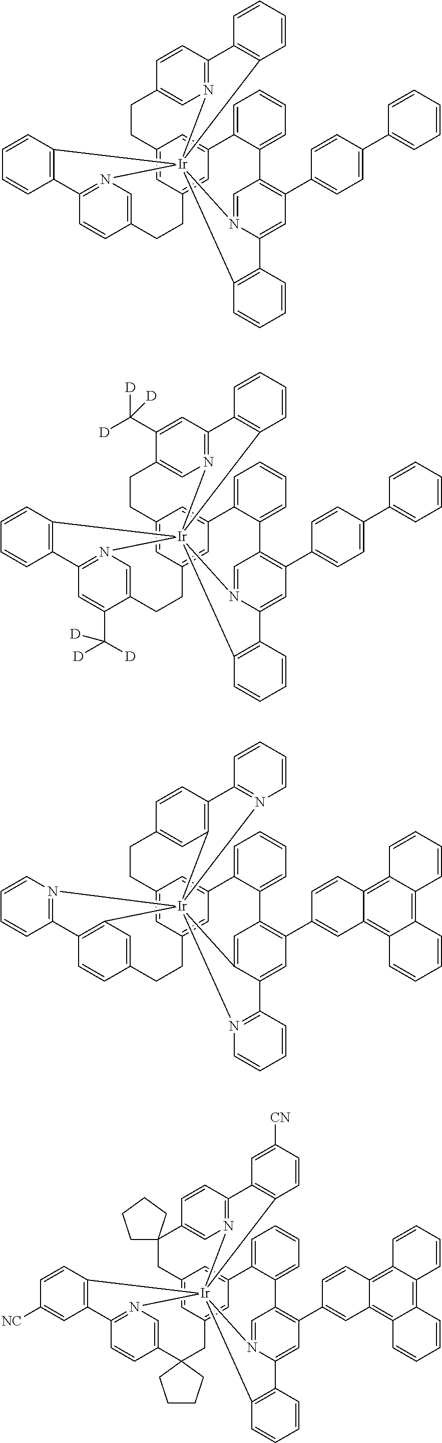

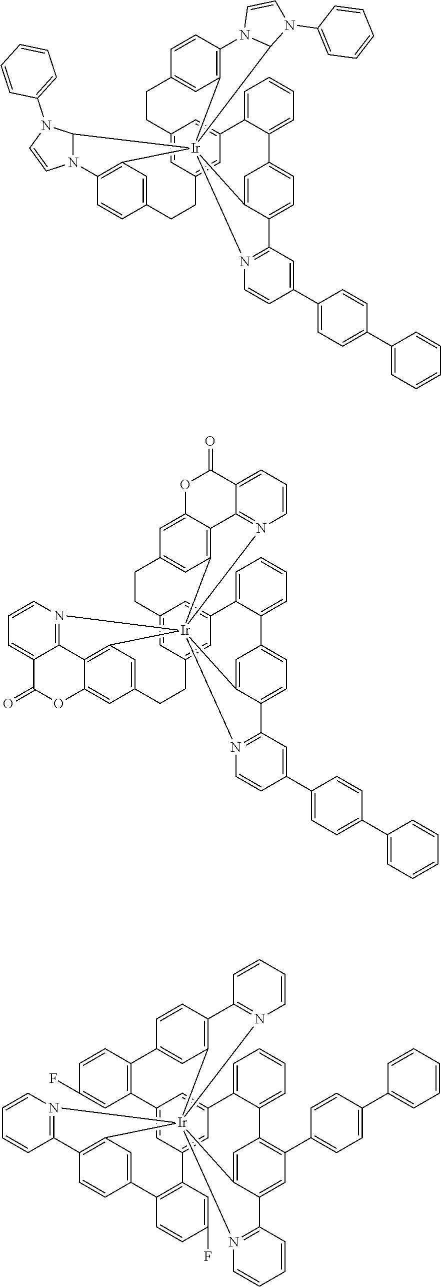

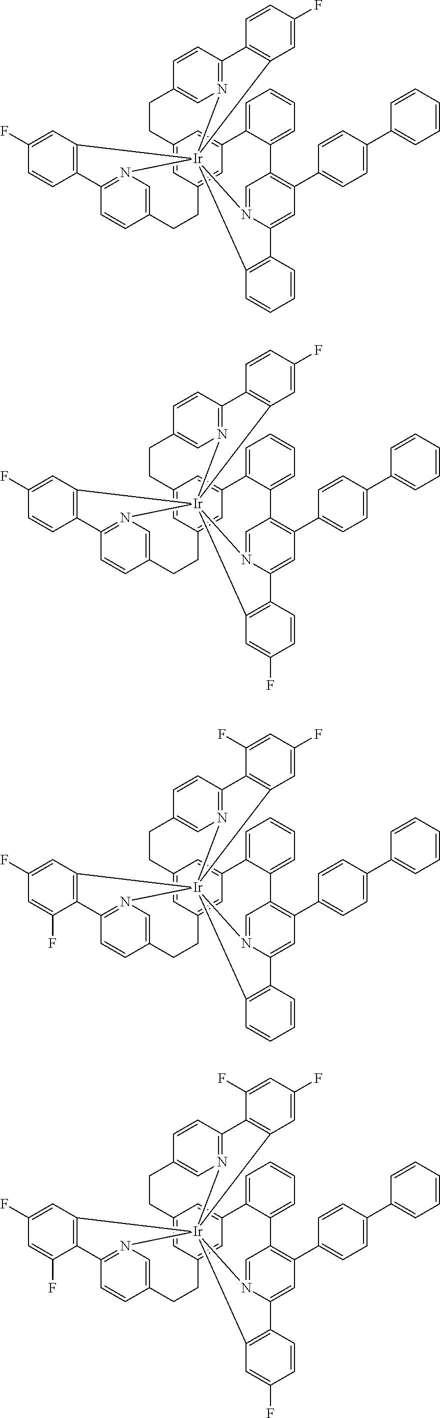

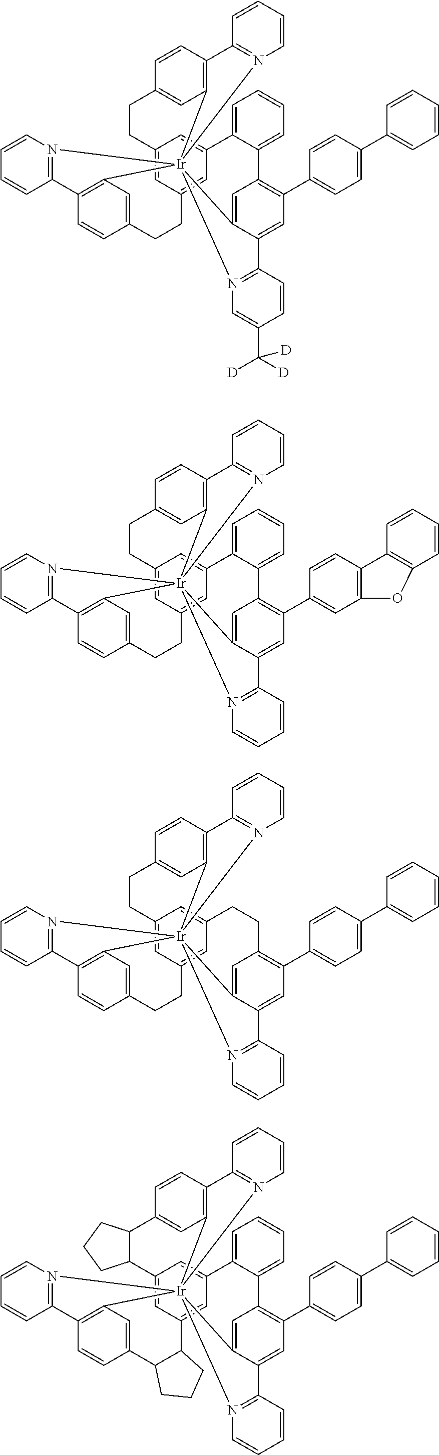

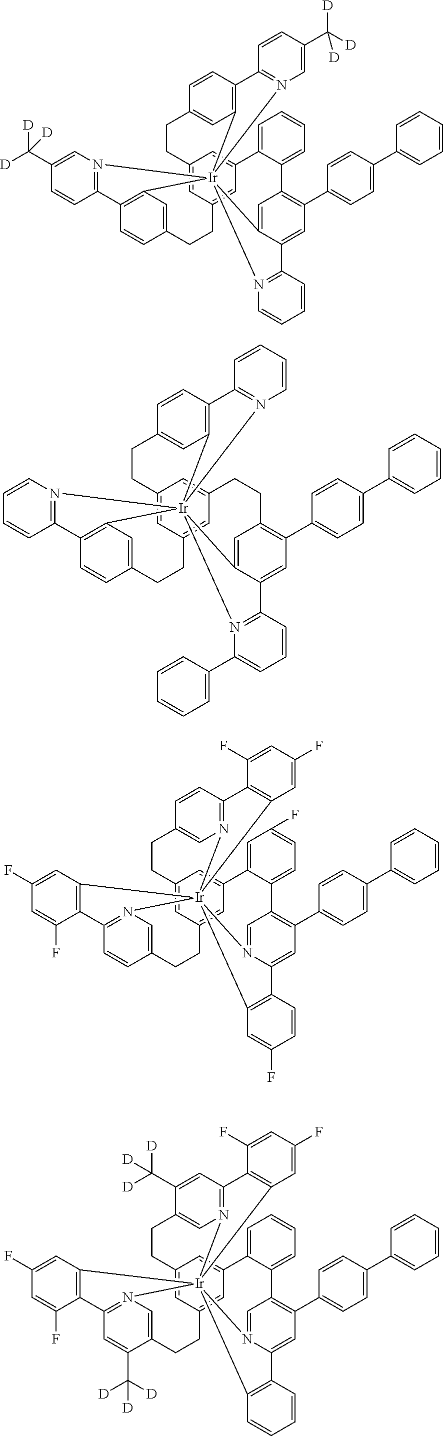

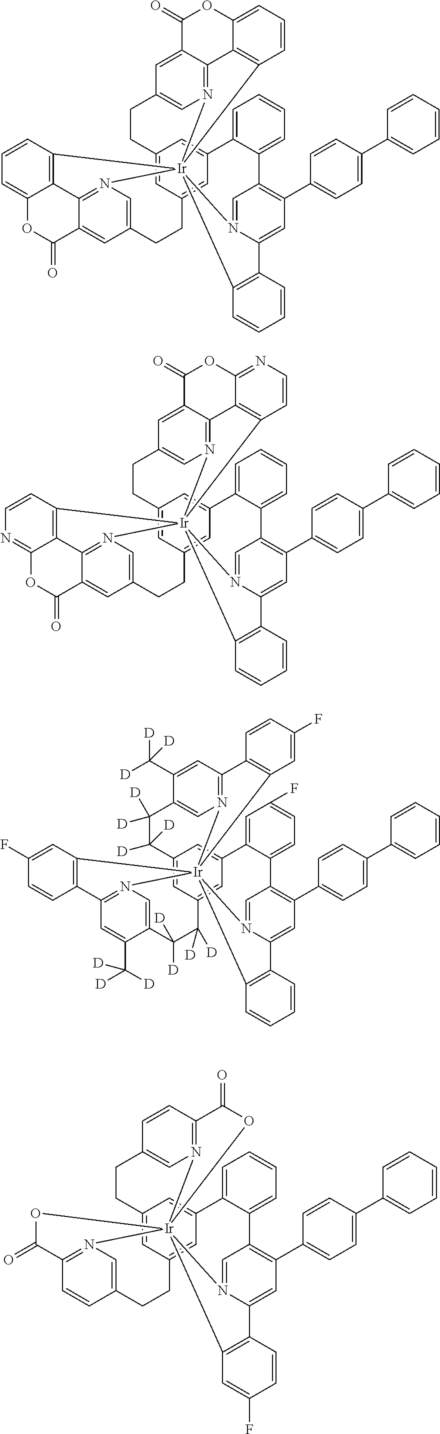

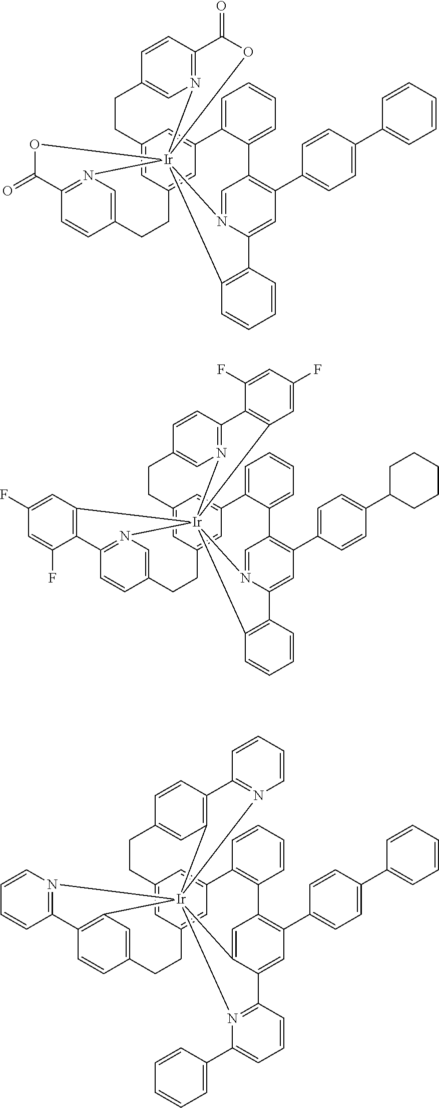

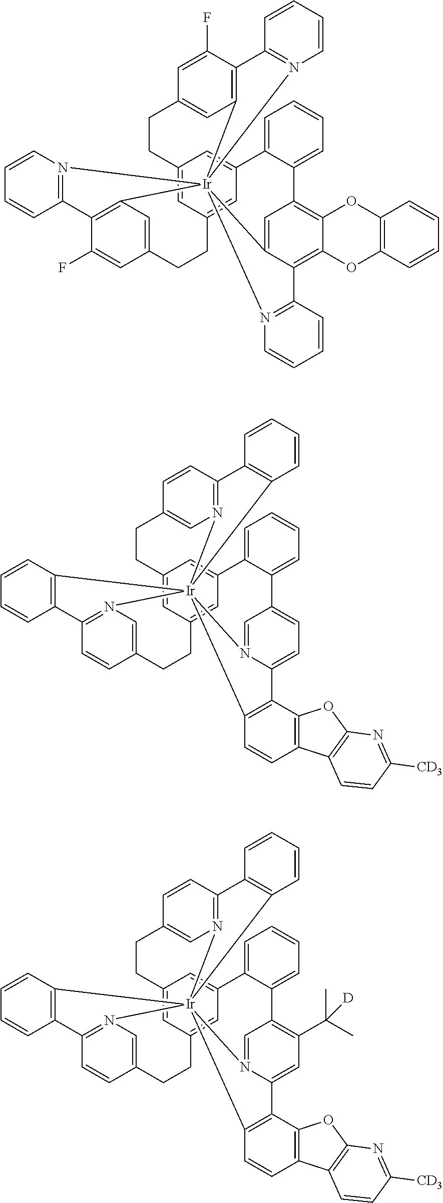

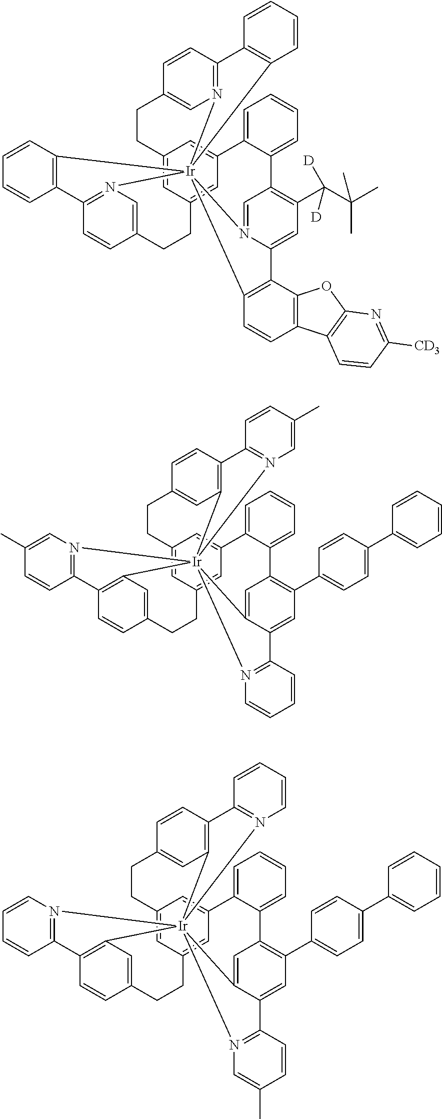

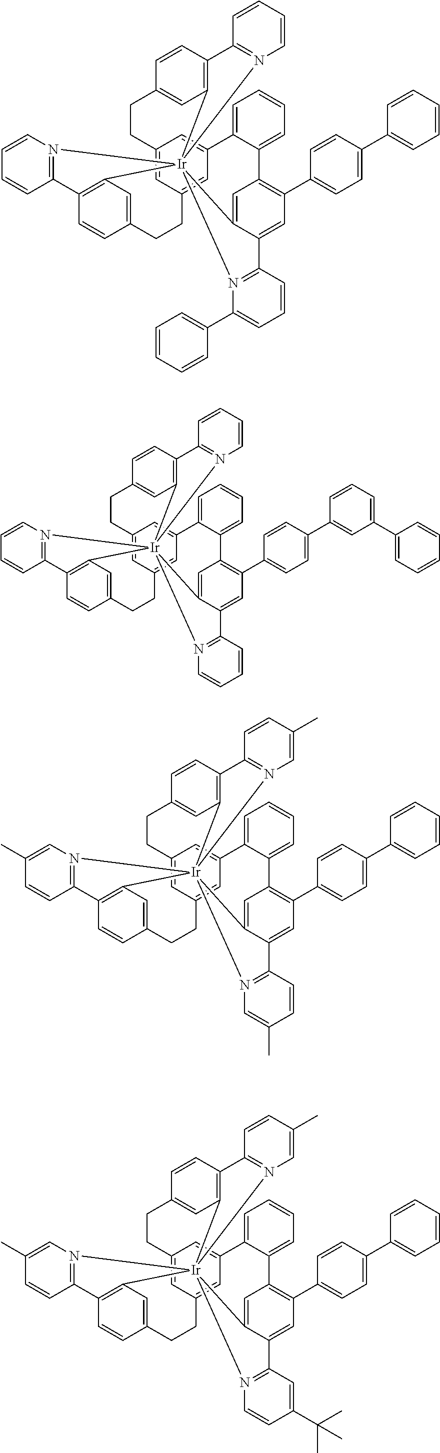

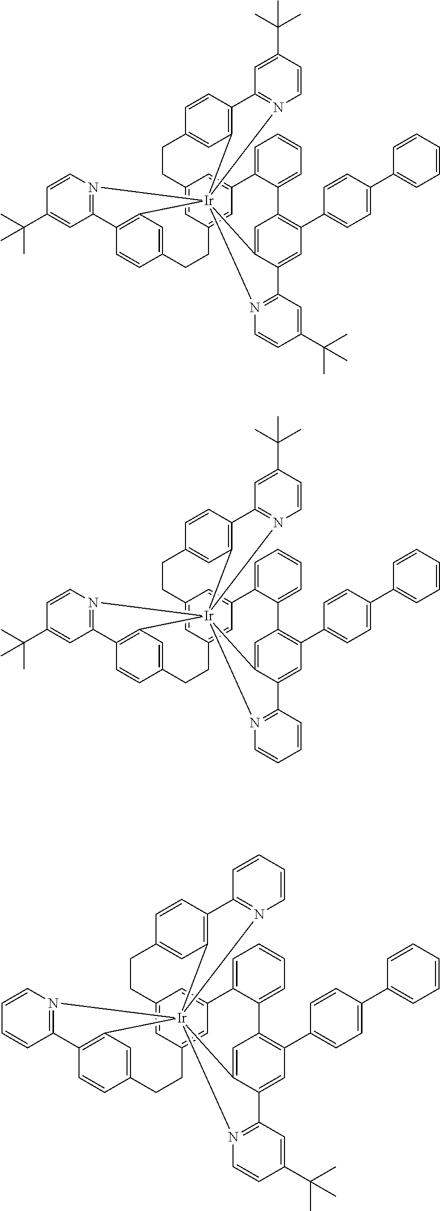

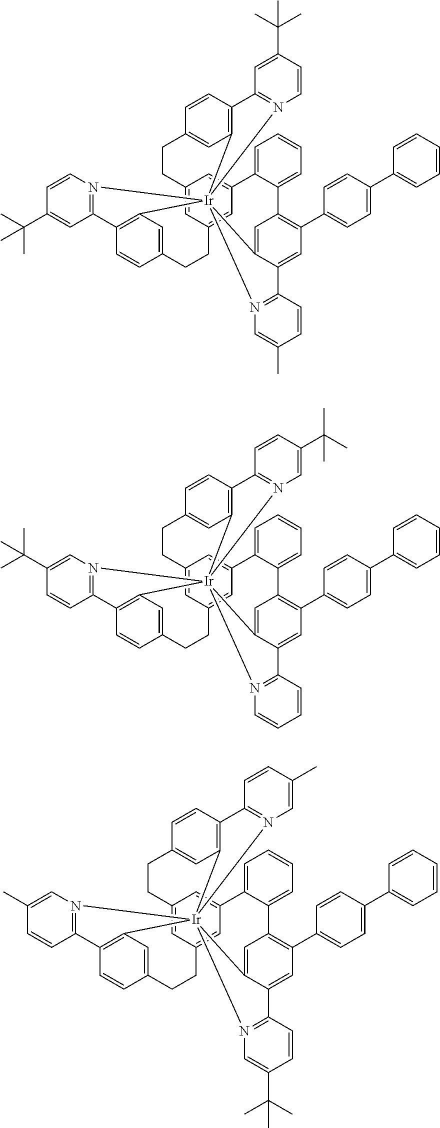

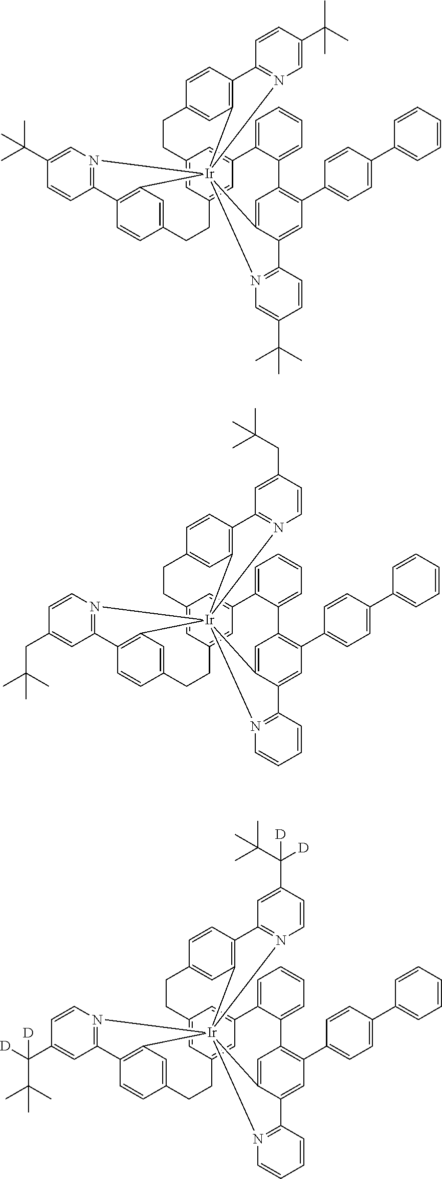

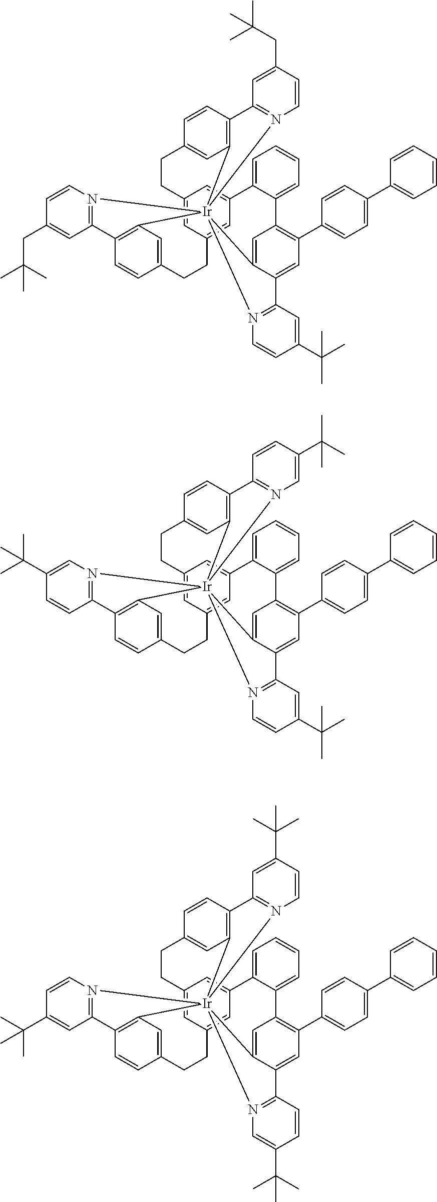

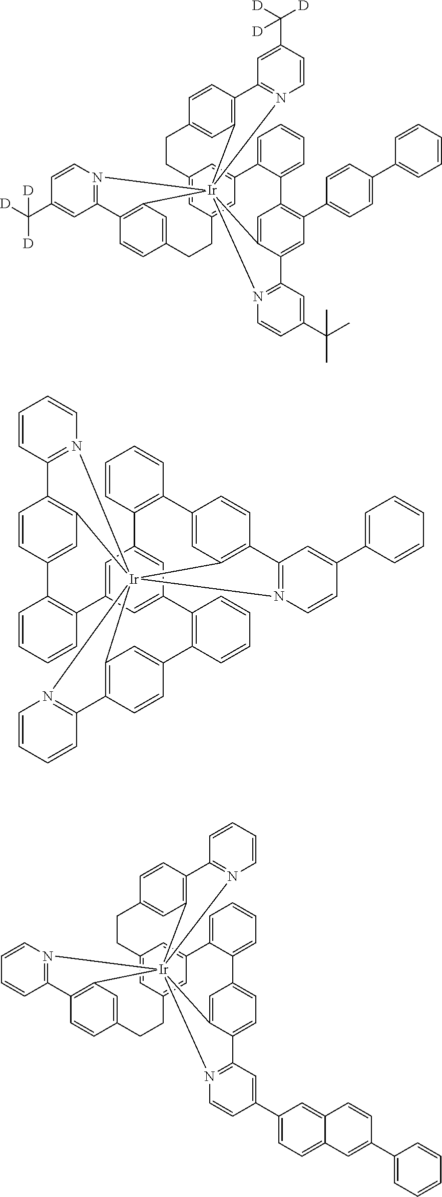

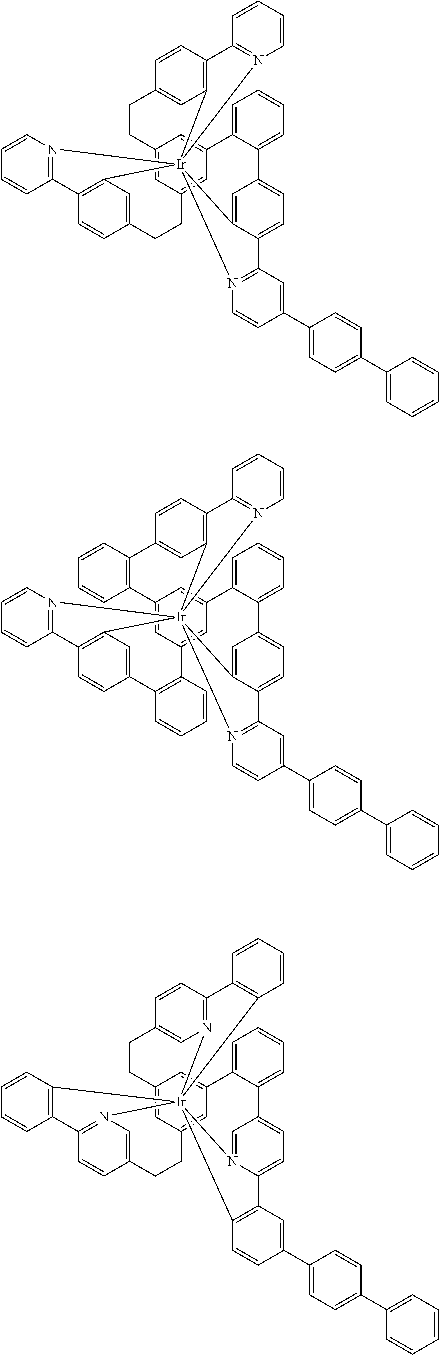

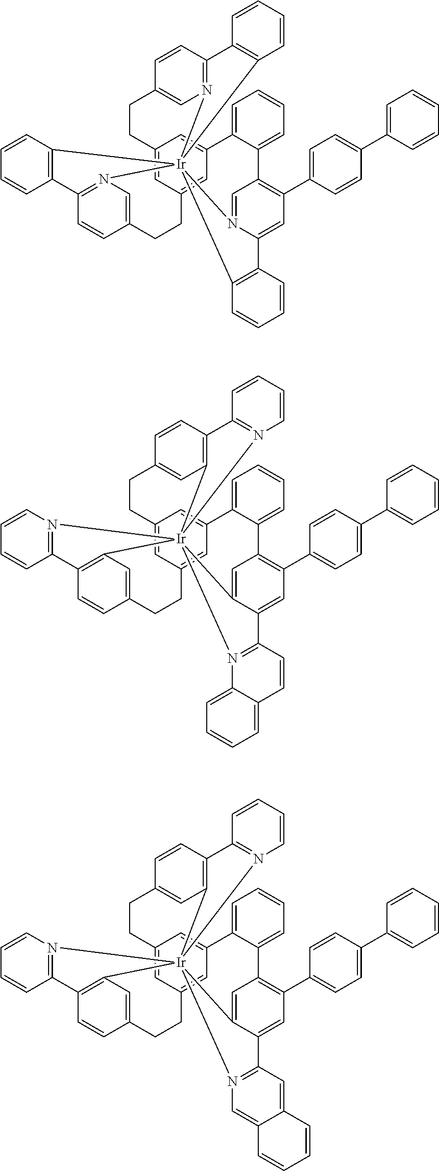

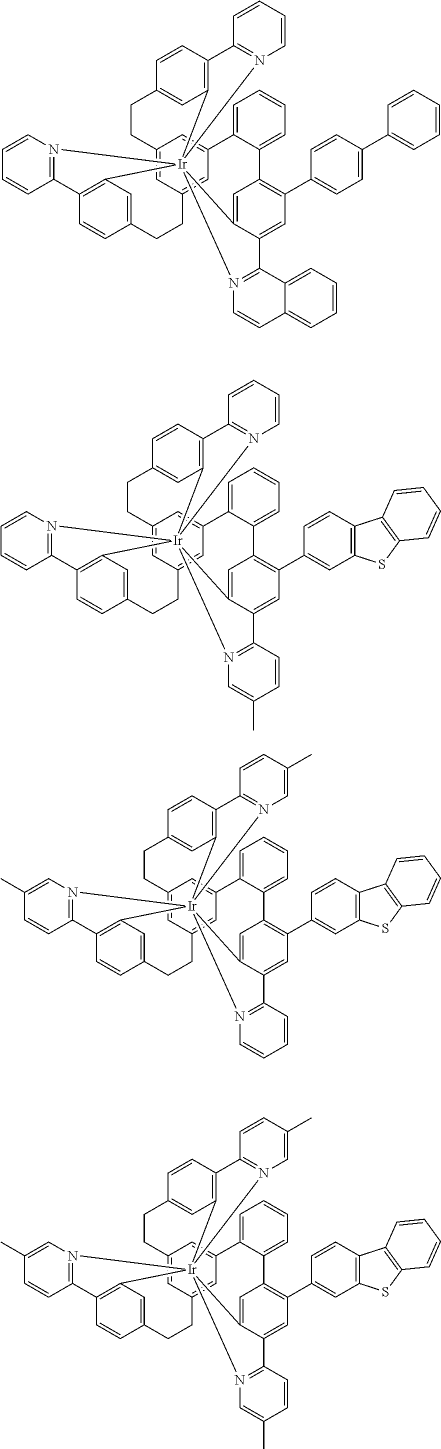

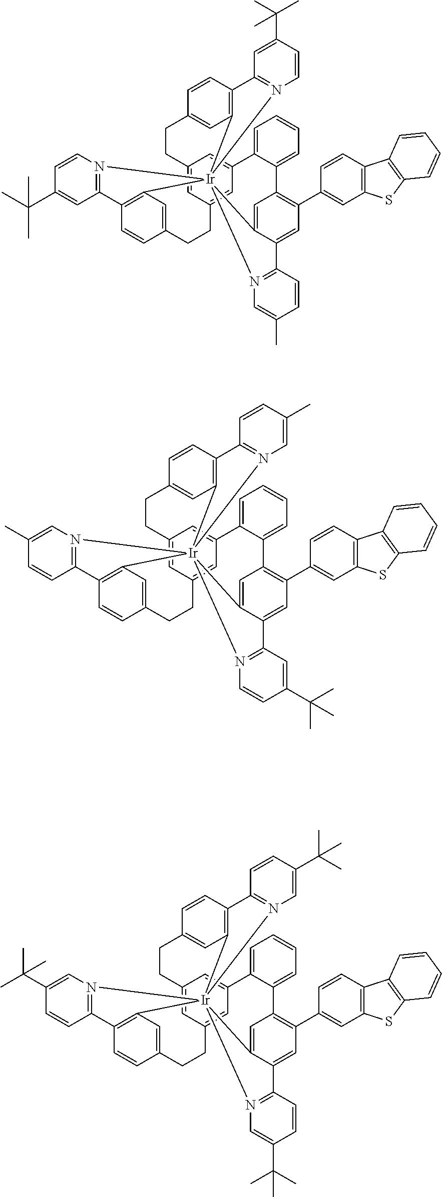

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

The compounds of the invention and the organic electroluminescent devices of the invention are notable for one or more of the following surprising properties:
1. The compounds of the invention, used as matrix material for phosphorescent emitters, lead to long lifetimes.
2. The compounds of the invention lead to high efficiencies, especially to a high EQE. This is especially true when the compounds are used as matrix material for a phosphorescent emitter.
3. The compounds of the invention lead to low operating voltages. This is especially true when the compounds are used as matrix material for a phosphorescent emitter.

The invention is illustrated in more detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased, for example, from Sigma-ALDRICH or ABCR. For the compounds known from the literature, the corresponding CAS numbers are also reported in each case.

### Example a) 8-Bromo-3-phenylbenzimidazolo[2,1-b][1,3]benzothiazin-12-one

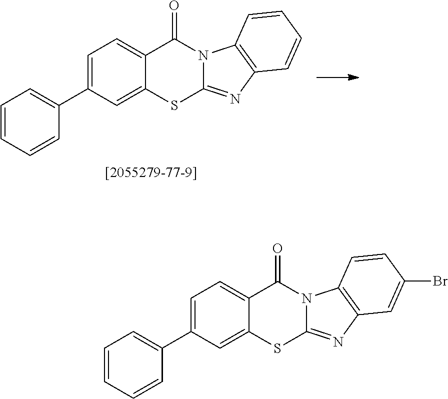 [SMILES: O=c1c2ccc(-c3ccccc3)cc2sc2nc3ccccc3n12 ; O=c1c2ccc(-c3ccccc3)cc2sc2nc3cc(Br)ccc3n12]

7.6 g (23 mmol) of 3-phenylbenzimidazolo[2,1-b][1,3]benzothiazin-12-one is initially charged in 150 ml of DMF. Subsequently, a solution of 4 g (22.5 mmol) of NBS in 100 ml of DMF is added dropwise in the dark at room temperature, the mixture is allowed to come to room temperature and stirring is continued at this temperature for 4 h. Subsequently, 150 ml of water are added to the mixture and extraction is effected with CH₂Cl₂. The organic phase is dried over MgSO₄ and the solvents are removed under reduced pressure. The product is subjected to extractive stirring with hot hexane and filtered off with suction. Yield: 7.9 g (19 mmol), 85% of theory, purity by ¹H NMR about 98%.

The following compounds are obtained in an analogous manner:

| Ex. | Reactant | Product | Yield |
|---|---|---|---|
| 1a | 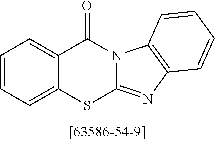 [SMILES: O=c1c2ccccc2sc2nc3ccccc3n12] | 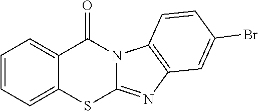 [SMILES: O=c1c2ccccc2sc2nc3cc(Br)ccc3n12] | 69% |
| 2a | 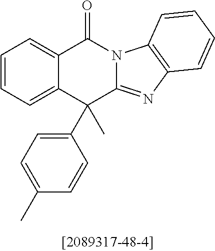 [SMILES: Cc1ccc(C2(C)c3ccccc3C(=O)n3c2nc2ccccc23)cc1] | 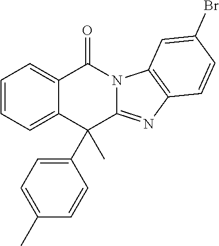 [SMILES: Cc1ccc(C2(C)c3ccccc3C(=O)n3c2nc2ccc(Br)cc23)cc1] | 60% |

### b) 8-(2-Chloroanilino)benzimidazolo[2,1-b][1,3]benzothiazin-12-one

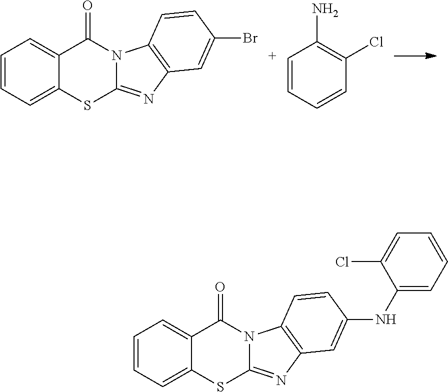 [SMILES: O=c1c2ccccc2sc2nc3cc(Br)ccc3n12 ; NC1=C(Cl)C=CC=[C+]1 ; O=c1c2ccccc2sc2nc3cc(Nc4ccccc4Cl)ccc3n12]

46 g (140 mmol) of 8-bromobenzimidazolo[2,1-b][1,3]benzothiazin-12-one, 17.9 g (140 mmol) of 2-chloroaniline, 68.2 g (710 mmol) of sodium tert-butoxide, 613 mg (3 mmol) of palladium(II) acetate and 3.03 g (5 mmol) of dppf are dissolved in 1.3 l of toluene and stirred under reflux for 5 h. The reaction mixture is cooled down to room temperature, extended with toluene and filtered through Celite. The filtrate is concentrated under reduced pressure and the residue is crystallized from toluene/heptane. The product is isolated as a colorless solid. Yield: 42.7 g (113 mmol), 81% of theory.

The following compounds can be prepared analogously:

| Ex. | Reactant 1 | Reactant 2 | Product | Yield |
|---|---|---|---|---|
| 1b | 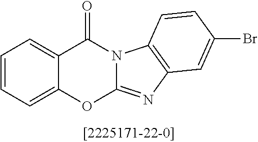 [SMILES: O=c1c2ccccc2oc2nc3cc(Br)ccc3n12] | 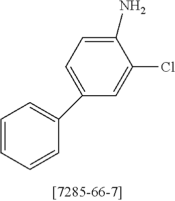 [SMILES: Nc1ccc(-c2ccccc2)cc1Cl] | 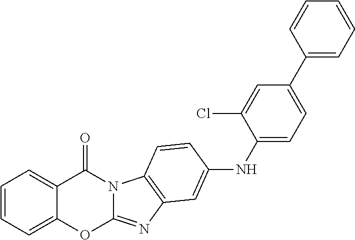 [SMILES: O=c1c2ccccc2oc2nc3cc(Nc4ccc(-c5ccccc5)cc4Cl)ccc3n12] | 80% |
| 2b | 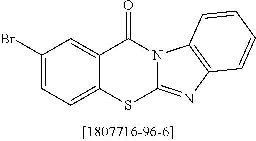 [SMILES: O=c1c2cc(Br)ccc2sc2nc3ccccc3n12] | 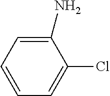 | 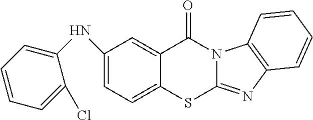 [SMILES: O=c1c2cc(Nc3ccccc3Cl)ccc2sc2nc3ccccc3n12] | 83% |
| 3b | 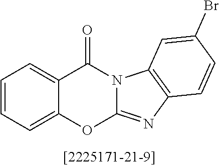 [SMILES: O=c1c2ccccc2oc2nc3ccc(Br)cc3n12] | 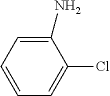 | 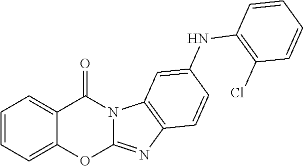 [SMILES: O=c1c2ccccc2oc2nc3ccc(Nc4ccccc4Cl)cc3n12] | 79% |
| 4b | 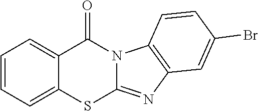 [SMILES: O=c1c2ccccc2sc2nc3cc(Br)ccc3n12] | 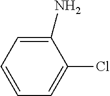 | 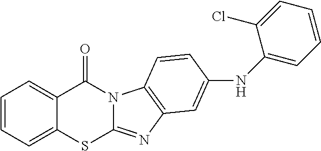 [SMILES: O=c1c2ccccc2sc2nc3cc(Nc4ccccc4Cl)ccc3n12] | 77% |
| 5b | 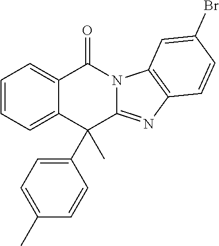 [SMILES: Cc1ccc(C2(C)c3ccccc3C(=O)n3c2nc2ccc(Br)cc23)cc1] | 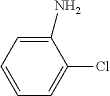 | 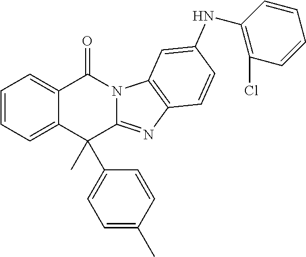 [SMILES: Cc1ccc(C2(C)c3ccccc3C(=O)n3c2nc2ccc(Nc4ccccc4Cl)cc23)cc1] | 86% |
| 6b | 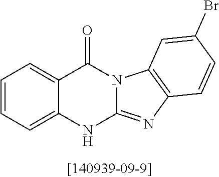 [SMILES: O=c1c2ccccc2[nH]c2nc3ccc(Br)cc3n12] | 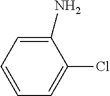 | 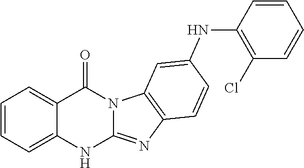 [SMILES: O=c1c2ccccc2[nH]c2nc3ccc(Nc4ccccc4Cl)cc3n12] | 88% |

c) Cyclization

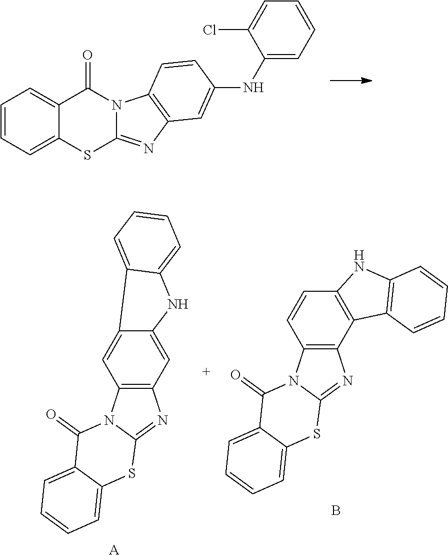

37 g (100 mmol) of 8-(2-chloroanilino)benzimidazolo[2,1-b][1,3]benzothiazin-12-one, 56 g (409 mmol) of potassium carbonate, 4.5 g (12 mmol) of tricyclohexylphosphine tetrafluoroborate, 1.38 g (6 mmol) of palladium(II) acetate are suspended in 500 ml of dimethylacetamide and stirred under reflux for 6 h. After cooling, the reaction mixture is diluted with 300 ml of water and 400 ml of ethyl acetate. The mixture is stirred for a further 30 min, the organic phase is separated off and filtered through a short Celite bed, and then the solvent is removed under reduced pressure. The crude product is subjected to hot extraction with toluene and recrystallized from toluene. Yield: 22.8 g (88 mmol) of the A+B mixture; 88% of theory; purity: 98.0% by HPLC. After recrystallization from EA/toluene (1:3) and subsequent workup, 69% A and 19% B are obtained.

The following compounds can be prepared analogously:

| | Reactant | Product A | Product B | Yield |
|---|---|---|---|---|
| 1c | 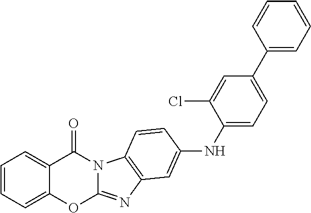 [SMILES: O=c1c2ccccc2oc2nc3cc(Nc4ccc(-c5ccccc5)cc4Cl)ccc3n12] | 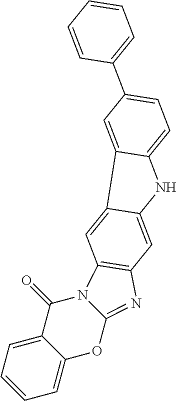 [SMILES: O=c1c2ccccc2oc2nc3cc4[nH]c5ccc(-c6ccccc6)cc5c4cc3n12] | 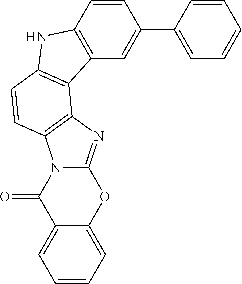 [SMILES: O=c1c2ccccc2oc2nc3c4c(ccc3n12)[nH]c1ccc(-c2ccccc2)cc14] | 57%, 21% |
| 2c | 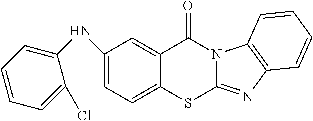 [SMILES: O=c1c2cc(Nc3ccccc3Cl)ccc2sc2nc3ccccc3n12] | 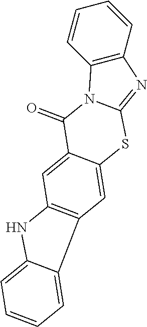 [SMILES: O=c1c2cc3[nH]c4ccccc4c3cc2sc2nc3ccccc3n12] | 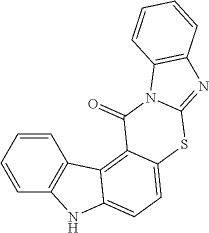 [SMILES: O=c1c2c(ccc3[nH]c4ccccc4c32)sc2nc3ccccc3n12] | 65%, 14% |
| 3c | 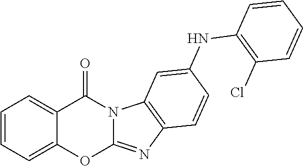 [SMILES: O=c1c2ccccc2oc2nc3ccc(Nc4ccccc4Cl)cc3n12] | 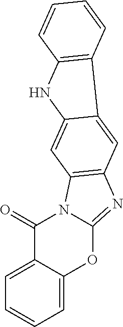 [SMILES: O=c1c2ccccc2oc2nc3cc4c(cc3n12)[nH]c1ccccc14] | 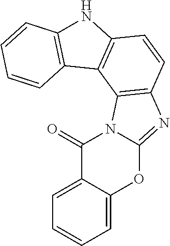 [SMILES: O=c1c2ccccc2oc2nc3ccc4[nH]c5ccccc5c4c3n12] | 66%, 18% |
| 4c | 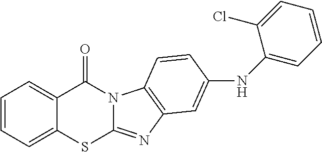 [SMILES: O=c1c2ccccc2sc2nc3cc(Nc4ccccc4Cl)ccc3n12] | 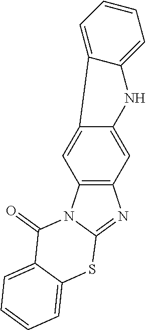 [SMILES: O=c1c2ccccc2sc2nc3cc4[nH]c5ccccc5c4cc3n12] | 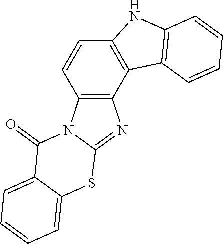 [SMILES: O=c1c2ccccc2sc2nc3c4c(ccc3n12)[nH]c1ccccc14] | 61%, 19% |
| 5c | 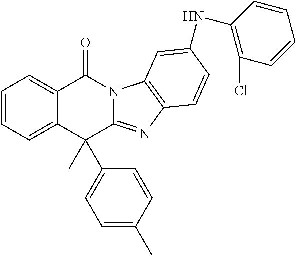 [SMILES: Cc1ccc(C2(C)c3ccccc3C(=O)n3c2nc2ccc(Nc4ccccc4Cl)cc23)cc1] | 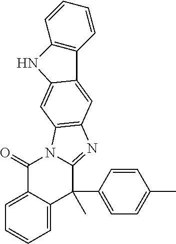 [SMILES: Cc1ccc(C2(C)c3ccccc3C(=O)n3c2nc2cc4c(cc23)[nH]c2ccccc24)cc1] | 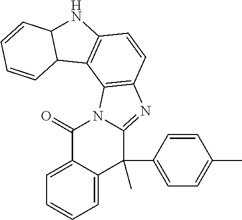 [SMILES: Cc1ccc(C2(C)c3ccccc3C(=O)n3c2nc2ccc4c(c23)C2C=CC=CC2N4)cc1] | 55%, 17% |
| 6c | 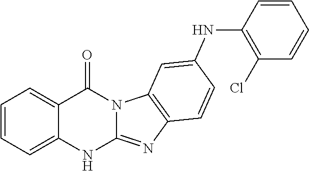 [SMILES: O=c1c2ccccc2[nH]c2nc3ccc(Nc4ccccc4Cl)cc3n12] | 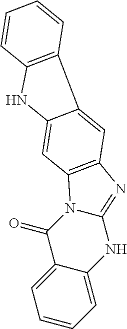 [SMILES: O=c1c2ccccc2[nH]c2nc3cc4c(cc3n12)[nH]c1ccccc14] | 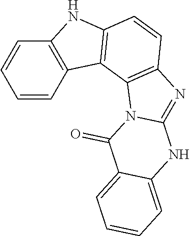 [SMILES: O=c1c2ccccc2[nH]c2nc3ccc4[nH]c5ccccc5c4c3n12] | 54%, 15% |

d) Ullmann Reaction

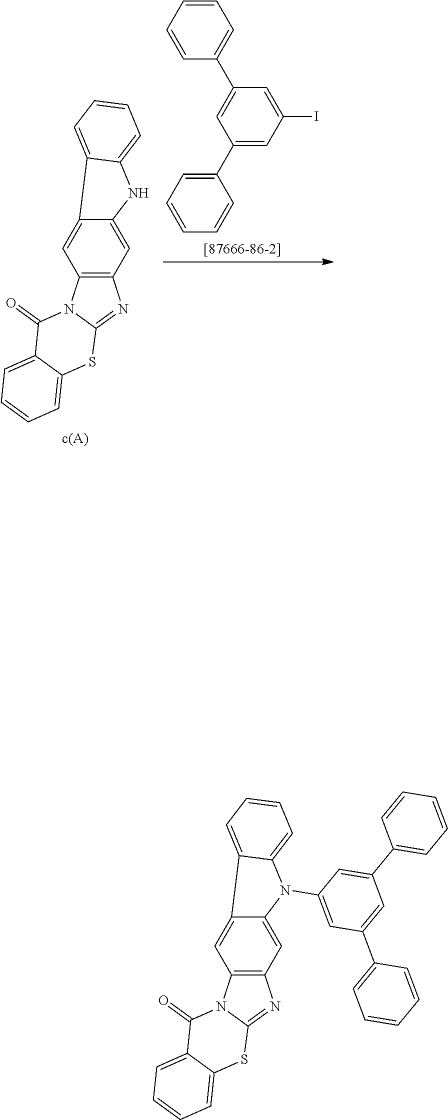

68 g (200 mmol) of compound c (A), 106 g (300 mmol) of 5′-iodo-[1,1′;3′,1″ ]terphenyl and 2.3 g (20 mmol) of L-proline are stirred in 100 ml of 1,2-dichlorobenzene at 150° C. for 30 h. The solution is diluted with water and extracted twice with ethyl acetate, and the combined organic phases are dried over Na₂SO₄ and concentrated by rotary evaporation. The residue is purified by chromatography (EtOAc/hexane: 2/3). The yield is 71 g (125 mmol), 63% of theory.

The following compounds are obtained in an analogous manner:

| Ex. | Reactant 1 | Reactant | Product | Yield |
|---|---|---|---|---|
| 1d | 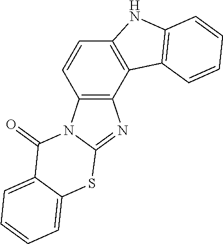 [SMILES: O=c1c2ccccc2sc2nc3c4c(ccc3n12)[nH]c1ccccc14] | 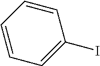 | 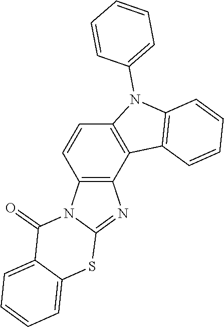 [SMILES: O=c1c2ccccc2sc2nc3c4c5ccccc5n(-c5ccccc5)c4ccc3n12] | 59% |
| 2d | 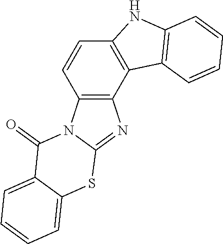 [SMILES: O=c1c2ccccc2sc2nc3c4c(ccc3n12)[nH]c1ccccc14] | 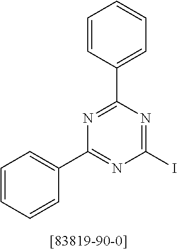 [SMILES: Ic1nc(-c2ccccc2)nc(-c2ccccc2)n1] | 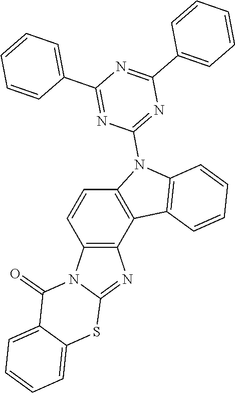 [SMILES: O=c1c2ccccc2sc2nc3c4c5ccccc5n(-c5nc(-c6ccccc6)nc(-c6ccccc6)n5)c4ccc3n12] | 65% |
| 3d | 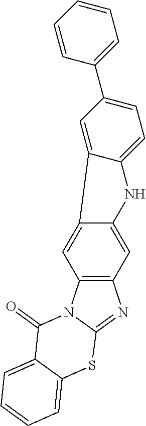 [SMILES: O=c1c2ccccc2sc2nc3cc4[nH]c5ccc(-c6ccccc6)cc5c4cc3n12] | 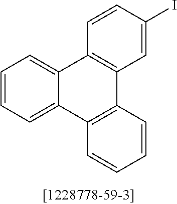 [SMILES: Ic1ccc2c3ccccc3c3ccccc3c2c1] | 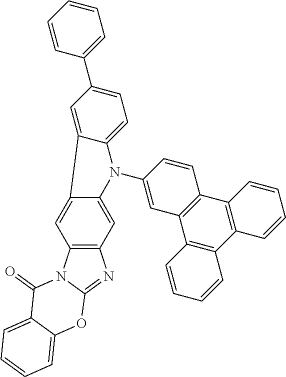 [SMILES: O=c1c2ccccc2oc2nc3cc4c(cc3n12)c1cc(-c2ccccc2)ccc1n4-c1ccc2c3ccccc3c3ccccc3c2c1] | 66% |
| 4d | 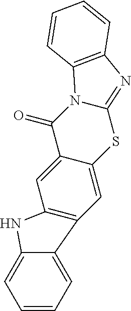 [SMILES: O=c1c2cc3[nH]c4ccccc4c3cc2sc2nc3ccccc3n12] | 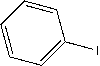 | 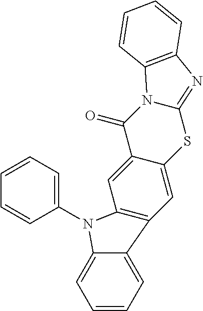 [SMILES: O=c1c2cc3c(cc2sc2nc4ccccc4n12)c1ccccc1n3-c1ccccc1] | 66% |
| 5d | 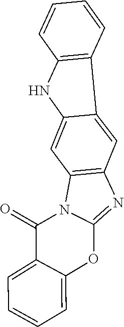 [SMILES: O=c1c2ccccc2oc2nc3cc4c(cc3n12)[nH]c1ccccc14] | 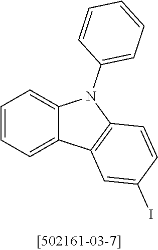 [SMILES: Ic1ccc2c(c1)c1ccccc1n2-c1ccccc1] | 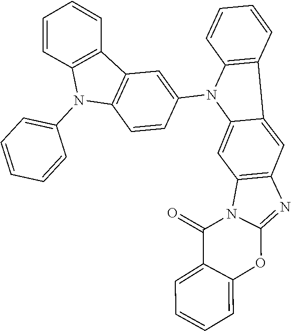 [SMILES: O=c1c2ccccc2oc2nc3cc4c5ccccc5n(-c5ccc6c(c5)c5ccccc5n6-c5ccccc5)c4cc3n12] | 64% |
| 6d | 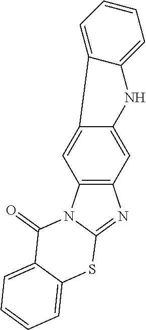 [SMILES: O=c1c2ccccc2sc2nc3cc4[nH]c5ccccc5c4cc3n12] | 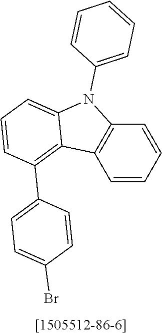 [SMILES: Brc1ccc(-c2cccc3c2c2ccccc2n3-c2ccccc2)cc1] | 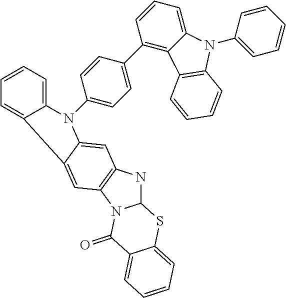 [SMILES: O=C1c2ccccc2SC2Nc3cc4c(cc3N12)c1ccccc1n4-c1ccc(-c2cccc3c2c2ccccc2n3-c2ccccc2)cc1] | 55% |
| 7d | 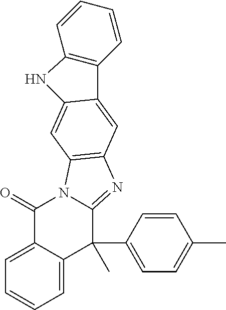 [SMILES: Cc1ccc(C2(C)c3ccccc3C(=O)n3c2nc2cc4c(cc23)[nH]c2ccccc24)cc1] | 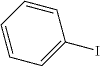 | 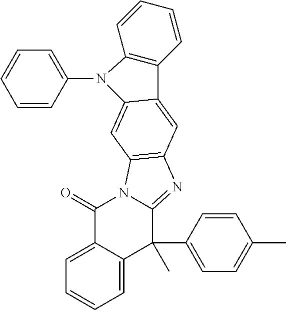 [SMILES: Cc1ccc(C2(C)c3ccccc3C(=O)n3c2nc2cc4c5ccccc5n(-c5ccccc5)c4cc23)cc1] | 71% |
| 8d | 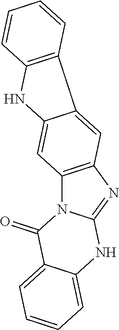 [SMILES: O=c1c2ccccc2[nH]c2nc3cc4c(cc3n12)[nH]c1ccccc14] | 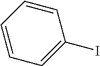 | 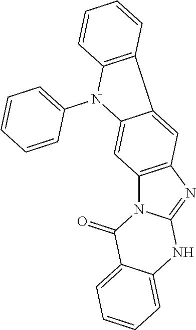 [SMILES: O=c1c2ccccc2[nH]c2nc3cc4c5ccccc5n(-c5ccccc5)c4cc3n12] | 70% |
| 9d | 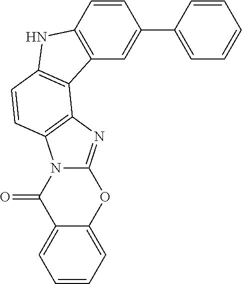 [SMILES: O=c1c2ccccc2oc2nc3c4c(ccc3n12)[nH]c1ccc(-c2ccccc2)cc14] | 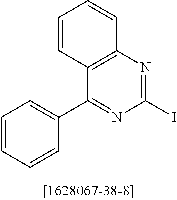 [SMILES: Ic1nc(-c2ccccc2)c2ccccc2n1] | 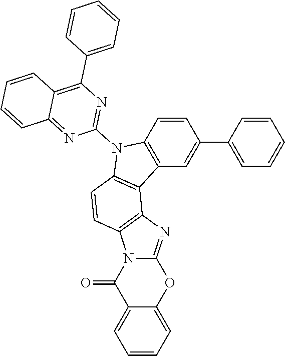 [SMILES: O=c1c2ccccc2oc2nc3c4c5cc(-c6ccccc6)ccc5n(-c5nc(-c6ccccc6)c6ccccc6n5)c4ccc3n12] | 65% |
| 10d | 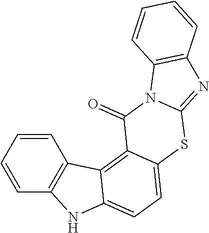 [SMILES: O=c1c2c(ccc3[nH]c4ccccc4c32)sc2nc3ccccc3n12] | 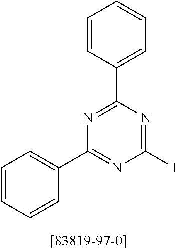 [SMILES: Ic1nc(-c2ccccc2)nc(-c2ccccc2)n1] | 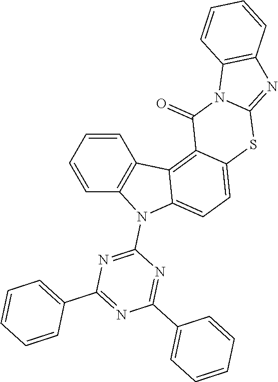 [SMILES: O=c1c2c(ccc3c2c2ccccc2n3-c2nc(-c3ccccc3)nc(-c3ccccc3)n2)sc2nc3ccccc3n12] | 53% |
| 11d | 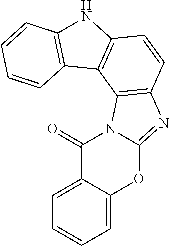 [SMILES: O=c1c2ccccc2oc2nc3ccc4[nH]c5ccccc5c4c3n12] | 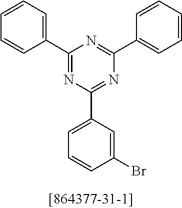 [SMILES: Brc1cccc(-c2nc(-c3ccccc3)nc(-c3ccccc3)n2)c1] | 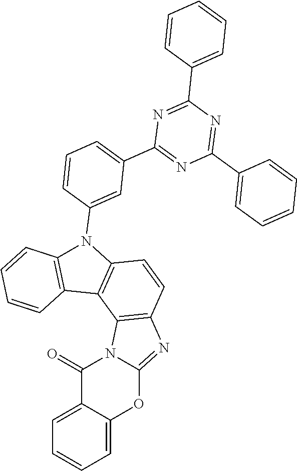 [SMILES: O=c1c2ccccc2oc2nc3ccc4c(c5ccccc5n4-c4cccc(-c5nc(-c6ccccc6)nc(-c6ccccc6)n5)c4)c3n12] | 52% |
| 12d | 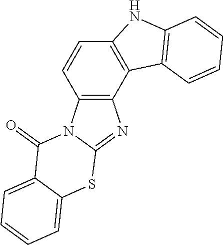 [SMILES: O=c1c2ccccc2sc2nc3c4c(ccc3n12)[nH]c1ccccc14] | 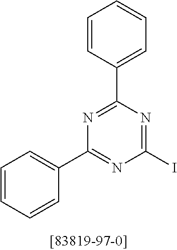 [SMILES: Ic1nc(-c2ccccc2)nc(-c2ccccc2)n1] | 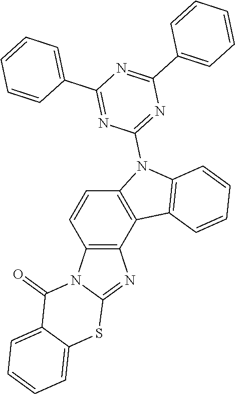 [SMILES: O=c1c2ccccc2sc2nc3c4c5ccccc5n(-c5nc(-c6ccccc6)nc(-c6ccccc6)n5)c4ccc3n12] | 60% |
| 13d | 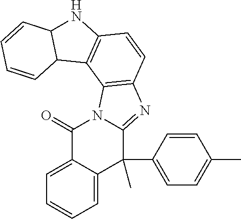 [SMILES: Cc1ccc(C2(C)c3ccccc3C(=O)n3c2nc2ccc4c(c23)C2C=CC=CC2N4)cc1] | 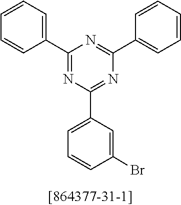 [SMILES: Brc1cccc(-c2nc(-c3ccccc3)nc(-c3ccccc3)n2)c1] | 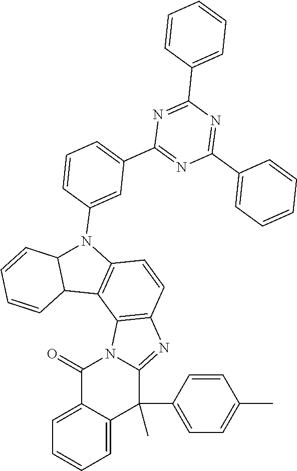 [SMILES: Cc1ccc(C2(C)c3ccccc3C(=O)n3c2nc2ccc4c(c23)C2C=CC=CC2N4c2cccc(-c3nc(-c4ccccc4)nc(-c4ccccc4)n3)c2)cc1] | 47% |
| 14d | 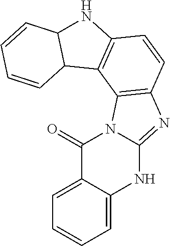 [SMILES: O=c1c2ccccc2[nH]c2nc3ccc4c(c3n12)C1C=CC=CC1N4] | 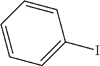 | 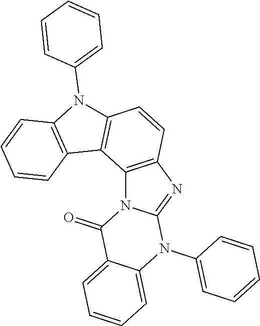 [SMILES: O=c1c2ccccc2n(-c2ccccc2)c2nc3ccc4c(c5ccccc5n4-c4ccccc4)c3n12] | 60% |
| 15d | 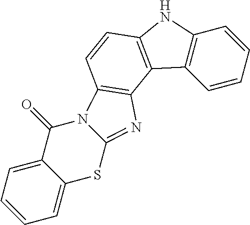 [SMILES: O=c1c2ccccc2sc2nc3c4c(ccc3n12)[nH]c1ccccc14] | 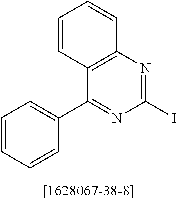 [SMILES: Ic1nc(-c2ccccc2)c2ccccc2n1] | 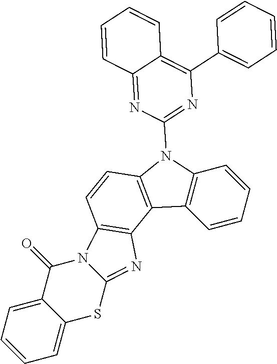 [SMILES: O=c1c2ccccc2sc2nc3c4c5ccccc5n(-c5nc(-c6ccccc6)c6ccccc6n5)c4ccc3n12] | 63% |
| 16d | 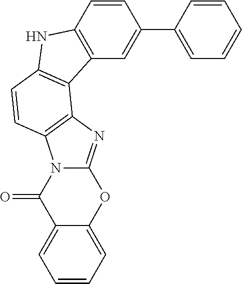 [SMILES: O=c1c2ccccc2oc2nc3c4c(ccc3n12)[nH]c1ccc(-c2ccccc2)cc14] | 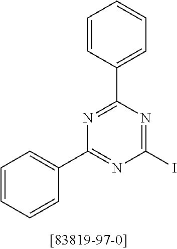 [SMILES: Ic1nc(-c2ccccc2)nc(-c2ccccc2)n1] | 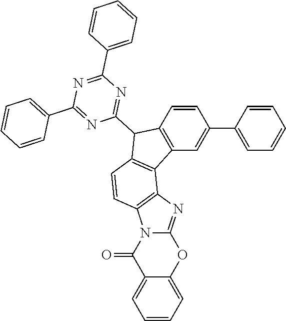 [SMILES: O=c1c2ccccc2oc2nc3c4c(ccc3n12)C(c1nc(-c2ccccc2)nc(-c2ccccc2)n1)c1ccc(-c2ccccc2)cc1-4] | 70% |
| 17d | 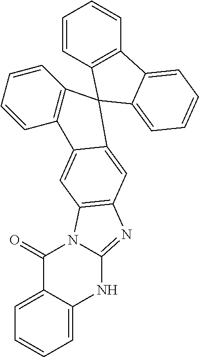 [SMILES: O=c1c2ccccc2[nH]c2nc3cc4c(cc3n12)-c1ccccc1C41c2ccccc2-c2ccccc21] | 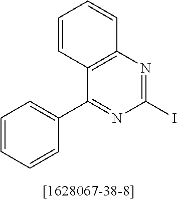 [SMILES: Ic1nc(-c2ccccc2)c2ccccc2n1] | 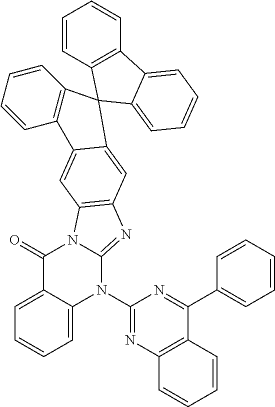 [SMILES: O=c1c2ccccc2n(-c2nc(-c3ccccc3)c3ccccc3n2)c2nc3cc4c(cc3n12)-c1ccccc1C41c2ccccc2-c2ccccc21] | 65% |
| 18d | 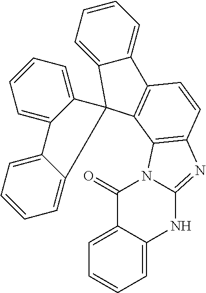 [SMILES: O=c1c2ccccc2[nH]c2nc3ccc4c(c3n12)C1(c2ccccc2-c2ccccc21)c1ccccc1-4] | 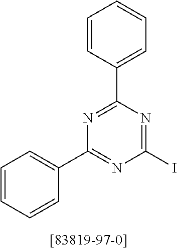 [SMILES: Ic1nc(-c2ccccc2)nc(-c2ccccc2)n1] | 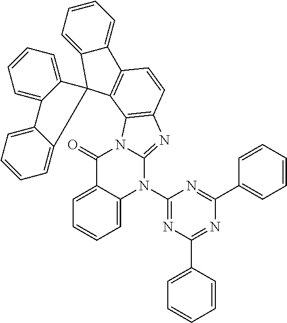 [SMILES: O=c1c2ccccc2n(-c2nc(-c3ccccc3)nc(-c3ccccc3)n2)c2nc3ccc4c(c3n12)C1(c2ccccc2-c2ccccc21)c1ccccc1-4] | 63% |

### e) 2-(12-Oxobenzimidazolo[2,1-b][1,3]benzoxazin-8-yl)methylbenzoic acid

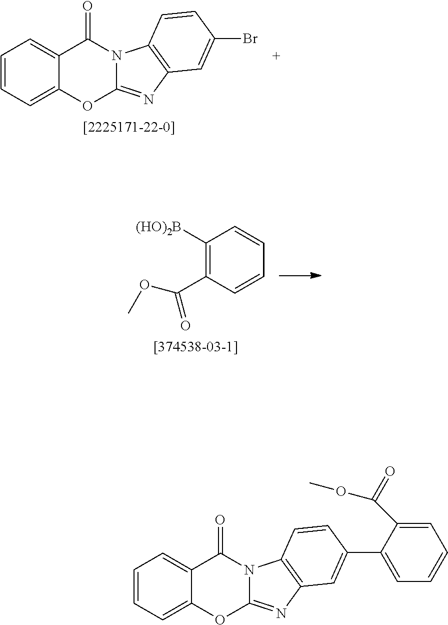 [SMILES: O=c1c2ccccc2oc2nc3cc(Br)ccc3n12 ; COC(=O)c1ccccc1B(O)O ; COC(=O)c1ccccc1-c1ccc2c(c1)nc1oc3ccccc3c(=O)n12]

To a well-stirred, degassed suspension of 10.6 g (71 mmol) of [2-(methoxycarbonyl)phenyl]boronic acid, 22.3 g (72 mmol) of 8-bromobenzimidazolo[2,1-b][1,3]benzoxazin-12-one and 18.9 g (6.6 mmol) of tripotassium phosphate in a mixture of 350 ml of water and 350 ml of THE is added 1.55 g (0.1 mmol) of Pd(PPh₃)₄, and the mixture is heated under reflux for 60 h. After cooling, the organic phase is removed, washed three times with 200 ml of water and once with 200 ml of saturated sodium chloride solution and then dried over magnesium sulphate. The organic phase is concentrated to dryness by rotary evaporation under reduced pressure. The grey residue thus obtained is recrystallized from dioxane. The precipitated crystals are filtered off with suction, washed with 50 ml of ethanol and then dried under reduced pressure. Yield: 22.1 g (59 mmol), 83% of theory.

The following compounds can be prepared analogously:

| | Reactant 1 | Reactant 2 | Product | Yield |
|---|---|---|---|---|
| 1e | 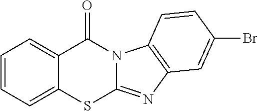 [SMILES: O=c1c2ccccc2sc2nc3cc(Br)ccc3n12] | 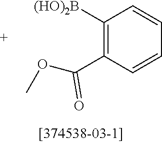 | 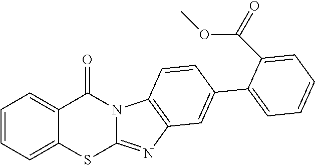 [SMILES: COC(=O)c1ccccc1-c1ccc2c(c1)nc1sc3ccccc3c(=O)n12] | 70% |
| 2e | 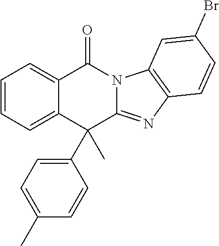 [SMILES: Cc1ccc(C2(C)c3ccccc3C(=O)n3c2nc2ccc(Br)cc23)cc1] | 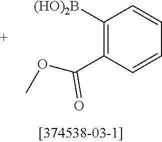 | 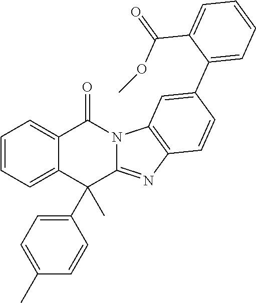 [SMILES: COC(=O)c1ccccc1-c1ccc2nc3n(c2c1)C(=O)c1ccccc1C3(C)c1ccc(C)cc1] | 73% |
| 3e | 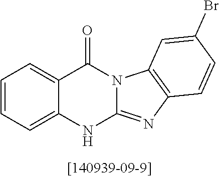 [SMILES: O=c1c2ccccc2[nH]c2nc3ccc(Br)cc3n12] | 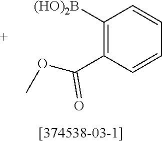 | 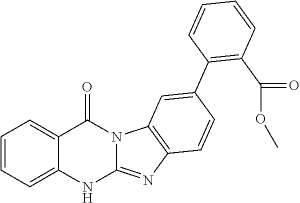 [SMILES: COC(=O)c1ccccc1-c1ccc2nc3[nH]c4ccccc4c(=O)n3c2c1] | 75% |
| 4e | 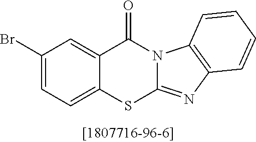 [SMILES: O=c1c2cc(Br)ccc2sc2nc3ccccc3n12] | 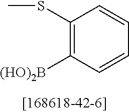 | 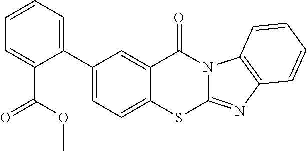 [SMILES: COC(=O)c1ccccc1-c1ccc2sc3nc4ccccc4n3c(=O)c2c1] | 69% |
| 5e | 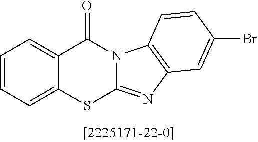 [SMILES: O=c1c2ccccc2sc2nc3cc(Br)ccc3n12] | 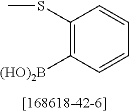 | 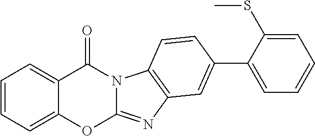 [SMILES: CSc1ccccc1-c1ccc2c(c1)nc1oc3ccccc3c(=O)n12] | 70% |

f) Cyclization

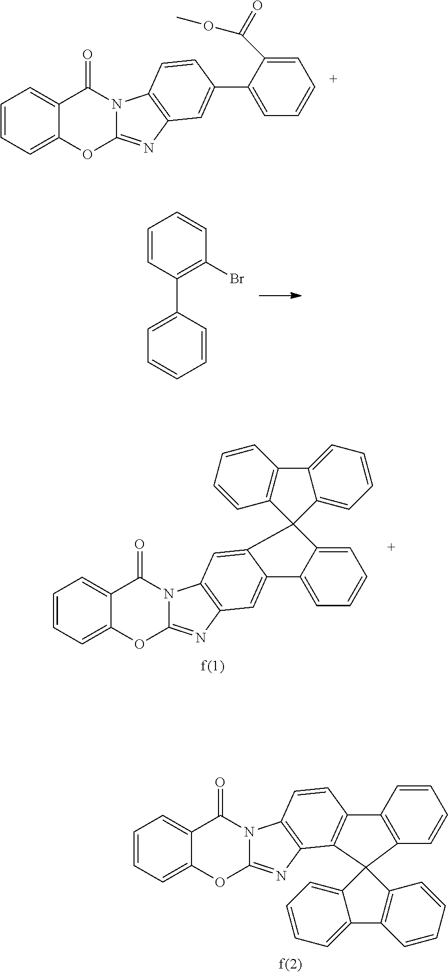 [SMILES: COC(=O)c1ccccc1-c1ccc2c(c1)nc1oc3ccccc3c(=O)n12 ; Brc1ccccc1-c1ccccc1 ; O=c1c2ccccc2oc2nc3cc4c(cc3n12)C1(c2ccccc2-c2ccccc21)c1ccccc1-4 ; O=c1c2ccccc2oc2nc3c4c(ccc3n12)-c1ccccc1C41c2ccccc2-c2ccccc21]

63 g (166 mmol) of 2-bromobiphenyl is initially charged in 700 ml of THE at −78° C. At this temperature, 70 ml of BuLi (2.5 M in hexane) is added dropwise. After 1 h, 99 g (166 mmol) of methyl 2-(12-oxobenzimidazolo[2,1-b][1,3]benzoxazin-8-ylbenzoate in 200 ml of THE is added dropwise. The mixture is left to stir at room temperature overnight, added to ice-water and extracted with dichloromethane. The combined organic phases are washed with water and dried over sodium sulphate. The solvent is removed under reduced pressure and the residue, without further purification, is heated with 90 ml of HCl and 1 l of AcOH at 75° C. overnight. After cooling, the precipitated solid is filtered off with suction and washed twice with 150 ml of water and three times with 150 ml each time of ethanol, and finally recrystallized from heptane. The residue is subjected to hot extraction with toluene and recrystallized from toluene and finally sublimed under high vacuum. Yield: 96 g (60 mmol) of f1 and f2, 87%. After recrystallization from toluene/EA (2:1), 51% e1 and 18% e2 are obtained with a purity of about 99.9% by HPLC.

The following compounds can be prepared analogously:

| | Reactant 1 | Reactant | Product 1 | Product 2 | Yield |
|---|---|---|---|---|---|
| 1f | 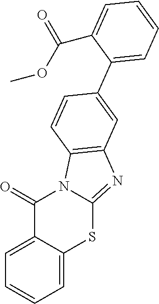 [SMILES: COC(=O)c1ccccc1-c1ccc2c(c1)nc1sc3ccccc3c(=O)n12] | 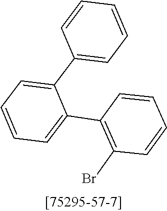 [SMILES: Brc1ccccc1-c1ccccc1-c1ccccc1] | 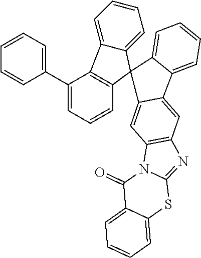 [SMILES: O=c1c2ccccc2sc2nc3cc4c(cc3n12)C1(c2ccccc2-4)c2ccccc2-c2c(-c3ccccc3)cccc21] | 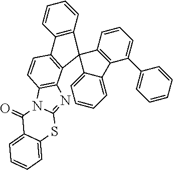 [SMILES: O=c1c2ccccc2sc2nc3c4c(ccc3n12)-c1ccccc1C41c2ccccc2-c2c(-c3ccccc3)cccc21] | 53%/ 17% |
| 2f | 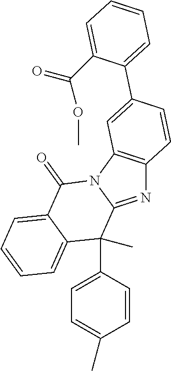 [SMILES: COC(=O)c1ccccc1-c1ccc2nc3n(c2c1)C(=O)c1ccccc1C3(C)c1ccc(C)cc1] | 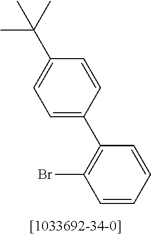 [SMILES: CC(C)(C)c1ccc(-c2ccccc2Br)cc1] | 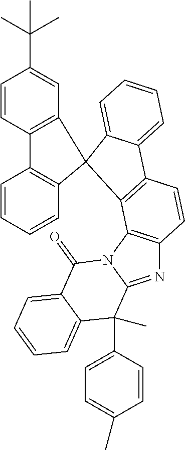 [SMILES: Cc1ccc(C2(C)c3ccccc3C(=O)n3c2nc2ccc4c(c23)C2(c3ccccc3-c3ccc(C(C)(C)C)cc32)c2ccccc2-4)cc1] | 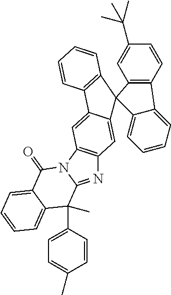 [SMILES: Cc1ccc(C2(C)c3ccccc3C(=O)n3c2nc2cc4c(cc23)-c2ccccc2C42c3ccccc3-c3ccc(C(C)(C)C)cc32)cc1] | 52%/ 17% |
| 3f | 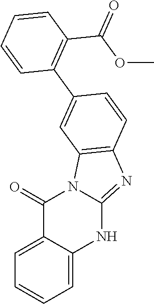 [SMILES: COC(=O)c1ccccc1-c1ccc2nc3[nH]c4ccccc4c(=O)n3c2c1] | 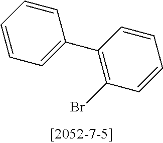 [SMILES: Brc1ccccc1-c1ccccc1] | 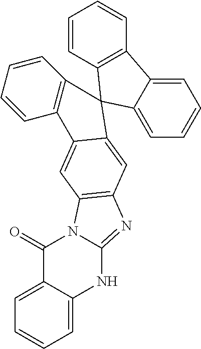 [SMILES: O=c1c2ccccc2[nH]c2nc3cc4c(cc3n12)-c1ccccc1C41c2ccccc2-c2ccccc21] | 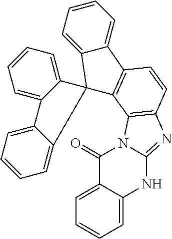 [SMILES: O=c1c2ccccc2[nH]c2nc3ccc4c(c3n12)C1(c2ccccc2-c2ccccc21)c1ccccc1-4] | 46%/ 14% |
| 4f | 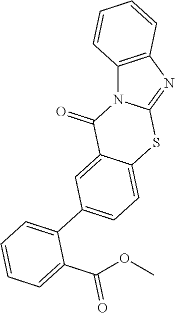 [SMILES: COC(=O)c1ccccc1-c1ccc2sc3nc4ccccc4n3c(=O)c2c1] | 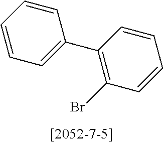 [SMILES: Brc1ccccc1-c1ccccc1] | 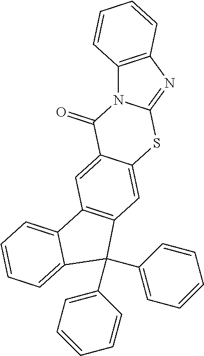 [SMILES: O=c1c2cc3c(cc2sc2nc4ccccc4n12)C(c1ccccc1)(c1ccccc1)c1ccccc1-3] | 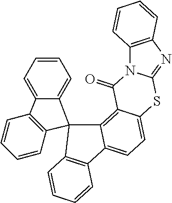 [SMILES: O=c1c2c3c(ccc2sc2nc4ccccc4n12)-c1ccccc1C31c2ccccc2-c2ccccc21] | 51%/ 15% |

### g) 8-(2-Methylsulfinylphenyl)benzimidazolo[2,1-b][1,3]benzoxazin-12-one

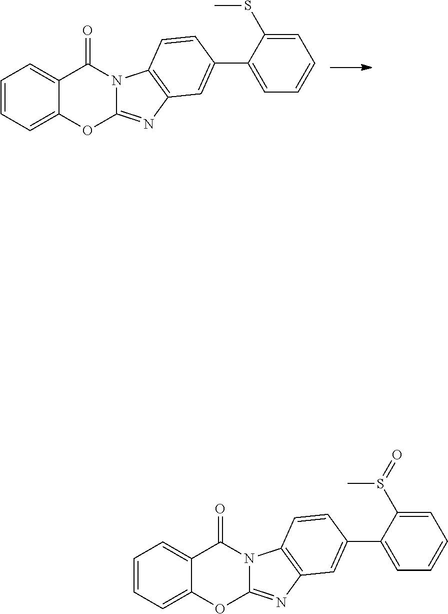 [SMILES: CSc1ccccc1-c1ccc2c(c1)nc1oc3ccccc3c(=O)n12 ; CS(=O)c1ccccc1-c1ccc2c(c1)nc1oc3ccccc3c(=O)n12]

Under protective gas, 107 g (300 mmol) of 8-(2-methylsulfanylphenyl)benzimidazolo[2,1-b][1,3]benzoxazin-12-one is initially charged in 1.1 l of glacial acetic acid and 125 ml of dichloromethane, and the mixture is cooled to 0° C. Added dropwise to this solution is 500 ml (309 mmol) of 30% H₂O₂ solution, and the mixture is stirred overnight. The mixture is admixed with Na₂SO₃ solution, the phases are separated and the solvent is removed under reduced pressure. Yield: 95 g (255 mmol), 85% of theory; purity: 92% by HPLC.

h) Cyclization

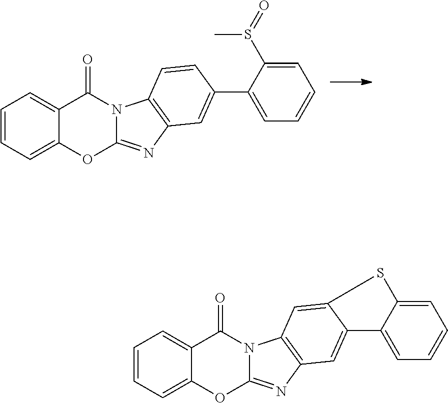 [SMILES: CS(=O)c1ccccc1-c1ccc2c(c1)nc1oc3ccccc3c(=O)n12 ; O=c1c2ccccc2oc2nc3cc4c(cc3n12)sc1ccccc14]

A mixture of 112 g (275 mmol) of 8-(2-methylsulfinylphenyl)benzimidazolo[2,1-b][1,3]benzoxazin-12-one and 737 ml (8329 mol) of trifluoromethanesulfonic acid is stirred at 5° C. for 48 h. Subsequently, the mixture is admixed with 2.4 l of water/pyridine 5:1 and heated under reflux for 20 min. After cooling to room temperature, 500 ml of water and 1000 ml of dichloromethane are added cautiously. The organic phase is washed with 4×50 ml of H₂O and dried over MgSO₄, and the solvents are removed under reduced pressure. The pure product is obtained by recrystallization. Yield: 81 g (240 mmol), 80% of theory; purity: 96% by HPLC.

Production of the OLEDs

Examples E1 to E14 which follow (see table 1) present the use of the materials of the invention in OLEDs.

Pretreatment for examples E1 to E14: Glass plates coated with structured ITO (indium tin oxide) of thickness 50 nm are treated prior to coating with an oxygen plasma, followed by an argon plasma. These plasma-treated glass plates form the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the OLEDs are shown in table 2.

All materials are applied by thermal vapor deposition in a vacuum chamber. In this case, the emission layer always consists of at least one matrix material (host material) and an emitting dopant (emitter) which is added to the matrix material(s) in a particular proportion by volume by co-evaporation. Details given in such a form as EG1:IC2:TEG1 (49%:44%:7%) mean here that the material EG1 is present in the layer in a proportion by volume of 49%, IC2 in a proportion of 44% and TEG1 in a proportion of 7%. Analogously, the electron transport layer may also consist of a mixture of two materials.

The OLEDs are characterized in a standard manner. For this purpose, electroluminescence spectra, current efficiency (CE, measured in cd/A) and external quantum efficiency (EQE, measured in %) are determined as a function of luminance, calculated from current-voltage-luminance characteristics assuming Lambertian emission characteristics. Electroluminescence spectra are determined at a luminance of 1000 cd/m², and these are used to calculate the CIE 1931 x and y color coordinates. The results thus obtained can be found in table 3.

Use of the Materials of the Invention in OLEDs

The inventive compounds EG1 to EG8 and EG11 can be used in examples E1 to E9 and E12 to E13 as matrix material in the emission layer of phosphorescent green OLEDs. The inventive compounds EG9 to EG10 can be used in examples E10 to E11 as matrix material in the emission layer of phosphorescent red OLEDs. The inventive compound EG5 can be used in example E14 as electron transport in the ETM layer of phosphorescent green OLEDs.

TABLE 1
Structure of the OLEDs

| | HIL | HTL | EBL | EML | HBL | ETL | EIL |
|---|---|---|---|---|---|---|---|
| Ex. | thickness | thickness | thickness | thickness | thickness | thickness | thickness |
| E1 | HATCN | SpMA1 | SpMA2 | EG1:IC2:TEG1 | ST2 | ST2:LiQ (50%:50%) | LiQ |
| | 5 nm | 230 nm | 20 nm | (49%:44%:7%) 40 nm | 5 nm | 30 nm | 1 nm |
| E2 | HATCN | SpMA1 | SpMA2 | EG2:IC2:TEG1 | ST2 | ST2:LiQ (50%:50%) | LiQ |
| | 5 nm | 230 nm | 20 nm | (49%:44%:7%) 40 nm | 5 nm | 30 nm | 1 nm |
| E3 | HATCN | SpMA1 | SpMA2 | EG3:IC3:TEG1 | ST2 | ST2:LiQ (50%:50%) | LiQ |
| | 5 nm | 230 nm | 20 nm | (49%:44%:7%) 40 nm | 5 nm | 30 nm | 1 nm |
| E4 | HATCN | SpMA1 | SpMA2 | EG4:IC3:TEG1 | ST2 | ST2:LiQ (50%:50%) | LiQ |
| | 5 nm | 230 nm | 20 nm | (49%:44%:7%) 40 nm | 5 nm | 30 nm | 1 nm |
| E5 | HATCN | SpMA1 | SpMA2 | EG5:IC3:TEG1 | ST2 | ST2:LiQ (50%:50%) | LiQ |
| | 5 nm | 230 nm | 20 nm | (45%:45%:10%) 30 nm | 10 nm | 30 nm | 1 nm |
| E6 | HATCN | SpMA1 | SpMA2 | EG6:IC3:TEG1 | ST2 | ST2:LiQ (50%:50%) | LiQ |
| | 5 nm | 230 nm | 20 nm | (44%:44%:12%) 30 nm | 10 nm | 30 nm | 1 nm |
| E7 | HATCN | SpMA1 | SpMA2 | EG7:IC3:TEG1 | ST2 | ST2:LiQ (50%:50%) | LiQ |
| | 5 nm | 230 nm | 20 nm | (44%:44%:12%) 30 nm | 10 nm | 30 nm | 1 nm |
| E8 | HATCN | SpMA1 | SpMA2 | EG8:IC3:TEG1 | ST2 | ST2:LiQ (50%:50%) | LiQ |
| | 5 nm | 230 nm | 20 nm | (46%:47%:7%) 30 nm | 10 nm | 30 nm | 1 nm |
| E9 | HATCN | SpMA1 | SpMA2 | EG8:IC3:TEG1 | ST2 | ST2:LiQ (50%:50%) | LiQ |
| | 5 nm | 230 nm | 20 nm | (46%:47%:7%) 30 nm | 10 nm | 30 nm | 1 nm |
| E10 | HATCN | SpMA1 | SpMA2 | EG9:TER5 | ST2 | ST2:LiQ (50%:50%) | LiQ |
| | 5 nm | 125 nm | 10 nm | (97%:3%) 35 nm | 10 nm | 30 nm | 1 nm |
| E11 | HATCN | SpMA1 | SpMA2 | EG10:TER5 | ST2 | ST2:LiQ (50%:50%) | LiQ |
| | 5 nm | 125 nm | 10 nm | (97%:3%) 35 nm | 10 nm | 30 nm | 1 nm |
| E12 | HATCN | SpMA1 | SpMA2 | IC1:EG11:TEG1 | ST2 | ST2:LiQ (50%:50%) | LiQ |
| | 5 nm | 230 nm | 20 nm | (46%:47%:7%) 30 nm | 10 nm | 30 nm | 1 nm |
| E13 | HATCN | SpMA1 | SpMA2 | EG4:TEG1 | ST2 | ST2:LiQ (50%:50%) | LiQ |
| | 5 nm | 230 nm | 20 nm | (49%:44%:7%) 40 nm | 5 nm | 30 nm | 1 nm |
| E14 | HATCN | SpMA1 | SpMA2 | IC1:TEG1 | — | EG5 | LiQ |
| | 5 nm | 70 nm | 15 nm | (90%:10%) 25 nm | | 45 nm | 3 nm |

TABLE 2
Structural formulae of the materials for the OLEDs

| | |
|---|---|
| 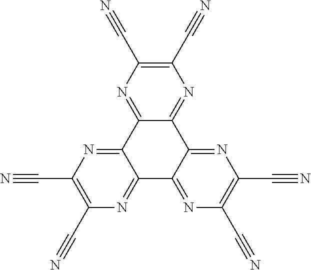 [SMILES: N#Cc1nc2c3nc(C#N)c(C#N)nc3c3nc(C#N)c(C#N)nc3c2nc1C#N] | HATCN |
| 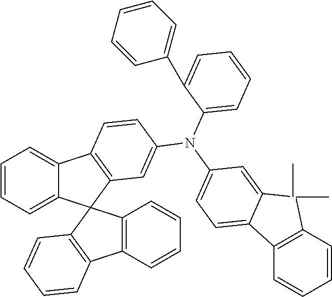 | SpMA1 |
| 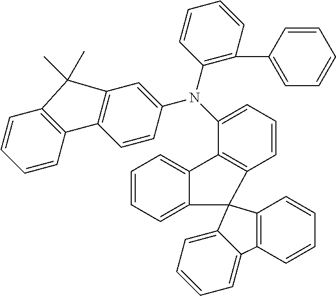 [SMILES: CC1(C)c2ccccc2-c2ccc(N(c3ccccc3-c3ccccc3)c3cccc4c3-c3ccccc3C43c4ccccc4-c4ccccc43)cc21] | SpMA3 |
| 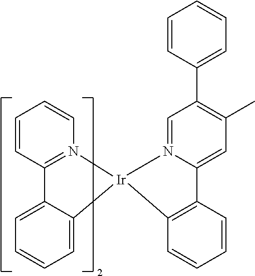 | TEG1 |
| 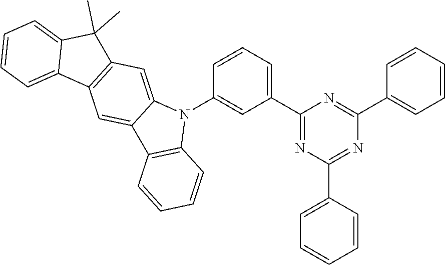 [SMILES: CC1(C)c2ccccc2-c2cc3c4ccccc4n(-c4cccc(-c5nc(-c6ccccc6)nc(-c6ccccc6)n5)c4)c3cc21] | IC1 |
| 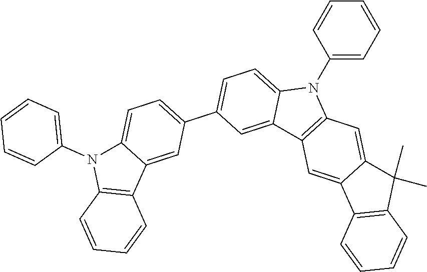 [SMILES: CC1(C)c2ccccc2-c2cc3c4cc(-c5ccc6c(c5)c5ccccc5n6-c5ccccc5)ccc4n(-c4ccccc4)c3cc21] | IC2 |
| 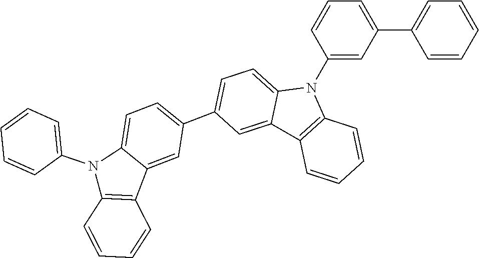 [SMILES: c1ccc(-c2cccc(-n3c4ccccc4c4cc(-c5ccc6c(c5)c5ccccc5n6-c5ccccc5)ccc43)c2)cc1] | IC3 |
| 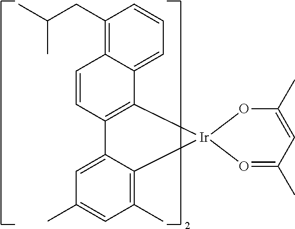 | TER5 |
| 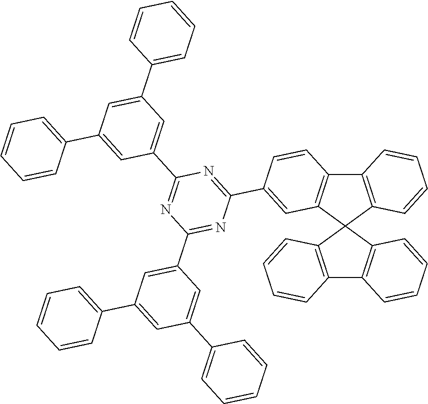 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ST2 |
| 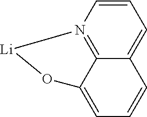 | LIQ |
| 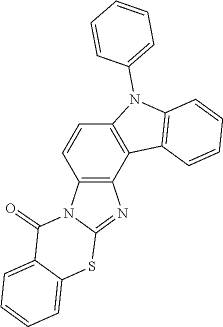 [SMILES: O=c1c2ccccc2sc2nc3c4c5ccccc5n(-c5ccccc5)c4ccc3n12] | EG1 (1d) |
| 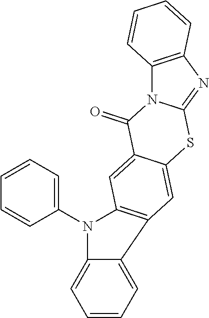 [SMILES: O=c1c2cc3c(cc2sc2nc4ccccc4n12)c1ccccc1n3-c1ccccc1] | EG2 (4b) |
| 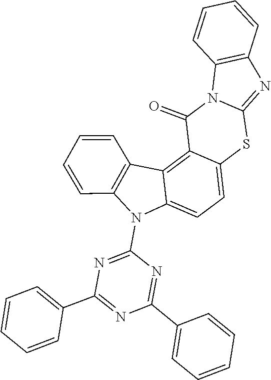 [SMILES: O=c1c2c(ccc3c2c2ccccc2n3-c2nc(-c3ccccc3)nc(-c3ccccc3)n2)sc2nc3ccccc3n12] | EG3 (10d) |
| 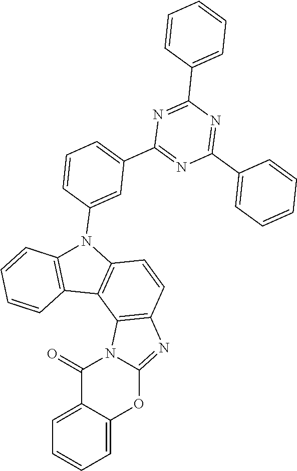 [SMILES: O=c1c2ccccc2oc2nc3ccc4c(c5ccccc5n4-c4cccc(-c5nc(-c6ccccc6)nc(-c6ccccc6)n5)c4)c3n12] | EG4 (11d) |
| 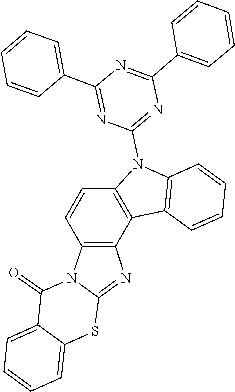 [SMILES: O=c1c2ccccc2sc2nc3c4c5ccccc5n(-c5nc(-c6ccccc6)nc(-c6ccccc6)n5)c4ccc3n12] | EG5 (12d) |
| 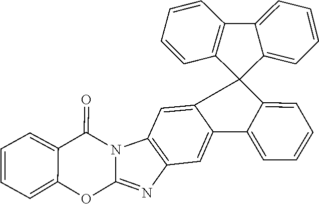 [SMILES: O=c1c2ccccc2oc2nc3cc4c(cc3n12)C1(c2ccccc2-c2ccccc21)c1ccccc1-4] | EG6 (f(1)) |
| 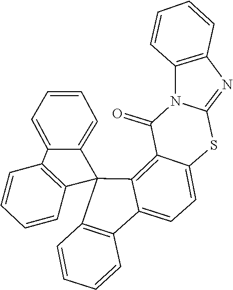 [SMILES: O=c1c2c3c(ccc2sc2nc4ccccc4n12)-c1ccccc1C31c2ccccc2-c2ccccc21] | EG7 (4f) |
| 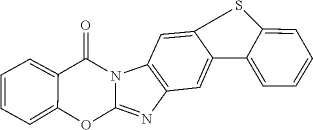 [SMILES: O=c1c2ccccc2oc2nc3cc4c(cc3n12)sc1ccccc14] | EG8 (g) |
| 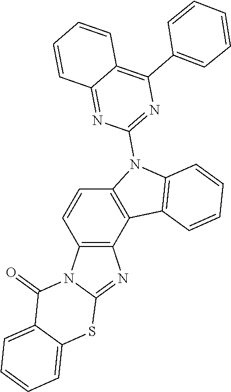 [SMILES: O=c1c2ccccc2sc2nc3c4c5ccccc5n(-c5nc(-c6ccccc6)c6ccccc6n5)c4ccc3n12] | EG9 (15d) |
| 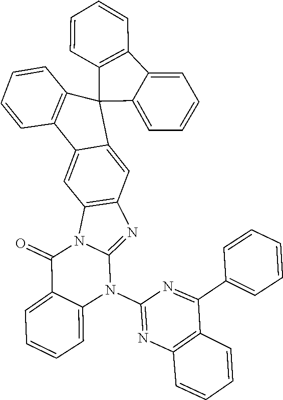 [SMILES: O=c1c2ccccc2n(-c2nc(-c3ccccc3)c3ccccc3n2)c2nc3cc4c(cc3n12)-c1ccccc1C41c2ccccc2-c2ccccc21] | EG10(12b) |
| 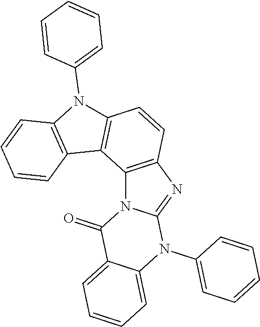 [SMILES: O=c1c2ccccc2n(-c2ccccc2)c2nc3ccc4c(c5ccccc5n4-c4ccccc4)c3n12] | EG11 (14d) |

TABLE 3
Data of the OLEDs

| | U1000 | SE1000 | EQE 1000 | CIE x/y at |
|---|---|---|---|---|
| Ex. | (V) | (cd/A) | (%) | 1000 cd/m² |
| E1 | 3.4 | 65 | 16.2 | 0.35/0.61 |
| E2 | 3.4 | 66 | 17.3 | 0.33/0.62 |
| E3 | 3.3 | 64 | 18.1 | 0.35/0.61 |
| E4 | 3.0 | 69 | 18.2 | 0.35/0.62 |
| E5 | 3.0 | 72 | 18.4 | 0.33/0.63 |
| E6 | 3.3 | 70 | 17.7 | 0.33/0.62 |
| E7 | 3.3 | 62 | 17.6 | 0.32/0.64 |
| E8 | 3.2 | 73 | 18.0 | 0.32/0.63 |
| E9 | 3.1 | 71 | 17.3 | 0.33/0.62 |
| E10 | 3.9 | 24 | 20 | 0.66/0.34 |
| E11 | 3.6 | 22 | 22 | 0.65/0.33 |
| E12 | 3.5 | 80 | 16 | 0.35/0.61 |
| E13 | 3.4 | 75 | 17 | 0.35/0.61 |
| E14 | 3.6 | 63 | 16.9 | 0.33/0.62 |

1.-11. (canceled)

12. A compound of formula (1) 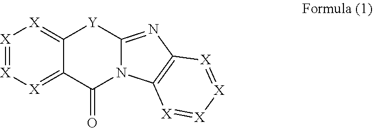
where
X is the same or different at each instance and is CR or N, where there is at least one instance of two adjacent X groups that are a group of the following formula (2), and the other symbols X are the same or different at each instance and are CR or N: 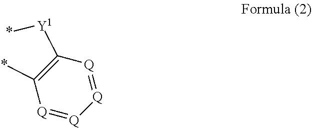
Y, Y¹ is the same or different at each instance and is an NR, NAr, CR₂, SiR₂, BAr, C═O, O or S;
Q is the same or different at each instance and is N or CR;
Ar is the same or different at each instance and is an aromatic or heteroaromatic ring system which has 5 to 60 aromatic ring atoms and may be substituted in each case by one or more R radicals;
R is the same or different at each instance and is H, D, F, Cl, Br, I, B(OR¹)₂, CHO, C(═O)R¹, CR¹═C(R¹)₂, CN, C(═O)OR¹, C(═O)N(R¹)₂, Si(R¹)₃, N(R¹)₂, NO₂, P(═O)(R¹)₂, OSO₂R¹, OR¹, S(═O)R¹, S(═O)₂R¹, SR¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may be substituted in each case by one or more R¹ radicals, where one or more nonadjacent CH₂ groups may be replaced by —R¹C═CR¹—, —C≡C—, Si(R¹)₂, C═O, C═S, C═NR¹, —C(═O)O—, —C(═O)NR¹—, NR¹, P(═O)(R′), —O—, —S—, SO or SO₂, or an aromatic or heteroaromatic ring system which has 5 to 60 aromatic ring atoms, preferably 5 to 40 aromatic ring atoms, and may be substituted in each case by one or more R¹ radicals, where two or more R radicals may be joined to one another and may form a ring;
R¹ is the same or different at each instance and is H, D, F, Cl, Br, I, B(OR²)₂, CHO, C(═O)R², CR²═C(R²)₂, CN, C(═O)OR², C(═O)N(R²)₂, Si(R²)₃, N(R²)₂, NO₂, P(═O)(R²)₂, OSO₂R², OR², S(═O)R², S(═O)₂R², a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R² radicals and where one or more CH₂ groups in the abovementioned groups may be replaced by —R²C═CR²—, —C≡C—, Si(R²)₂, C═O, C═S, C═NR², —C(═O)O—, —C(═O)NR²—, NR², P(═O)(R²), —O—, —S—, SO or SO₂ and where one or more hydrogen atoms in the abovementioned groups may be replaced by D, F, Cl, Br, I, CN or NO₂, or an aromatic or heteroaromatic ring system which has 5 to 30 aromatic ring atoms and may be substituted in each case by one or more R² radicals, where two or more R¹ radicals may be joined to one another and may form a ring;
R² is the same or different at each instance and is H, D, F or an aliphatic, aromatic or heteroaromatic organic radical having 1 to 20 carbon atoms, in which one or more hydrogen atoms may also be replaced by D or F; at the same time, two or more R² substituents may be joined to one another and may form a ring.

13. The compound as claimed in claim 12, selected from the compounds of the formulae (3) to (6): 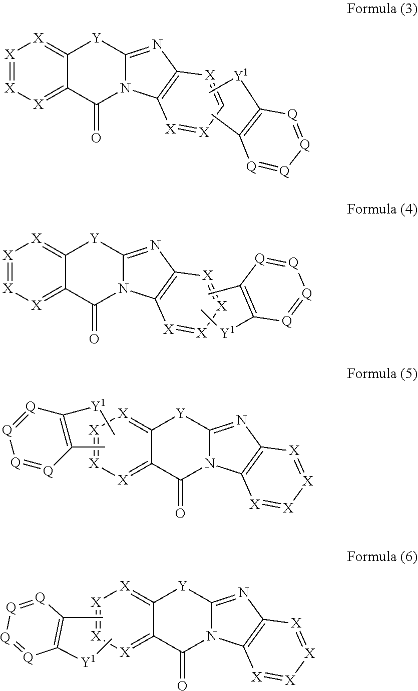
where the symbols used have the definitions given in claim 12.

14. The compound as claimed in claim 12, wherein not more than two symbols X per cycle are N.

15. The compound as claimed in claim 12, selected from the compounds of the formulae (7) to (10): 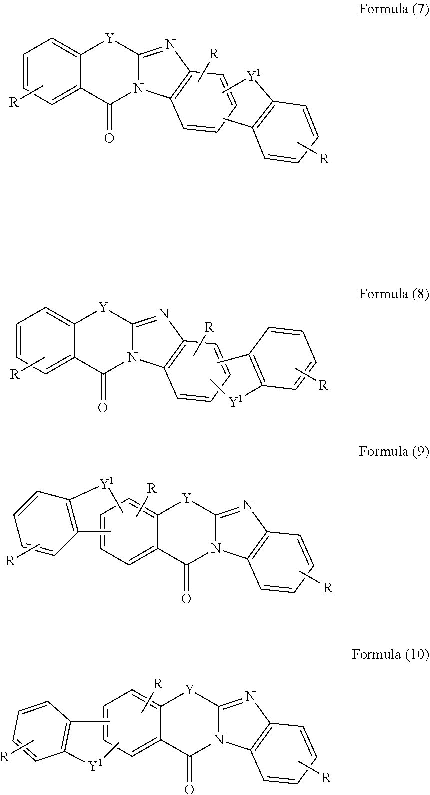
where the symbols used have the definitions given in claim 12.

16. The compound as claimed in claim 13, wherein the Y¹ group in the formulae (3) and (4) or (7) and (8) is in the para position to the nitrogen atoms, and in formulae (5) and (6) or (9) and (10) is in the para position to the keto group or to Y.

17. The compound as claimed in claim 12, selected from the compounds of the formulae (11) to (14) 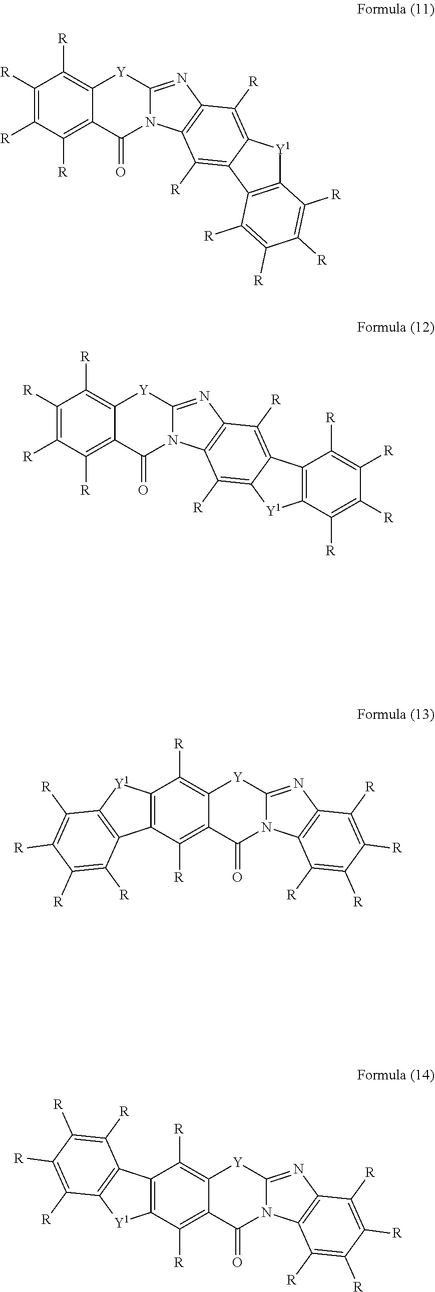
where the symbols used have the definitions given in claim 12.

18. A process for preparing a compound as claimed in claim 12, comprising the following steps:
(A) synthesizing the base skeleton of formula (1) that does not yet bear a group of the formula (2), and
(B) introducing the group of the formula (2) by at least one coupling reaction.

19. A formulation comprising at least one compound as claimed in claim 12 and at least one further compound and/or at least one solvent.

20. A method comprising providing the compound as claimed in claim 12 and including the compounds in an electronic device.

21. An electronic device comprising at least one compound as claimed in claim 12.

22. The electronic device as claimed in claim 21, wherein the device is an organic electroluminescent device, and wherein the compound is used in an emitting layer as matrix material for phosphorescent emitters or for emitters that exhibit TADF (thermally activated delayed fluorescence), or in an electron transport layer and/or in a hole blocker layer and/or in a hole transport layer and/or in an exciton blocker layer.
